(19) United States
(12) Patent Application Publication
(10) Pub. No.: US 2006/0263806 A1
(43) Pub. Date: Nov. 23, 2006

(54) BIOMARKERS FOR BREAST CANCER

(75) Inventors: Xiao-Jun Ma, San Diego, CA (US); Dennis C. Sgroi, Winchester, MA (US); Mark G. Erlander, Encinitas, CA (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: AviaraDx, Inc., Carlsbad, CA (US)

(21) Appl. No.: 11/381,353

(22) Filed: May 2, 2006

Related U.S. Application Data
(63) Continuation of application No. 10/282,596, filed on Oct. 28, 2002, now abandoned.
Application No. 10/282,596 is a continuation-in-part of application No. 10/211,015, filed on Aug. 1, 2002, now abandoned.
Application No. 10/282,596 is a continuation-in-part of application No. 10/028,018, filed on Dec. 21, 2001, now abandoned.

(51) Int. Cl. C12Q 1/68; G06F 19/00

(52) U.S. Cl. 435/6; 702/20

(57) ABSTRACT

Methods and compositions are provided for the detection of breast cancer based upon the identification of three biomarkers for non-normal breast cells. The biomarkers were identified based upon multiple sampling of reference breast tissue samples from independent cases of breast cancer. Two biomarkers display increased expression in non-normal cells while the third biomarker displays decreased expression in non-normal cells.

### RELATED APPLICATIONS

This application is a continuation of U.S. patent application Ser. No. 10/282,596, filed Oct. 28, 2002, which is a continuation-in-part of U.S. patent application Ser. No. 10/028,018, filed Dec. 21, 2001, and Ser. No. 10/211,015, filed Aug. 1, 2002. All three of these applications are hereby incorporated in their entireties as if fully set forth.

### FIELD OF THE INVENTION

The invention relates to the identification and use of gene sequences which are differentially expressed in breast cancer. In particular, the invention provides the identities of three sets of sequences that may be used to identify the presence of breast cancer in tissue and cell samples. The expression of these sequences, whether embodied in nucleic acid expression, protein expression, nucleic acid amplification and/or activation, or other formats, are used in the diagnosis and/or treatment of breast cancer as well as for the study and/or determination of prognosis of a patient. When used for diagnosis, the expression levels of these sequences are used to identify the presence of breast cancer and provide guidance as to the treatment thereof.

### BACKGROUND OF THE INVENTION

Breast cancer is by far the most common cancer among women. Each year, more than 180,000 and 1 million women in the U.S. and worldwide, respectively, are diagnosed with breast cancer. Breast cancer is the leading cause of death for women between ages 50-55, and is the most common non-preventable malignancy in women in the Western Hemisphere. An estimated 2,167,000 women in the United States are currently living with the disease (National Cancer Institute, Surveillance Epidemiology and End Results (NCI SEER) program, Cancer Statistics Review (CSR), www-seer.ims.nci.nih.gov/Publications/CSR1973 (1998)). Based on cancer rates from 1995 through 1997, a report from the National Cancer Institute (NCI) estimates that about 1 in 8 women in the United States (approximately 12.8 percent) will develop breast cancer during her lifetime (NCI's Surveillance, Epidemiology, and End Results Program (SEER) publication SEER Cancer Statistics Review 1973-1997). Breast cancer is the second most common form of cancer, after skin cancer, among women in the United States. An estimated 250,100 new cases of breast cancer are expected to be diagnosed in the United States in 2001. Of these, 192,200 new cases of more advanced (invasive) breast cancer are expected to occur among women (an increase of 5% over last year), 46,400 new cases of early stage (in situ) breast cancer are expected to occur among women (up 9% from last year), and about 1,500 new cases of breast cancer are expected to be diagnosed in men (Cancer Facts & Figures 2001 American Cancer Society). An estimated 40,600 deaths (40,300 women, 400 men) from breast cancer are expected in 2001. Breast cancer ranks second only to lung cancer among causes of cancer deaths in women. Nearly 86% of women who are diagnosed with breast cancer are likely to still be alive five years later, though 24% of them will die of breast cancer after 10 years, and nearly half (47%) will die of breast cancer after 20 years.

Every woman is at risk for breast cancer. Over 70 percent of breast cancers occur in women who have no identifiable risk factors other than age (U.S. General Accounting Office. Breast Cancer, 1971-1991: Prevention, Treatment and Research. GAO/PEMD-92-12; 1991). Only 5 to 10% of breast cancers are linked to a family history of breast cancer (Henderson I C, Breast Cancer. In: Murphy G P, Lawrence W L, Lenhard R E (eds). Clinical Oncology. Atlanta, Ga.: American Cancer Society; 1995:198-219).

The relationship between the expression of cellular factors and breast cancer has been an area of interest. STK15, a centrosomal protein kinase, has been observed as frequently amplified in breast cancer, and its quantitative expression levels positively correlate with tumor grade (Zhou, H. et al. Tumour amplified kinase STK15/BTAK induces centrosome amplification, aneuploidy and transformation. Nat Genet 20, 189-93, (1998)). RAD51 has recently been shown to interact with the tumor suppressor BRCA1 (Chen, J. J., et al. BRCA1, BRCA2, and Rad51 operate in a common DNA damage response pathway. Cancer Res 59, 1752s-1756s (1999)), and its expression also positively correlates with tumor grade in breast cancer (Maacke, H. et al. Over-expression of wild-type Rad51 correlates with histological grading of invasive ductal breast cancer. Int J Cancer 88, 907-13 (2000)).

Citation of documents herein is not intended as an admission that any is pertinent prior art. All statements as to the date or representation as to the contents of documents is based on the information available to the applicant and does not constitute any admission as to the correctness of the dates or contents of the documents.

### SUMMARY OF THE INVENTION

The present invention relates to the identification and use of gene sequences identified as differentially expressed in breast cancer. The sequences of two of the genes display increased expression in non-normal (or abnormal) breast cells, such as those that would be identified as atypical ductal hyperplasia (ADH), ductal carcinoma in situ (DCIS), and invasive ductal carcinoma (IDC) by standard pathology techniques based upon cytological criteria. The sequences of the third gene display decreased expression the same non-normal cells.

The first set of sequences found to be more highly expressed in non-normal breast cells are those of human cysteine-rich intestinal protein 1 (CRIP1 or hCRIP1, also known as human cysteine-rich heart protein or HCRHP). CRIP1 has been mapped to human chromosomal segment 7q11.23 (see Garcia-Barcelo et al. Genomics, 47(3):419-422, 1998).

The second set of sequences found to be more highly expressed in non-normal breast cells are those of the “hematological and neurological expressed sequence 1” (HN1 or Hn1). Murine HN1 has been identified as being expressed in mouse hemopoietic and brain tissues (see Tang et al. Mamm. Genome, 8:695-696, 1997).

The set of sequences found to be expressed at lower levels in non-normal breast cells are those expressed with a “second epithelium restricted Ets transcription factor” termed ESE-2 (see Oettgen et al., J. Biol. Chem., 274(41):29439-52, 1999). The coding region sequence of ESE-2b is identical to that of the E74 like factor 5, termed ELF5 (see Zhou et al., Oncogene, 17(21):2719-32, 1998).

The identified sequences may thus be used in methods of detecting the presence of non-normal breast cells in a tissue or cell containing sample from a subject. The presence of non-normal breast cells may also be used in methods of diagnosing the presence of breast cancer in a tissue or cell containing sample from a subject. A subject, from which a sample is taken, may be one afflicted with, or suspected of having, breast cancer.

The present invention provides a non-subjective means for detecting the presence of non-normal breast cells. This provides advantages over the use of histomorphological or cytological criteria in standard pathology techniques, which requires some level of interpretation by a pathologist trained in assessing the presence and/or progression of breast cancer. The expression levels of these sequences may also be used as a means to assay small, node negative tumors that are not readily assessed by conventional means.

The expression levels of the identified sequences may be used alone or in combination with other sequences capable of identifying the presence of non-normal cells or of various stages and/or grades of breast cancer. Preferably, the sequences of the invention are used alone or in combination with each other.

The present invention provides means for correlating a molecular expression phenotype with a physiological (cellular) stage or state of a non-normal or abnormal breast cell. This correlation provides a way to molecularly diagnose and/or monitor a cell's status in comparison to normal breast cell phenotypes as disclosed herein. Additional uses of the sequences are in the classification of cells and tissues; determination of diagnosis and/or prognosis. Use of the sequences to identify cells of a sample as non-normal or abnormal may also be used to determine the choice, or alteration, of therapy used to treat such cells in the subject from which the sample originated.

The ability to identify non-normal and abnormal breast cells is provided by the recognition of the relevancy of the level of expression of the identified sequences and not by the form of the assay used to determine the actual level of expression. An assay may utilize a means related to the expression level of the sequences disclosed herein as long as the assay reflects, quantitatively or qualitatively, expression of the sequence. Preferably, however, a quantitative assay means is preferred. Identifying features of the sequences include, but are not limited to, unique nucleic acid sequences used to encode (DNA), or express (RNA), the disclosed sequences or epitopes specific to, or activities of, proteins encoded by the sequences. Alternative means include detection of nucleic acid amplification as indicative of increased expression levels (CRIP1 and HN1 sequences) and nucleic acid inactivation, deletion, or methylation, as indicative of decreased expression levels (ESE-2 and ELF5 sequences). Stated differently, the invention may be practiced by assaying one or more aspect of the DNA template(s) underlying the expression of the disclosed sequence(s), of the RNA used as an intermediate to express the sequence(s), or of the proteinaceous product expressed by the sequence(s). As such, the detection of the amount of, stability of, or degradation (including rate) of, such DNA, RNA and proteinaceous molecules may be used in the practice of the invention.

The practice of the present invention is unaffected by the presence of minor mismatches between the disclosed sequences and those expressed by cells of a subject's sample. A non-limiting example of the existence of such mismatches are seen in cases of sequence polymorphisms between individuals of a species, such as individual human patients within Homo sapiens. Knowledge that expression of the disclosed sequences (and sequences that vary due to minor mismatches) is correlated with the presence of non-normal or abnormal breast cells and breast cancer is sufficient for the practice of the invention with an appropriate cell containing sample via an assay for expression.

In one aspect, the invention provides for the identification of the expression levels of the disclosed sequences by analysis of their expression in a sample containing breast cells. In one preferred embodiment, the sample contains single cells or homogenous cell populations which have been dissected away from, or otherwise isolated or purified from, contaminating cells beyond that possible by a simple biopsy. Multiple means for such analysis are available, including detection of expression within an assay for global, or near global, gene expression in a sample (e.g. as part of a gene expression profiling analysis such as on a microarray) or by specific detection, such as quantitative PCR or real time quantitative PCR.

Preferably, the sample is isolated via non-invasive means. The expression of the disclosed sequence(s) in the sample may be determined and compared to the expression of said sequence(s) in reference data of non-normal breast cells. Alternatively, the expression level may be compared to expression levels in normal cells, preferably from the same sample or subject.

When individual breast cells are isolated in the practice of the invention, one benefit is that contaminating, non-breast cells (such as infiltrating lymphocytes or other immune system cells) are not present to possibly affect detection of expression of the disclosed sequence(s). Such contamination is present where a biopsy is used to generate gene expression profiles.

While the present invention has been described mainly in the context of human breast cancer, it may be practiced in the context of breast cancer of an animal known to be potentially afflicted by breast cancer by use of the corresponding sequences of the animal. Preferred animals for the application of the present invention are mammals, particularly those important to agricultural applications (such as, but not limited to, cattle, sheep, horses, and other “farm animals”) and for human companionship (such as, but not limited to, dogs and cats).

### BRIEF DESCRIPTION OF THE FIGURES

FIG. 1 shows log₂ plots of the ratio of expression in ADH, DCIS, and IDC cells to normal cells for CRIP1 and ELF5 sequences. The horizontal line is at “0” such that the ratio is “1” and all points above the line represent increases in expression relative to normal breast cells while all points below the line represent decreases in expression relative to normal breast cells.

FIG. 2 shows the results of in situ hybridization with sense and anti-sense CRIP1 sequences to locate its expression at the cellular level in normal versus IDC cells of the same sample. CRIP1 signal localized to the epithelial cells, and its intensity was markedly increased in the IDC compartment of the same biopsy.

FIG. 3 shows the results of in situ hybridization with sense and anti-sense CRIP1 sequences to locate its expression at the cellular level in normal versus DCIS and IDC cells. CRIP1 signal again localized to the epithelial cells, and its intensity was markedly increased in non-normal cells.

### DETAILED DESCRIPTION OF THE SPECIFIC EMBODIMENTS

Definitions of terms as used herein:

A “sequence” or “gene sequence” as used herein is a nucleic acid molecule or polynucleotide composed of a discrete order of nucleotide bases. The term includes the ordering of bases that encodes a discrete product (i.e. “coding region”), whether RNA or proteinaceous in nature, as well as the ordered bases that precede or follow a “coding region”. Non-limiting examples of the latter include 5′ and 3′ untranslated regions of a gene. It is appreciated that more than one polynucleotide may be capable of encoding a discrete product. It is also appreciated that alleles and polymorphisms of the disclosed sequences may exist and may be used in the practice of the invention to identify the expression level(s) of the disclosed sequences or the allele or polymorphism. Identification of an allele or polymorphism depends in part upon chromosomal location and ability to recombine during Mitosis.

The terms “correlate” or “correlation” or equivalents thereof refer to an association between expression of one or more sequences and a physiologic state of a breast cell to the exclusion of one or more other states by use of the methods as described herein. The invention provides for the correlation between increases in CRIP1 and HN1 sequences and non-normal or abnormal breast cells. Similarly, the invention provides for the correlation between decreases in ESE-2/ELF5 sequences and non-normal or abnormal breast cells. Increases and decreases may be readily expressed in the form of a ratio between expression in a non-normal cell and a normal cell such that a ratio of one (1) indicates no difference while ratios of two (2) and one-half indicate twice as much, and half as much, expression in the non-normal cell versus the normal cell, respectively. Expression levels can be readily determined by quantitative methods as described below.

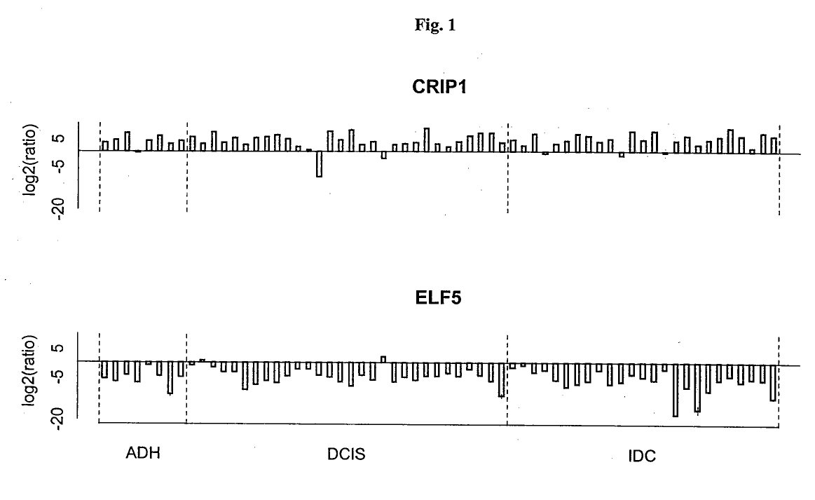

For example, increases in CRIP1 expression can be indicated by ratios of or about 1.1, of or about 1.2, of or about 1.3, of or about 1.4, of or about 1.5, of or about 1.6, of or about 1.7, of or about 1.8, of or about 1.9, of or about 2, of or about 2.5, of or about 3, of or about 3.5, of or about 4, of or about 4.5, of or about 5, of or about 5.5, of or about 6, of or about 6.5, of or about 7, of or about 7.5, of or about 8, of or about 8.5, of or about 9, of or about 9.5, of or about 10, of or about 15, of or about 20, of or about 30, of or about 40, of or about 50, of or about 60, of or about 70, of or about 80, of or about 90, of or about 100, of or about 150, of or about 200, of or about 300, of or about 400, of or about 500, of or about 600, of or about 700, of or about 800, of or about 900, or of or about 1000. A ratio of 2 is a 100% (or a twofold) increase in expression. Similar ratios can be used with respect to increases in HN1 expression. Decreases in ESE-2/ELF5 expression can be indicaed by ratios of or about 0.9, of or about 0.8, of or about 0.7, of or about 0.6, of or about 0.5, of or about 0.4, of or about 0.3, of or about 0.2, of or about 0.1, of or about 0.05, of or about 0.01, of or about 0.005, of or about 0.001, of or about 0.0005, of or about 0.0001, of or about 0.00005, of or about 0.00001, of or about 0.000005, or of or about 0.000001. Non-limiting examples of such ratios are shown in FIG. 1.

A “polynucleotide” is a polymeric form of nucleotides of any length, either ribonucleotides or deoxyribonucleotides linked by phosphodiester bonds and encompasses the strand of a given sequence as disclosed herein as well as the complementary strand of a given sequence. The term refers only to the primary structure of the molecule. Thus, this term includes double- and single-stranded DNA and RNA as well as analogs thereof comprising a non-phosphodiester backbone. It also includes known types of modifications including labels known in the art, methylation, “caps”, substitution of one or more of the naturally occurring nucleotides with an analog, and internucleotide modifications such as uncharged linkages (e.g., phosphorothioates, phosphorodithioates, etc.), as well as unmodified forms of the polynucleotide.

The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Amplification” may also be used in the context of DNA amplification wherein copies of coding sequences within the cellular genome are increased. “Multiple copies” mean at least 2 copies. A “copy” does not necessarily mean perfect sequence complementarity or identity to the template sequence. Methods for amplifying mRNA are generally known in the art, and include reverse transcription PCR (RT-PCR) and those described in U.S. patent application Ser. No. 10/062,857 entitled “Nucleic Acid Amplification” filed on Oct. 25, 2001 as well as U.S. Provisional Patent Application 60/298,847 (filed Jun. 15, 2001) and 60/257,801 (filed Dec. 22, 2000), all of which are hereby incorporated by reference in their entireties as if fully set forth.

By corresponding is meant that a nucleic acid molecule shares a substantial amount of sequence identity with another nucleic acid molecule. Substantial amount means at least 95%, usually at least 98% and more usually at least 99%, and sequence identity is determined using the BLAST algorithm, as described in Altschul et al. (1990), J. Mol. Biol. 215:403-410 (using the published default setting, i.e. parameters w4, t=17). Alternatively, RNA may be directly labeled as the corresponding cDNA by methods known in the art.

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of a sample polynucleotides can be determined based on their binding to a particular position in the microarray.

Because the invention relies upon the identification of sequences that are over- or under-expressed, one embodiment of the invention involves determining expression by hybridization of mRNA, or an amplified or cloned version thereof, of a sample cell to a polynucleotide of a disclosed sequence. Preferred polynucleotides of this type contain at least about 20, at least about 22, at least about 24, at least about 26, at least about 28, at least about 30, at least about 32, at least about 34, at least about 36, at least about 38, at least about 40, at least about 42, at least about 44, or at least about 46 consecutive bases of a sequence that is not found in other human sequences. The term “about” as used in the previous sentence refers to an increase or decrease of 1 from the stated numerical value. Longer polynucleotides may of course contain minor mismatches (e.g. via the presence of mutations) which do not affect hybridization to the nucleic acids of a sample. Such polynucleotides may be label to assist in their detection; alternatively, the nucleic acids to which such polynucleotides will hybridize may be labeled. Such polynucleotides may also be immobilized, such as by attachment to a solid support.

Even more preferred are polynucleotides of at least or about 50, at least or about 100, at least about or 150, at least or about 200, at least or about 250, at least or about 300, at least or about 350, at least or about 400, at least or about 450, or at least or about 500 consecutive bases of a sequence that is not found in other sequences in the human genome. The term “about” as used in the preceding sentence refers to an increase or decrease of 10% from the stated numerical value. The polynucleotides may of course contain minor mismatches which do not affect hybridization to the nucleic acids of a sample.

In another embodiment of the invention, all or part of a disclosed sequence may be amplified and detected by methods such as the polymerase chain reaction (PCR) and variations thereof, such as, but not limited to, quantitative PCR (QPCR), reverse transcription PCR (RT-PCR), and real-time PCR, optionally real-time RT-PCR. Such methods would utilize one or two primers that are complementary to portions of a disclosed sequence, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) of the invention under conditions which allow for their hybridization.

Alternatively, and in another embodiment of the invention, expression of a sequence may be determined by analysis of expressed protein encoded by said sequence in a cell sample of interest by use of one or more antibodies specific for one or more epitopes of the individual products (proteins) in said cell sample. Such antibodies are preferably labeled to permit their easy detection after binding to the gene product. In the case of a protein that may be found in the blood, serum or other bodily fluid, the assay may modified to use such materials in place of a breast cell containing sample.

The term “label” refers to a composition capable of producing a detectable signal indicative of the presence of the labeled molecule. Suitable labels include radioisotopes, nucleotide chromophores, enzymes, substrates, fluorescent molecules, chemiluminescent moieties, magnetic particles, bioluminescent moieties, and the like. As such, a label is a composition detectable by spectroscopic, photochemical, biochemical, immunochemical, electrical, optical or chemical means.

The term “support” refers to conventional supports such as beads, particles, dipsticks, fibers, filters, membranes and silane or silicate supports such as glass slides.

As used herein, a “breast tissue sample” or “breast cell sample” refers to a sample of breast tissue or fluid isolated from an individual, preferably suspected of being afflicted with, or at risk of developing, breast cancer. Such samples are primary isolates (in contrast to cultured cells) and may be collected by a non-invasive means, including, but not limited to, ductal lavage, fine needle aspiration, needle biopsy, the devices and methods described in U.S. Pat. No. 6,328,709, or another suitable means recognized in the art. Alternatively, the “sample” may be collected by an invasive method, including, but not limited to, surgical biopsy.

“Expression” and “gene expression” include transcription and/or translation of nucleic acid material, such as the sequences of the invention.

As used herein, the term “comprising” and its cognates are used in their inclusive sense; that is, equivalent to the term “including” and its corresponding cognates.

Conditions that “allow” an event to occur or conditions that are “suitable” for an event to occur, such as hybridization, strand extension, and the like, or “suitable” conditions are conditions that do not prevent such events from occurring. Thus, these conditions permit, enhance, facilitate, and/or are conducive to the event. Such conditions, known in the art and described herein, depend upon, for example, the nature of the nucleotide sequence, temperature, and buffer conditions. These conditions also depend on what event is desired, such as hybridization, cleavage, strand extension or transcription.

Sequence “mutation,” as used herein, refers to any sequence alteration in the sequence of a gene disclosed herein interest in comparison to a reference sequence. A sequence mutation includes single nucleotide changes, or alterations of more than one nucleotide in a sequence, due to mechanisms such as substitution, deletion or insertion. Single nucleotide polymorphism (SNP) is also a sequence mutation as used herein. Because the present invention is based on the relative level of sequence expression, mutations in non-coding regions of genes as disclosed herein may also be assayed in the practice of the invention.

“Detection” includes any means of detecting, including direct and indirect detection of gene expression and changes therein. For example, “detectably less” products may be observed directly or indirectly, and the term indicates any reduction (including the absence of detectable signal). Similarly, “detectably more” product means any increase, whether observed directly or indirectly.

Increases and decreases in expression of the disclosed sequences are defined in the following terms based upon percent or fold changes over expression in normal cells. Increases may be of 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 10, 20, 30, 40, 50, 55, 60, 65, 70, 75, 80, 82, 84, 86, 88, 90, 92, 94, 96, 98, 99 or 100% relative to expression levels in normal cells.

Unless defined otherwise all technical and scientific terms used herein have the same meaning as commonly understood to one of ordinary skill in the art to which this invention belongs.

### Specific Embodiments

The present invention relates to the identification and use of three sets of sequences for the detection of non-normal and cancerous breast cells. The differential expression of these sequences in non-normal or abnormal breast cells relative to normal breast cells is used to identify a breast cancer cell as being non-normal or otherwise abnormal. The invention is advantageously used to identify breast cells as being those of ADH, DCIS, and IDC as otherwise determinable standard pathological techniques. The invention may also be applied to the identification of breast cells as being those of atypical lobular hyperplasia (ALH), lobular carcinoma in situ (LCIS), and invasive lobular carcinoma (ILC) as otherwise determinable by standard pathological techniques.

Other non-limiting examples of non-normal or abnormal cells include malignant cells, atypical cells (including reactive and pre-neoplastic), neoplastic cells, tumor cells, and cancer or cancerous cells.

The sequences(s) identified by the present invention are expressed in correlation with non-normal breast cells, and thus negatively correlated with normal breast cells. For example, CRIP1, identified by I.M.A.G.E. Consortium CloneID 1323448 and cluster NM_{—}001311 (“The I.M.A.G.E. Consortium: An Integrated Molecular Analysis of Genomes and their Expression,” Lennon et al., 1996, Genomics 33:151-152; see also image.llnl.gov) has been found to be useful in discriminations between normal and ADH or DCIS or IDC breast cells.

In preferred embodiments of the invention, any sequence, or unique portion thereof, of the CRIP1 sequences identified by the cluster, as well as UniGene Homo sapiens cluster Hs.17409, may be used. The consensus sequence of the I.M.A.G.E. Consortium cluster is as follows, with the assigned coding region (ending with a termination codon) underlined and preceded by the 5′ untranslated and/or non-coding region and followed by the 3′ untranslated and/or non-coding region:

(consensus sequence for CRIP1)

SEQ ID NO:1

CGCACGAGGGCCCGTGCCGCCCCAGCCGCTGCCGCCTGCACCGGACCCGG
AGCCGCCATGCCCAAGTGTCCCAAGTGCAACAAGGAGGTGTACTTCGCCG
AGAGGGTGACCTCTCTGGGCAAGGACTGGCATCGGCCCTGCCTCAAGTGC
GAGAAATGTGGGAAGACGCTGACCTCTGGGGGCCACGCTGAGCACGAAGG
CAAACCCTACTGCAACCACCCCTGCTACGCAGCCATGTTTGGGCCTAAAG
GCTTTGGGCGGGGCGGAGCCGAGAGCCACACTTTCAAGTAAACCAGGTGG
TGGAGACCCCATCCTTGGCTGCTTGCAGGGCCACTGTCCAGGCAAATGCC
AGGCCTTGTCCCCAGATGCCCAGGGCTCCCTTGTTGCCCCTAATGCTCTC
AGTAAACCTGAACACTTGGAAAAAAAAAAAAAAAAAAA

The sequences identified as belonging to the I.M.A.G.E. Consortium and UniGene clusters, with the assigned coding region underlined, follow below. The 5′ and 3′ untranslated and/or non-coding regions are by reference to the assigned coding region, which is presented as the complementary strand in the 3′ to 5′ direction for some of the sequences.

(CloneID 5103334)

SEQ ID NO:2

GGGGATTGGAGATGTTCCCCTCATGGAGGGTGCTGAGGACCTTAGGGTGG
GCTGCCAGGCTGGGCGGATGCGGGCTAAGTGCACAGGGCCTTGGGCAGAG
CTGGCTGCAAGAGGCGGGTACGCCAGTGGTGGGTAGGCGCCGCGTCCTGC
AGCGTCTCACCGGGGCCTGTCTGTGCCTCTGCAGCCGAGAGGGTGACCTC
TCTGGGCAAGGACTGCCATCGGCCCTGCCTCAAGTGCGAGAAATGTGGGA
ACACGCTGACCTCTGGGGGCCACGCTGAGCACGAAGGCAAACCCTACTGC
AACCACCCCTGCTACGCAGCCATGTTTGGGCCTAAAGGCTTTGGGCGGGG
CGGAGCCGAGAGCCACACTTTCAAGTAAACCAGGTGGTGGAGACCCCATC
CTTGGCTGCTTGCAGGGCCACTGTCCAGGCAAATGCCAGGCCTTGTCCCC
AGATGCCCAGGGCTCCCTTGTTGCCCCTAATGCTCTCAGTAAACCTGAAC
ACTTGGAAAAAAAAAAAA
(CloneID 5777677)

SEQ ID NO:3

GCACGAGCGCTGGGCTAGGGGCGCGGCTTGAACTCGCCTAAAGAGCTGCG
CCCTCTCATCTCGCGCCTGCAGCCCGTGCCGCCCCAGCCGCTGCCGCCTG
CACCGGACCCGGAGCCGCCATGCCCAAGTGTCCCAAGTGCAACAAGGAGG
TGTACTTCGCCGAGAGGGTGACCTCTCTGGGCAAGGACTGGCATCGGCCC
TGCCTGAAGTGCGAGAAATGTGGGAAGACGCTGACCTCTGGGGGCCACGC
TGAGCACGAAGGCAAACCCTACTGCAACCACCCCTGCTACGCAGCCATGT
TTGGGCCTAAAGGCTTTGGGCGGGGCGGAGCCGAGAGCCACACTTTCAAG
TAAACCAGGTGGTGGAGACCCCATCCTTGGCTGCTTGCAGGGCCACTGTC
CAGGCAAATGCCAGGCCTTGTCCCCAGATGCCCAGGGCTCCCTTGTTGCC
CCTAATGCTCTCAGTAAACCTGAACACTTCG
(CloneID 563289)

SEQ ID NO:4

GCACGAGCGCTGGGCTAGGGGCGCGGCTTGAACTCGCCTAAAGAGCTGCG
CCCTCTCATCTCGCGCCTGCAGCCCGTGCCGCCCCAGCCGCTGCCGCCTG
CACCGGACCCGGAGCCGCCATGCCCAAGTGTCCCAAGTGCAACAAGCAGG
TGTACTTCGCCGAGAGGGTGACCTCTCTGGGCAAGGACTGGCATCGGCCC
TGCCTGAAGTGCGAGAAATGTGGGAAGACGCTGACCTCTGGGGGCCACGC
TGAGCACGAAGGCAAACCCTACTGCAACCACCCCTGCTACGCAGCCATGT
TTGGGCCTAAAGGCTTTGGGCGGGGCGGAGCCGAGAGCCACACTTTCAAG
TAAACCAGGTGGTGGAGACCCCATCCTTGGCTGCTTGCAGGGCCACTGTC
CAGGCAAATGCCAGGCCTTGTCCCCAGATGCCCAGGGCTCCCTTGTTGCC
CCTAATGCTCTCAGTAAACCTGAACACT
(CloneID 1627147 in 3′ to 5′ orientation)

SEQ ID NO:5

TGGTTTTCCAAGTGTTCAGGTTTACTGAGAGCATTAGGGGCAACAAGGGA
GCCCTGGGCATCTGGGGACAAGGCCTGGCATTTGCCTGGACAGTGGCCCT
GCAAGCAGCCAAGGATGGGGTCTCCACCACCTGCTTTACTTGAAAGTGTG
GCTCTCGGCTCCGCCCCGCCCAAAGCCTTTAGGCCCAAACATGGCTACGT
AGCAGGGGTGGTTGCAGTAGGGTTTGCCTTCGTGCTCAGCGTGGCCCCCA
GAGGTCAGCGTCTTCCCACATTTCTCGCACTTCAGGCAGGGCCGATGCCA
GTCCTTGCCCAGAGAGGTCACCCTCTCGGCGAAGTACACCTCCTTGTTGC
ACTTGGGACACTTGGGCATGGCGGCTCCGGGTCCGGTGCAGGCGGCAGCG
GCTGGGGCGGCACGGGCTGCAGGCGCGAGACTGGGTGGATCCGGCCTGGT
CCGCGCCTTTCAGGGACCCCGGGACCCCGCCCCTTGCAGACACCGCCCCT
C
(CloneID 1627139 in 3′ to 5′ orientation)

SEQ ID NO:6

TGGTTTTCCAAGTGTTCAGGTTTACTGAGAGCATTAGGGGCAACAAGGGA
GCCCTGGGCATCTGGGGACAAGGCCTGGCATTTGCCTGGACAGTGGCCCT
GCAAGCAGCCAAGGATGGGGTCTCCACCACCTGGTTTACTTGAAAGTGTG
GCTCTCGGCTCCGCCCCGCCCAAAGCCTTTAGGCCCAAACATGGCTACGT
AGCAGGGGTGGTTGCAGTAGGGTTTGCCTTCGTGCTCAGCGTGGCCCCCA
GAGGTCAGCGTCTTCCCACATTTCTCGCACTTCAGGCAGGGCCGATGCCA
GTCCTTGCCCAGAGAGGTCACCCTCTCGGCGAAGTACACCTCCTTGTTGC
ACTTGGGACACTTGGGCATGGCGGCTCCGGGTCCGGTGCAGGCGGCAGCG
GCTGGGGCGGCACGGGCTGCAGGCGCGAGACTGGGTGGATCCGGCCTGGT
CCGCGCCTTTCA
(CloneID 3296101 in 3′ to 5′ orientation)

SEQ ID NO:7

TTGGTTTTCCAAGTGTTCAGGTTTACTGAGAGCATTAGGGGCAACAAGGG
AGCCCTGGGCATCTGGGGACAAGGCCTGGCATTTGCCTGGACAGTGGCCC
TGCAAGCAGCCAAGGATGGGGTCTCCACCACCTGGTTTACTTGAAAGTGT
GGCTCTCGGCTCCGCCCCGCCCAAAGCCTTTAGGCCCAAACATGGCTGCG
TAGCAGGGGTGGTTGCAGTAGGGTTTGCCTTCGTGCTCAGCGTGGCCCCC
AGACGTCAGCGTCTTCCCACATTTCTCGCACTTCAGGCAGGGCCGATGCC
AGTCCTTGCCCAGAGAGGTCACCCTCTCGGCGAAGTACACCTCCTTGTTG
CACTTGGGACACTTGGGCATGGCGGCTCCGGGTCCGGTGCAGGCGGCAGC
GGCTGGGGCGGCACGGGCTGCATGCGCGAGACTGGGTGGATCCGGCCTGG
TCCGCGCCTTTCAGGGACCCCGGGACCCCG
(CloneID 3631097)

SEQ ID NO:8

GCCGCCTGCACCGGACCGGAGCCGCCATGCCCAAGTGTCCCAAGTGCAAC
AAGGAGGTGTACTTCGCCGAGAGGGTGACCTCTCTGGGCAAAGGACTGGC
ATCGGCCCTGCCTGAAGTGCGAGAAATGTGGGAAGACGCTGACCTCTGTG
GGGCCACGCTGAGCACGAAGGCAAACCCTACTGCAACCACCCCTGCTACG
CAGCCATGTTTGGGCCTAAAGGCTTTGGGCGGGGCGGAGCCGAGGAGCCA
CACTTTCAAGTAAAACCAGGGTGGTGGAGACCCCATCCTTGGCTGCTTG
CAGGGCCCACTTGTCCAGGGCAAAATTGCCAGGCCTTTGTCCCCCAGAAT
GCCCAGGGGCTCCCTTTGTTTGGCCCCCTTAATTGCTCTCAGTAAACCCT
TGAACACTTGGA
(CloneID 2782288)

SEQ ID NO:9

CAGCCCGTGCCGCCCCAGCCGCTGCCGCCTGCACCGGACCCGGAGCCGCC
ATGCCCAAGTGTCCCAAGTGCAACAAGGAGGTGTACTTCGCCAAGAGGGT
GACCTCTCTGGGCAAGCACTGGCATCGGCCCTGCCTGAAGTGCGAGAAAT
GTCGGAAGACGCTGACCTCTGGGGGCCACGCTGAGCACGAAGGCAAACCC
TACTGCAACCACCCCTGCTACGCAGCCATGTTTGGGCCTAAAGGCTTTGG
GCGGGGCGGAGCCGAGAGCCACACTTTCAAGTAAACCAGGTGGTGGAGAC
CCCATCCTTGGCTGCTTGCAGGGCCACTGTCCAGGCAAATGCCAGGCCTT
GTCCCCAGATGCCCAGGGCTCCCTTGTTGCCCCTAATGCTCTCAGT
(CloneID 3903337)

SEQ ID NO:10

CACCGGACCCGGAGCCGGCCATGCCCAAGTGTCCCAAGTGCAACAAGGAG
GTGTACTTCGCCGAGAGGGTGACCTCTCTGGGCAAGGACTGGCATCGGCC
CTGCCTGAAGTGCGAGAAATGTGGGAAGACGCTGACCTCTGGGGGCCACG
CTGAGCACGAAGGCAAACCCTACTGCAACCACCCCTGCTACGCAGCCATG
TTTGGGCCTAAAGGCTTTGGGCGGGGCGGAGCCGAGAGCCACACTTTCAA
GTAAACCAGGTGGTGGAGACCCCATCCTTGGCTGCTTGCAGGGCCACTGT
CCAGGCAAATGCCAGGCCTTGTCCCCAGATGCCCAGGGCTCCCCTTGTTG
CCCCCTAAATGCTCTCCAGGTAAAACCTGAAACACTTGGAAAAAAAAAAC
AAAAAAAAAAA
(CloneID 2063820 in 3′ to 5′ orientation)

SEQ ID NO:11

TCCAAGTGTTCAGGTTTACTGAGAGCATTAGGGGCAACAAGGGAGCCCTG
GGCATCTGGGGAAAAGGCCTGGCATTTGCCTGGACAGTGGCCCTGCAAGC
AGCCAAGGATGGGGTCTCCACCACCTGGTTTACTTGAAAGTGTGGCTCTC
GGCTCCGCCCCGCCCAAAGCCTTTAGGCCCAAACATGGCTACGTAGCAGG
GGTGGTTGCAGTAGGGTTTGCCTTCGTGCTCAGCGTGGCCCCCAGAGGTC
AGCGTCTTCCCACATTTCTCGCACTTCAGGCAGGGCCGATGCCAGTCCTT
GCCCAGAGAGGTCACCCTCTCGGCTGCAGAGGCACAGACAGGCCCCGGTG
AGACGCTGCAGGACGCGGCGCCTACCCACCACTGGCGTACCCGCCTCTTG
CAGCCAGCTCTGCCCAAGGCCCTGTGCACTTAGCCCGCATC
(CloneID 2568304 in 3′ to 5′ orientation)

SEQ ID NO:12

TTTTTTTTTTTTTTTTTTTTTTTTTTTTTCCAAGGGTTCAGGTTTACTGA
AAGCATTAGGGGCAACAAGGGAGCCCTGGGCATTTGGGGACAAGGCCTGG
CATTTGCCTGGACAGGGGCCCTGCAAGCAGCCAAGGATCGGGTCTCCACC
ACCTGGTTTACTTGAAAGTGTGGCTCTCGGCTCCGCCCCGCCCAAAGCCT
TTAGGCCCAAACATGGCTGCGTAACAGGGGGGGGTTGCANTACGGTTTGC
CTTCGGGCTCAGCGTGGCCCCCAAAGGTCAGCGTCTTCCCACATTTTTCG
CACTTCAGGCAGGGACGATGCCAGTCCTTGCCCAAAAAGGTCACCCTCTC
GGCAAAGTACACCTCCTTGTTGCACTTGGGACACTTGGGCATGGCGGCTC
CGGGTCCGGTGCAGGCGGCAACGGCTGGGGCGGCA
(CloneID 2329226 in 3′ to 5′ orientation)

SEQ ID NO:13

CCAAGTGTTCAGGTTTACTGAGAGCATTAGGGGCAACAAGGGAGCCCTGG
GCATCTGGGGACAAGGCCTGGCATTTGCCTGGACAGTGGCCCTGCAAGCA
GCCAAGGATGGGGTCTCCACCACCTGGTTTACTTGAAAGTGTGGCTCTCG
GCTCCGCCCCGCCCAAAGCCTTTAGGCCCAAACATGGCTACGTAGCAGGG
GTGGTTGCAGTAGGGTTTGCCTTCGTGCTCAGCGTGGCCCCCAGAGGTCA
GCGTCTTCCCACATTTCTCGCACTTCAGGCAGGGCCGATGCCAGTCCTTG
CCCAGAGAGGTCACCCTCTCGGCGAAGTACACCTCCTTGTTGCACTTGGG
ACACTTGGGCATGGCGGCTCCGGGTCCGGTGCAGGCGGCAGCGGCTGGGG
CGGCACGGGCTGCAGGCGCGAGA
(CloneID 5433206)

SEQ ID NO:14

GGCCCGTGCCGCCCCAGCCGCTGCCGCCTGCACCGGACCCGGAGCCGCC
ATGCCCAAGTGTCCCAAGTGCAACAAGGAGGTGTACTTCGCCGAGAGGG
TGACCTCTCTGGGCAAGGACTGGCATCGGCCCTGCCTGAAGTGCGAGAA
ATGTGGGAAGACGCTGACCTCTGGGGGCCACGCTGAGCACGAAGGCAAA
CCCTACTGCAACCACCCCTGCTACGCACCCATGTTTGGGCCTAAAGGCT
TTGGGCGGGGCGGAGCCGAGAGCCACACTTTCAAGTAAACCAGGTGGTG
GAGACCCCATCCTTGGCTGCTTGCAGGGCCACTGTCCAGGCAAATGCCA
GGCCTTGTCCCCAGATGCCCAGGGCTCCCTTGTTGCCCCTAATGCTCTC
AGTAAACCTGAACACTTGGAAACAAACACACAAAAAAAAAAACAAAACA
AAAAAAACACAAACCAAAAAACACGAGAAAAGCACAAAAAAAGAGGCAA
AGAGAAAAACGCGCGCGGGATATCAGAGGCAGGAGGGGCGAAAAGGGGG
GAGACAGAGGAGGAACAGCGACAACCGGCACCGCGCGC
(CloneID 5220536)

SEQ ID NO:15

GCCGTGCCGCCCAGCCGCTGCCGCTGCACCGGACCCGGAGCCGCATGCCC
AAGTGTCCAAGTGCAACAAGGAGGTGTACTTCGCCGAGAGGGTGACCTCT
CTGGGCAAGGACTGGCATCGGCCCTGCCTGAAGTGCGAGAAATGTGGGAA
GACGCTGACCTCTGGGGGCCACGCTGAGCACGAAGGCAAACCCTACTGCA
ACCACCCCTGCTACGCAGCCATGTTTGGGCCTAAAGGCTTTGGGCGGGGC
GGAGCCCAGAGCCACACTTTCAAGTAAACCAGGTGGTGGAGACCCCATCC
TTGGCTGCTTGCAGGGGCCACTGTCCAGGCAAATGCCAGGCCTTGTCCCC
CAGATGCCCAGGGCTTCCCTTTGTTGCCCCTAATGGCTCTCAGTAAACCT
TGAACAACTTGGAAACACCACACCAAAAACCAACACACAGGG
(CloneID 2387987 in 3′ to 5′ orientation)

SEQ ID NO:16

CAAGTGTTCAGGTTTACTGAGAGCATTAGGGGCAACAAGGGAGCCCTGGG
CATCTGGGGACAAGGCCTGGCATTTGCCTGGACAGTGGCCCTGCAAGCAG
CCAAGGATGGGGTCTCCACCACCTGGTTTACTTGAAAGTGTGGCTCTCGG
CTCCGCCCCGCCCAAAGCCTTTAGGCCCAAACATGGCTGCGTAGCAGGGG
TGGTTGCAGTACGGTTTGCCTTCGTGCTCAGCGTGGCCCCCAGAGGTCAG
CGTCTTCCCACATTTCTCGCACTTTAGGCAGGGCCGATGCCAGTCCTTGC
CCAGAGAGGTCACCCTCTCGGCGAAGTACACCTCCTTGTTGCACTTGGGA
CACTTGGGCATGGCGGCTCCGGGTCCGGTGCAGGCGGCAGCGGCTGCGGCG
GCACGGGCTGCAGGCGCGAGATGAGAGGGCGCAGCTCTTTAGGCGAGTTC
AAGCCGCGCCCCTAGCCCAGCG
(CloneID 5186252)

SEQ ID NO:17

TGCAGCCGTCCGCCCACCCTGGTGCGCTGCACGGACCCGCAGCCGCAT
GCGACGAGTGTCCAATGTGCAACAAGGAGGTGTACTTCGCCGAGAGGGTG
ACCTCTCTGGGCAAAGACTGGCATCTGGCCCTGCCTGAAGTGCGAGAAAT
GTGGGAAGACGCTGACCTCTGGGGGCCACGCTGAGCACGAAGGCAAACCC
TACTGCAACCACCCCTGCTACGCAGCCATGTTTGGGCCTAAAGGCTTTGG
GCGGGGCGGAGCCGAGAGCCACACTTTCAAGTAAACCAGGTGGTGGAGAC
CCCATCCTTGGCTGCTTGCAGCGCCACTGTCCAGGCAAATTGCCAGGGCC
TTGTCCCCCAGATTGCCCAGGGCTCCCTCTTGTTGCCCCCTAAATTGCTC
TCAGGTTAGAACCTTGAAACAGCTTTGCGAACAAGCAAAAGAAGAGAAGG
AGGTGCTGATAATAGCAGACGAAGTGAGAAGAGAACACCAATCACATGAA
CACGAATAGGCAGGCCATACAACGCAGGAGATATCGAACCCGGCGATAGT
GCCCGGT
(CloneID 3609948)

SEQ ID NO:18

CGGACCCGGAGCCGCCATGCCCAAGTGTCCCAAGTGCAACAAGGAGGTGT
ACTTCGCCGAGAGGGTGACCTCTCTGGGCAAGGACTGGCATCGGCCCTGC
CTGAAGTGCGAGAAATGTGGGAAGACGCTGACCTCTGGGGGCCACGCTGA
GCCAAGGCAAACCCTACTGCAACCACCCCTGCTACGCAGCCATGTTTGGG
CCTAAAGGCTTTGGGCGGGGCGGAGCCGAGAGCCACACTTTCAAGTAAAC
CAGGTGGTGGAGACCCCATCCTTGGCTGCTTGCAGGGCCACTGTCCAGGC
AAATGCCAGGCCTTGTCCCCAGATGCCCAGGGCTCCCTTGTTGCCCCTAA
TGCTCTCAGTAAACCTGAACACTTGGA
(CloneID 1584777 in 3′ to 5′ orientation)

SEQ ID NO:19

CCAAGTGTTCAGGTTTACTGAGAGCATTAGGGGCAACAAGGGAGCCCTGC
GCATCTGGGGACAAGGCCTGGCATTTGCCTGGACAGTGGCCCTGCAAGCA
GCCAAGGATGGGGTCTCCACCACCTGGTTTACTTGAAAGTGTGGCTCTCG
GCTCCGCCCGCCCAAAGCCTTTAGGCCAAACATGGCTGCGTAAGAGAGTG
GTTGAAGTAGGGTTTGCCTTCGTGCTCAGCGTGGCCCCCAGAGGTCAGCG
TCTTCCCACATTTCTCGCAGTTCAGGCAGGGCAGAGTGCAGTCCTTGCCC
AGAGAGGGGACCTGTCGGCGAAGAGAACTCCTTGTTGCACTTGGGACACT
TGGCCATGGCGGCTCCGGGTCCGGTGCAGGCGGC
(CloneID 2130246 in 3′ to 5′ orientation)

SEQ ID NO:20

AGCGGCCGCCCTTTTTTTTTTTTTTTTTTCCAAGTGTTCAGGTTTACTGA
GAGCATTAGGGGCAACAAGGGAGCCCTGGGCATCTGGGGACAAGGCCTGG
CATTTGCCTGGACAGTGGCCCTGCAAGCAGCCAAGGATGGGGTCTCCACC
ACCTGGTTTACTTGAAAGTGTGGCTCTCGGCTCCGCCCCGCCCAAAGCCT
TTAGGCCCAAACATGGCTGCGTAGCAGGGGTGGTTGCAGTAGGGTTTGCC
TTCGTGCTCAGCGTGGCCCCCAGAGGTCAGCGTCTTCCCACATTTCTCGC
ACTTCAGGCAGGGCCGATGCCAGTCCTTGCCCAGAGAGGTCACCCTCTCG
GCGAAGTACACCTCCTTGTTGCACTTGGGACAC
(CloneID 1908782 in 3′ to 5′ orientation)

SEQ ID NO:21

CCAAGTGTTCAGGTTTACTGAGAGCATTAGGGGCAACAAGGGAGCCCTGG
GCATCTGGGGACAAGGCCTGGCATTTGCCTGGACAGTGGCCCTGCAAGCA
GCCAAGGATGGGGTCTCCACCACCTGGTTTACTTGAAAGTGTGGCTCTCG
GCTCCGCCCCGCCCAAAGCCTTTAGGCCCAAACATGGCTGCGTAGCAGGG
GTGGTTGCAGTAGGGTTTGCCTTCGTGCTCAGCGTGGCCCCCAGAGGTCA
GCGTCTTCCCACATTTCTCGCACTTCAGGCAGGGCCGATGCCAGTCCTTG
CCCACAGAGGTCACTTCTCTCGGCGAAGTCCACCCTCCTTGTTGCACCTT
GGGACACCTT
(CloneID 1323448 in 3′ to 5′ orientation)

SEQ ID NO:22

GAGCATTAGGGGCAACAAGGGAGCCCTGGGCATCTGGGGACAAGGCCTGG
CATTTGCCTGGACAGTGGCCCTGCAAGCAGCCAAGGATGGGGTCTCCACC
ACCTGGTTTACTTGAAAGTGTGGCTCTCGGCTCCGCCCGCCCAAAGCCTT
TAGCCCAAAACATGGCTGCGTACGAGGGTGGTTGCAGTAGGGTTTGCCTT
CGTGCTCAGCGTGGCCCCCAGAGGTCAGCGTCTTCCCACATTTCTCGCAC
TTCAGGCAGGGCGANTGCCAGTCCTTGCCCAGAGAGGTCACCCTCTCGGC
GAAGTACACCTCCTTGTTGC
(CloneID 1837171 in 3′ to 5′ orientation)

SEQ ID NO:23
TTTGGTTTTCCAAGTGTTCAGGTTTACTGAGAGCATTAGGGGCAACAAGG
GAGCCCTGGGCATCTGGGGACAAGGCCTGGCATTTGCCTGGACAGTGGCC
CTGCAAGCAGCCAAGGATGGGGTCTCCACCACCTGGTTTACTTGAAAGTG
TGGCTCTCGGCTCCGCCCCGCCCAAAGCCTTTAGGCCCAAACATGGCTGC
GTAGCAGGGGTGGTTGCAGTAGGGTTTGCCTTCCTGCTCAGCGTGGCCCC
CAGACGTCAGCGTCTTCCCACATTTCTCGCACTTCAGGCAGGGCCGATGC
CAGTCCTTGCCCAGAGAGGTCACCCTCTCGGCGAAGTACACCTCCTTGTT
GCACTTGGGACACTTGGG
(CloneID 2802267)
SEQ ID NO:24

GCGTCGACCGCCGAGAGGGTGACCTCTCTGGGCAAGGACTGGCATCGGCC
CTGCCTGAAGTGCGAGAAATGTGGGAAGACGCTGACCTCTGGGGGCCACG
CTGAGCACGAAGGCAAACCCTACTGCAACCACCCCTGCTACGCAGCCATG
TTTGGGCCTAAAGGCTTTGGGCGGGGCGGAGCCGAGAGCCACACTTTCAA
GTAAACCAGGTGGTGGAGACCCCATCCTTGGCTGCTTGCAGGGCCGCTGT
CCAGGCAAATGCCAGGCCTTGTCCCCAGATGCCCAGGGCTCCCTTGTTGC
CCCTAATGCTCT
(CloneID 2847309)

SEQ ID NO:25

AAGGTAGCGAAGCGAGAGGGTGACCTCTCTGGGCAAGGACTGGCATCGGC
CCTGCCTGAAGTGCGAGAAATGTGGGAAGACGCTGACCTCTGGGGGCCAC
GCTGAGCACGAAGGCAAACCCTACTGCAACCACCCCTGCTACGCAGCCAT
GTTTGGGCCTAAAGGCTTTGGGCGGGGCGGAGCCGAGAGCCACACTTTCA
AGTAAACCAGGTGGTGGAGACCCCATCCTTGGCTGCTTGCAGGGCCACTG
TCCAGGCAAATGCCAGGCCTTGTCCCCAGATGCCCAGGGCTCCCTTGTTG
CCCCTAATGCTCTCAGTAAACCTGAACACTTGGAAAACCAAAAAAAAAAA
AAAAAAAAAAAAAAAAAAAAAAAAAAAAAAAAAAAAAAAAAAAAATTTCN
(CloneID 5018693)

SEQ ID NO:26

GCCCAAGTGTCCCAAGTGCAACAAGGAGGTGTACTTCGCCGAGAGGGTGA
CCTCTCTGGGCAAGGACTGGCATCGGCCCTGCCTGAAGTGCGAGAAATGT
GGGAAGACGCTGACCTCTGGGGGCCACGCTGAGCACGAAGGCAAACCCTA
CTGCAACCACCCCTGCTACGCAGCCATGTTTGGGCCTAAAGGCTTTGGGC
GGGGCGGAGCCGAGAGCCACACTTTCAAGTAAACCAGGTGGTGGAGACCC
CATCCTTGGCTGCTTGCAGGGCCACTGTCCAGGCAAATGCCAGGCCTTGT
CCCCAGATGCCCAGGGCTCCCTTGTTGCCCCTAATGCTCTCAGTAAACCT
GAACACTTGGAAAAGCAAAAAAAAAAAAAAAAAA
(CloneID 155219 in 3′ to 5′ orientation wherein
the Coding and 3′ untranslated and/or non-Coding
regions are present or similar to that in CloneID
563289)

SEQ ID NO:27

GCATTAGGGGCAACAAGGGAGCCCTGGGCATCTGGGGACAAGGCCTGGCA
TTTGCCNGGACAGNGGCCCTGCAAGCAGCCAAGGATGGGGNCTCCACCAC
CTGGNTNACTTGAAAGTGTGGCTCTCGGCTCCGCCCGCCCAAAGCCTTNA
GGNCCAAACATGGCTGCGNANAGGNGNTGGTTGCAGNAGGGTTTGCCTTC
GTGCTCAGCGTGGCCCCCAGAGGTCAGCGTCTTCCCACATTTCTCGCACT
TCAGGCAGGGCCANTGCCANTCCTTNCCCAGAGAGGTCACCCTCTCGGCC
AAGTACACC
(CloneID 307716 in 3′ to 5′ orientation wherein
the Coding and 3′ untranslated and/or non-Coding
regions are present or similar to that in CloneID
563289) 307716 (3′)

SEQ ID NO:28

TTTTTTTTTTTTTTCCAAGTGTTCAGGTTTACTGAGAGCATTAGGGGCAA
CAAGGGAGCCCTGGNATCTGGGGACAAGGCCTGGCATTTGCTGGACAGTG
GCCCTGCAAGCAGCCAAGGATGGGGTCTCCACCACCTGGTTTACTTGAAA
GTGTGGCTCTCGGCTCCGCCCGCCCAAAGCCTTTAGNCNCAAACATGGAT
ATGGCTACGTAGCAGGGGTGGTTGCANTAGGGTTTCCCTTCGTGCTCAGC
GTGGCCCCCAGAGGTCAGCGTCTTCCCACATTTCTCGCACTTCAGGCAGG
GCNAATGCCANTCCTTGCCCAGAGAGGTCACCCTCTCGGG

In another set of preferred embodiments of the invention, any sequence, or unique portion thereof, of the HN1 sequences identified by the I.M.A.G.E. Consortium CloneID 471568 and cluster NM_{—}016185, as well as UniGene Homo sapiens cluster Hs.109706, may be used. HN1 sequences are useful in discriminations between normal and DCIS or IDC (of grades I to III) breast cells. Its expression is also increased in grade III relative to grade I breast cancer cells.

The consensus sequence of the I.M.A.G.E. Consortium cluster is as follows, with the assigned coding region (ending with a termination codon) underlined and preceded by the 5′ untranslated and/or non-coding region and followed by the 3′ untranslated and/or non-coding region:

(Consensus sequence for HN1)

SEQ ID NO:29

TGCAGCGGTGGTCGGCTGTTGGGTCTGGAGTTTCCCAGCGCCCCTCGGGT
CCGACCCTTTGAGCGTTCTGCTCCGGCGCCAGCCTACCTCGCTCCTCGGC
GCCATGACCACAACCACCACCTTCAAGGGAGTCGACCCCAACAGCAGGAA
TAGCTCCCGAGTTTTGCGGCCTCCAGGTGGTGGATCCAATTTTTCATTAG
GTTTTGATGAACCAACAGAACAACCTGTGAGGAAGAACAAAATGGCCTCT
AATATCTTTGGGACACCTGAAGAAAATCAAGCTTCTTGGGCCAAGTCAGC
AGGTGCCAAGTCTAGTGGTGGCAGGGAAGACTTGGAGTCATCTGGACTGC
AGAGAAGGAACTCCTCTGAAGCAAGCTCCGGAGACTTCTTAGATCTGAAG
GGAGAAGGTGATATTCATGAAAATGTGGACACAGACTTGCCAGGCAGCCT
GGGGCAGAGTGAAGAGAAGCCCGTGCCTGCTGCGCCTGTGCCCAGCCCGG
TGGCCCCGGCCCCAGTGCCATCCAGAAGAAATCCCCCTGGCGGCAAGTCC
AGCCTCGTCTTGGGTTAGCTCTGACTGTCCTGAACGCTGTCGTTCTGTCT
GTTTCCTCCATGCTTGAGAACTGCACAACTTGAGCCTGACTGTACATCTT
CTTGGATTTGTTTCATTAAAAAGAAGCACTTTATGTAAAAAAAAAAAAAA
AAAA

The sequences identified as belonging to the I.M.A.G.E. Consortium and UniGene clusters, with the assigned coding region underlined, follow below. The 5′ and 3′ untranslated and/or non-coding regions are by reference to the assigned coding region, which is presented as the complementary strand in the 3′ to 5′ direction for some of the sequences.

(CloneID 4795778)

SEQ ID NO:30

AGCGGCGCCGCTCCTGCAGCGGTGGTCGGCTGTTGGGTGTGGAGTTTCCC
AGCGCCCCTCGGGTCCGACCCTTTGAGCGTTCTGCTCCGGCGCCAGCCTA
CCTCGCTCCTCGGCGCCATGACCACAACCACCACCTTCAAGGGAGTCGAC
CCCAACAGCAGGAATAGCTCCCGAGTTTTGCGGCCTCGAGGTGGTGGATC
CAATTTTTCATTAGGTTTTGATGAACCAACAGAACAACCTGTGAGGAAGA
ACAAAATGGCCTCTAATATCTTTGGGACACCTGAAGAAAATCAAGCTTCT
TGGGCCAAGTCAGCAGGTGCCAAGTCTAGTGGTGGCAGGGAAGACTTGGA
GTCATCTGGACTGCAGAGAAGGAACTCCTCTGAAGCAAGCTCCGGAGACT
TCTTAGATCTGAAGGGAGAAGGTGATATTCATGAAAATGTGGACACAGAC
TTGCCAGGCAGCCTGGGGCAGAGTGAAGAGAAGCCCGTGCCTGCTGCGCC
TGTGCCCAGGCCCCAACCCACAGGGAGTCTCTCCGCAGAGCCTTCTTGGT
GTTGCCCTAACTTGCCAGTGGCCTTTGCTCAGAGCCTCCTCCTGTGACAT
GTGAACAATGAAGAGGCCTGCGCCTCCTGCCTTGCCGCCTGCAAAGCAAA
GAAACTGCCTTTTATTTTTTAACCTTAACAAGTAGCCAGATAGTAACAAG
ACTGGCCTGGAACTAAAGCAGTGAAGCAGGGGAGGCCATCACACTGAAGC
GGGTCTTCCTCCAGGAACGGGTCCACAAGGGTGTGTCTGAATTACCTGAT
GCTGTGTGCTGATGCTGGCTCTTGACCATGGACGGCAAGTTCATCTTAAC
CGTGCTGTCCTCACACCTGACTGTGCTCCCTTAACATTTCCC
(CloneID 5239921)

SEQ ID NO:31

CTCCTGCAGCGGTGGTCGGCTGTTGGGTGTGGAGTTTCCCAGCGCCCCTC
GGGTCCGACCCTTTGAGCGTTCTGCTCCGGCGCCAGCCTACCTCGCTCCT
CGGCGCCATGACCACAACCACCACCTTCAAGGGAGTCGACCCCAACAGCA
GGAATAGCTCCCGAGTTTTGCGGCCTCCAGGTGGTGGATCCAATTTTTCA
TTAGGTTTTGATGAACCAACAGAACAACCTGTGAGGAAGAACAAAATGGC
CTCTAATATCTTTGGGACACCTGAAGAAAATCAAGCTTCTTGGGCCAAGT
CAGCAGGTGCCAAGTCTAGTGGTGGCAGGGAAGACTTGCAGTCATCTGGA
CTGCAGAGAAGGAACTCCTCTGAAGCAAGCTCCGGAGACTTCTTAGATCT
GAAGGGAGAAGGTGATATTCATGAAAATGTGGACACAGACTTGCCAGGCA
GCCTGGGGCAGAGTGAAGAGAAGCCCGTGCCTGCTGCGCCTGTGCCCAGC
CCGGTGGCCCCGGCCCCAGTGCCATCCAGAAGAAATCCCCCTGGCGGCAA
GTCCAGCCTCGTCTTGGGTTAGCTCTGACTGTCCTGAACGCTGTCGTTCT
GTCTGTTTCCTCCATGCTTGTGAACTGCACAACTTGAGCCTGACTGTACA
TCTCTTGGATTTGTTTCATTAAAAAGAAGCACTTTATGTACTGCTGTCTT
TTCTTATCCCTTTGGAAGAACAGGTTTCTCTCTCTCCTTGACTCTTGGGT
CTGTGGGCCATGGCATGAGTGTTCCTAGTAGTAGATTGGAGGGAAAAGTT
TGTGACCCTTAGTACGCGGTTTTAAGACGAAATAATTGCGTTCCA
(CloneID 4623018)

SEQ ID NO:32

GGGGTGTGGAGTTTCCCAGCGCCCCTCGGGTCCGACCCTTTGAGCGTTCT
GCTCCGGCGCCAGCCTACCTCGCTCCTCGGCGCCATGACCACAACCACCA
CCTTCAAGGGAGTCGACCCCAACAGCAGGAATAGCTCCCGAGTTTTGCGG
CCTCCAGGTGGTGGATCCAATTTTTCATTAGGTTTTGATGAACCAACAGA
ACAACCTGTGAGGAAGAACAAAATGGCCTCTAATATCTTTGGGACACCTG
AAGAAAATCAAGCTTCTTGGGCCAAGTCAGCAGGTGCCAAGTCTAGTGGT
GGCAGGGAAGACTTGGAGTCATCTGGACTGCAGAGAAGGAACTCCTCTGA
AGCAAGCTCCGGAGACTTCTTAGATCTGAAGGGAGAAGGTGATATTCATG
AAAATGTGGACACAGACTTGCCAGGCAGCCTGGGGCAGAGTGAAGAGAAG
CCCGTGCCTGCTGCGCCTGTGCCCAGCCCGGTGGCCCCGGCCCCAGTGCC
ATCCAGAAGAAATCCCCCTGGCGGCAAGTCCAGCCTCGTCTTGGGTTAGC
TCTGACTGTCCTGAACGCTGTCGTTCTGTCTGTTTCCTCCATGCTTGTGA
ACTGCACAACTTGAGCCTGACTGTACATCTCTTGGATTTGTTTCATTAAA
AAGAAGCACTTTATGTACTGCTGTCTTTATTTTTTGCTTTGTGAAGACCA
GGTTTCTCTCTGTCCTTCACCTCTGTGGGTCTGTGGGCCATGGCATGAGT
GTTTCCTAGTAGTAGATGGGAGGGAAAGCTTGTGACCCCTTAGTACTGTG
TTTTTACAC
(CloneID 5729213)

SEQ ID NO:33

CGGTCCGGAATTCTCCGGATGCTGTTGGGTGTGGAGTTTCTACCGCCCCT
CGGGTCCGACCCTTTGAGCGTTCTGCTCCGGCGCCAGCCTACCTCGCTCC
TCGGCGCCATGACCACAACCACCACCTTCAAGGGAGTCGACCCCAACAGC
AGGAATAGCTCCCGAGTTTTGCGGCCTCCAGGTGGTGGATCCAATTTTTC
ATTAGGTTTTGATGAACCAACAGAACAACCTGTGAGGAAGAACAAAATGG
CCTCTAATATCTTTGGGACACCTGAAGAAAATCAAGCTTCTTGGGCCAAG
TCAGCAGGTGCCAAGTCTAGTGGTGGCAGGGAAGACTTGGAGTCATCTGG
ACTGCAGAGAAGGAACTCCTCTGAAGCAAGCTCCGGAGACTTCTTAGATC
TGAAGGGAGAAGGTGATATTCATGAAAATGTGGACACAGACTTGCCAGGC
AGCCTGGGGCAGAGTGAAGAGAAGCCCGTGCCTGCTGCGCCTGTGCCCAG
CCCGGTGGCCCCGGCCCCAGTGCCATCCAGAAGAAATCCCCCTGGCGGCA
AGTCCAGCCTCGTCTTGGGTTAGCTCTGACTGTCCTGAACGCTGTCGTTC
TGTCTGTTTCCTCCATGCTTGTGAACTGCACAACTTGAGCCTGACTGTAC
ATCTCTTGGATTTGTTTCATTAAAAAGAAGCACTTTATGTACTGCTGTCT
TTTTTTTTTTCTTTTGAAGAACAGGTTTCTCTCTGTCCTTGACTCTTGGG
TCTGTGGGCCATGGCATGAGTGTTTTCTAGTAGTAGATTGGAGGGAAAGC
TTTGTGACACTTAGTACTGTGTTTTTAAGAAGAAATAATTTGGTTCCAGA
TGTGTTANAAGATCTTTTGTACTGGAGTTTTTTACACTTTTACTTGGGTT
TAACCAGCCTCAACTGGGACGACCATAAACAGTCCACCAGGCACCGTTTC
CTGCCAGGCCCCAAACCCACGGGAAGTCTCTCGCAAAAACCCCTTCCTGG
GGGTTGCCCCTAATTTGCCAAGGGGCCTTTTGCTCCGGAGCCTCCCTCCC
GGGGAATTGGGGAAACAATGAAAAAGGCCTGTGCCCTCCGTGCCTTG
(CloneID 5192505)

SEQ ID NO:34

CTGCAGCGGTGGTCGGCTGTTGGGTGTGGAGTTTCCCAGCGCCCCTCGGG
TCCGACCCTTTGAGCGTTCTGCTCCGGCGCCAGCCTACCTCGCTCCTCGG
CGCCATGACCACAACCACCACCTTCAAGGGAGTCGACCCCAACAGCAGGA
ATAGCTCCCGAGTTTTGCGGCCTCCAGGTGGTGGATCCAATTTTTCATTA
GGTTTTGATGAACCAACAGAACAACCTGTGAGGAAGAACAAAATGGCCTC
TAATATCTTTGGGACACCTGAAGAAAATCAAGCTTCTTGGGCCAAGTCAG
CAGGTGCCAAGTCTAGTGGTGGCAGGGAACACTTGGACTCATCTGGACTG
CAGAGAAGGAACTCCTCTGAAGCAAGCTCCGGAGACTTCTTAGATCTGAA
GGGAGAAGGTGATATTCATGAAAATGTGGACACAGACTTGCCAGGCAGCC
TGGGGCAGAGTGAAGAGAAGCCCGTGCCTGCTGCGCCTGTGCCCAGCCCG
GTGGCCCCGGCCCCAGTGCCATCCAGAAGAAATCCCCCTGGCGGCAAGTC
CAGCCTCGTCTTGGGTTAGCTCTGACTGTCCTGAACGCTGTCGTTCTGTC
TGTTTCCTCCATGCTTGTGAACTGCACAACTTGAGCCTGACTGTACATCT
CTTGGATTTGTTTCATTAAAACAGAAGCACTTTATGTACTGCTGTCTTAT
ATCCTTTTGAAGAACAGGGTTTCTCTCTTGTCCTTGACCTCTGGGGTCTG
TGGGCCCATGGCATGAAGTGGTTTCTAGTAGTTAGATGGCGCGCAAAGCT
TTTGTGGACCCTATGTAACTGGTGGTTTTTACGACAGACACTACTTTGGC
TACCAGATGTGTTAGAGGAACCTTGGCACTGGAGGTTTAACCACTTTACT
GGGTTTACCAGGCCTTAGTGGCACGGCCCATAACGTCCACAGCCCCGATC
CTCCCAGCCCAA
(CloneID 3945044)

SEQ ID NO:35

TTGGGTGTGGAGTTTCCCAGCGCCCCTCGGGTCCGACCCTTTGAGCGTTC
TGCTCCGGCGCCAGCCTACCTCGCTCCTCGGCGCCATGACCACAACCAC
CACCTTCAAGGGAGTCGACCCCAACAGCAGGAATAGCTCCCGAGTTTGCG
GCCTCCAGGTGGTGGATCCAATTTTTCATTAGGTTTGATGAACCAACAGA
ACAACCTGTGAGGAAGAACAAAATGGCCTCTAATATCTTGGGACACCTGA
AGAAAATCAAGCTTCTTGGGCCAAGTCAGCAGGTGCCAAGTCTAGTGGTG
GCAGGGAAGACTTGGAGTCATCTGGACTGCAGAGAAGGAACTCCTCTGAA
GCAAGCTCCGGAGACTTCTTAGATCTGAAGGGAGAAGGTGATATTCATGA
AAATGTGGACACAGACTTGCCAGGCAGCCTGGGGCAGAGTGAAGAGAAGC
CCGTGCCTGCTGCGCCTGTGCCCAGCCCGGTGGCCCCGGCCCCAGTGCCA
TCCAGAAGAAATCCCCCTGGCGGCAAGTCCAGCCTCGTCTTGGGTTAGCT
CTGACTGTCCTGAACGCTGTCGTTCTGTCTGTTTCCTCCATGCTTGTGAA
CTGCACAACTTGAGCCTGACTGTACATCTCTGTGGATTGTTCTCATTAAA
AAGAAGCACTTTATGTACTGCTGTCTTTTTTTTTCCTTTTTGAAGAACCA
GGTTTCTCTCTGTCTTGACTCTGGGGTCTGTGGGGCCATGGCATGAGTGT
TTCTAGTAGTACATTGGCGGGAAACGCTTTGGGACCCTTTAGTACCTGTG
TTTCCACGGAACAAATTACTTTGGGGTCCCCATGTTGTTAA
(CloneID 5240530)

SEQ ID NO:36

CTGCAGCGGTGGTCGGCTGTTGGGTGTGGAGTTTCCCAGCGCCCCTCGGG
TCCGACCCTGTGGGAGCGTTCTGCTCCGGCGCCAGCCTACCTCGCTCCTC
GGCGCCATGACCACAACCACCACCTTCAAGGGAGTCGACCCCAACAGCAG
GAATAGCTCCCGAGTTTTGCGGCCTCCAGGTGGTGGATCCAATTTTTCAT
TAGGTTTGGATGAACCAACAGAACAACCTGTGAGGAAGAACAAAATGGCC
TCTAATATCTTTGGGACACCTGAAGAAAATCAAGCTTCTTGGGCCAAGTC
AGCAGGTGCCAAGTCTAGTGGTGGCAGGGAAGACTTGGAGTCATCTGGAC
TGCAGAGAAGGAACTCCTCTGAAGCAAGCTCCCGAGACTTCTTAGATCTG
AAGGGAGAAGGTGATATTCATGAAAATGTGGACACAGACTTGCCAGGCAG
CCTGGGGCAGAGTGAAGAGAAGCCCGTGCCTGCTGCGCCTGTGCCCAGCC
CGGTGGCCCCGGCCCCAGTGCCATCCAGAAGAAATCCCCCTGGCGGCAAG
TCCAGCCTCGTCTTGGGTTACCTCTGACTGTCCTGAACGCTGTCGTTCTG
TCTGTTTCCTCCATGCTTGTGAACTGCACAACTTGAGCCTGACTGTACAT
CTCTTGGATTNGTTTCATTAAAAAGAAGCACTTTATGTACTGCTGTCTTT
TAATTTTACTTTTGAAGAACAGGTTCTCTCTGTCCTTGACTCTGGGTCTG
GGGCCATGG
(CloneID 3617626)

SEQ ID NO:37

TGGCTAGGTACGAGGCTGGGTTTGGGCGGACAGGCGGCAGCGGCGGCTCC
TGCAGCGGTGGTCGGCTGTTGGGTGTGGAGTTTCCCAGCGCCCCTCGGGT
CCGACCCTTTGAGCGTTCTGCTCCGCCGCCAGCCTACCTCGCTCCTCGGC
GCCATGACCACAACCACCACCTTCAAGGGAGTCGACCCCAACAGCAGGAA
TAGCTCCCGAGTTTTGCGGCCTCCAGGTGGTGGATCCAATTTTTCATTAG
GTTTTGATGAACCAACAGAACAACCTGTGAGGAAGAACAAAATGGCCTCT
AATATCTTTGGGACACCTGAAGAAAATCAAGCTTCTTGGGCCAAGTCAGC
AGGTGCCAAGTCTAGTGGTGGCAGGGAAGACTTGGAGTCATCTGGACTGC
AGAGAAGGAACTCCTCTGAAGCAAGCTCCGGAGACTTCTTAGATCTGAAG
GGAGAAGGTGATATTCATGAAAATGTGGACACAGACTTGCCAGGCAGCCT
GGGGCAGAGTGAAGAGAAGCCCGTGCCTGCTGCGCCTGTGCCCAGCCCGG
TGGCCCCGGCCCCAGTGCCATCCAGAAGAAATCACCCTGGCGCCAGTCCA
GCCTCGTCTTGGGTATAGCTCTGACTGTCCTGAACGCTGTCGTTCTGTCT
GTTTCCTCCATGCTTGTGAACTGCACAACTGAGCCTGACTGTCATCTCTG
GGATTTGTTCATTAACAGAGCCTTATGTAAAAAAAAAAAAAAAACGCGGA
TTCTTCGGGGGGGAACAAGAAGAGGCCAGGGATA
(CloneID 4814114)

SEQ ID NO:38

AGCGGGGGGGCGGCTCCTGCAGCGGTGGTCGGCTGTTGGGTGTGGAGTTT
CCCAGCGCCCCTCGGGTCCGACCCTTTGAGCGTTCTGCTCCGGCGCCAGC
CTACCTCTCTCCTCGGCGCCATGACCACAACCACCACCTTCAAGGGAGTC
GACCCCAACAGCAGGAATAGCTCCCGAGTTTTGCGGCCTCCAGGTGGTGG
ATCCAATTTTTCATTAGGTTTTGATGAACCAACAGAACAACCTGTGAGGA
AGAACAAAATGGCCTCTAATATCTTTGGGACACCTGAAGAAAATCAAGCT
TCTTGGGCCAAGTCAGCAGGTGCCAAGTCTAGTGGTGGCAGGGAAGACTT
GGACTCATCTGGACTGCAGAGAAGGAACTCCTCTGAAGCAAGCTCCGGAG
ACTTCTTAGATCTGAAGGGAGAAGGTGATATTCATGAAAATGTGGACACA
GACTTGCCAGGCAGCCTGGGGCAGAGTGAAGAGAAGCCCGTGCCTGCTGC
GCCTGTGCCCAGCCCGGTGGCCCCGGCCCCAGTGCCATCCAGAAGAAATC
ACCCTGGCGGCAAGTCCAGCCTCGTCTTGGGTTAGCTCTGACTGTCCTGA
ACGCTGTCGTTCTGTCTGTTTCCTCCATGCTTGTGAACTGCACAACTTGA
GCCTGACTGTACATCTCTTGGATTTGTTTCATTAAAAAGAAGCACTTTAT
GTACTGCTGTCTTTTTTTCTTTCGGAAGAACAGGTTTCTCTCTGTCCTTG
ACTCTTGGGTCTGTGGGCCATGGCATTAGT
(CloneID 4894047)

SEQ ID NO:39

TGATGGCAGTAATCTCGGGGTATAAGGTAGCGAGGCTGGGTGGCGCCTGC
AGCGGTGGTCGGCTGTTGGGTGTGGAGTTTCCCAGCGCCCCTCGGGTCCG
ACCCTTTGAGCGTTCTGCTCCGGCGCCAGCCTACCTCGCTCCTCGGCGCC
ATGACCACAACCACCACCTTCAAGGGAGTCGACCCCAACAGCAGGAATAG
CTCCCGAGTTTTGCGGCCTCCAGGTGGTGGATCCAATTTTTCATTAGGTT
TTGATGAACCAACAGAACAACCTGTGAGGAAGAACAAAATGGCCTCTAAT
ATCTTTGGGACACCTGAAGAAAATCAAGCTTCTTGGGCCAAGTCAGCAGG
TGCCAAGTCTAGTGGTGGCAGGGAAGACTTGGAGTCATCTGGACTGCAGA
GAAGGAACTCCTCTGAAGCAAGCTCCGGAGACTTCTTAGATCTGAAGGGA
GAAGGTGATATTCATGAAAATGTGGACACACGACTTGCCAGGCAGCCTGG
GGCAGAGTGAAGAGAAGCCGTGCCTGCTGCGCCTGTGCCCAGCCCGGTGG
CCCCGNGCCCAGTGCCATCCAGAAGAAATCCCCCTGGCGGCAAGTCCAGC
CTCGTCTTGGGTTAGTTGAGCCTGACTGTACATCTCTTGGATTTGTTTCA
TTAAAAAGAAGCACTTTATGTACTGCTGTCTATTCAGACCTTTTGAAAGA
CCAGGTTTCTCTCTGTCCTTGACTCTGGGGCCTGTCGGCCATGGACTGAG
TGTTTCCTAGATAGCAGAT
(CloneID 5531619)

SEQ ID NO:40

AATTTTCCGAAATCGTCGACCCACGCGTCCGTTTCCAGCGGTGGTCGGCT
GTTGGGTGTGGAGTTTCCCAGCGCCCCTCGGGTCCGACCCTTTGAGCGTT
CTGCTCCGGCGCCAGCCTACCTCGCTCCTCGGCGCCATGACCACAACCAC
CACCTTCAAGGGAGTCGACCCCAACAGCAGGAATAGCTCCCGAGTTTTGC
GGCCTCCAGGTGGTGGATCCAATTTTTCATTAGGTTTTGATGAACCAACA
GAACAACCTGTGAGGAAGAACAAAATGGCCTCTAATATCTTTGGGACACC
TGAAGAAAATCAAGCTTCTTGGGCCAAGTCAGCAGGTGCCAAGTCTAGTG
GTGGCAGGGAAGACTTGGAGTCATCTGGACTGCAGAGAAGGAACTCCTCT
GAAGCAAGCTCCGGAGACTTCTTAGATCTGAAGGGAGAAGGTGATATTCA
TGAAAATGTGGACACAGACTTGCCAGGCAGCCTGGGGCAGAGTGAAGAGA
AGCCCGTGCCTGCTGCGCCTGTGCCCACCCCGGTGGCCCCGGCCCCAGTG
CCATCCAGAAGAAATCCCCCTGGCGGCAAGTCCAGCCTCGTCTTGGGTTA
GCTCTGACTGTCCTGAACGCTGTCGTTCTGTCTGTTTCCTCCATGCTTGT
GAACTGCACAACTTGAGCCTGACTGTACATCTCTTGGATTTGTTTCATTA
AAAAGAAGCACTTTAAAAAAAAAAAAAA
(CloneID 5588318)

SEQ ID NO:41

CGGTCCGGAATTCTCCGGATGCTGTTGGGTGTGGAGTTTCCCTGCGCCCC
TCGGGTCCGACCCTTTGAGCGTTCTGCTCCGGCGCCAGCCTACCTCGCTC
CTCGGCGCCATGACCACAACCACCACCTTCAAGGGAGTCGACCCCAACAG
CAGGAATAGCTCCCGAGTTTTGCGGCCTCCAGGTGGTGGATCCAATTTTT
CATTAGGTTTTGATGAACCAACAGAACAACCTGTGAGGAAGAACAAAATG
GCCTCTAATATCTTTGGGACACCTGAAGAAAATCAAGCTTCTTGGGCCAA
GTCAGCAGGTGCCAAGTCTAGTGGTGGCAGGGAAGACTTGGAGTCATCTG
GACTGCAGAGAAGGAACTCCTCTGAAGCAAGCTCCGGAGACTTCTTAGAT
CTGAAGGGAGAAGGTGATATTCATGAAAATGTGGACACAGACTTGCCAGG
CAGCCTGGGGCAGAGTGAAGAGAAGCCCGTGCCTGCTGCGCCTGTGCCCA
GCCCGGTGGCCCCGGCCCCAGTGCCATCCAGAAGAAATCCCCCTGGCGGC
AAGTCCAGCCTCGTCTTGGGTTAGCTCTGACTGTCCTGAACGCTGTCGTT
CTGTCTGTTTCCTCCATGCTTGTGAACTGCACAACTTGAGCCTGACTGTA
CATCTCTTGGATTTGTTTCATTAAAAAGAAGCACTTTATGTACTGCTGTC
TCTTTTTTTTTTCTTTTGAAGAACAGGTTTCTCTCTGTCCTTGACTCTTG
NGTCTGTGGGCCATGGCATGAGTGTTTTCTAGTAGTAGATTGGAGGGAAA
GCTTTGTGACACTTAGTACTGTGTTTTTAAGAGGAAATAATTTGGTTCCA
GATGTGTTAGAGGATCTTTTGTACTGAAGGTTTTACACCTTTACTTTGGG
TTTACCAGCCTCCACTGGAACAGACATAAACAGTCCACAGGCAACCGTTC
TGCCCAGCCCCAACCCCAGGGAAGTCTCTCCGCAAAACCTTTCTTGGGGT
TGCCCTAAACTTGCCAGGGGCCTTTGCTTCAAACCTCCCTCCTGTGAAAT
GTGTAACAAATGAAAAAGGCGTGGGCCTCTCTGCCTTTGCCCCCTTGAAA
ACAAAAAACCGGGCCTTTTTATTTTTTAACCTTAAAAAGAACCCCGATAT
TAAAAAAAAATGGGTTGGGTGTTGAACAAAACCCTGTCCTTCCC
(CloneID 5768283)

SEQ ID NO:42

TGCAGCGGTGGTCGGCTGTTGGGTGTGGAGTTTCCCAGCGCCCCTCGGGT
CCGACCCTTTGAGCGTTCTGCTCCGCCGCCAGCCTACCTCGCTCCTCGGC
GCCATGACCACAACCACCACCTTCAAGGGAGTCGACCCCAACAGCAGGAA
TAGCTCCCGAGTTTTGCGGCCTCCAGGTGGTGGATGCCAATTTTTCATTA
GGTTTTGATGAACCAACAGAACAACCTGTGAGGAAGAACAAAATGGCCTC
TAATATCTTTGGGACACCTGAAGAAAATCAAGCTTCTTGGGCCAAGTCAG
CAGGTGCCAAGTCTAGTGGTGGCAGGGAAGACTTGGAGTCATCTGGACTG
CAGAGAAGGAACTCCTCTGAAGCAAGCTCCGGAGACTTCTTAGATCTGAA
GGGAGAAGGTGATATTCATGAAAATGTGGACACAGACTTGCCAGGCAGCC
TGGGGCAGAGTGAAGAGAAGCCCGTGCCTGCTGCGCCTGTGCCCAGCCCG
GTGGCCCCGGCCCCAGTGCCATCCAGAAGAAATCCCCCTGGCGGCAAGTC
CAGCCTCGTCTTGGGTTAGCTCTGACTGTCCTGAACGCTGTCGTTCTGTC
TGTTTCCTCCATGCTTGTGAACTGCACAACTTGAGCCTGACTGTACATCT
CTTGGATTTGTTTCATTAAAAAGAAGCACTTTATGTACTGCTGTCTTTTT
TTTTTTTCTTTTGAAGAACAGGTTTCTCTCTGTCCTTGACTCTTGGGTCT
GTGGGCCATGGGCATGAGTGTTTTCTAGAAGTAGATTGGAGGGGAAAGCT
TTGTGGACACTTAAAACCGGGGTTTTTAAAAAAAATTAACTTGGGTTCCC
GAATGGGTTAGAAGAATCTTTTGGACCTGAGGTTTTTTAAACCTTTTAAT
TGGGGCTTAACCAAGCCTCTACTGGACCGAAATCATTAACAGGCCCCCCG
GCTCCGCGTTCCTTGCCAGGGCCCCAACCCACAGGGGGAGTTCTCCTCGG
CCCAAGCCCTTTTTTGGGGGCTCGCCCCCAAAATGTGCAGGGGGGCCTTT
GCGCTCAAAACCTCCC
(CloneID 5396994)

SEQ ID NO:43

GTGGAGTTTCCCAGCGCCCCTCGGGTCCGACCCTTTGAGCGTTCTGCTCC
GGCGCCAGCCTACCTCGCTCCTCGGCGCCATGACCACAACCACCACCTTC
AAGGGAGTCGACCCCAACAGCAGGAATAGCTCCCGAGTTTTGCGGCCTCC
AGGTGGTGGATCCAATTTTTCATTAGGTTTGATGAACCAACAGAACAACC
TGTGAGGAAGAACAAAATGGCCTCTAATATCCTTGGGACACCTGAAGAAA
ATCAAGCTTCTTGGGCCAAGTCAGCAGGTGCCAAGTCTAGTGGTGGCAGG
GAAGACTTGGAGTCATCTGGACTGCAGAGAAGGAACTCCTCTGAAGCAAG
CTCCGGAGACTTCTTAGATCTGAAGGGAGAAGGTGATATTCATGAAAATG
TGGACACAGACTTGCCAGGCAGCCTGGGGCAGAGTGAAGAGAAGCCCGTG
CCTGCTGCGCCTGTGCCCAGCCCGGTGGCCCCGGCCCCAGTGCCATCCAG
AAGAAATCCCCCTGGCGGCAACTCCAGCCTCGTCTTGGGTTAGCTCTGAC
TGTCCTGAACGCTGTCGTTCTGGTCTGTTTCCTCCATGCTTTGTGAACTG
GCACAACTTGAGCCTGACTGGTACATCTCTTTGGATTTGTTTCCATTAAA
AAGAAGCACTTTATGTACTGCTGTCTTTATTAGCCCTTTGGAAGAACCGG
TTTCTCTCTGTCCTGTGACTCCTGGGGTCTGTGGGCCATGGCATGAGGGT
CTCTAGTAGTAGATTGAGGGAAAGCTTGTGGCCACTTAATACTGGGTTTA
AAGACGAAATACTTGTGGTCCAGATGTGTTACAAGGAACCTTGGCACGGA
GGTTATAACACCTTTATGGGGTGTCCCAGGCTCCCTGGACGACCCTAAGC
GTCCACGGCGCCGGTCCGCCGGCCCAC
(CloneID 3941190)

SEQ ID NO:44

TTGGGTGTGGAGTTTCCCAGCGCCCCTCGGGTCGACCCTTTGAGCGTTCT
GCTCCGGCGCCAGCCTACCTCGCTCCTCGGCGCCATGACCACAACCACCA
CCTTCAAGGGAGTCGACCCCAACAGCAGGAATAGCTCCCGAGTTTTGCGG
CCTCCAGGTGGTGGATCCAATTTTTCATTAGGTTTTGATGAACCAACAGA
ACAACCTGTGAGGAAGAACAAAATGGCCTCTAATATCTTTGGGACACCTG
AAGAAAATCAAGCTTCTTGGGCCAAGTCAGCAGGTGCCAAGTCTAGTGGT
GGCAGGGAAGACTTGGAGTCATCTGGACTGCAGAGAAGGAACTCCTCTGA
AGCAAGCTCCGGAGACTTCTTAGATCTGAAGGGAGAAGGTGATATTCATG
AAAATGTGGACACAGACTTGCCAGGCAGCCTGGGGCAGAGTGAAGAGAAG
CCCGTGCCTGCTGCGCCTGTGCCCAGCCCGGTGGCCCCGGCCCCAGTGCC
ATCCAGAAGAAATCCCCCTGGCGGCAACTCCAGCCTCGTCTTGGGTTAGC
TCTGACTGTCCTGAACGCTGTCGTTCTGTCTGTTTCCTCCATGCTTGTGA
ACTGCACAACTTGAGCCTGACTGTACATCTCTTGGATTTGTTTCATTAAA
AAAGAAGCACTTTATGTACTGCTGTCTTTTTTTTCCTTTCGAAGAACAGG
TTGCTCTCTGCCTTGACTCTGTGGGTCTGTGGCGCATGGATGAGTGTTCC
TAGCTAGTAGATGGAGGGCAACTTTGGGGACCCTTAGCGTGGTTTACCAG
AAATACCTGGGTCCAGTGGCCCAGGCCTTGGCCGGGGTTCCACCTTCTGG
GTTTCAGCTCACGGGAACCATAAACCAAGGCGCCGAGGCCACAGGATCCC
GCCTTGTGGCATCCGGGGTGACCCGTATGAAAAAGCGCCTCCAAATTTTT
AAACACAAGCGCCCGGAGACCAT
(CloneID 5928395)

SEQ ID NO:45

GCAGCGGTGGTCGGCTGTTGGGTGTGGAGTTTCCCAGCGCCCCTCGGGTC
CGACCCTTTGAGCGTTCTGCTCCGGCGCCAGCCTACCTCGCTCCTCGGCG
CCATGACCACAACCACCACCTTCAAGGGAGTCGACCCCAACAGCAGGAAT
AGCTCCCGAGTTTTGCGGCCTCCAGGTGGTGGATCCAATTTTTCATTAGG
TTTTGATGAACCAACAGAACAACCTGTGAGGAAGAACAAAATGGCCTCTA
ATATCTTTGGGACACCTGAAGAAAATCAAGCTTCTTGGGCCAAGTCAGCA
GGTGCCAAGTCTAGTGGTGGCAGGGAAGACTTGGAGTCATCTGGACTGCA
GAGAAGGAACTCCTCTGAAGCAAGCTCCGGAGACTTCTTAGATCTGAAGG
GAGAAGGTGATATTCATGAAAATGTGGACACAGACTTGCCAGGCAGCCTG
GGGCAGAGTGAAGAGAAGCCCGTGCCTGCTGCGCCTGTGCCCAGCCCGGT
GGCCCCGGCCCCAGTGCCATCCAGAAGAAATCCCCCTGGCGGCAAGTCCA
GCCTCGTCTTGGGTTAGCTCTGACTGTCCTGAACGCTGTCGTTCTGTCTG
TTTCCTCCATGCTTGTGAACTGCACAACTTGAGCCTGACTGTACATCTCT
TGGATTTGTTTCATTAAAAAGAAGCACTTTATGTACTGCTGTCTTTTTTT
TTTTTCTTTTGAAGAACAGGTTTCTCTCTGTCCTTGACTCTTGGGTCTGT
GGGCCATGGCATGAGTGTTTTCTAGTAATAGATTGGAGGGAAACCTTTGT
GACACTTAATACTGTGTTTTTAAGAAGAAATAATTTGGTTCCAAATGTGT
TAGGAGGATCTTTTTGTACTGAAGTTTTTAAAACTTTACTTGGGTTTTAC
CAAGCCCTCACCTGGGACAGACCCTTAACCGGTCCAACGGGCCACCGTTT
CTTGCCAGGGCCCCAAACCCACAGGGGAAGTCTTCTCGCAAAAACCCTTC
CTGGGGGGTTCCCCCTAACTTGCCCAGGGGCCCCTTTGCTCCAAAAC
(CloneID 4750262)

SEQ ID NO:46

CGATCGGCCGGACAGGCGGCAGCGGCGGCTCCTGCAGCGGTGGTCGGCTG
TTGGGTGTGGAGTTTCCCAGCGCCCCTCGGGTCCGACCCTTTGAGCGTTC
TGCTCCGGCGCCAGCCTACCTCGCTCCTCGGCGCCATGACCACAACCACC
ACCTTCAAGGGAGTCGACCCCAACAGCAGGAATAGCTCCCGAGTTTTGCG
GCCTCCAGGTGGTGGATCCAATTTTTCATTAGGTTTCGATGAACCAACAG
AACAACCTGTGAGGAAGAACAAAATGGCCTCTAATATCTTTGGGACACCT
GAAGAAAATCAAGCTTCTTGGGCCAAGTCAGCAGGTGCCAAGTCTAGTGG
TGGCAGGGAAGACTTGGAGTCATCTGGACTGCAGAGAAGGAACTCCTCTG
AAGCAAGCTCCGGAGACTTCTTAGATCTGAAGGGAGAAGGTGATATTCAT
GAAAATGTGGACACAGACTTGCCAGGCAGCCTGGGGCAGAGTGAAGAGAA
GCCCGTGCCTGCTGCGCCTGTGCCCAGCCCGGTGGCCCCGGCCCCAGTGC
CATCCAGAAGAAATCCCCCTGGCGGCAAGTCCAGCCTCGTCTTGGGTTAG
CTCTGACTGTCCTGAACGCTGTCGTTCTGTCTGTTTCCTCCATGCTTGTG
AACTGCACAACTTGAGCCTGACTGTACATCTCTTGGATTGGTGTCATTAC
ACAAGAAGCACTTTATGTACTGCTGGTCTATAAATATCCTTTGCGACGAA
CAGGTTCCTCTGTGCCTTGACTCTGGGGCCTGTGGGCCACTGGCAGTGAG
TTGTGACTAGCTAGTAGATTGGGCGGAAACGCTTGGTGACCCTTAGCCCC
TGGTTTCTACGACGCCATACTTGGTCCCGATGTGTTACAGGCCTCTTGCG
CCGGAGGCTTTACACCCTTTACTGCGCTCCCAAGGCTCAGGGGCGACCTA
A
(CloneID 5274910)

SEQ ID NO:47

AGCGGAGCGGCGGCTCCTGCAGCGGTGGTCGGCTGTTGGGTGTGGAGTTT
CCCAGCGCCCCTCGGGTCCGACCCTTTGAGCGTTCTGCTCCGGCGCCAGC
CTACCTCGCTCCTCGGCGCCATGACCACAACCACCACCTTCAAGGGAGTC
GACCCCAACAGCAGGAATAGCTCCCGAGTTTTGCGGCCTCCAGGTGGTGG
ATCAAATTTTTCATTAGGTTTTGATGAACCAACAGAACAACCTGTGAGGA
AGAACAAAATGGCCTCTAATATCTTTGGGACACCTGAAGAAAATCAAGCT
TCTTGGGCCAAGTCAGCAGGTGCCAAGTCTAGTGGTGGCAGGGAAGACTT
GGAGTCATCTGGACTGCAGAGAAGGAACTCCTCTGAAGCAAGCTCCGGAG
ACTTCTTAGATCTGAAGGGAGAAGGTGATATTCATGAAAATGTGGACACA
GACTTGCCAGGCAGCCTGGGGCAGAGTGAAGAGAAGCCCGTGCCTGCTGC
GCCTGTGCCCAGCCCGGTGGCCCCGGCCCCAGTGCCATCCAGAAGAAATC
CCCCTGGCGGCAAGTCCAGCCTCGTCTTGGGTTAGCTCTGACTGTCCTGA
ACGCTGTCGTTCTGTCTGTTTCCTCCATGCTTGTGAACTGCACAACTTGA
GCCTGACTGTACATCTCTTGGATTTGTTTCATTCACAAAGAAGCACTTTA
TGTAACTGCTGTCTTTTTTACCTTTTGAAGAACCGGTTCCTCTCTGTCCT
TGACTCTGGGGTCTGGGGGCCAGGCATT
(CloneID 3140086)

SEQ ID NO:48

TTGGGTGTGGAGTTTCCCAGCGCCCCTCGGGTCCGACCCTTTGAGCGTTC
TGCTCCGGCGCCGGCCTACCTCGCTCCTCGCCGCCATGACCACAACCACC
ACCTTCAAGGGAGTCGACCCCAACAGCAGGAATAGCTCCCGAGTTTTGCG
GCCTCCAGGTGGTGGATCCAATTTTTCATTAGGTTNTGATGAACCAACAG
AACAAGGCGGTGAGGAAGAACAAAATGGCCTCTAATATCTTTGGGACACC
TGAAGAAAATCAAGCTTCTTGGGCCAAGTCAGCAGGTGCCAAGTCTAGTG
GTGGCAGGGAAGACTTGGAGTCATCTGGACTGCAGAGAAGGAACTCCTCT
GAAGCAAGCTCCGGAGACTTCTTAGATCTGAAGGGAGAAGGTGATATTCA
TGAAAATGTGGACACAGACTTGCCAGGCAGCCTGGGGCAGAGTGAAGAGA
AGCCCGTGCCTTGCTGCGCCTGTGCCCAGCCCGGTGGCCCCGGCCCCAGT
GCCATCCAGAAGAAATCCCCCTGGCGGCAAGTCCAGCCTCGTCTTGGGTT
AGCTCTGACTGTCCTGAACGCTGTCGTTCTGTCTGGTTTCCTCCATGCTT
GTGAACTGGACAACTTGAGCCTGACTGTACATCTCTTGGATTGTGTTTCA
TTCAAAAGACGCACTTTATGTAACACAAA
(CloneID 5286160)

SEQ ID NO:49

AGCGGAGGGGCGGCTCCTGCAGCGGTGGTCGGCTGTGGGTGTGGAGTTTC
CGAGCGCCCCTCGGGTCCGACCCTTTGAGCGTCTGCTCCGGCGCCAGCCT
ACCTCGCTCCTCGGCGCGATGACCACAACCACCACCTTCAAGGGAGTCGA
CCCCAACAGCAGGAATAGCTCCCGAGTTTTGCGGCCTCCAGGTGGTGGAT
CCAATTTTTCATTAGGTTTTGATGAACCAACAGAACAACCTGTGAGGAAG
AACAAAATGGCCTCTAATATCTTTGGGACACCTGAAGAAAATCAAGCTTC
TTGGGCCAACTCAGCAGGTGCCAAGTCTAGTGGTGGCAGGGAAGACTTGG
AGTCATCTGCACTGCAGAGAAGGAACTCCTCTGAAGCAAGCTCCGGAGAC
TTCTTAGATCTGAAGGGAGAAGGTGATATTCATGAAAATGTGGACACAGA
CTTGCCAGGCAGCCTGGGGCAGAGTGAAGAGAAGCCCGTGCCTGCTGCGC
CTGTGCCCAGCCCGGTGGCCCCGGCCCCAGTGCCATCCAGAAGAAATCCC
CCTGGCGGCAAGTCCAGCCTCGTCTTGGGTTAGCTCTGACTGTCCTGAAC
GCTGTCGTTCTGTCTGTTTCCTCCATGCTTGTGAACTGCACAACTTGAGC
CTGACTGTACATCTCTTGGATTTGTTTCATTACAAAGAAGCACTTTATGT
ACTGCTGTCTTTTTTTCCTTTTGAAGAAC
(CloneID 5215141)

SEQ ID NO:50

GGTACCGGTCCCGAGTTCTTTGGATGCAGCGGTGGTCGGCTGTTGGGTGT
GGAGTTTCCCAGCGCCCCTCGGGTCCGACCCTTTGGAGCGTTCTGCTCCG
GCGCCAGCCTACCTCGCTCCTCGGCGCCATGACCACAACCACCACCTTCA
AGGGAGTCGACCCCAACAGCAGGAATAGCTCCCGAGTTTTGCGGCCTCCA
GGTGGTGGATCCAATTTTTCATTAGGTTTTGATGAACCAACAGAACAACC
TGTGAGGAAGAACAAAATGGCCTCTAATATCTTTGGGACACCTGAAGAAA
ATCAAGCTTCTTGGGCCAAGTCAGCAGGTGCCAAGTCTAGTGGTGGCAGG
GAAGACTTGGAGTCATCTGGACTGCAGAGAAGGAACTCCTCTGAAGCAAG
CTCCGGAGACTTCTTAGATCTGAAGGGAGAACGGTGATATTCATGAAAAT
GTGGACACAGACTTGCCAGGCAGCCTGGGGCAGAGTGAAGAGAAGCCCGT
GCCTGCTGCGCCTGTGCCCAGACCCGGTGGACCCGGACCCAGTTGCCATC
CAGAAGAAATCCCCCTGGCGGCAAGTCCAGCCTCGTCTAGGGTTAGCTCT
GACTGTCCTGAACGCTGTCGTCTGTCTGTTTCCTCCATGCTTGTGAACTG
CACAACTTGAGCCTGACTGTTACATCTCTTGGATAAGTTTCATTAAAAAG
AAGCACTTTATGTACTG
(CloneID 4845679)

SEQ ID NO:51

GAGCGGTGGTCGGCTGTGGGTGTGGAGTTTCCCAGCGCCCCTCGGGTCCG
ACCCTTTGAGCGTTCTGCTCCGGCGCCAGCCTACCTCGCTCCTCGGCGCC
ATGACCACAACCACCACCTTCAAGGGAGTCGACCCCAACAGCAGGAATAG
CTCCCGAGTTTGCGGCCTCCAGGTGGTGGATCCAATTTTTCATTAGGTTT
GATGAACCAACAGAACAACCTGTGAGGAAGAACAAAATGGCCTCTAATAT
CTTTGGGACACCTGAAGAAAATCAAGCTTCTTGGGCCAAGTCAGCAGGTG
CCAACTCTACTGGTGGCAGGGAAGACTTGGAGTCATCTGGACTGCAGAGA
AGGAACTCCTCTGAAGCAAGCTCCGGAGACTTCTTAGATCTGAAGGGAGA
AGGTGATATTCATGAAAATGTGGACACAGACTTGCCAGGCAGCCTGGGGC
AGAGTGAAGAGAAGCCCGTGCCTGCTGCGCCTGTGCCCAGCCCGGTGGCC
CCGGCCCCAGTGCCATCCAGAAGAAATCCCCCTGGCGGCAAGTCCAGCCT
CGTCTTGGGTTAGCTCTGACTGTCCTGAACGCTGTCGTTCTGTCTGTTTC
CTCCATGCTTGTGAACTGCACAACTTGAGCCTGACTGTACATCTCTTGGA
TTTGTTTCATTAAAAAGAAGCACTCTATGTACTGCTGTCTTTTTTTTTCT
TTGGAAGAA
(CloneID 5297866)

SEQ ID NO:52

AGCGGAGCGTCGGCTCCTGCAGCGGTGGTCCGCTGTTGGGTGTGGAGTTT
CCCAGCGCCCCTCGGGTCCGACCCTTTCAGCGTTCTGCTCCGGCGCCAGC
CTACCTCGCTCCTCGGCGCCATGACCACAACCACCACCTTCAAGGGAGTC
GACCCCAACAGCAGGAATAGCTCCCGAGTTTTGCGGCCTCCAGGTGGTGG
ATCCAATTTTTCATTAGGTTTTGATGAACCAACAGAACAACCTGTGAGGA
AGAACAAAATGGCCTCTAATATCTTTGGGACACCTGAAGAAAATCAAGCT
TCTTGGGCCAAGTCAGCAGGTGCCAAGTCTAGTGGTGGCAGGGAAGACTT
GGAGTCATCTGGACTGCAGAGAAGGAACTCCTCTGAAGCAAGCTCCGGAG
ACTTCTTAGATCTGAAGGGCGACGGTGATATTCATGAACAATGTGGACAC
CAGACTTGCCAGGCAGCCTGGGGCAGAGTGAAGAGAAGCCCGTGCCTGCT
GCGCCTTGTGCCCAGACCCGGTTGGCCCCGGCCCCCAGTGCCATCCAGAA
GAAATCCCCCTGGCGGCATGTCAGCCTCGTCTTGGGTAAGCTCTGACTGT
CCTGAACGCTGTCGTTCTGTCTGTTTACCTCCATGCTTGTTGAACTTGAC
AACTTGGAGCCTGACGTGTACATCTCTTGGATTGGTTTCATTAAAAAAGA
AGCACTTTATGTACTGGCTGTCTTAAATAACTTTTGAAAGAAC
(CloneID 5194208)

SEQ ID NO:53

CGAATCCCTGGATCTGCAGCGGTGGTCGGCTGTTGGGTGTGGAGTTTCCC
AGCGCCCCTCGGGTCCCACCCTTTGAGCGTTCTGCTCCGGCGCCAGCCTA
CCTCGCTCCTCGGCGCCATGACCACAACCACCACCTTCAAGGGAGTCGAC
CCCAATAGCAGGAATAGCTCCCGAGTTTTGCGGCCTCCAGGTGGTGGATC
CAATTTTTCATTAGGTTTTGATGAACCAACAGAACAACCTGTGAGGAAGA
ACAAAATGGCCTCTAATATCTTTGGGACACCTGAAGAAAATCAAGCTTCT
TGGGCCAAGTCAGCAGGTGCCAAGTCTAGTGGTGGCAGGGAAGACTTGGA
GTCATCTGGACTGCAGAGAAGGAACTCCTCTGAAGCAAGCTCCGGAGACT
TCTTAGATCTGAAGGGAGAAGGTGATATTCATGAAAATGTGGACACAGAC
TTGCCAGGCAGCCTGGGGCAGAGTGAAGAGAAGCCCGTGCCTGCTGCGCC
TGTGCCCAGCCCGGTGGCCCCGGCCCCAGTGCCATCCAGAAGAATCCCCC
CTGGCGGCAAGTCCAGCCTCGTCTTGGGTTAGCTCTGACTGTCCTGAACG
CTGTCGTTCTGTCTGTTTCCTCCATGCTGTGTGAAACTGGACAACTTGAG
CCTGACTGTACATCTCGTTGGATTGGTTTCATTAACAAAGAAGCACTTTA
TGTACTTGCTGTCTTTTGATGCTTTGGAGAGAACAGGTTACTACTCTGGC
CCTTGACTCTGGGGTCTGTGGGCCATGGCATGAGTGTTTACTACGTAGCA
CAACTGGCGCGGAAACGCTCGGGTGAGACCCATTAGCTACGGGGGTTTTA
AGAACGCAAATTAATCGGGTACGCGAATGTGGTACAAGGACACTGTAGGA
CACGGACGCTGTAACACACTTAACAAGCGGTTTCACCAGGACCTCAACGG
AGACCGACCATATAACGGCCACAGGAGCACGGACATGACAGGGCCAAGCC
(CloneID 5017113)

SEQ ID NO:54

GCCTGCAGCGGTGGTCGGCTGTGGGTGTGGAGTTTCCCAGCGCCCCTCGG
GTCCGACCCTTTCAGCGTTCTGCTCCGGCGCCAGCCTACCTCGCTCCTCG
GCGCCATGACCACAACCACCACCTTCAAGGGAGTCGACCCCAACAGCAGG
AATAGCTCCCGAGTTTTGCGGCCTCCAGGTGGTGGATCCAATTTTTCATT
AGGTTTTGATGAACCAACAGAACAACCTGTGAGGAAGAACAAAATGGCCT
CTAATATCTTTGGGACACCTGAAGAAAATCAAGCTTCTTGGGCCAAGTCA
GCAGGTGCCAAGTCTAGTGGTGGCAGGGAAGACTTGGAGTCATCTGGACT
GCAGAGAAGGAACTCCTCTGAAGCAAGCTCCGGAGACTTCTTAGATCTGA
AGGGAGAAGGTGATATTCATGAAAATGTGGACACAGACTTGCCAGGCAGC
CTGGGGCAGAGTGAAGAGAAGCCCGTGCCTGCTGCGCCTGTGCCCAGCCC
GGTGGCCCCGGCCCAGTGCCATCCAGAAGAAATCCCCCTGGCGGCAAGTC
CAGCCTCGTCTGTGGGTTAGCTCTGACTGTCCTGAACGCTGTCGTTCTGT
CTGTTTCCTCCATGCTTGTGAACTGACAATTGAGCCTGACTGTACATCTC
TTGGATTGGGTTTCATTAAAAAGAAGCACTTTAAAAAAAAAAAAGAAAAA
AAAAAAAAT
(CloneID 5483414)

SEQ ID NO:55

TTGGGTGTGGAGTTTCCCAGCGCCCCTCGGGTCCGACCCTTTGAGCGTTC
GCTCCGGCGCCAGCCTACCTCGCTCCTCGGCGCCATGACCACAACCACC
ACCTTCAAGGGAGTCGACCCCAACAGCAGGAATAGCTCCCGAGTTTTGCG
GCCTCCAGGTGGTGGATCCAATTTTTCATTAGGTTTTGATGAACCAACAG
AACAACCTGTGAGGAAGAACAAAATGGCCTCTAATATCTTTGGGACACCT
GAAGAAAATCAAGCTTCTTGGGCCAAGTCAGCAGGTGCCAAGTCTAGTGG
TGGCAGGGAAGACTTGGAGTCATCTGGACTGCAGAGAAGGAACTCCTCTG
AAGCAAGCTCCGGAGACTTCTTAGATCTGAAGGGAGAAGGTGATATTCAT
GAAAATGTGGACACAGACTTGCCAGGCAGCCTGGGGCAGAGTGAAGAGAA
GCCCGTGCCTGCTGCGCCTGTGCCCAGCCCGGTGGCCCCGGCCCAGTGCC
ATCCAGAAGAAATCCCCCTGGCGGCAAGTCCAGCCTCGTCTGTGGGTTAG
CTCTGACTGTCCTGAACGCTGTCGTTCTGTCTGTTTCCTCCATGCTTGTG
AACTGCACAACTTGAGCCTGACTGTACATCTCTTGGATTTGTTTCATTAA
AAAGAAGCACTTTATGTACTGCTGTCTTTTTTTTTTTTCTTTTGAAGAAC
AGGTTTCTCTCTGTCCTTGACTCTTGGGTCTGTGGGCCATGGCATGAGTG
TTTTCTAGTAGTAGATTGGAGGGAAAGCTTTGTGACACTTAGTACTGTGT
TTTTAGAAGAAATAATTTGGTTCCAAATGTGTTAAAGGATCTTTTGAACT
GAGGTTTTTAAACACTTTACTTGGGTTTCCCAAGCCTCAACTGGACAGAA
CATAAACAGTCCACAGGCACCGGTTCCTGCCAGGGCCCAAACCCACAGGA
AATCCTTTCGCCAAAAACCCCTTCTTGGGGGTGGCCCCTACTTGGCCAGT
GGCCCTTTGCTCCAAAACCTCCTCCTGGGAACCGGGGGAAAACATTGAAA
AGGCCCTGGGGCCTCCCT
(CloneID 5778829)

SEQ ID NO:56

GCACGAGGTCGGCTGTTGGGTGTGGAGTTTCCCAGCGCCCCTCGGGTCCG
ACCCTTTGAGCGTTCTGCTCCGGCGCCAGCCTACCTCGCTCCTCGGCGCC
ATGACCACAACCACCACCTTCAAGGGAGTCGACCCCAACAGCAGGAATAG
CTCCCGAGTTTTGCGGCCTCCAGGTGGTGGATCCAATTTTTCATTAGGTT
TTGATGAACCAACAGAACAACCTGTGAGGAAGAACAAAATGGCCTCTAAT
ATCTTTGGGACACCTGAAGAAAATCAAGCTTCTTGGGCCAAGTCAGCAGG
TGCCAAGTCTAGTGGTGGCAGGGAAGACTTGGAGTCATCTGGACTGCAGA
GAAGGAACTCCTCTGAAGCAAGCTCCGGAGACTTCTTAGATCTGAAGGGA
GAAGGTGATATTCATGAAAATGTGGACACAGACTTGCCAGGCAGCCTGGG
GCAGAGTGAAGAGAAGCCCGTGCCTGCTGCGCCTGTGCCCAGCCCGGTGG
CCCCGGCCCCAGTGCCATCCAGAAGAAATCCCCCTGGCGGCAAGTCCAGC
CTCG
(CloneID 5778829 in 3′ to 5′ orientation)

SEQ ID NO:57

TTTTTTTTTTTTTTTTTTTTTTTACATAAAGTGCTTCTTTTTAATGAAAC
AAATCCAAGAGATGTACAGTCAGGCTCAAGTTGTGCAGTTCACAAGCATG
GAGGAAACAGACAGAACGACAGCGTTCAGGACAGTCAGAGCTAACCCAAG
ACGAGGCTGGACTTGCCGCCAGGGGGATTTCTTCTGGATGGCACTGGGGC
CGGGGCCACCGGGCTGGGCACAGGCGCAGCAGGCACGGGCTTCTCTTCAC
TCTGCCCCAGGCTGCCTGGCAAGTCTGTGTCCACATTTTCATGAATATCA
CCTTCTCCCTTCAGATCTAAGAAGTCTCCGGAGCTTGCTTCAGAGGAGTT
CCTTCTCTGCAGTCCAGATGACTCCAAGTCTTCCCTGCCACCACTAGACT
TGGCACCTGCTGACTTGGCCCAAGAAGCTTGATTTTCTTCAGGTGTCCCA
AAGATATTAGAGGCCATTTTGTTCTTCCTCACAGGTTGTTCTGTTGGTTC
ATCAAAACCTAATGAAAAATTGGATCCACCACCTGGAGGCCGCAAAACTC
GGGAGCTATTCCTGCTGTTGGGGTCGACTCCCTTGAAGGT
(CloneID 2781859)

SEQ ID NO:58

GACCGGCGGCTCCTGCAGCGGTGGTCGGCTGTTGGGTGTGGACTTTCCCA
GCGCCCCTCGGGTCCGACCCTTTGAGCGTTCTGCTCCGGCGCCAGCCTAC
CTCGCTCCTCGGCGCCATGACCACAACCACCACCTTCAAGGGAGTCGACC
CCAACAGCAGGAATAGCTCCCGAGTTTTGCGGCCTCCAGGTGGTGGATCC
AATTTTTCATTAGGTTTTGATGAACCAACAGAACAACCTGTGAGGAAGAA
CAAAATGGCCTCTAATATCTTTGGGACACCTGAAGAAAATCAAGCTTCTT
GGGCCAAGTCAGCAGGTGCCAAGTCTAGTGGTGGCAGGGAAGACTTGGAG
TCATCTGGACTGCAGAGAAGGAACTCCTCTGAAGCAAGCTCCGGAGACTT
CTTAGATCTGAAGGGAGAAGGTGATATTCATGAAAATGTGGACACAGACT
TGCCAGGCAGCCTGGGGCAGAGTGAAGAGAAGCCCGTGCCTGCTGCGCCT
GTGCCCAGCCCGGTGGCCCCGGCCGCAGTGCCATCCAGAAGAAATCCCCC
TGGCGGCAAGTCCAGCCTCGTCTTG
(CloneID 2781859 in 3′ to 5′ orientation)

SEQ ID NO:59

TTTTTTTACATAAAGTGCTTCTTTTTAATGAAACAAATCCAAGAGATGTA
CAGTCAGGCTCAAGTTGTGCAGTTCACAAGCATGGAGGAAACAGACAGAA
CGACAGCGTTCAGGACAGTCAGAGCTAACCCAAGACGAGGCTGGACTTGC
CGCCAGGGGGATTTCTTCTGGATGGCACTGGGGCCGGGGCCACCGGGCTG
GGCACAGGCGCAGCAGGCACGGGCTTCTCTTCACTCTGCCCCAGGCTGCC
TGGCAAGTCTGTGTCCACATTTTCATGAATATCACCTTCTCCCTTCAGAT
CTAAGAAGTCTCCGGAGCTTGCTTCAGAGGAGTTCCTTCTCTGCAGTCCA
GATGACTCCAAGTCTTCCCTGCCACCACTAGACTTGGCACCTGCTGACTT
GGCCCAAGAAGCTTGATTTTCTTCAGGTGTCCCAAAGATATTAGAGGCCA
TTTTGTTCTTCCTCACAGGTTGTTCTGTTGGTTCATCAAAACCTAATGAA
AAATTGGATCCACCACCTGGAGGCCGCANAACTCGGGAGCTATTCCTGCT
GTTGGGGTCGACTCCCTTGAAGGTGGTGGTTGTGGTCAT
(CloneID 3940455)

SEQ ID NO:60

GGGTGTGGAGTTTCCCAGCGCCCCTCGGGTCGACCCTTTGAGCGTTCTGC
TCCGGCGCCAGCCTACCTCGCTCCTCGGCGCCATGACCACAACCACCACC
TTCAAGGGAGTCGACCCCAACAGCAGGAATAGCTCCCGAGTTTGCGGCCT
CCAGGTGGTGGATCCAATTTTTCATTAGGTTTTGATGAACCAACAGAACA
ACCTGTGAGGAAGAACAAAATGGCCTCTAATATCTTTGGGACACCTGAAG
AAAATCAAGCTTCTTGGGCCAAGTCAGCAGGTGCCAAGTCTAGTGGTGGC
AGGGAAGACTTGGAGTCATCTGGACTGCAGAGAAGGAACTCCTCTGAAGC
AAGCTCCGGAGACTTCTTAGATCTGAAGGGAGAAGGTGATATTCATGAAA
ATGTGGACACAGACTTGCCAGGCAGCCTGGGGCAGAGTGAAGAGAAGCCC
GTGCCTGCTTGCGCCTGTGCCCAGCCCGGTGGCCCCGGCCCAGTGCCATC
CAGAAGAAATCCCCCTGGCGGCAAGTCCCAGCCTCGTCTTGGGTTAGCTC
TGACTGTCCTGAACGCTGTCGTTCTGTCTGTNTCCTCCATGCTTGGTGGA
ACTGACAATTTGAGCCTGGACTGTACATCTCTGGGAATTGGTTTCATTAA
AAAGAAGCACTTTAAAAAAAAAAAAAAAAAAAAAAAAAAAAAAAAAAAAA
AAAACCGGAGAGATAAAAAGGGGGGGGGGAAAAGTGGAAAAAAAGACCAG
CGGCAAACATTTGCCGGACCCTTTTTGGCGACACAGGGGAAAACGGGAGC
CCCAAAGGAGGTAAATGAAACGGTTCTTTTTTTCTTATTTCAGAAAGCAG
AAGAAAAAGAAATAAAGAAAAGAAAGAAAAAAAAAGAATGATAAAAGAGG
AAAAATAAAAAAAAAGAAAAAGAGAGAGAATAGAAACTTAAAAAATAAAC
ACAAAAAAGAA
(CloneID 5459503)

SEQ ID NO:61

GCTGCAGCGGTGGTCGCCTGTTGGGTGTGGAGTTTCCCAGCGCCCCTCGG
GTCCGACCCTTTGAGCGTTCTGCTCCGGCGCCAGCCTACCTCGCTCCTCG
GCGCCATGACCACAACCACCACCTTCAAGGGAGTCGACCCCAACAGCAGG
AATAGCTCCCGAGTTTTGCGGCCTCCAGGTGGTGGATCCAATTTTTCATT
AGGTTTTGATGAACCAACAGAACAACCTGTGAGGAAGAACAAAATGGCCT
CTAATATCTTTGGGACACCTGAAGAAAATCAAGCTTCTTGGGCCAAGTCA
GCAGGTGCCAAGTCTAGTGGTGGCAGGGAAGACTTGGAGTCATCTGGACT
GCAGAGAAGGAACTCCTCTGAAGCAAGCTCCGGAGACTTCTTAGATCTGA
AGGGAGAAGGTGATATTCATGAACAATGTGGACACAGACTTGCCAGGCAG
CCTGGGGCAGAGTGAAGAGAAGCCCGTGCCTGCTGCGCCTGTCCCCAGCC
CGGTGGCCCGGCCCCAGTGCCATCCAGAAGAAATCCCCCTGGCGGCAAGT
CCAGCCTCGTCTTGGGTTAGCTCTGACTGTCCTGAACGCTGTCGTTCTGT
CTGTTTCCTCCATGCTTGTGAACTGCACAACTTGAGCCTGACTGTACATC
TCTTGGATTTGTCCCCTTCCCAAACGAAGCACTTTATGTACTGCTGTCTT
TTTTTTTTCCCTTTGGAACGCCCCCCGGTTCCTCTCTGTCCCTTGACTCC
TGGGGTCTGGGGGCCTG
(CloneID 4539292)

SEQ ID NO:62

TGCAGCGGTGGTCGGCTGTGGGTGTGGAGTTTCCCAGCGCCCCTCGGCTC
CGACCCTTTGAGCGTTCTGCTCCGGCGCCAGCCTACCTCGCTCCTCGGCG
CCATGACCACAACCACCACCTTCAAGGGAGTCGACCCCAACAGCAGGAAT
AGCTCCCGAGTTTTGCGGCCTCCAGGTGGTGGATCCAATTTTTCATTAGG
TTTTGATGAACCAACAGAACAACCTGTGAGGAAGAACAAAATGGCCTCTA
ATATCTTTGGGACACCTGAAGAAAATCAAGCTTCTTGGGCCAAGTCACCA
GGTGCCAAGTCTAGTGGTGGCAGGGAAGACTTGGAGTCATCTGGACTGCA
GAGAAGGAACTCCTCTGAAGCAAGCTCCGGAGACTTCTTAGATCTGAAGG
GAGAAGGTGATATTCATGAAAATGTGGACACAGACTTGCCAGGCAGCCTG
GGGCAGAGTGAAGAGAAGCCCGTCCCTGCTGCGCCTGTGCCCAGCCCGGT
GGCCCCGGCCCCAGTGCCATCCAGAAGAAATCCCCCTGGCGGCAAGTCCA
GCCTCGTCTTGCGTTAGCTCTGACTGTCCTGAACGCTGTCGTTCTGTCTG
TTTCCTCCATGCTTGTGAACTGCACAACTTGAGCCTGACTGTACATCTCT
TGGATTGTTTCATTAAAAAGAAGCACTTTATGTAAAAAAAAAAAAAA
(CloneID 5247820)

SEQ ID NO:63

CTCCTGCAGCGGTGGTCGGCTGTTGGGTGTGGAGTTTCCACCGCCCCTCG
GGTCGACCCTTTGAGCGTTCTGCTCCGGCGCCAGCCTACCTCGCTCCTCG
GCGCCATGACCACAACCACCACCTTCAAGGGAGTCGACCCCAACAGCAGG
AATAGCTCCCGAGTTTTGCGGCCTCCAGGTGGTGGATCCAATTTTTCATT
AGGTTTTGATGAACCAACAGAACAACCTGTGAGGAAGAACAAAATGGCCT
CTAATATCTTTGGGACACCTGAAGAAAATCAAGCTTCTTGGGCCAAGTCA
GCACGTGCCAAGTCTAGTGGTGGCAGGGAAGACTTGGAGTCATCTGGACT
GCAGAGAAGGAACTCCTCTGAAGCAAGCTCCGGAGACTTCTTAGATCTGA
AGGGAGAAGGTGATATTCATGAAAATGTGGACACAGACTTGCCAGGCAGC
CTGGGGCAGAGTGAACGAGAAGCCCGTGCCTGCTGCGCCTGTGCCCAGCC
CGGTGGCCCCGGCCCCAGTGCCATCCAGAAGAAATCCCCCTGGCGGCAAG
TCCAGCCTCGTCTGGGGTTAGCTCTGACTGTCCTGAACGCTGTCGTTCTG
TCTGTTTCCTCCATGCTTGTGAACTGCACAACTTGAGCCTGACTGTACAT
CTCTTGGATTTGTTTCATTACAAAGAAGCACTTTATGTACTGCTGTCTTT
ATATTTTGTCCTTTGTGAGAACAGGTCGTCTCGTGTCCTTGACTCTGGGG
TCTGTGGC
(CloneID 5397495)

SEQ ID NO:64

CTCCTGCAGCGGTGGTCGGCTCTTGGGTGTGGAGTTTCCCAGCGCCCCTC
GGGTCCGACCCTTTGAGCGTTCTGCTCCGGCGCCAGCCTACCTCGCTCCT
CGGCGCCATGACCACAACCACCACCTTCAAGGGAGTCGACCCCAACAGCA
GGAATAGCTCCCGAGTTTTGCGGCCTCCAGGTGGTGGATCCAATTTTTCA
TTAGGTTTTGATGAACCAACAGAACAACCTGTGAGGAAGAACAAAATGGC
CTCTAATATCTTTGGGACACCTGAACGAAAATCAAGCTTCTTGGGCCAAG
TCAGCAGGTGCCAAGCTCTAGTGGTGGCAGGGAAGACTTGGAGTCATCTG
GACTGCAGAGAAGGAACTCCTCTGAAGCAAGCTCCGGAGACTTCTTAGAT
CTGAAGGGCACGAAGGTGATACTTCATGAAAACTGTGGACACAGACTTGC
CAGGCAGCCTGGGGCAGAGTGAACAGAAGCCCGTGCCTGCTGCGCCTCTG
CCCAGCCCGGTGGGCCCTGGCCCAGTGCCATCCACGAACGAAATCCCCCT
GGCTGGCAATGTCCAGCCTCGTCTTGGGTTACGCTCTGACTGTCCTGAAC
GCTGTCGTACTGTCTGTCTCCTCCATGCTCGTGAACTGCACAACTTGAGC
CTGACTGTACATCTCTTGGATTCGTATCATTAACCAAGAAGCACTTTATG
TACTGCTGTCTTTATTTACGCCTTTCGAAGACCG
(CloneID 5200412)

SEQ ID NO:65

CTGCAGCGGTGGTCGGCTGTTGGGTGTGGAGTTTCCCAGCGCCCCTCGGG
TCCGACCCTTTGACCGTTCTGCTCCGGCGCCAGCCTACCTCGCTCCTCGG
CGCCATGACCACAACCACCACCTTCAAGGGAGTCGACCCCAACAGCAGGA
ATAGCTCCCGAGTTTTGCGGCCTCCAGGTGGTGGATCCAATTTTTCATTA
GGTTTTGATGAACCAACAGAACAACCTGTGAGGAAGAACAAAATGGCCTC
TAATATCTTTGGGACACCTGAAGAAAATCAAGCTTCTTGGGCCAAGTCAG
CAGGTGCCAAGTCTAGTGGTGGCAGGGAAGACTTGGAGTCATCTGGACTG
CAGAGAAGGAACTCCTCTCAAGCAAGCTCCGGAGACTTCTTAGATCTGAA
GGGAGAAGGTGATATTCATGAAAATGTGGACACAGACTTGCCAGGCAGCC
TGGGGCAGAGTGAAGAGAAGCCCGTGCCTGCTGCGCCTGTGCCCAGCCCG
GTGGCCCCGGCCCCAGTGCCATCCAGAAGAAATCCCCCTGGCGGCAAGTC
CAGCCTCGTCTTGGGTTAGCTCTGACTGTCCTGAACGCTGTCGTTCTGTC
TGTGTCCTCCATGCTTGTGAACTGCACAACTTGAGCCTGACTGGTACATC
TCTTGGATCCGTTTCATTAAAAAGAAGCACTTTATGTACTGCTGTCTTTA
TTTTCACTTTAGACGAACCAGGTGTCTCTCGTGTCCTGGACTCCTGGGAC
CTGTGGGCCATG
(CloneID 5208934)

SEQ ID NO:66

CTGCAGCGGTGGTCGGCTGTTGGGTGTGGAGTTTCCCAGCGCCCCTCGGG
TCCGACCCTTTGAGCGTTCTGCTCCGGCGCCAGCCTACCTCGCTCCTCGG
CGCCATGACCACAACCACCACCTTCAAGGGAGTCGACCCCAACAGCAGGA
ATAGCTCCCGAGTTTTGCGGCCTCCAGGTGGTGGATCCAATTTTTCATTA
GGTTTTGATGAACCAACAGAACAACCTGTGAGGAAGAACAAAATGGCCTC
TAATATCTTTGGGACACCTGAAGAAAATCAAGCTTCTTGGGCCAAGTCAG
CAGGTGCCAAGTCTAGTGGTGGCAGGGAAGACTTGGAGTCATCTGGACTG
CAGAGAAGGAACTCCTCTGAAGCAAGCTCCGGAGACTTCTTAGATCTGAA
GGGAGAAGGTGATATTCATGAAAATGTGGACACAGACTTGCCAGGCAGCC
TGGGGCAGAGTGAAGAGAAGCCCGTGCCTGCTGCGCCTGTGCCCAGCCCG
GTGGCCCCGGCCCCAGTGCCATCCAGAAGAAATCCCCCTGGCGGCAAGTC
CAGCCTCGTCTTCGGTTAGCTCTGACTGTCCTGAACGCTGTCGTTCTGTC
TGTTTCCTCCATGCTTGTGAACTGCACAACTTGAGCCTGACTGTACATCT
CTTGGATTTGTTTCATTAAAAAGAAGCACTTTATGTACTGCTGTCTTTTT
TTTCCTTTTGAAGACCAGGTTCTCTCTGTCCTTGACTCTTGGGCTCTGTG
GGCCCG
(CloneID 4156130)

SEQ ID NO:67

CGGACGCGTGGGCGGCTGTTGGGTGTGGAGTTTCCCAGCGCCCCTCGGGT
CCGACCCTTTGAGCGTTCTGCTCCGGCGCCAGCCTACCTCGCTCCTCGGC
GCCATGACCACAACCACCACCTTCAAGGGAGTCGACCCCAACAGCAGGAA
TAGCTCCCGAGTTTTGCGGCCTCCAGGTGGTGGATCCAATTTTTCATTAG
GTTTGATGAACCAACAGAACAACCTGTTGAGGAAGAACAAAATGGCCTCT
AATATCTTTGGGACACCTGAAGAAAATCAAGCTTCTTGGCCAAGTCAGCA
GGTGCCAAGTCTAGTGGTGGCAGGGAAGACTTGGAGTCATCTGGACTGCA
GGAGAAGGAACTCCTCTGAAGCAAGCTCCGGAGACTTCTTAGATCTGAAG
GGAGAAGGTGATATTCATGAAAATGTGGACACAGACTTGCCAGGCAGCCT
GGGGCAGAGTGAAGAGAAGCCCGTGCCTGCTGCGCCTGTGCCCAGCCCGG
TGGCCCCGGCCCCAGTGCCATCCAGAAGAAATCCCCCGGCGGCAAGTCCA
GCCTCGTCTTGGGTTAGCTCTGACTGTCCTGAACGCTGTCGTTCTGTCTG
TTTCCTCCATGCTTCTGAACTGCACAACTTGAGCCTGACTGTACATCTCT
GTGGATTGTTTCATTAAAAAGAAGCCCTTTATGTACTGCTGTCTTTATCT
TTCCTTTGGAGGACCCGGTTTCCCCCGCGTCCTGGCCTCTGGGTCCTGGG
GCCCTGGCTTGGGTGTTC
(CloneID 4123030)

SEQ ID NO:68

GCTCCTGCAGCGGTGGTCGGCTGTGGGGTGTGGAGTTTCCCAGCGCCCCT
CGGGTCCGACCCTTTGAGCGTTCTGCTCCGGCGCCAGCCTACCTCGCTCC
TCGGCGCCATGACCACAACCACCACCTTCAAGGGAGTCGACCCCAACAGC
AGGAATAGCTCCCGAGTTTGCGGCCTCCAGGTGGTGGATCCAATTTTTCA
TTAGGTTTGATGAACCAACAGAACAACCTGTGAGGAAGAACAAAATGGCC
TCTAATATCTTGGGACACCTGAAGAAAATCAAGCTTCTTGGGCCAAGTCA
GCAGGTGCCAAGTCTAGTGGTGGCAGGGAAGACTTGGAGTCATCTGGACT
GCAGAGAAGGAACTCCTCTGAAGCAAGCTCCGGAGACTTCTTAGATCTGA
AGGGAGAAGGTGATATTCATGAAAATGTGGACACAGACTTGCCAGGCAGC
TGGGGCAGAGTGAAGAGAAGCCCGTGCCTGCTGCGCCTGTGCCCAGCCCG
GTGGCCCGGCCCAGTGCCATCCAGAAGAAATCCCCCTGGGCGGCAAGTCC
AGCCTCGTCGTTGGGTTAGCTCTGACTGTCCTGAACGCTGTCGTTCTGTC
TGTTTCCTCCATGCTTGTGAACTGACAACTTGAGCCTGACTGTACATCTC
TCGGATTGTTCATTAAAAGAAGCACTTTATGTACTGCTGTCTTTTTTTTC
TTTTGAGGACCGTTCTCCCTGTCCTGACTCTGGTTGGGGGGCAGGGTGAG
GGTTCTAAAAAGCCCC
(CloneID 5459215)

SEQ ID NO:69

GGCACCCGTGGTCGCCTCTTGGGTGTCGAGTTTCCCACCGCCCCTCGCCT
CGACCCTTTGAGCGTTCTGCTCCGGCGCCAGCCTACCTCGCTCCTCGGCG
CCATGACCACAACCACCACCTTCAAGGGAGTCGACCCCAACAGACAGGAA
TAGCTCCCGAGTTTTGCGGCCTCCAGGTGGTGGATCCAATTTTTCATTAG
GTTTTGATGAACCAACAGAACAACCTGTGAGGAAGAACAAAATGGCCTCT
AATATCTTTGGGACACCTGAAGAAAATCAAGCTTCTTGGGCCAACTCACC
AGGTGCCAAGTCTAGTCCTCCCACCCAAGACTTCCAGTCATCTGGACTCC
AGAGAAGGAACTCCTCTGAAGCAAGCTCCGGAGACTTCTTAGATCTGAAG
GGAGAAGGTGATATTCATGAAAATGTGGACACAGACTTGCCAGGCAGCCT
GGGGCAGAGTGAAGAGAAGCCCGTGCCTGCTGCGCCTCTCCCCACCCCGG
TCCCCCGGCCCAGTGCCATCCACAAGAAATCCCCCTCCCGGCAACTCCAG
CCTCCTCTTCGCTTAGCTCTGACTGTCCTGAACGCTGTCGTTCTGTCTGT
TTTCCTCCATGCTTGTGAACTGCACAATTGAGACCTGACTGCTACATCTC
TGGATTTGTTTCATTAAAAAGAAGCACTTTATGTACTGCTCTCTTTATTT
TTCCTTGTGAACA
(CloneID 4337230)

SEQ ID NO:70

GCCTCCAGCCCTGGTCGCCTCTTCGCTCTGGACTTTCCCAGCGCCCCTCG
GCTCCCACCCTTTGAGCCTTCTGCTCCGGCGCCAGCCTACCTCCCTCCTC
CGCGCCATGACCACAACCACCACCTTCAAGGGAGTCGACCCCAACAGCAG
GAATAGCTCCCGAGTTTTGCGGCCTCCAGGTGGTGGATCCAATTTTTCAT
TAGGTTTTGATGAACCAACAGAACAACCTGTGAGGAAGAACAAAATGGCC
TCTAATATCTTTGGGACACCTGAAGAAAATCAAGCTTCTTGGCCCAACTC
ACCACCTCCCAACTCTACTCCTCCCACCCAACACTTCCACTCATCTCCAC
TCCAGAGAAGGAACTCCTCTGAAGCAAGCTCCGGAGACTTCTTAGATCTG
AAGGGAGAAGGTGATATTCATGAAAATGTGGACACAGACTTGCCAGGCAG
CCTGGGGCAGAGTGAAGAGAAGCCCGTGCCTGCTGCGCCTGTGCCCAGCC
CGGTGGCCCCGGCCCAGTGCCATCCAGAAGAAATCCCCCTGGCGCCAAGT
CCAGCCTCGTCTTGGGTTAGCTCTGACTGTCCTGAACGCTGTCGTTCTGT
CTGTTTCCTCCATGCTTGTGAACTGACAATTGAGCCTGACTGTACATCTC
TTGGATTTGTTTCATTAAAAAGAAGCACTTTAAAAAAAAAAAAAAGAGAA
AAGAAAAACCGGGGGTTTTTTGGGGGGGGAAAAGAGGGTTAAAGGCGGGG
TAGGTTTTTGCCCTTTTTGAAAGGGGAGACGGGGGCCCAGGGGGGGTAAT
GGAAGGGGACG
(CloneID 4649355)

SEQ ID NO:71

GCAGCGGTGGTCGGCTGTTGGGTGTGGAGTTTCCCAGCGCCCCTCGGGTC
CGACCCTTTGAGCGTTCTCCTCCGGCGCCAGCCTACCTCGCTCCTCGGCG
CCATGACCACAACCACCACCTTCAAGGGAGTCGACCCCAACAGCAGGAAT
AGCTCCCGAGTTTTGCGGCCTCCAGGTGGTGGATCCAATTTTTCATTAGG
TTTTGATGAACCAACAGAACAACCTGTGAGGAAGAACAAAATGGCCTCTA
ATATCTTTGGGACACCTGAAGAAAATCAAGCTTCTTGGGCCAAGTCAGCA
GGTGCCAAGTCTAGTGGTGGCAGGGAAGACTTGGAGTCATCTGGACTGCA
GAGAAGGAACTCCTCTGAAGCAAGCTCCGGAGACTTCTTAGATCTGAAGG
GAGAAGGTGATATTCATGAAAATGTGGACACAGACTTGCCAGGCAGCCTG
GGGCAGAGTGAAGAGAAGCCCGTGCCTGCTGCGCCTGTGCCCAGCCCGGT
GGCCCCGGACCCAGTCCCATCCAGAAGAAATCCCCCTGGCGGCAAGTCCA
GCCTCGTCTTGGGTTAGCTCTGACTGTCCTGAACGCTGTCGTTCTGTCTG
TTTCCTCCATGCTTGTGAACTGCACAACTTGAGCCTGACTGTACATCTCT
TGGATCTGTTTCATTAAACAGAAGCACTTTATGTACTGCTGTCTCACTAC
AAATACACTATCGAACAACAGGTTACTACTCTGTCCTTGAN
(CloneID 4126162)

SEQ ID NO:72

GCTGCAGCGGTGGTCGGCTGTTGGGTGTCGAGTTTCCCAGCGCCCCTCGG
GTCCGACCCTTTGAGCGTTCTGCTCCGGCGCCAGCCTACCTCGCTCCTCG
GCGCCATGACCACAACCACCACCTTCAAGGGAGTCGACCCCAACAGCAGG
AATAGCTCCCGAGTTTTGCGGCCTCCAGGTGGTGGATCCAATTTTTCATT
AGGTTTGATGAACCAACAGAACAACCTGTGAGGAAGAACAAAATGGCCTC
TAATATCTTTGGGACACCTGAAGAAAATCAAGCTTCTTGGGCCAAGTCAG
CAGGTGCCAAGTCTAGTGGTGGCAGGGAAGACTTGGAGTCATCTGGACTG
CAGAGAAGGAACTCCTCTGAAGCAAGCTCCGGAGACTTCTTAGATCTGAA
GGGAGAAGGTGATATTCATGAAAATGTGGACACAGACTTGCCAGGCAGCC
TGGGGCAGAGTGAAGAGAAGCCCGTGCCTGCTGCGCCTGTGCCCAGCCCG
GTGGCCCCGGCCCCAGTGCCATCCAGAAGAAATCCCCCTGGCGGCAAGTC
CAGCCTCGTCTTGCGTTAGCTCTGACTGTCCTGAACGCTGTCGTTTCTGT
CTGTTTCCTCCATGCTGTGAACTGCACAACTTGAGCCTGACTGTACATCT
CTGGGATTGTTCATTCAAAAGAAGCACTTTATGTACTGCTGTCTTTTTTC
CCTTGGACACCAGGTTCCCCTCCGTCCTTGACTCCTGGCTTTGGGGCCCG
GCCAGAGTGTTCCTATAACAATGGGGGGAAAACCTTGGGCACCTAGCCGG
GTTAAAGAAAAATGGGCCACTGGTAAGC
(CloneID 2783676 in 3′ to 5′ orientation)

SEQ ID NO:73

TTTTTTTACATAAAGTGCTTCTTTTTAATGAAACAAATCCAAGAGATGTA
CAGTCAGGCTCAAGTTGTGCAGTTCACAAGCATGGAGGAAACAGACAGAA
CGACAGCGTTCAGGACAGTCAGAGCTAACCCAAGACGAGGCTGGACTTGC
CGCCAGGGGGATTTCTTCTGGATCGCACTGGGGCCGGGGCCACCGGGCTG
GGCACAGGCGCAGCAGGCACGGGCTTCTCTTCACTCTGCCCCAGGCTGCC
TGGCAAGTCTGTGTCCACATTTTCTTCAGATCTAAGAAGTCTCCGGAGCT
TGCTTCAGAGGAGTTCCTTCTCTGCAGTCCAGATGACTCCAAGTCTTCCC
TGCCACCACTAGACTTGGCACCTGCTGACTTGGCCCAAGAAGCTTGATTT
TCTTCAGGTGTCCCAAAGATATTAGAGGCCATTTTGTTCTTCCTCACAGG
TTGTTCTGTTGGTTCATCAAAACCTAATGAAAAATTGGATCCACCACCTG
GAGGCCGCAAAACTCGGGAGCTATTCCTGCTGTTGGGGTCGACTCCCTTG
AAGGTGGTGGT
(CloneID 2783676)

SEQ ID NO:74

GCAGCGGTGGTCGGCTGTTGGGTGTGGAGTTTCCCAGCGCCCCTCGGGTC
CGACCCTTTGAGCGTTCTGCTCCGGCGCCAGCCTACCTCGCTCCTCGGCG
CCATGACCACAACCACCACCTTCAAGGGAGTCGACCCCAACAGCAGGAAT
AGCTCCCGAGTTTTGCGGCCTCCAGGTGGTGGATCCAATTTTTCATTAGG
TTTTGATGAACCAACAGAACAACCTGTGAGGAAGAACAAAATGGCCTCTA
ATATCTTTGGGACACCTGAAGAAAATCAAGCTTCTTGGGCCAAGTCAGCA
GGTGCCAAGTCTAGTGGTGGCAGGGAAGACTTGGAGTCATCTGGACTGCA
GAGAAGGAACTCCTCTGAAGCAAGCTCCGGAGACTTCTTAGATCTGAAGA
AAATGTGGACACAGACTTGCCAGGCAGCCTGGGGCAGAGTGAAGAGAAGC
CCGTGCCTGCTGCGCCTGTGCCCAGCCCGGTGGCCCCGGCCCCAGTGCC
(CloneID 2822776 in 3′ to 5′ orientation)

SEQ ID NO:75

TTTTTTTTTTACATAAAGTGCTTCTTTTTAATGAAACAAATCCAAGAGAT
GTACAGTCAGGCTCAAGTTGTGCAGTTCACAAGCATGGAGGAAACAGACA
GAACGACAGCGTTCAGGACAGTCAGAGCTAACCCAAGACGAGGCTGGACT
TGCCGCCAGGGGGATTTCTTCTGGATGGCACTGGGGCCGGGGCCACCGGG
CTGGGCACAGGCGCAGCAGGCACGGGCTTCTCTTCACTCTGCCCCAGGCT
GCCTGGCAAGTCTGTGTCCACATTTTCATGAATATCACCTTCTCCCTTCA
GATCTAAGAAGTCTCCGGAGCTTGCTTCAGAGGAGTTCCTTCTCTGCAGT
CCAGATGACTCCAAGTCTTCCCTGCCACCACTAGACTTGGCACCTGCTGA
CTTGGCCCAAGAAGCTTGATTTTCTTCAGGTGTCGCAAAGATATTAGAGG
CCATTTTGTTCTTCCTCACAGGTTGTTCTGTTGGTTCATCAAAACCTAAT
GAAAAATTGGATCCACCACCTGGAGGCCGCAAAACTCGGGAAGCATTCCT
GCTGTTGG
(CloneID 2822776)

SEQ ID NO:76

GGCACGAGGGGCTGTTGGGTGTGGAGTTTCCCAGCGCCCCTCGGGTCCGA
CCCTTTGAGCGTTCTGCTCCGGCGCCAGCCTACCTCGCTCCTCGGCGCCA
TGACCACAACCACCACCTTCAAGGGAGTCGACCCCAACAGCAGGAATAGC
TCCCGAGTTTTGCGGCCTCCAGGTGGTGGATCCAATTTTTCATTAGGTTT
TGATGAACCAACAGAACAACCTGTGAGGAAGAACAAAATGGCCTCTAATA
TCTTTGGGACACCTGAAGAAAATCAAGCTTCTTGGGCCAAGTCAGCAGGT
GCCAAGTCTAGTGGTGGCAGGGAAGACTTGGAGTCATCTGGACTGCAGAG
AAGGAACTCCTCTGAAGCAAGCTCCGGAGACTTCTTAGATCTGAAGGGAG
AAGGTGATATTCATGAAAATGTGGACACAGACTTGCCAGGCAGCCTGGGG
CAGAGTGAAGAGAAGCCCGTGCCTGCTGCGCCTGTGCCCAGCCCCGTGGC
CCCGGCCCCAGTGCCATCCAGAAAAAATCCCCCTGGCGGCAAGTCCAGC
(CloneID 2781579)

SEQ ID NO:77

GCAGCGGTGGTCGGCTGTTGGGTGTGGAGTTTCCCAGCGCCCCTCGGGTC
CGACCCTTTGAGCGTTCTGCTCCGGCGCCAGCCTACCTCGCTCCTCGGCG
CCATGACCACAACCACCACCTTCAAGGGAGTCGACCCCAACAGCAGGAAT
AGCTCCCGAGTTTTGCGGCCTCCAGGTGGTGGATCCAATTTTTCATTAGG
TTTTGATGAACCAACAGAACAACCTGTGAGGAAGAACAAAATGGCCTCTA
ATATCTTTGGGACACCTGAAGAAAATCAAGCTTCTTGGGCCAAGTCAGCA
GGTGCCAAGTCTAGTGGTGGCAGGGAAGACTTGGAGTCATCTGGACTGCA
GAGAAGGAACTCCTCTGAAGCAAGCTCCGCAGACTTCTTAGATCTGAAGG
GAGAAGGTGATATTCATGAAAATGTGGACACAGACTTGCCAGGCAGCCTG
GGGCAGAATGAAAAAAACCCGTGCCTGCTGCCCTGTGCCCAGCCCGTGGC
CCCCGGCCCATGCCATCAAAAAAATCCCCCTGCCGCGAACCAACCTTTGC
TG
(CloneID 2781579 in 3′ to 5′ orientation)

SEQ ID NO:78

TTTTTTTACATAAAGTGCTTCTTTTTAATGAAACAAATCCAAGAGATGTA
CAGTCAGGCTCAAGTTGTGCAGTTCACAAGCATGGAGGAAACAGACAGAA
CGACAGCGTTCAGGACAGTCAGAGCTAACCCAAGACGAGGCTGGACTTGC
CGCCAGGGGGATTTCTTCTGGATGGCACTGGGGCCGGGGCCACCGGGCTG
GGCACAGGCGCAGCAGGCACGGGCTTCTCTTCACTCTGCCCCAGGCTGCC
TGGCAAGTCTGTGTCCACATTTTCATCAATATCACCTTCTCCCTTCAGAT
CTAAGAAGTCTCCGGAGCTTGCTTCAGAGGAGTTCCTTCTCTGCAGTCCA
GATGACTCCAAGTCTTCCCTGCCACCACTAGACTTGGCACCTGCTGACTT
GGCCCAAGAAGCTTGATTTTCTTCAGGTGTCCCAAAGATATTAGAGGCCA
TTTTGTTCTTCCTCACAGGTTGTTCTGTTGGTTCATCAAAACCTAATGAA
AAATTGGATCCACCACCTGGAGGCCGCAAAACTCGGGAGCTATTCCTGCT
GTTGGGGTCGACTCCCTTGAAAGTGGTGGT
(CloneID 5419867)

SEQ ID NO:79

GCAGCGGTGGTCGGCTGTTGGGTGTGGAGTTTCCCAGCGCCCCTCGGGTC
CGACCCTTTGAGCGTTCTGCTCCGGCGCCAGCCTACCTCGCTCCTCGGCG
CCATGACCACAACCACCACCTTCAAGGGAGTCGACCCCAACAGCAGGAAT
AGCTCCCGAGTTTTGCGGCCTCCAGGTGGTGGATCCAATTTTTCATTAGG
TTTTGATGAACCAACAGAACAACCTGTGAGGAAGAACAAAATGGCCTCTA
ATATCTTTGGGACACCTGAAGAAAATCAAGCTTCTTGGGCCAAGTCAGCA
GGTGCCAAGTCTAGTGGTGGCAGGGAAGACTTGGAGTCATCTGGACTGCA
GAGAAGGAACTCCTCTGAAGCAAGCTCCGCAGACTTCTTAGATCTGAAGG
GAGAAGGTGATATTCATGTTTTCATAGAAAATGTGGACACAGACTTGCCA
GGCAGCCTGGGGCAGAGTGAAGAGAAGCCCGTGCCTTGCTGCGCCTGTGC
CCAGCCCGGTGGCCCCGGACCCAGTGCCATCCAGAAGAAATCCCCCTGGC
GGCAAGTCCAGCCTCGTCTTGGGTTAGCTCTGACTGTCCTGAACGCTGTC
GTTCTGTCTGTTTCCTCCATGCTTGTGAACTGCACAACTAGAGCCTGACT
GTACATCTCTATGGATATGTTTCATTAAAACAGAAGCACTTTATGTACTG
GTGTCTTTATTATTTCTTTGGAAGACCAGGTTCTCCTCTGGCCTAGAATC
CTGGGGGTCTGAGG
(CloneID 4328715)

SEQ ID NO:80

GGCTGATTGGGTGTGGAGTTTCCCAGCGCCCCTCGGGTCCGACCCTTTGA
GCGTTCTGCTCCGGCGCCAGCCTACCTCCTCCTCGGGCGCCATGACCACA
ACCACCACCTTCAAGGGATTCGATCCCAACAGCAGGAATAGCTCCCGAGT
TGTGCGGACTCCAGCGTGGTGGATCCAATTTTATCATTAGGTTTTGATGA
ACCAACAGAGCAACCTGTGAGGAAGAACAAAATGGCCTCTAAATATCTTT
GGGACACCTGAAGAAGAATCAAGCGTTCTTGGGCCAGAGTCAGTCAGCGT
GCCAAGTCTAGATGGTGGCAGGGAACGACTTGGAGTCATCTGGACTGCAT
GAGAAGGAACTCCTCTGAAGCAAGCTCCGGAGACTTCTTAGATCTGAAGC
GAGACCGGTGATATTCATGAAAATGTGGACACAGACTTGCCAGGCAGCCT
GTGGCAGAGTGATGAGAAGCCCGTGCCTGCTTGCGCCTGTGCCCAGCCCG
GTGGCCCCGGCCCCAGTGCCATCCAGAAGAAATCCCCCTGGCGGCAGTCC
AGCCTCGTCTTGGGTTAGCTCTGACTGTCCTGAACGCTGTCGTTCTGTC
TGTTTCCTCCATGCTTGTGAACTGCACAACTTGAGCCTGACTGTACATCT
CTTGGGATTTGGTTCCCTTTAAAAAGAAGCACTTTATGTTACTGCTGTCT
TTTTTTTTTCCTTTTGACGGCACGGGTTTCCTCTCGGTCCTTGACTCTGG
GGGTCTGGGGGCCCAGGGCATGAGTGTTCCTTATATGATGGGGGGAGAGC
TTGGGGCCCTTGCACCGGTTTTTCGCGAGCAATATTGGGCCCAGTGGTTA
GGCTCTTTGGCCGGGGTTCACCTCTTGGGTTCCCCCCCGGGGGCCAAGGA
CGCCGTTTGGTC
(CloneID 4138051)

SEQ ID NO:81

GCTGCAGCGGTGGTCGGCTGTGGGTGTGGAGTTTCCCAGCGCCCCTCGGG
TCCGACCCTTTGAGCGTTCTGCTCCGGCGCCAGCCTACCTCGCTCCTCGG
CGCCATGACCACAACCACCACCTTCAAGGGAGTCGACCCCAACAGCAGGA
ATAGCTCCCGAGTTTGCGGCCTCCACGTGGTGGATCCAATTTTTCATTAG
GTTTTGATGAACCAACAGAACAACCTGTGAGGAAGAACAAAATGGCCTCT
AATATCTTTGGGACACCTGAAGAAAATCAAGCTTCTTGGGCCAAGTCAGC
AGGTGCCAAGTCTAGTGGTGGCAGGGAAGACTTGGAGTCATCTGGACTGC
AGAGAAGGAACTCCTCTGAAGCAAGCTCCGGAGACTTCTTAGATCTGAAG
GGAGAAGGTGATATTCATGAAAATGTGGACACAGACTTGCCAGGCAGCCT
GGGGCAGAGTGAAGAGAAGCCCGTGCCTGCTGCGCCTGTGCCCAGCCCGG
TGGCCCCGGCCCAGTGCCATCCAGAAGAAATCCCCCTGGCGGCAGTCCAG
CCTCGTCTTGGGTTAGCTCTGACTGTCCTGAACGCTGTCGTATCTGTCTG
TTGTCCTCCATGCTTGTGAACTGCACAAGTTGAGCCTGACTGTACATCTC
TTGGATTCGTTCATTAACAAGAAGCACTTTATGTACTGCTGTCTTTTATT
TTCCCTTTGAAGACCCGGTCCCCCCAGGCCTGACCTGGGCTGGGGCCAGG
AAGAGGTTCTCATAAAAAAGGGGGGAACACTTGGGACACTAGACTCGGGT
TTCGCAAAAATCTGGGTCCAATGTGAAAAGATGGGAGG
(CloneID 4766330)

SEQ ID NO:82

GCTGCAGCGGTGGTCGGCTGTTGGGTGTGGAGTTTCCCAGCGCCCCTCGG
GTCCGACCCTTTGAGCGTTCTGCTCCGGCGCCAGCCTACCTCGCTCCTCG
GCGCCATGACCACAACCACCACCTTCAAGGGAGTCGACCCCAACAGCAGG
AATAGCTCCCGAGTTTTGCGGCCTCCAGGTGGTGGATCCAATTTTTCATT
AGGTTTTGATGAACCAACAGAACAACCTGTGAGGAAGAACAAAATGGCCT
CTAATATCTTTGGGACACCTGAAGAAAATCAAGCTTCTTGGGCCAAGTCA
GCAGGTGCCAAGTCTAGTGGTGGCAGGAAGACTTGGAGTCATCTGGACTG
CAGAGAAGGAACTCCTCTGAAGCAAGCTCCGGAGACTTCTTAGATCTGAA
GGGAGAAGGTGATATTCATGAACAATGTGGACACAGACTTGCCAGGCAGC
CTGGGGCAGAGTGAAGAGAAGCCCGTGCCTGCTGCGCAATGTGCCCAGCC
CGGTGGCCCCGGCCCCAGTGCCATCCAGAAGAATCCCCCTGGCGGCAAGT
CCAGCCTCGTCTTGGGTTAGCTCTGACTGTCCCTGAACGCTGTCGTTCTG
TCTGTTTCCTCCATGCTTGTGAACTGCACAATTGAGCCTGACTGTACATC
TCTTGGATTTGTTTCATTAAACAAGAAGCACCTTTAAACACAAAAAAAAA
A
(CloneID 3958097)

SEQ ID NO:83

GTTGGGTGTGGAGTTTCCCAGCGCCCCTCGGGTCAGACCCTTTGAGCGTT
CTGCTCCGGCGCCAGTCCTATCCTCGCTCCTCGGCGCCATGACCACAACC
ACCACCTTCAAGGGAGTCGACCCCAACAGCAGGAATAGCTCCCGAGTTTT
GCGGCTCCAGGTGGTGGATCCAATTTTTCATTAGGTTTTGATGAACCAAC
AGAACAACCTGTGAGGCAAAGAACAAAATGGCCTCTAATATCTTTGGGAC
ACCTGAAGAAAATCAAGCTTCTTGGGCCAAGTCACGCAGGTGCCAAGTCT
AGTGGTGGCAGGGAAGACTTGGAGTCATCTGGACTGCAGAGAAGGAACTC
CTCTGAAGCAGCTCCGGAGACTTCTTAGATCTGAAGGGCAGAAAGCTGAT
ATTCATGAAAATGTGGACACAGACTTGCCAGGCAGCCTGGGGCAGCAGTG
AACGAGAAGCCCGTGCCTGCTGCGCCTGTGCCCAGCCCGGTGGCCCCGGC
CCCAGTGCCATCCAGAAGAAATCCCCCTGGCGGGAAGTCCAGCCTCGTCT
TGGGTTAGCTCTGACTGTCCTGAACGCTGTCGTTCTGTCTGTTTCCTCCA
TGCTTGTGAGCTGCACAACTTGAGTCCTGACTGTACATCTCTTGGATTCG
TCTCATTGAGAAGAGAAGCACTTTATGTACTGTGTCTTTTCTGTCCTTTG
GCGGCCCGGTTCTCTCTGTCTTGACCCTGGGGCGGTGGGN
(CloneID 346318 in 3′ to 5′ orientation)

SEQ ID NO:84

TTTTTTTTTTTTTTTTTTTTAAAGTGCTTCTTTTTAATGAAACAAATCCA
AGAGATGTACAGTCAGGCTCAAGTTGTGCAGTTCACAAGCATGGAGGAAA
CAGACAGAACGACAGCGTTCAGGACAGTCAGAGCTAACCCAAGACGAGCC
TGGACTTGCCGCCAGGGGATTTCTTCTGGATGGCACTGGGGCCGGGGCAC
CGGGCTCGGGCACAGGCGCANAGGCACGGGCTTCTCTTCACTCTGCCCCA
GGCTGCCTGGCAAGTCTGTGTCCACATTTTCATGAATATCACCTTCTCCC
TTCAGATCTAAGAAGTCTCCGGAGCTTGCTTCAGAGGACTTCCTTCTCTG
CAGTCCAGATGACTCCAAGTCTTCCCTGCCACCACTNAGACTTGGCACCT
GCTGACTTGGCCCAAGAAGCTTGATTTTCTTCAGGTGTCCCAAAGATATT
AGAGGCCATT
(CloneID 346318)

SEQ ID NO:85

CGTTGGGTGTGGAGTTTCCCAGCGCCCCTCGGTCCGACCCTTTGAGCGTT
CTGCTCCGGCGCCACTANCCTCGCTCCTCGGCGCCATGACCACAACCACC
ACCTTCAAGGGAGTCGACCCCAACAGCAGGAATAGCTCCCGAGTTTTGCG
GCCTCCAGGTGGTGGATCCAATTTTTCATTAGGTTTTGATGAACCAACAG
AACAACCTGTGAGGAAGAACAAAATGGCCTCTAATATCTTTGGGACACCT
GAAGAAAATCAAGCTTCTTGGGCCAAGTCAGCAGGTGCCAAGTCTAGTGG
TGGCAGGGAAGACTTCGAGTCATCTGGGACTNGCAGAGAAGCAACTCCTC
TGAAGCAAGCTCCGGAGACTTCTTAGATCTGAAGGGAAGAAAGGTGATAT
TCATG
(CloneID 487018 in 3′ to 5′ orientation)

SEQ ID NO:86

AAAGTGCTTCTTTTTAATGAAACAAATCCAAGAGATGTACAGTCAGGCTC
AAGTTGTGCAGTTCACAAGCATGGAGGAAACAGACAGAACGACAGCGTTC
AGGACAGTCAGAGCTAACCCAAGACGAGGCTGGACTTGCCGCCAGGGGGA
TTTCTTCTGGATGGGCACTGGGGGCCGGGGCACCGGGCTGNGGCACAGGC
GCANAGGCACGGGCTTCTCTTCACTCTGCCCCAGGCTGCCTGGCAAGTCT
GTGTCCACATTTTCATGAATATCACCTTCTCCCTTCAGATCTAAGAAGTC
TCCGGAGCTTGCTTCAGAGGAGTTCCTTCTCTGCAGTCCAGATGACTCCA
AGTCTTCCCTGCCACCACTAGACTTGGCACCTGCTGACTTGGCCCAAGAA
GCTTGATTTTCTTCNGGTGTCCCAA
(CloneID 487018)

SEQ ID NO:87

CTCCTGCAGCGGTGGTCGGCTGTTGGGTGTGGAGTTTCCCAGCGCCCCTC
GGGTCCGACCCTTTGAGCGTTCTGCTCCGGCGCCACTACCTCGCTCCTCG
GCGCCATGACCACAACCACCACCTTCAAGGGAGTCGACCCCAACAGCAGG
AATAGCTCCCGAGTTTTGCGGCCTCCAGGTGGTGGATCCAATTTTTCATT
AGGTTTTGATGAACCAACAGAACAACCTGTGAGGAAGAACAAAATGGCCT
CTAATATCTTTGGGACACCTGAAGAAAATCAAGCTTCTTGGGCCAAGTCA
GCAGGTGCCAAGTCTAGTGGTGGCAGGGAAGACTTTGGAGTCATCTTGCA
CTTGCAGAGAAGGAACTCCTCTTGAAGCAAAGCTTCCGGAGACTTCTTTA
GATCTTGAAGGGAGAAGGTGATATTTG
(CloneID 3509038)

SEQ ID NO:88

AGCTAGGTACGAGGCCTGGGTCTTGCTGCAGCGGTGGTCGGCTGTTGGGT
GTGGAGTTTCCCAGCGCCCCTCGGGTCCGACCCTTTGAGCGTTCTGCTCC
GGCGCCAGCCTACCTCGCTCCTCGGCGCCATGACCACAACCACCACCTTC
AAGGGAGTCGACCCCAACAGCAGGAATAGCTCCCGAGTTTTGCGGCCTCC
AGGTGGTGGATCCAATTTTTCATTAGGTTTTGATGAACCAACAGAACAAC
CTGTGAGGAAGAACAAAATGGCCTCTAATATCTTTGGGACACCTGAAGAA
AATCAAGCTTCTTGGGCCAAGTCAGCAGGTGCCAAGTCTAGTGGTGGCAG
GGAAGACTTGGAGCTCATCTGGACTGCAGAGAAGGAACTCCTCTGAAGCA
AGCTCCGGAGACTTCTTAGATCTGAACGGGCAGAACGGTGATATTCATGA
AACATGTGGACACAGACTTGCCAGGCAGCCTGGGGCAGAGTGAAGAGAAG
CCCGTGCCTGCTGCGCCTGTGCCCAGCCCGGTGGCCCCGGCCCCAGTGCC
ATCCAGAAGAAATCCCCCTCGCGGCAAGTCCAGCCTCGTCTTCGGTTAGC
TCTGACTGTCCTGAACGCTGTCGTTCTGTCTGTTTCCTCCATGCTTGTGA
ACTGCACAACTTGAGCCTGACTGTCATCTCTTGGATTCTTCAC
(CloneID 5094728)

SEQ ID NO:89

GCAGCGGTGGTCGCCTGTTGGGTGTGGAGTTTCCCAGCGCCCCTCGGGTC
CGACCCTTTGACCGTTCTGCTCCGGCGCCAGCCTACCTCGCTCCTCGGCG
CCATGACCACAACCACCACCTTCAAGGGAGTCGACCCCAACAGCAGGAAT
AGCTCCCGAGTTTTGCGGCCTCCAGGTGGTGGATCCAATTTTTCATTAGG
TTTCGATGAACCAACAGAACAACCTGTGAGGAAGAACAAAATGGCCTCTA
ATATCTTTGGGACACCTGAAGAAAATCAAGCTTCTTGGGCCAAGTCAGCA
GGTGCCAAGTCTAGTGGTGGCAGGGAAGACTTGGAGTCATCTGGACTGCA
GAGAAGGAACTCCTCTGAAGCAAGCTCCGGAGACTTCTTAGATCTGAAGG
GAGAAGGTGATATTCATGTTTTCATAGAAAATGTGGACACAGACTTGCCA
GGCAGCCTGGGGCAGAGTGAAGAGAAGCCCGTGCCTGCTGCGCCTGTGCC
CAGCCCGGTGGCCCCGGACCCAGTGCCATCCAGAAGAAAACCCCCTGGCG
GCAAGTCCAGCCTCGTCTTGGGTTAGCTCTGACTGTCCTGAACGCTGTCG
TTCTGTCTGTTTCCTCCATGCTTGTGAACTGCACAACTTGAGCCTGACTG
TACATCTCTTGGATATGTTTCATTAAACAAGAAGCACTTTATGTAC
(CloneID 5590830)

SEQ ID NO:90

GTCCGGAATTCTCCGCATCTCCTGCAGCGGTGGTCGGCTTTTGGGTGTGC
AGTTTCCCAGCGCCCCTCGGGTCCGACCCTTTGAGCGTTCTGCTCCGGCG
CCAGCCTACCTCGCTCCTCGGCGCCATGACCACAACCACCACCTTCAAGG
GAGTCGACCCCAACAGCAGGAATAGCTCCCGAGTTTTGCGGCCTCCAGGT
GGTGGATCCAATTTTTCATTAGGTTTTGATGAACCAACAGAACAACCTGT
GAGGAAGAACAAAATGGCCTCTAATATCTTTGGGACACCTGAAGAAAATC
AAGCTTCTTGGGCCAAGTCAGCAGGTGCCAAGTCTAGTGGTGGCAGGGAA
GACTTGGAGTCATCTGGACTGCAGAGAAGGAACTCCTCTGAAGCAAGCTC
CGGAGACTTCTTAGATCTGAAGGGAGAAGGTGATATTCATGAAAATGTGG
ACACAGACTTGCCAGGCAGCCTGGGGCAGAGTGAAGAGAAGCCCGTGCCT
GCTGCGCCTGTGCCCAGCCCGGTGGCCCCGGCCCCAGTGCCATCCAGAAG
AAATCCCCCTGGCGGCAAGTCCAGCCTCGTCTTGGGTTAGCTCTGACTGT
CCTGAACGCTGTCGTTCTGTCTGTTTCCTCCATGCTTGTGAACTGCACAA
CTTGAGCCTGACTGTACATCTCTTGGAATTGTTTCATTAAAAAGAAGCAC
TTTATGTACTGCCTGTCTTTTTTTTTTTTCTTTTGAAAGAACAGGGTTTC
TCTCTGCCCTTGACTCTTGGGGTCCGTGGGCCATGGGCATGAGGGGTTTT
CCCAGTAGTTACATTGGGAGGGGAAACGTCTCTGTGACCACTTAAATCAC
CGGGGCTTTTAAACAACAAATTAATTTCGGCTCCCTCAAGGGCGGTTATA
AGGTACCCTTTTGTCTCTCTGAGGGCCCTGAAAACTCCTTCACCCCGGGC
ATCAACGCAGGTTCCCTACTTGGTTCAGCACCTCAAGCGCTCTGTACCCG
CCAGGGGACGGCTGTCTCCGCCCGAGGTGCCCCTCCCTCCCCCCCGGGCG
ATAATCTTTTCTTCAGCCCAACGAAACTGCCA
(CloneID 4550156)

SEQ ID NO:91

GCGGCTGTTGGGTCTGGAGTTTCCCAGCGCCCCTCGGGTCGACCCTTTGA
GCGTTCTGCTCCGGCGCCAGCCTACCTCGCTCCTCGGCGCCATGACCACA
ACCACCACCTTCAAGGGAGTCGACCCCAACAGCAGGAATAGCTCCCGAGT
TTTGCGGCCTCCAGGTGGTGGATCCAATTTTTCATTAGGTTTTGATGAAC
CAACAGAACAACCTGTGAGGAAGAACAAAATGGCCTCTAATATCTTTGGG
ACACCTGAAGAAAATCAAGCTTCTTGGGCCAAGTCAGCAGGTGCCAAGTC
TAGTGGTGGCAGGGAAGACTTGGAGTCATCTGGACTGCAGAGAAGGAACT
CCTCTGAAGCAAGCTCCGGAGACTTCTTAGATCTGAAGGGAGAAGGTGAT
ATTCATGAAAATCTGGACACAGACTTGCCAGGCAGCCTGCGGCAGAGTGA
AGAGAAGCCCGTGCCTGCTGCGCCTGTGCCCAGCCCGGTGGCCCCGGCCC
CAGTGCCATCCAGAAGAAATCCCCCTGGCGGCAAGTCCAGCCTCGTCTTG
GGTTAGCTCTGACTGTCCTGAACGCTGTCGTTCTGTCTGTTTCCTCCATG
CTTGTTGAACTGGCACAACTTGGAGCCCTGACTGTACATCTCTTGGATTG
TTTCATTAAAAAGAAGCCACTTTAAAAAAAAAAAAACCCCTGGAGCAGCT
ACGCAGAGACACGAGACAAAAAAAATCACAACGCGGGGCGGCCCTTTTGC
GGGGGGGGGCAAAACGAGTAAAAAAGGGGCGGGGTCTACAGTGTTGTCCG
GACACCCACTTTGGTCACAAGGGGGAACGCGGCGCCCAGAACGGGGGGAA
ATAAGCAAGCGGGGGATCCTA
(CloneID 5258724)

SEQ ID NO:92

GCTGCAGCGGTGGTCGGCTGTTGGGTGTGGAGTTTCCCAGCGCCCCTCGG
GTCCGACCCTTTGAGCGTTCTGCTCCGGCGCCAGCCTACCTCGCTCCTCG
GCGCCATGACCACAACCACCACCTTCAAGGGAGTCGACCCCAACAGCAGG
AATAGCTCCCGAGTTTGCGGCCTCCAGGTGGTGGATCCAATTTTTCATTA
GGTTTGATGAACCAACAGAACAACCTGTGAGGAAGAACAAAATGGCCTCT
AATATCTTTGGGACACCTGAAGAAAATCAAGCTTCTTGGGCCAAGTCAGC
AGGTGCCAAGTCTAGTGGTGGCAGGGAAGACTTGGAGTCATCTGGACTGC
AGAGAAGGAACTCCTCTGAAGCAAGCTCCGGAGACTTCTTAGATCTGAAG
GGAGAAGGTGATATTCATGAAAATGTGGACACAGACTTGCCAGGCAGCCT
GGGGCAGAGTGAAGAGAAGCCCGTGCCTGCTGCGCCTGTGCCCAGCCCGG
TGGCCCCGGCCCCAGTGCCATCCAGAAGAAATCCCCCTGGCGGCAAGTCC
AGCCTCGTCTTGGGTTAGCTCTGACTGTCCTGAACGCTGTCGTTCTGGTC
TGTTTCCTCCATGCTTGTGAACTGCACAACTTGAGCCTGACTGTACATCT
CTTGGATCTGTTCATTAAAAAGAAGCACTTTAAGAAAACCACACAAAAAA
ACTCCGAGGCCACTA
(CloneID 3458066)

SEQ ID NO:93

TGGGTGTGGAGTTTCCCAGCGCCCCTCGGGTCCGACCCTTTGAGCGTTCT
GCTCCGGCGCCAGCCTACCTCGCTCCTCGGCGCCATGACCACAACCACCA
CCTTCAAGGGAGTCGACCCCAACAGCAGGAATAGCTCCCGAGTTTGCGGC
CTCCAGGTGGTGGATCCAATTTTTCATTAGGTTTGATGAACCAACAGAAC
AACCTGTGAGGAAGAACAAAATGGCCTCTAATATCTTGGGACACCTGAAG
AAAATCAAGCTTCTGGGCCAAGTCAGCAGGTGCCAAGTCTAGTGGTGGCA
GGGAAGACTTGGAGTCATCTGGACTGCAGAGAAGGAACTCCTCTGAAGCA
AGCTCCGGAGACTTCTTAGATCTGAAGGGAGAAGGTGATATTCATGAAAA
TGTGGACACAGACTTGCCAGGCAGCCTGGGGCAGAGTGAAGAGAAGCCCG
TGCCTGCTGCGCCTGTGCCCAGCCCGGTGGCCCCGGCCCAGTGCCATCCA
GAAGAAATCCCCCTGGCGGCAAGTCCAGCCTCGTCTTGGGTTAGCTCTGA
CTGTCCTGAACGCTGTCGTTCTGTCTGTTTCCTCCATGCTTTGTGAACTG
GCACAACTTTGAGCCTGAGTGTACATCTCCTGTGGTTGTTTCATTAAACA
GAAGCACTTTATGTACTGTGTCTTTTTTTTGTCCTTCGGAGACCGCTTCT
CTCCGGTCCTGACTCTGGGTCTGGGGGCCGGCTGGGTGTCTAGCGGCACG
GCGGGAGCTTGGCCCCTGCGGGCT
(CloneID 3940211)

SEQ ID NO:94

TGGAGTTTCCCAGCGCCCCTCGGGTCCGACCCTTTGAGCGTTCTGCTCCG
GCGCCAGCCTACCTCGCTCCTCGGCGCCATGACCACAACCACCACCTTCA
AGGGAGTCGACCCCAACAGCAGGAATAGCTCCCGAGTTTGCGGCCTCCAG
GTGGTGGATCCAATTTTTCATTAGGTTTGATGAACCAACAGAACAACCTG
TGAGGAAGAACAAAATGGCCTCTAATATCTTGGGACACCTGAAGAAAATC
AAGCTTCTTGGGCCAAGTCAGCAGGTGCCAAGTCTAGTGGTGGCAGGGAA
GACTTGGAGTCATCTGGACTGCAGAGAAGGAACTCCTCTGAAGCAAGCTC
CGGAGACTTCTTAGATCTGAAGGGAGAAGGTGATATTCATGAAAATGTGG
ACACAGACTTGCCAGGCAGCCTGGGGCAGAGTGAAGAGAAGCCCGTGCCT
GCTGCGCCTGTGCCCAGCCCGGTGGCCCCGGCCCAGTGCCATCCAGAAGA
AATCCCCCTGGCGGCAAGTCCAGCCTCGTCTTGGGTTAGCTCTGACTGTC
CTGAACGCTGTCGTTCTGTCTGTTTCCTCCATGCTTGTGAACTGACAATT
GAGCCTGACTGTACATCTCTTGGATTTCTTTCATTAAAAAGAAGCACTTT
ATGTACTGCTGTCTTTTTTTTCTTTGGAAGAACAGCTGCCTCTCTGTCCT
GACCCTGGGTCTGGGGCATGCATGGGTGGTTCTAATAGTAATGGGGGAAC
TGGGACCTTAGACGGGTGTCAGAGACACACGGGTCCAGGTCAGGCCTCGG
CGGGGTTACACTCAGGGTGCACCGCATGGGGCACAAAGCCCGGGCGCTCC
GGCCCACAGGGGTCCCGCACCGGGGGCCAACGGGGCGGCACCCGGGAAAA
AGAGCGCCTCGCGGAAAAGTGTATACAAGGGGGGAGCGGCGAAGAACTAG
ACTCCGCGC
(CloneID 5015022)

SEQ ID NO:95

GGTCGGCTGTTGGGTGTGGAGTTTCCCAGCGCCCCTCGGGTCCGACCCTT
TGAGCGTTCTGCTCCGGCGCCAGCCTACCTCGCTCCTCGGCGCCATGACC
ACAACCACCACCTTCAAGGGAGTCGACCCCAACAGCAGGAATAGCTCCCG
AGTTTTGCGGCCTCCAGGTGGTGGATCCAATTTTTCATTAGGTTTTGATG
AACCAACAGAACAACCTGTGAGGAAGAACAAAATGGCCTCTAATATCTTT
GGGACACCTGAAGAAAATCAAGCTTCTTGGGCCAAGTCAGCAGGTGCCAA
GTCTAGTGGTGGCAGGGAAGACTTGGAGTCATCTGGACTGCAGAGAAGGA
ACTCCTCTGAAGCAAGCTCCGGAGACTTCTTAGATCTGAAGGGACGACGG
TGATATTCATGAACAATGTGGACACAGACTTGCCAGGCAGCCTGGGGCAG
AGTGAAGAGAAGCCCGTGCCTGCTGCGCCTGTGCCCAGCCCGGTGGCCCC
GGACCCAGTGCCATCCAGAAGAAATCCCCCTGGCGGCAAGTCCAGCCTCG
TCTTGGGTTAGCTCTGACTGTCCTGAACGCTGTCGTTCTGTCTGTTTCCT
CCATCCTTGTGAACTGCACAACTTGCACGCCTGACTGTACATCTCTTGGG
ATTTGTTTCATTACAAAGAAGCACTTTATGTACAAACACCATAAAAAAC
(CloneID 489924)

SEQ ID NO:96

GGCTGTTGGGTGTGGAGTTTCCCAGCGCCCCTCGGGTCCGACCCTTTGAG
CGTTCTGCTCCGGCGCCACTAGNCTCCCTCCTCGGCGCCATGACCACAAC
CACCACCTTCAAGGGAGTCGACCCCAACAGCAGGAATAGCTCCCGAGTTT
TGCGGCCTCCAGGTGGTGGATCCAATTTTTCATTAGGTTTTGATGAACCA
ACAGAACAACCTGTGAGGAAGAACAAAATGGCCTCTAATATCTTTGGGAC
ACCTGAAGAAAATCAAGCTTCTTGGGCCAAGTCAAGCAGGTGCCAAGTCC
AGTNGTTGCAAGGNAGGACTTTGGAGTCATTCTGGGACTGCAGAGANGGA
ACTCCTCTGAAGGCAAGCTCCCGGAGACTTTCTTAGGATCTTGAAAGGGA
GAAGGTGATATNCCATGAAAATGTGGACACAGACTTGCCAGGCAGCCTGG
GGAAGAGTGAAGAGAAGCCCGTGCCTGCTGCGCCTTGTGCCCAGCCCGGT
GCCCCGGGCCCAGTGCCATCCAGNAAAATCCCCCTTGGCGGAANGTCC
AGCTNGTTTNGGGTTAGCNCTGATTTCCTAACGNGGT
(CloneID 489924 in 3′ to 5′ orientation)

SEQ ID NO:97

ACATAAAGTGCTTCTTTTTAATGAAACAAATCCAAGAGATGTACAGTCAG
GCTCAAGTTGTGCAGTTCACAAGCATGGAGGAAACAGACAGAACGACAGC
GTTCAGGACAGTCAGAGCTAACCCAAGACGAGGCTGGACTTGCCGCCAGG
GGATTTCTTCTGGATGGCACTGGGGCCGGGGCACCGGGCTGGGCACAGGC
GCANAGGCACGGGCTTCTCTTCACTCTGCCCCAGGCTGCCTGGCAAGTCT
GTGTCCACATTTTCATGAATATCACCTTCTCCCTTCAGATCTAAGAAGTC
TCCGGAGCTTCCTTCAGAGGAGTTCCTTCTCTGCAGTCCAGATGACTCCA
AGTCTTCCCTGCCACCACTAGACTTGGCACCTGCTGACTTGGCCCAAGAA
GCTTGATTTTCTTCAGGTGTCCCAAAGATATTAGAGGCCATTTTGTTCTT
CCTCACAGGTTGTTCTGTTGGTTCATCAAAACCCTAATGAAAATTGGATC
CANCACCTNGAAGCCGCAAACTTCGGGAGCTANTCCTGCTGNTGGGG
(CloneID 3940896)

SEQ ID NO:98

GCTGTTGGGTGTGGAGTTTCCCAGCGCCCCTCGGGTCGACCCTTTGAGCG
TTCTGCTCCGGCGCCAGCCTACCTCGCTCCTCGGCGCCATGACCACAACC
ACCACCTTCAAGGGAGTCGACCCCAACAGCAGGAATAGCTCCCGAGTTTT
GCGGCCTCCAGGTGGTGGATCCAATTTTTCATTAGGTTTTGATGAACCAA
CAGAACAACCTGTGAGGAACAACAAAATGGCCTCTAATATCTTTGGGACA
CCTGAAGAAAATCAAGCTTCTTGGGCCAAGTCAGCAGGTGCCAAGTCTAG
TGCTGGCAGGGAAGACTTGGAGTCATCTGGACTGCAGAGAAGGAACTCCT
CTGAAGCAAGCTCCGGAGACTTCTTAGATCTGAAGGGAGAAGGTGATATT
CATGAAAATGTGGACACAGACCTTGCCAGGCAGCCTGGGGCAGAGTGAAG
AGAAGCCCGTGCCTGCTGCGCCTGTGCCCAGCCCGGTGGCCCCGGCCCCA
GTGCCATCCAGAAGAAATCCCCCTGGCGGCAAGTCCAGCCTCGTCTTGGG
TTAGCTCTGACTGTCCTGAACGCTGTCGTTCTGTCTGTTTCCTCCATGCT
TGTGAACTGCACAACTTGAGCCTGACTGTACATCTCTTGGATNTGTTTCA
TTAAAAAGAAGCACTTTATGT
(CloneID 4857040)

SEQ ID NO:99

GGAGTTTCCCAGCGCCCCTCGGGTCCGACCCTTTGAGCGTTCTGCTCCGG
CGCCAGCCTACCTCGCTCCTCGGCGCCATGACCACAACCACCACCTTCAA
GGGAGTCGACCCCAACAGCAGGAATAGCTCCCGAGTTTTGCGGCCTCCAG
GTGGTGGATCCAATTTTTCATTAGGTTTTGATGAACCAACAGAACAACCT
GTGAGGAAGAACAAAATGGCCTCTAATATCTTTGGGACACCTGAAGAAAA
TCAAGCTTCTTGGGCCAAGTCAGCAGGTGCCAAGTCTAGTGGTGGCAGGG
AAGACTTGGAGTCATCTGGACTGCAGAGAAGGAACTCCTCTGAAGCAAGC
TCCGGAGACTTCTTAGATCTGAAGGGAGAAGGTGATATTCATGAAAATGT
GGACACAGACTTGCCAGGCAGCCTGGGGCAGAGTGAAGAGAAGCCCGTGC
CTGCTGCGCCTGTGCCCAGCCCGGTGGCCCCGGCCCCAGTGCCATCCAGA
AGAAATCCCCCTGGCCGCAAGTCCAGCCTCGTCTTGGGTTAGCTCTGACT
GTCCTGAACGCTGTCGTTCTGTCTGTTTCCTCCATGCTTGTGAACTGCAC
AACTTGAGCCTGACTGTACATCTCTTGGATTCGTTTCATTAAAAAGAAGC
ACTTTATGTACTGCTGTCTTTTTTAATCCCTTTGGAACGACCAGGTTTCT
CCTCTGTCCTTGACTCTAGGGTCCTGTGGGCCATGG
(CloneID 3844688)

SEQ ID NO:100

TGTGGGTGTGGAGTTTCCCAGCGCCCCTCGGGTCCGACCCTTTGAGCGTT
CTGCTCCGGCGCCAGCCTACCTCGCTCCTCGGCGCCATGACGACAACCAC
CACCTTCAAGGGAGTCGACCCAACAGCAGGAATAGCTCCCGAGTTTGCGG
CCTCCAGGTGGTGGATCCAATTTTTCATTAGGTTTTGATGAACCAACAGA
ACAACCTGTGAGGAAGAACAAAATGGCCTCTAATATCTTTGGGACACCTG
AAGAAAATCAAGCTTCTTGGGCCAAGTCAGCAGGTGCCAAGTCTAGTGGT
GGCAGGGAAGACTTGGAGTCATCTGGACTGCAGAGAAGGAACTCCTCTGA
AGCAAGCTCCGGAGACTTCTTAGATCTGAAGGGAGAAGGTGATATTCATG
AAAATGTGGACACAGACTTGCCAGGCAGCCTGGGGCAGAGTGAAGAGAAG
CCCCTGCCTGCTGCGCCTGTGCCCAGCCCGGTGGCCCGGCCCAGTGCCAT
CCAGAAGAAATCCCCCTGGCGGGCAAGTCCAGCCTCGTCTTGGGTTAGCT
CTGACTGTCCTGAACGCTGTCGTTCTGTCTGTTTCCTCCATGCTTGTGAA
CTGCACAACTTGAGCCTGACTGTACATCTCTTGGATTGTCTCATTGAAAC
CAGAAGCACTTTATGTACTGCTGTCTTTCTTTACCCTTTGGACGGAACAG
GTTCCTCTGTCCTGA
(CloneID 4797137)

SEQ ID NO:101

AGCGGAGCGGCGGCTCCTGCAGCGGTGGTCGGCTGTTGGGTGTGGAGTTT
CCCAGCGCCCCTCGGGTCCGACCCTTTGAGCGTTCTGCTCCGGCGCCAGC
CTACCTCGCTCCTCGGCGCCATGACCACAACCACCACCTTCAAGGGAGTC
GACCCCAACAGCAGGAATAGCTCCCGAGTTTTGCGGCCTCCAGGTGGTGG
ATCCAATTTTTCATTAGGTTTTGATGAACCAACAGAACAACCTGTGAGGA
AGAACAAAATGGCCTCTAATATCTTTGGGACACCTGAAGAAAATCAAGCT
TCTTGGGCCAAGTCAGCAGGTGCCAAGTCTAGTGGTGGCAGGGAAGACTT
GGAGTCATCTGGACTGCAGAGAAGGAACTCCTCTGAAGCAAGCTCCGGAG
ACTTCTTAGATCTGAAGGGAGAACGGTGATATTCATGAAAATGTGGACAC
AGACTTGCCAGGCAGCCTGGGGCAGAGTGAAGAGAAGCCCGTGCCTGCTG
CGCCTGTGCCCAGCCCGGTGGCCCCGGCCCCAGTGCCATCCAGAAGAAAT
CCCCCTGGCGGCAAGTCCAGCCTCGTCTTGGGTTAGCTCTGACTGTCCTG
AACGCTGTCGTTCTGTCTGTTTCCTCCATGCTTGTGAACTGCACAACTTG
AGCCTGACTGTACATCTCTTGGCATTTGTCTCCATTACCAAGAAGCACTT
TTATGTACCTGCTGTCTTTCATCCATTATCACTTTCTGAAGAAACAGGTT
TTCTCCTCTGTCCTTGACTCCTGGGGCTCTGTGGGCCCTGGCATGACGTG
TCCCTAGCAGCTCCCTTGGCCCGCGAACGCTTTGTGACACCTAAGTACTG
AGGATCTCACGAACCA
(CloneID 4304080)

SEQ ID NO:102

GGTTGGGTGTGGAGTTTCCCAGCGCCCCTCGGGTCCGACCCTTTGAGCGT
TCTGCTCCGGCGCCAGCCTACCTCGCTCCTCGGCGCCATGACCACAACCA
CCACCTTCAAGGGAGTCGACCCCAACAGCAGGAATAGCTCCCGAGTTTGC
GGCCTCCAGGTGGTGGATCCAATTTTTCATTAGGTTTGATGAACCAACAG
AACAACCTGTGAGGAAGAACAAAATGGCCTCTAATATCTTTGGGACACCT
GAAGAAAATCAAGCTTCTTGGGCCAAGTCAGCAGGTGCCAAGTCTAGTGG
TGGCAGGGAAGACTTGGAGTCATCTGGACTGCAGAGAAGGAACTCCTCTG
AAGCAAGCTCCGGAGACTTCTTAGATCTGAAGGGAGAAGGTGATATTCAT
GAAAATGTGGACACAGACTTGCCAGGCAGCCTGGGGCAGAGTGAAGAGAA
GCCCGTGCTGCTGCGCCTGTGCCCAGCCCGGTGGCCCCGGCCCAGTGCCA
TCCAGAAGAAATCCCCCTGGCGGCAAGTCCAGCCTCGTCTTGGGTTAGCT
CTGACTGTACTGAACCGCTGTCGTTCCTGTCTGTTTCCTCCATGCTTGGT
GAACTGACAACTTGAGCCTGATGTACAATCTCTGGGGATTTGTTTCATTA
AAAAGAAGCACTTTATGTAAAAAAAAGTTCACAAAAAAAAATCGGGGCAC
AAAGAAGGGGGGGAGAAAAGGGGAGGAAGGGGACATGGACATAGGTGTTC
TGGACAAGTTTTGGCCAGGGGAAATGGGGCCCCAAAGGGGGGATATGAAA
GAAGTAAGTA
(CloneID 5928314)

SEQ ID NO:103

GCGGTGGTCGGCTGTTGGGTGTGGACTTTCCCAGCGCCCCTCGGGTCCGA
CCCTTTGAGCGTTCTGCTCCGGCGCCAGCCTACCTCGCTCCTCGGCGCCA
TGACCACAACCACCACCTTCAAGGGAGTCGACCCCAACAGCAGGAATAGC
TCCCGAGTTTTGCGGCCTCCAGGTGGTGGATCCAATTTTTCATTAGGTTT
TGATGAACCAACAGAACAACCTGTGAGGAAGAACAAAATGGCCTCTAATA
TCTTTGGGACACCTGAAGAAAATCAAGCTTCTTGGGCCAAGTCAGCAGGT
GCCAAGTCTAGTGGTGGCAGGGAAGACTTGGAGTCATCTGGACTGCAGAG
AAGGAACTCCTCTGAAGCAAGCTCCGGAGACTTCTTAGATCTGAAGGGAG
AAGGTGATATTCATGAAAATGTGGACACAGACTTGCCAGGCAGCCTGGGG
CAGAGTGAAGAGAAGCCCGTGCCTGCTGCGCCTGTGCCCAGCCCGGTGGC
CCCGGCCCCAGTGCCATCCAGAAGAAATCCCCCTGGCGGCAAGTCCAGCC
TCGTCTTGGGTTAGCTCTGACTGTCCTGAACGCTGTCGTTCTGTCTGTTT
CCTCCATGCTTGTGAACTGCACAACTTGAGCCTCACTGTACATCTCTTGG
ATTTGTTTCATTAAAAAGAAGCACTTTATGTACTGCTGTCTTTTTTTTTT
TTCTTTTGAAAACAAGTTTCTCTCTGTCCTTGACTCTTGGGTCTGTGGGG
CCATGGCATGAGTGGTTTTCTAGTAGTAGATTGGAGGGAAAAGCTTTGTG
GACACTTTAAGAACTGGGGTTTTTTAGAAGAAAATAATTTGGGTTTCCGA
AGGTGGGTAAAAAGGATCTTTTTGGGAACTGGAGGGGTTTTTAACAACTT
TAATTTGGGGGTTTTACCCAAGCCCTCAAACTTGGGACCGAACCCTTTAA
ACAGGTG
(CloneID 6159735)

SEQ ID NO:104

GGACGCGTGGGCGGCTGTTGGGTGTGGAGTTTCCCAGCGCCCCTCGGGTC
CGACCCTTTGAGCGTTCTGCTCCGGCGCCAGCCTACCTCGCTCCTCGGCG
CCATGACCACAACCACCACCTTCAAGGGAGTCGACCCCAACAGCAGGAAT
AGCTCCCGAGTTTTGCGGCCTCCAGGTGGTGGATCCAATTTTTCATTAGG
TTTTGATGAACCAACAGAACAACCTGTGAGGAAGAACAAAATGGCCTCTA
ATATCTTTGGGACACCTGAAGAAAATCAAGCTTCTTGGGCCAAGTCAGCA
GGTGCCAAGTCTAGTGGTGGCAGGGAAGAGTTGGAGTCATCTGGACTGCA
GAGAAGGAACTCCTCTGAAGCAAGCTCCGGAGACTTCTTAGATCTGAAGG
GAGAAGGTGATATTCATGAAAATGTGGACACAGACTTGCCAGGCAGCCTG
GGGCAGAGTGAAGAGAAGCCCGTGCCTGCTGCGCCTGTGCCCAGCCCGGT
GGCCCCGGCCCCAGTGCCATCCAGAAGAAATCCCCCTGGCGGCAAGTCCA
GCCTCGTCTTGGGTTAGCTCTGACTGTCCTGAACGCTGTCGTTCTGTCTG
TTTCCTCCATGCTTGTGAACTGCACAACTTGAGCCTGACTGTACATCTCT
TGGATTTGTTTCATTAAAAAGAAGCACTTTATGTACAACACCCCACCCNC
CCACCACCCCCCCACACCATCAAACAAAGGGGGGGCCGGCTCTAAGAATA
TCCCCTCGAGGGGGCCCAAAGCCTTACCGCGTAACCCCATCTTTTTCTTG
GAACAAAAGGGGTTCCCCTTATAGGGGGAGGCCGGAAATTATAAAAGCTT
AGGGCACCCTGGGGCCGCTCCCCTTTTTTTAACAAAACGTCTCCTCGGAA
TCGGGGG
(CloneID 308502)

SEQ ID NO:105

GCTGTTGGGTGTGGAGTTTCCCAGCGCCCCTCGGGTCCGACCCTTTGAGC
GTTCTGCTCCGGCGCCACTANCCTCGCTCCTCGGCGCCATGACCACAACC
ACCACCTTCAAGGGAGTCGACCCCAACAGCAGGAATAGCTCCCGAGTTTT
GCGGCCTCCAGGTGGTGGATNCAATTTTTCAATTAGGTTTTGATGAACCA
ACAGGACCANCTGTTAAGAAGGACCAAATTGGGCTCCTAATAATCTTTGG
GACACCTGAAGAAAATCAAGCTTCTTGGGGCCAAGTCAGCAGGTGCCAAG
TCTAGTGGTGGCAGGGGAAAGACTTGGAGTCATCTGGACTTGCAGAGAAG
GAACTCCTCTTGAAGCAAGCTTCCGGAGACTTTCTTTAGATCTTGAAAGG
GGAGAAAGGTTGATATTCCATGGAAAATGTTGGACACAGACTTTTN
(CloneID 308502 in 3′ to 5′ orientation)

SEQ ID NO:106

AAAGTGCTTCTTTTTAATGAAACAAATCCAAGAGATGTACACTCAGGCTC
AAGTTGTGCAGTTCACAAGCATGGAGGAAACAGACAGAACGACAGCGTTC
AGGACAGTCAGAGCTAACCCAAGACGAGGCTGGACTTGCCGCCAGGGGAT
TTCTTCTGGATGGCACTGGGGCCGGGGCACCGGGCTGGGCACAGGCGCAC
AGGCACGGGCTTCTCTTCACTCTGCCCCAGGCTGCCTGGCAAGTCTGTGT
CCACATTTTCATGAATATCACCTTCTCCCTTCAGATCTAAGAAGTCTCCG
GAGCTTGCTTCAGAGGAGTTCCTTCTCTGCAGTCCAGATGACTCCAAGTC
TTCCCTGCCACCACTAGACTTGGCACCTGCTGACTTGGCCCAAGAAGCTT
GATTTTCTTCAGGTGTCCCA
(CloneID 4302544)

SEQ ID NO:107

GGTTGGGTGTGGAGTTTCCCAGCGCCCCTCGGGTCCGACCCTTTGAGCGT
TCTGCTCCGGCGCCAGCCTACCTCGCTCCTCGGCGCCATGACCACACCAC
CACCTTCAAGGGAGTCGACCCCAACAGCAGGAATAGCTCCCGAGTTTTGC
GGCCTCCAGGTGGTGGATCCAATTTTTCATTAGGTTTGATGAACCAACAG
AACAACCTGTGAGGAAGAACAAAATGGCCTCTAATATCTTTGGGACACCT
GAAGAAAATCAAGCTTCTTGGGCCAAGTCAGCAGGTGCCAAGTCTAGTGG
TGGCAGGGAAGACTTGGAGTCATCTGGACTGCAGAGAAGGAACTCCTCTG
AAGCAAGCTCCGGAGACTTCTTAGATCTGAAGGGAGAAGGTGATATTCAT
GAAAATGTGGACACAGACTTGCCAGGCAGCCTGGGGCAGAGTGAAGAGAA
GCCCGTGCCTGCTGCGCCTGTGCCCAGCCCGGTGGCCCCGGCCCACTGCC
ATCCAGAAGAAATCCCCCTGGCGGCAAGTCCAGCCTCGTCTTGGGTTAGC
TCTGACTGTCCTGAACGCTGTCGTTCTGTCTGTTTCCTCCATGCTTGTGA
ACTGCACAACTTGAGCCCTGACTGTACATCTCTTGGATTGTCTCATTAAA
AAGAAGCACTTTATGTTAAACAAAAACAAAAAAAAAA
(CloneID 3937350)

SEQ ID NO:108

TTGGGTGTGGAGTTTCCCAGCGCCCCTCGGGTCCGACCCTTTGAGCGTTC
TGCTCCGGCGCCAGCCTACCTCGCTCCTCGGCGCCATGACCACAACCACC
ACCTTCAAGGGAGTCGACCCCAACAGCAGGAATAGCTCCCGAGTTTTGCG
GCCTCCAGGTCGTGGATCCAATTTTTCATTAGGTTTTGATGAACCAACAG
AACAACCTGTGAGGAAGAACAAAATGGCCTCTAATATCTTTGGGACACCT
GAAGAAAATCAAGCTTCTTGGGCCAAGTCAGCAGGTGCCAAGTCTAGTGG
TGGCAGGGAAGACTTGGAGTCATCTGGACTGCAGAGAAGGAACTCCTCTG
AAGCAAGCTCCGGAGACTTCTTAGATCTGAAGGGAGAAGGTGATATTCAT
GAAAATGTGGACACAGACTTGCCAGGCAGCCTGGGGCAGAGTGAAGAGAA
GCCCGTGCCTGCTGCGCCTGTGCCCAGCCCGGTGGCCCCGGCCCCAGTGC
CATCCAGAAGAAATCCCCCTGGCGGCAAGTCCAGCCTCGTCTTGGGTTAG
CTCTGACTGTCCTGAACGCTGTCGTTCTGTCTGTTTCCTCCATGCTTGTG
AACTGCACAACTTGAGCCTGACTGTACATCTCTTGGATTTGTTTCATTAA
AAACAAGCACTTTATGT
(CloneID 3835958)

SEQ ID NO:109

TTCCCAGCGCCCCTCGGGTCAGGACCCTTTGAGCGTTCTGCTCCGGCGCC
AGCCTACCTCGCTCCTCGGCGCCATGACCACAACCACCACCTTCAAGGGA
GTCGACCCCAACAGCAGGAATAGCTCCCGAGTTTGCGGCCTCCAGGTGGT
GGATCCAATTTTTCATTAGCTTTGATGAACCAACAGAACAACCTGTGAGG
AAGAACAAAATGGCCTCTAATATCTTTGGGACACCTGAAGAAAATCAAGC
TTCTTGGGCCAAGTCAGCAGGTGCCAAGTCTAGTGGTGGCAGGGAAGACT
TGGAGTCATCTGGACTGCAGAGAAGGAACTCGTCTGAAGCAAGCTCCGGA
GACTTCTTAGATCTGAAGGGAGAAGGTGATATTCATGAAAATGTGGACAC
AGACTTGCCAGGCAGCCTGGGGCAGAGTGAAGAGAAGCCCGTGCCTGCTG
CGCCTGTGCCCAGCCCGGTGGCCCCGGCCCAGTGCCATCCAGAAGAAATC
CCCCTGGCGGCAAGTCCAGCCTCGTCTTGGGTTAGCTCTGACTGTCCTGA
ACGCTGTCGTTCTGTCTGTTTCCTCCATGCTTGTGAACTGACAACTTGAG
CCTGACTGTACATCTCTTGGATTTGTTTCATTAAAAAGAAGCACTTTAAA
AAAAAAAAAAAAAAAAAAAAAAACCGGGGGACACAAGGGGGGGGGGGAAA
AAG
(CloneID 5925943)

SEQ ID NO:110

TCGGCTGTTGGGTGTGGAGTTTCCCAGCGCCCCTCGGGTCCGACCCTTTG
AGCGTTCTGCTCCGGCGCCAGCCTACCTCGCTCCTCGGCGCCATGACCAC
AACCACCACCTTCAAGGGAGTCGACCCCAACAGCAGGAATAGCTCCCGAG
TTTTGCGGCCTCCAGGTGGTGGATCCAATTTTTCATTAGGTTTTGATGAA
CCAACAGAACAACCTGTGAGGAAGAACAAAATGGCCTCTAATATCTTTGG
GACACCTGAAGAAAATCAAGCTTCTTGGGCCAAGTCAGCAGGTGCCAAGT
CTAGTGGTGGCAGGGAAGACTTGGAGTCATCTGGACTGCAGAGAAGGAAC
TCCTCTGAAGCAAGCTCCGGAGACTTCTTAGATCTGAAGGGAGAAGGTGA
TATTCATCAAAATGTGGACACAGACTTGCCAGGCAGCCTGGGGCAGAGTG
AAGAGAAGCCCGTGCCTGCTGCGCCTGTGCCCAGCCCGGTGGCCCCGGCC
CCAGTGCCATCCAGAAGAAATCCCCCTGGCGGCAAGTCCACCCTCGTCTT
GGGTTAGCTCTGACTGTCCTCAACGCTGTCGTTCTGTCTGTTTCCTCCAT
GCTTGTGAACTGCACAACTTGAGCCTGACTGTACATCTCTTGGATTTGTT
TCATTANAAAGAAGCACTTTATGTAAAAAAAAAAAAAAAAAA
(CloneID 3936327)

SEQ ID NO:111

GTTGGGTGTGGAGTTTCCCAGCGCCCCTCGGGTCCGACCCTTTGAGCGTT
CTGCTCCGGCGCCAGCCTACCTCGCTCCTCGGCGCCATCACCACAACCAC
CACCTTCAAGGGAGTCGACCCCAACAGCAGGAATAGCTCCCGAGTTTTGC
GGCCTCCAGGTGGTGCATCCAATTTTTCATTAGGTTTTGATGAACCAACA
GAACAACCTGTGAGGAAGAACAAAATGGCCTCTAATATCTTTGGGACACC
TGAAGAAAATCAAGCTTCTTGGGCCAAGTCACCAGGTGCCAAGTCTAGTC
GTGGCAGGGAAGACTTGGAGTCATCTGGACTGCAGAGAAGGAACTCCTCT
GAAGCAAGCTCCGGAGACTTCTTAGATCTGAAGGGAGAAGGTGATATTCA
TGAAAATGTGGACACAGACTTGCCAGGCAGCCTGGGGCAGAGTGAAGAGA
AGCCCGTGCCTGCTGCGCCTGTGCCCAGCCCGGTGGCCCCGGCCCAGTGC
CATCCAGAAGAAATCCCCCTGGCGGCAAGTCCAGCCTCGTCTTGCGTTAG
CTCTGACTGTCCTGAACGCTGTCGTTCTGTCTGTTTCCTCCATGCTTGTG
AACTGCACAACTTGAGCCTGACTGTACATCTCTTGGATTTGTCTTCATTA
AAAAGAAGCACTT
(CloneID 4100301)

SEQ ID NO:112

GGCGCCTCCTGCAGCGGTGGTCGGCTGTTGGGTGTGGAGTTTCCCAGCGC
CCCTCGGGTCCGACCCTTTGAGCGTTCTGCTCCGGCGCCAGCCTACCTCG
CTCCTCGGCGCCATGACCACAACCACCACCTTCAAGGGAGTCGACCCCAA
CAGCAGGAATAGCTCCCGAGTTTTGCGGCCTCCAGGTGGTGGATCCAATT
TTTCATTAGGTTTTGATGAACCAACAGAACAACCTGTGAGGAAGAACAAA
ATGGCCTCTAATATCTTTGGGACACCTGAAGAAAATCAAGCTTCTTGGGC
CAAGTCAGCAGGTGCCAAGTCTAGTGGTGGCAGGGAAGACTTGGAGTCAT
CTGGACTGCAGAGAAGCAACTCCTCTGAAGCAAGCTCCGGAGACTTCTTA
GATCTGAAGGGAGAAGGTGATATTCATGAAAATGTGGACACAGACTTGCC
AGGCCGCTGGGGCAGAGTGAAGAGAAGCCCGTGCTGCTGCGCCCTCTGCC
CAGCCGGTGGCCCCGGCCCCAGTGCCATCCAGAAGAAATCCCCCTGGCGG
CAAGTCCAGCCTCGTCTGGGTTAGCCTCTGACTGTCCTGAACGCTGTCGT
TCTGTCTGTTTCCTCCATGCTTGTGAACTGCACAATTTGAGCTTGACTGT
ACATCTTCTGGATTTGTGTCATTACCACAGACGCCTTTACAAAGAGAAAA
AAAAATCCGGGGCTTCATTTTCGGGGGGGAACAGGGTATGACG
(CloneID 3903688)

SEQ ID NO:113

GTTGGGTGTGGGAGTGTCCCAGCGCCCCTCGGGTCCGACCCTTTGAGCGT
TCTGCTCCGGCGCCAGCCTACCTCGCTCCTCGGCGCCATGACCACAACCA
CCACCTTCAAGGGAGTCGACCCCAACAGCAGGAATAGCTCCCGAGTTTTG
CGGCCTCCAGGTGGTGGATCCAATTTTTCATTAGGTTTGATGAACCAACA
GAACAACCTGTGAGGAAGAACAAAATGGCCTCTAATATCTTTCGGACACC
TGAAGAAAATCAAGCTTCTTGGGCCAAGTCAGCAGGTGCCAAGTCTAGTG
GTGGCAGGGAAGACTTGGAGTCATCTGGACTGCAGAGAAGGAACTCCTCT
GAAGCAAGCTCCGGAGACTTCTTAGATCTGAAGGGAGAAGGTGATATTCA
TGAAAATGTGGACACAGACTTGCCAGGCAGCCTGGGGCAGAGTGAAGAGA
AGCCCGTGCCTGCTGCGCCTGTGCCCAGCCCGGTGGCCCCGGCCCCAGTG
CCATCCAGAAGAAATCCCCCTGGCGGCAAGTCCAGCCTCGTCTTGGGTTA
GCTCTGACTGTCCTGAACGCTGTCGTTCTGTCTGTTTCCTCCATGCTTGT
GAACTGACAATTGAGCCTGACTGTACATCTCTGGATTGTTTCATTAAAAA
AGAAGCAC
(CloneID 4797136)

SEQ ID NO:114

AGCGGAGTCCCCTGTCTTGCTCCGGTAGTTGGCTGTGGACTGAGCGCTTT
CCCAGCGCCCCTCGGGTCCGACCCTTTGATGCGTTGCTGCTCCTGGCAGC
CAGCCATACCTCGCTCCTCGGCGGCCATGACCACAAGCGACCACCTTCA
AGGGAGTCGACCCCAACAGCAGGAATATGCTCCCGAGTTTTGCGGCTCCA
GGTGGTGGATCCAATTTTTCATTAGGTTTTGATGAACCAACAGAACAACC
TGTGAGGAAGAACAAAATGGTCTCTAATATCTTTGGGACACCTGAAGAAA
CATCAAGCTTCTTGGGCCAAGTCAGCAGGTGCCAAGTCTAGTGGTGGCAG
GGAAGACTTGGAGTCATCTGGACTGCAGAGAAGGAACTCCTCTGAAGCAA
GCTCCGGAGACTTCTTAGATCTGAAGGGAGACGGTGATATTCATGAAAAC
TGTGGACACAGACTTGCCAGGCAGCCTGGGGCACGAGTGAAGAGAAGCCC
GTGCCTGCTGCGCCTGTGCCCAGCCCGGTGGCCCCGGGCCCAGTGCCATC
CAGAAGAAATCCCCCTGGCGGCAAGTCCAGCCTCGTCTATGGGTTAAGCT
CTGACTGTCCTGAACGCTGTCGTTCTGTCTGTTTCCTCCATGCTTGTGAA
CTGCACAACTTGAGCCTGACTGTACAATCTCTTGGATTGCTCTCATTACA
AACAGAAGCACTTTATGTACTGCTGTACTTCTCATTTTCGTTTTTGCACA
ACAGGGTTGCCTCTCTGCACTTTGACTCTTTGGGCTCTGTGCGCCCATGG
GCATGACTTT
(CloneID 4120864)

SEQ ID NO:115

GCTCCTGCAGCGGTGGTCGGCTGTGGGTGTGGAGTTTCCCAGCGCCCCTC
GGGTCCGACCCTTTGAGCGTTCTGCTCCGGCGCCAGCCTACCTCGCTCCT
CGGCGCCATGACCACAACCACCACCTTCAAGGGAGTCGACCCCAACAGCA
GGAATAGCTCCCGAGTTTGCGGCCTCCAGGTGGTGGATCCAATTTTTCAT
TAGGTTTGATGAACCAACAGAACAACCTGTGAGGAAGAACAAAATGGCCT
CTAATATCTTGGGACACCTGAAGAAAATCAAGCTTCTTGGGCCAAGTCAC
CAGGTGCCAAGTCTAGTGGTGGCAGGGAAGACTTGGACTCATCTGGACTG
CAGAGAAGGAACTCCTCTGAAGCAAGCTCCGGAGACTTCTTAGATCTGAA
GGGAGAAGCTGATATTCATGAAAATGTGGACACAGACTTGCCAGGCAGCC
TGGGGCAGAGTGAAGAGAAGCCCGTGCCTGCTGCGCCTGTGCCCAGCCCG
GTGGCCCCGGCCCAGTGCCATCCAGAAGAAATCCCCCTGGCGGCAAGTCC
AGCCTCGTCTTGGGTTAGCTCTGACTGTCCTGAACGCTGTCGTTCTGTCT
GTTTCCTCCATGCTTGTGAACTGCACAACTTGAGGCTGACTGTACATCTC
TGGATTGTTCTCCATTAACAAAGAAGCACTTTAGGCAACACCCACCAAAA
AC
(CloneID 5405894)

SEQ ID NO:116

CTCCTGCAGCGGTGGTCCGCTGTTGGGTGTGGAGTTTCCCAGCGCCCCTC
GGGTCCGACCCTTTGAGCGTTCTGCTCCGGCGCCAGCCTACCTCGCTCCT
CGGCGCCATGACCACAACCACCACCTTCAAGGGAGTCGACCCCAACAGCA
GGAATAGCTCCCGAGTTTTGCGGCCTCCAGGTGGTGGATCCAATTTTTCA
TTAGGTTTTGATGAACCAACAGAACAACCTGTGAGGAAGAACAAAATCGC
CTCTAATATCTTTGGGACACCTGAAGAAAATCAAGCTTCTTGGGCCAAGT
CAGCAGGTGCCAAGTCTAGTGGTGGCAGGGAAGACTTGGAGTCATCTGGA
CTGCAGAGAAGGAACTCCTCTGAAGCAAGCTCCGGAGACTTCTTAGATCT
GAAGGGAGAAGGTGATATTCATGAAAATGTGGACACAGACTTGCCAGGCA
GCCTGGGGCAGAGTGAAGAGAAGCCCCTGCCTGCTGCGCCTGTGCCCAGC
CCGGTGGCCCCGGCCCCAGTGCCATCCAGAAGAAATTCCCCTGGCGGCAA
GTCCAGCCTCGTCTTGGGTTAGCTCTGACTGTCCTGAACGCTGTCGTNCT
GTCTGTTTCCTCCATGCTTGTGAACTGCACAACTTGAGCCTGACTGTACA
TCTCTTGGATTCGTTCCCATTAACAAAGAAGCACTTTAAAACAACCCACA
CACACAACGCGG
(CloneID 4276593)

SEQ ID NO:117

GAGCGGCGGCTCCTGCAGCGGTGGTCGGCTGTTGGGTGTGGAGTTTCCCA
GCGCCCCTCGGGTCCGACCCTTTGAGCGTTCTGCTCCGGCGCCAGCCTAC
CTCGCTCCTCGGCGCCATGACCACAACCACCACCTTCAAGGGAGTCGACC
CCAACAGCAGGAATAGCTCCCGAGTTTTGCGGCCTCCAGGTGGTGGATCC
AATTTTTCATTAGGTTTTTGATGAACCAACAGAACAACCTGTGAGGAAGA
ACAAAAAATGGCTCTAATATCTTTGGGACACCTGAAGAAAATCAAGCTTC
TTGGGCCAAAGTCAGCAGGTGCCAAGTCTAGTGGTGGCAGGGAAGACTTG
GAGTCATCTGGACTGCAGAGAAGGAACTCCTCTGAAGCAAGCTCCGGAGA
CTTCTTAGATCTGAAGGGAGAAGGTGATATTCATGCACAATGTGGACACA
GACTTGCCAGGCAGCCTGGGGCAGAGTGAAGAGAAGCCCGTGCTGCTGCG
CCTGTGCCAGCCCGGTGGTCCGNGCCAGTGCCATCCAGAAGAAATCCCCT
GGCGGCAAGTCCAGCTCGTCTTTGGGTTAGCTCTGACTGTGCCTGAACGC
TGTCGTCTGTCTGTTCCTCCCATGCTTGTGAACTGACAATTGGGCAGACT
GTCATCTCTTGGTATTTGTTCCTTAACAAGAAGCTAAACAAACACAACAC
ACAAAAAATTTTGGCGGGCTGGGGCACGAGAAGTTTAACACCACTCTGGG
CGCGCGCCCCAGGTGGAGAAGGGCGCCGCGGTCACGGAACACCGGCAGAC
GCGGGGGGGGTTCCCACAAAAACGGGGGGCCGGGGGTGAACACCACAGGG
GGGGGTCGGTCACACGACCCCACTCAACCCCG
(CloneID 4127376)

SEQ ID NO:118

GGAGTTTCCCAGCGCCCCTCGGGTCCGACCCTTTGAGCGTTCTGCTCCGG
CGCCAGCCTACCTCGCTCCTCGGCGCCATGACCACAACCACCACCTTCAA
GGGAGTCGACCCCAACAGCAGGAATAGCTCCCGAGTTTTGCGGCCTCCAG
GTGGTGGATCCAATTTTTCATTAGGTTTTGATGAACCAACAGAACAACCT
GTGAGGAAGAACAAAATGGCCTCTAATATCTTTGGGACACCTGAAGAAAA
TCAAGCTTCTTGGGCCAAGTCAGCAGGTGCCAAGTCTAGTGGTGGCAGGG
AAGACTTGGAGTCATCTGGACTGCAGAGAAGGAACTCCTCTGAAGCAAGC
TCCGGAGACTTCTTAGATCTCAAGGGAGAAGGTGATATTCATGAAAATGT
GGACACAGACTTGCCAGGCAGCCTGGGGCAGAGTGAAGAGAAGCCCGTGC
CTGCTGCGCCTGTGCCCAGCCCGGTGGCCCCGGCCCCAGTGCCATCCAGA
AGAAATCCCCGTGGCGGCAAGTCCAGCCCTCGTCTTGGGTTAGCTCTGAC
TGTCCTGAACGCTGTCGTTCTGTCTGTTTCCTCCATGCTTGTGAACTGCA
CAACTTGAGCCTGACTGTACATCTCTTGGATTGGTTTCATTAAAAAGAAG
CACTTTATGTACACAACACACACACAAAAAC
(CloneID 5934901)

SEQ ID NO:119

CTGTTGGGTGTGGAGTTTCCCAGCGCCCCTCGGGTCCGACCCTTTGAGCG
TTCTGCTCCGGCGCCAGCCTACCTCGCTCCTCGGCGCCATGACCACAACC
ACCACCTTCAAGGCAGTCGACCCCAACAGCAGGAATAGCTCCCGAGTTTT
GCGGCCTCCAGGTCGTGGATCCAATTTTTCATTAGGTTTTGATGAACCAA
CAGAACAACCTGTGAGGAAGAACAAAATGGCCTCTAATATCTTTGGGACA
CCTGAAGAAAATCAAGCTTCTTGGGCCAAGTCAGCAGGTGCCAAGTCTAG
TGCTGGCAGGGAAGACTTGGAGTCATCTGGACTGCAGAGAAGGAACTCCT
CTGAAGCAAGCTCCGGAGACTTCTTAGATCTGAAGGGAGAAGGTGATATT
CATGAAAATGTGGACACAGACTTGCCAGGCAGCCTGGGGCAGAGTGAAGA
GAAGCCCGTGCCTGCTGCGCCTGTGCCCAGCCCGGTGGCCCCGGCCCCAG
TGCCATCCAGAAGAAATCCCCCTGGCGGCAAGTCCAGCCTCGTCTTGGGT
TAGCTCTGACTGTCCTGAACGCTGTCGTTCTGTCTGTTTCCTCCATGCTT
GTGAACTGCACAACTTGAGCCTGACTGTACATCTCTTGGATTTGTTTCAT
TANAAAGAAGCACTTTATGTAAAAAAAAAAAAAAAAAA
(CloneID 4622938)

SEQ ID NO:120

GCACGAGGGGGTGTGGAGTTTCCTAGCGCCCCTCGGGTCCGACCCTTTGA
GCGTTCTGCTCCGGCGCCAGCCTACCTCGCTCCTCGGCGCCATGACCACA
ACCACCACCTTCAAGGGAGTCGACCCCAACAGCAGGAATAGCTCCCGAGT
TTTGCGGCCTCCAGGTGGTGGATCCAATTTTTCATTAGGTTTTGATGAAC
CAACAGAACAACCTGTGAGGAACAACAAAATGGCCTCTAATATCTTTGGG
ACACCTGAAGAAAATCAAGCTTCTTGGGCCAAGTCAGCAGGTGCCAAGTC
TAGTGGTGGCAGGGAAGACTTGCAGTCATCTGGACTGCAGAGAAGGAACT
CCTCTGAAGCAAGCTCCGGAGACTTCTTAGATCTGAAGGGAGAAGGTGAT
ATTCATGAAAATGTGGACACAGACTTGCCAGGCAGCCTGGGGCAGACTGA
AGAGAAGCCCGTCCCTGCTCCGCCTGTGCCCAGCCCGGTGGCCCCGGCCC
CAGTGCCATCCAGAAGAAATCCCCCTGGCGGCAAGTCCAGCCTCGTCTTG
GGTTAGCTCTGACTGTCCTGAACGCTGTCGTTCTGTCTGTTTCCTCCAT
GCTTGTGAACTGCACAACTTGAGCCTGACTGTCATCTCTTGGATTTGTTT
CATTAAA
(CloneID 3909220)

SEQ ID NO:121

TGTGGAGTTTCCCAGCGCCCCTCGGGTCCGACCCTTTGACCGTTCTGCTC
CGGCCCCAGCCTACCTCGCTCCTCGGCGCCATGACCACAACCACCACCTT
CAAGGGAGTCGACCCCAACAGCAGGAATAGCTCCCGAGTTTTGCGGCCTC
CAGGTGGTGGATCCAATTTTTCATTAGGTTTTGATGAACCAACAGAACAA
CCTGTGAGGAAGAACAAAATGGCCTCTAATATCTTTGGGACACCTGAAGA
AAATCAAGCTTCTTGGGCCAAGTCAGCAGGTGCCAAGTCTAGTGGTGGCA
GGGAAGACTTGGAGTCATCTGGACTGCAGAGAAGGAACTCCTCTGAAGCA
AGCTCCGGAGACTTCTTAGATCTGAAGGGAGAAGGTGATATTCATGAAAA
TGTGGACACAGACTTGCCAGGCAGCCTGGGGCAGAGTGAAGAGAAGCCCG
TGCCTGCTGCGCCTGTGCCCAGCCCGGTGGCCCCGGCCCAGTGCCATCCA
GAAGAAATCCCCCTGGCGGCAAGTCCAGCCTCGTCTTGGGTTAGCTCTGA
CTGTCCTGAACGCTGTCGTTCTGTCTGTTTCTCCATGCTTGTGAACTGGA
CAACTTGAGCCTGACTGTACATCTCTTGGTTTGTTTCATTAAAAGAAGCA
CTTTATGGTACTGGCTGGCCTTTTTTTCTTCCTTTCGGACCAACCGGGTT
CCTCTTCTGTCCCTGTACTCTGGGGTCCTGGGGGCAGGCTTGGTTTTTCT
CGTTGCTTGGGGCGCACAGCTTGTGTCACCTTTTCTGGTCTTTCCAGACC
ACATTCGGGCCCCGTGTTGGGCTCTGTCGCGGTGTACTTTTGGTTCTGCC
CCCGGACTAACCGGTCTCGGCCCCGGGTTCTCCCTGTGGTCTCCGGGGGC
CCCTTAAAGCGTCGGCAGTTTAAGGG
(CloneID 6061001)

SEQ ID NO:122

TGCTGTTCGGTGTGGAGTTTCCCAGCGCCCCTCGGCTCCGACCCTTTGAG
CGTTCTGCTCCGGCGCCAGCCTACCTCGCTCCTCGGCGCCATGACCACAA
CCACCACCTTCAAGGGAGTCGACCCCAACAGCAGGAATAGGTCCCGAGTT
TTGCGGCCTCCAGGTGGTGGATCCAATTTTTCATTAGGTTTTGATGAACC
AACACAACAACCTGTGAGGAAGAACAAAATGGCCTCTAATATCTTTGGGA
CACCTGAAGAAAATCAAGCTTCTTGGGCCAAGTCAGAAGGTGCCAAGTCT
AGTGGTGGCAGGGAAGACTTGGAGTCATCTGGACTGCAGAGAAGGAACTC
CTCTGAAGCAAGCTCCGGAGACTTCTTAGATCTGAAGGGAGAAGGTGATA
TTCATGAAAATGTGGACACAGACTTGCCAGGCAGCCTGGGGCAGAGTCAA
GAGAAGCCCGTGCCTGCTGCGCCTGTGCCCAGCCCGGTGGCCCCGGCCCC
AGTGCCATCCAGAAGAAATCCCCCTGGCGGCAAGTCCAGCCTCGTCTTGG
GTTAGCTCTGACTGTCCTGAACGCTGTCGTTCTGTCTGTTTCCTCCATG
CTTGTGAACTGCACAACTTGAGCCTGACTGTACATCTCTTGGATTTGTTT
CATTAAAAAGAAGCACTTTATGTACTGCTGCCTTTTTTTTTTTTCTTTTG
AAGAACAGGGTTCCTCTCTGTCCTTGACTCTTGGGGTCTGTGGGGCCATG
GCATGAGTGGTTTTCTAGTCAGAAGATTGGAAGGGAAGCTTTTGGGACAC
TTAACCACTGGGGTTTTTTAAGAAGAAATAATTTTGGTTCCCAGAATGTG
TTTACAAGGATCT
(CloneID 4604404)

SEQ ID NO:123

GAGCGGCGGCTCCTGCAGCGGTGGTCGGCTGTTGGGTGTGGAGTTTCCCA
GCGCCCCTCGGGTCCGACCCTTTGAGCGTTCTGCTCCGGCGCCAGCCTAC
CTCGCTCCTCGGCGCCATGACCACAACCACCACCTTCAAGGGAGTCGACC
CCAACAGCAGGAATAGCTCCCGAGTTTTGCGGCCTCCAGGTGGTGGATCC
AATTTTTCATTAGGTTTTGATGAACCAACAGAACAACCTGTGAGGAAGAA
CAAAATGGCCTCTAATATCTTTGGGACACCTGAAGAAAATCAAGCTTCTT
GGGCCAAGTCAGCAGGTGCCAAGTCTAGTGGTGGCAGGGAAGACTTGGAG
TCATCTGGACTGCAGAGAAGGAACTCCTCTGAAGCAAGCTCCGGAGACTT
CTTAGATCTGAAGGGAGAAGGTGATATTCATGAACCTGTGGACACAGACT
TGCCAGGCAGCCTGGGGCAGAGTGACGAGAAGCCCGTGCCTGCTGCCGCC
TGTGCCCCAGCCCGGTGGACCCGGCCCAGTGCATCCAGAAGAAAATCCCC
TGGCGGCACTCCAGCCTCGTACTTGGGTTAGCTCTGACTGTCTGAACGCT
GTCGTACTGTCTGTTTCTCCATGCTTGTGAACTGACAATTGAGGCTGACT
GTACTTCTCCTGGACTTGGGTTGCATTCAAAGAGGAAAGCACCTTTTATT
GTACTTGGATGGGTATCTATACTATACATATCGTTAGTGAAGAGACAGGG
GTTCATATTAGTGTACCTGGACTTTCTGGGGCATTGTGGCGCACTGGATT
GAGGTGTATACTAAGAATAAATGAGAGGAAACGGTTGTGACAAGTGTGAT
GGGATTAGAAGGAGACAATAGATGGGTCCAGAATT
(CloneID 4805717)

SEQ ID NO:124

GAGCGGCGGCTCCTGCAGCGGTGGTCGGCTGTTGGGTGTGGAGTTTCCCA
CCGCCCCTCGGGTCCGACCCTTTGAGCGTTCTGCTCCGGCGCCAGCCTAC
CTCCCTCCTCGGCGCCATGACCACAACCACCACCTTCAAGGGAGTCGACC
CCAACAGCAGGAATAGCTCCCGAGTTTTGCGGCCTCCAGGTGGTGGATCC
AATTTTTCATTAGCTTTTGATGAACCAACAGAACAACCTGTGAGGAAGAA
CAAAATGGCCTCTAATATCTTTGGGACACCTGAAGAAAATCAAGCTTCTT
GGGCCAAGTCAGCAGGTGCCAAGTCTAGTGGTGGCAGGAAGACTTGGAGT
CATCTGGACTGCAGAGAAGGAACTCCTCTGAAGCAAGCTCCGGAGACTTC
TTAGATCTGAAGGGAGAAGGTGATATTTATGAAAAAATGTGGACACAGAC
TTGCCAGGCAGCCTGGGGCAGAGTGAAGAGAGGCCCGTGCCTGCTGCGCC
TGTGCCCAGCCCGGTGGCCCCGGCCCCAGTGCCATCCAGAAGAAATCCCC
CTGGCGGCAAGTCCAGCCTCGTCTTGGGTTAGCTCTGACTGTCCTGAACG
CTGTCGTTCTGTCTGTTTCCTCCATGCTTGTGAACTGCACAACTTGAGCC
TGACTGTAC
(CloneID 4109734)

SEQ ID NO:125

GGTCCGACCCTTTGAGCGTTCTGCTCCGGCGCCAGCCTACCTCGCTCCTC
GGCGCCATGACCACAACCACCACCTTCAAGGGAGTCGACCCCAACAGCAG
GAATAGCTCCCGAGTTTGCGGCCTCCAGGTGGTGGATCCAATTTTTCATT
AGGTTTGATGAACCAACAGAACAACCTGTGAGGAAGAACAAAATGGCCTC
TAATATCTTTGGGACACCTGAAGAAAATCAAGCTTCTTGGGCCAAGTCAG
CAGGTGCCAAGTCTAGTGGTGGCAGGGAAGACTTGGAGTCATCTGGACTG
CAGAGAAGGAACTCCTCTGAAGCAAGCTCCGGAGACTTCTTAGATCTGAA
GGCAGAAGGTGATATTCATGAAAATGTGGACACAGACTTGCCAGGCAGCC
TGGGGCAGAGTGAAGAGAAGCCCGTGCCTGCTGCGCCTGTGCCCAGCCCG
GTGGCCCCGGCCCCAGTGCCATCCAGAAGAAATCCCCCTGGCGGCAAGTC
CAGCCTCGTCTTGGGTTAGCTCTGACTGTCCTGAACGCTGTCGTTCTGTC
TGTTTCCTCCATGCTTGTGAACTGACAACTTGAGCCTGACTGTACATCTC
TTGGATTTGTTTCACTTAAAAAGAAGCACTTTATGTACTGTGTCTTTTTT
TTTTCCTTTGGACGACCGGGTTTCTCTCTGCCTTGACTCTTGGGTCTGTG
GGCCCTGGCTGAGTGTTCTCATCATGGGGGGCC
(CloneID 4553496)

SEQ ID NO:126

GCTGCAGCGGTGGTCGGCTGTTGCGTGTGGAGTTTCCCAGCGCCCCTCGG
GTCCACCCTTTGAGCGTTCTGCTCCGGCGCCAGCCTACCTCGCTCCTCGG
CGCCATGACCACAACCACCACCTTCAAGGGAGTCGACCCCAACAGCAGGA
ATAGCTCCCGAGTTTTGCGGCCTCCAGGTGGTGGATCCAATTTTTCATTA
GGTTTTGATGAACCAACAGAAGAACCTGTGAGGAAGAACAAAATGGCCTC
TAATATCTTTGGGACACCTGAAGAAAATCAAGCTTCTTGGGCCAAGTCAG
CAGGTGCCAAGTCTAGTGGTGGCAGGGAAGACTTGGAGTCATCTGGACTG
CAGAGAAGGAACTCCTCTGAAGCAAGCTCCGGAGACTTCTTAGATCTGAA
GGGACGGAAGGTGATATTCATGAAAATGTGGACACAGACTTGCCAGGCAG
CCTGGGGCAGAGTGAAGAGAAGCCCGTGCCTGCTGCGCCTGTGCCCAGCC
CGGTGGCCCCGGCCCCAGTGCCATCCAGAAGAAATCCCCCTGGCGCCAAG
TCCAGCCTCGTCTTGGGTTAGCTCTGACTGTCCTGAACGCTGTCGTTCTG
TCTGTTTCCTCCATGCTTGTGAACTGGCACAAGTTGAGCCTGACTGTACA
TCTCTGGATTGGTGTCATTAAACGAGAAGCACTTTATGTACTGTGTCTTT
ATTTAGCATTTGAGAACAGGGTGCCTCTTGTCCCTGAATCTGGGTTGGGG
GCAGGATGAGTGTTACTATAGTAATGCGGGAGGTTGGGCACTAGACGGGT
TGAGGAAGAATATTGGGGCGATGTGTAAGGATTGGGAGGGGTAACAGTAG
GGCGTGACAGCGTAATGGAGGCTATAGTCAGGAGCTGGAGGCCACCAGAG
TGGGATTGGTATATGAGGTGAAGCTTGGGTGAAAGGCTCGGACGAAGTTA
GCAGAA
(CloneID 4156131)

SEQ ID NO:127

CGGACGGCGTGGGCGGCTGTGGGTGTGGAGTTTCCCAGCGCCCCTCGGGT
CCGACCCTTTGAGCGTTCTGCTCCGGCGCCAGCCTACCTCGCTCCTCGGC
GCCATGACCACAACCACCACCTTCAAGGGAGTCGACCCCAACAGCAGGAA
TAGCTCCCGAGTTTGCGGCCTCCAGGTGGTGGATCCAATTTTTCATTAGG
TTTGATGAACCAACAGAACAACCTGTGAGGAAGAACAAAATGGCCTCTAA
TATCTTTGGGACACCTGAAGAAAATCAAGCTTCTTGGGCCAAGTCAGCAG
GTGCCAAGTCTAGTGGTGGCAGGGAAGACTTGGAGTCATCTGGACGTGCA
GAGAAGGAACTCCTCTGAAGCAAGCTCCGGAGACTTCTTAGATCTGAAGG
GAGAAGGTGCTATTTATGAAAATGTGGACACAGACTTGCCAGGCAGCCTC
GGGCAGAGTGAAGAGAAGCCCGTGCCTGCTGCGCCTGTGCCCAGCCGGTG
GCCCCGGCCCAGTGCCATCCAGAAGAAATCCCCCTGGCGGAAGTCCAGCC
TCGTCTTGGGTTAGCTCTGACTGTCCTGAACGCTGTCGTTCTGTCTGTTT
TCCTCCATGCTTGTGAACTGGACAAATTTGAGCCTGATGGTACATCTCTT
TGGATTGGTTCATTAACCAAGAAGCACTTTATGTACGCNGGCCTAGATTG
CTGTGAGGACCGGTTCTCTCGTGTCCTGGACCTGGTCGGGGGGCCGGATG
AGGGGTCTAGAGGGATCGGGGGGAACCTGGGGACCTAGACGGGGTCGAAC
AACACTTCGGGCCCACTGGGTGGACAGCTTGGCGGGGGTAACCCAACTGG
GGCCCGCCCCGGGGACAAAAAGCAAGGCGGCGGCGCGACCCAGGGCCCCC
ACGTGGCAAGAGGGGGACCCCAAGAAAAGACCCCGGCGACAGTATTACAA
CCGAGG
(CloneID 5014564)

SEQ ID NO:128

GCTGCAGCGGTGGTCGGCTGTTCGGTGTGGAGTTTCCCAGCGCCCCTCGG
GTCCGACCCTTTGAGCGTTCTGCTCCGGCGCCAGCCTACCTCGCTCCTCG
GCGCCATGACCACAACACCACCTTCAAGGGAGTCGACCCCAACAGCACGA
ATAGCTCCCGAGTTTTGCGGCCTCCAGGTGGTGGATCCAATTTTTCATTA
GGTTTTGATGAACCAACAGAACAACCTGTGAGGAAGAACAAAATGGCCTC
TAATATCTTTGGGACACCTGAAGAAAATCAAGCTTCTTGGGCCAAGTCAG
CAGGTGCCAAGTCTAGTGGTGGCACGGAAGACTTGGAGTCATCTGGACTG
CAGACAAGGAACTCCTCTGAAGCAACCTCCGGAGACTTCTTAGATCTGAA
GGGAGAAGGTGATATTCATGAAAATGTGGACACAGACTTGCCAGGCAGCC
TGGGGCAGAGTGAAGAGAAGCCCGTGCCTGCTGCGCCTGTGCCCAGCCCG
GTGGCCCCGGCCCCAGTCGCATCCAGAAGAAATCCCCCTGGCGGCAAGTC
CAGCCTCGCTCTTGGGTTAGTCTGACTGTCCTGAACGCTGTCGTTCTGTC
TGTTTCCTCCATGCTTGTGAACTGCCACCACTTGAGCCTGACTGTACATC
TCTGTGGATCGTTTCATTAACNACCGACGCACTTTCATGTCACTGCGTGG
TCGTATTGCTTTCCCCCTCTGCGCAGCAACCGGGTTCCTCCTCGGGGCCC
TGAACCTCTTGGGACCGTGTGGCCCATGGCCTGAGCGTTCCTACTCGCAA
CACTCGCCGGCACACCATCGGCGCACCCGCACCGGCCTTCTCCCGCACAA
CACACTCGGCTCCCAGGTCTCCACGACCTCCATCCGGCCCCACACACACC
ATGCCGTCCCCACG
(CloneID 3458462)

SEQ ID NO: 129

CCCCAGCGCCCCTCGGGTCCGACCCTTTGAGCGTTCTGCTCCGGCGCCAG
CCTACCTCGCTCCTCGGCGCCATGACCACAACCACCACCTTCAAGGGAGT
CGACCCCAACAGCAGGAATAGCTCCCGAGTTTTGCGGCCTCCAGGTGGTG
GATCCAATTTTTCATTAGGTTTGATGAACCAACAGAACAACCTGTGAGGA
AGAACAAAATGGCCTCTAATATCTTTGGCACACCTGAAGAAAATCAAGCT
TCTTGGGCCAAGTCAGCAGGTGCCAAGTCTAGTGGTGGCAGGGAAGACTT
GGAGTCATCTGGACTGCAGAGAAGGAACTCCTCTGAAGCAAGCTCCGGAG
ACTTCTTAGATCTGAAGGGAGAAGGTGATATTCATGAAAATGTGGACACA
GACTTGCCAGGCAGCCTGGGGCAGAGTGAAGAGAAGCCCGTGCCTGCTGC
GCCTGTGCCCAGCCCGGTGGCCCCGGCCCCAGTGCCATCCAGAAGAAATC
CCCCTGGCGGCAAGTCCAGCCTCGTCTTGGGTTAGCTCTGACTGTCCTGA
ACGCTGTCGTTCTGTCTGTTTCCTCCATGCTTGTGAACTGACAATTGAGC
CTGACTGTACATCTCTGGGATTGTTTCATTTACAAAAGAAGCACTT
(CloneID 3910873)

SEQ ID NO:130

TCCCAGCGCCCCTCGGGTCCGACCCTTTGAGCGTTCTGCTCCGGCGCCAG
CCTACCTCGCTCCTCGGCGCCATGACCACAACCACCACCTTCAAGGGAGT
CGACCCCAACAGCAGGAATAGCTCCCGAGTTTTGCGGCCTCCAGGTGGTG
GATCCAATTTTTCATTAGGTTTTGATGAACCAACAGAACAACCTGTGAGG
AAGAACAAAATGGCCTCTAATATCTTTGGGACACCTGAAGAAAATCAAGC
TTCTTGGGCCAAGTCAGCAGGTGCCAAGTCTAGTGGTGGCAGGGAAGACT
TGGAGTCATCTGGACTGCAGAGAAGGAACTCCTCTGAAGCAAGCTCCGGA
GACTTCTTAGATCTGAAGGGAGAAGGTGATATTCATGAAAATGTGGACAC
AGACTTGCCAGGCAGCCTGGGGCAGAGTGAAGAGAAGCCCGTGCCTGCTG
CGCCTGTGCCCAGCCCGGTGGCCCCGGCCCCAGTGCCATCCAGAAGAAAT
CCCCCTGGCGGCAAGTCCAGCCTCGTCTTGGGTTAGCTCTGACTGTCCTG
AACGCTGTCGTTCTGTCTGTTTCCTCCATGCTTGTGAACTGCACAACTTG
AGCCTGACTGTACATCTCTTGGATTTGTTTCATTCAAAAAGAAGCACTT
(CloneID 5928362)

SEQ ID NO:131

CCTGCAGCGGTGGTCGGCTGTTGGGTGTGGAGTTTCCCAGCGCCCCTCGG
GTCCGACCCTTTGAGCGTTCTGCTCCGGCGCCAGCCTACCTCGCTCCTCG
GCGCCATGACCACAACCACCACCTTCAAGGGAGTCGACCCCAACAGCAGG
AATAGCTCCGGAGTTTTGCGGCCTCCAGGTGGTGGATCCAATTTTTCATT
AGGTTTTGATGAACCAACAGAACAACCTCTGAGGAAGAACAAAATGGCGT
CTAATATCTTTGGGACACCTGAAGAAAATCAAGCTTCTTGGGCCAAGTCA
GCAGGTGCCAAGTCTAGTGGTGGCAGGGAAGACTTGCAGTCATGTGGACT
GCAGAGAAGGAACTCCTCTGAAGCAAGCTCCGGAGACTTCTTAGATCTGA
AGGGAGAAGGTGATATTCATGAAAATGTGGACACAGACTTGCCAGGCAGC
CTGGGGCAGAGTGAAGAGAAGCCCGTGCCTGCTGCGCCTGTGCCCAGCCC
GGTGGCCCCGGCCCCAGTGCCATCCAGAAGAAATCCCCCTGGCGGCAAGT
CCAGCCTCGTCTTGGGTTAGCTCTGACTGTCCTGAACGCTGTCGTTCTG
TCTGTTTCCTCCATGCTTGTGAACTGCACAACTTGAGCCTGACTGTACAT
CTCTTGGATTTGTTTCATTAAAAAGAAGCACTTTAAAAAAAAAAAAANAA
AAANTAA
(CloneID 3904371)

SEQ ID NO:132

CCCAGCGCCCCTCGGGTCCGACCCTTTGAGCGTTCTGCTCCGGCGCCAGC
CTACCTCGCTCCTCGGCGCCATGACCACAACCACCACCTTCAAGGGAGTC
GACCCCAACAGCAGGAATAGCTCCCGAGTTTTGCGGCCTCCAGGTGGTGG
ATCCAATTTTTCATTAGGTTTTGATGAACCAACAGAACAACCTGTGAGGA
AGAACAAAATGGCCTCTAATATCTTTGGGACACCTGAAGAAAATCAAGCT
TCTTGGGCCAAGTCAGCAGGTGCCAAGTCTAGTGGTGGCAGGGAAGACTT
GGAGTCATCTGGACTGCAGAGAAGGAACTCCTCTGAAGCAAGCTCCGGAG
ACTTCTTAGATCTGAAGGGAGAAGGTGATATTCATGAAAATGTGGACACA
GACTTGCCAGGCAGCCTGGGGCAGAGTGAAGAGAAGCCCGTGCCTGCTGC
GCCTGTGCCCAGCCCGGTGGCCCCGGCCCCAGTGCCATCCAGAAGAAATC
CCCCTGGCGGCAAGTCCAGCCTCGTCTTGGGTTAGCTCTGACTGTCCTGA
ACGCTGTCGTTCTGTCTGTTTCCTCCATGCTTGTGAACTGCACAACTTGA
GCCTGACTGTACATCTCTTGGATTTCGTTTCATTAAAAAAGAAGC
(CloneID 4578473)

SEQ ID NO:133

GCAGCGGTGGTCGCCTGTTGGGTGTGGAGTTTCCCAGCGCCCCTCGGGTC
CGACCCTTTGAGCGTTCTGCTCCGGCGCCAGCCTACCTCGCTCCTCGGCG
CCATGACCACAACCACCACCTTCAAGGGAGTCGACCCCAACAGCAGGAAT
AGCTCCCGAGTTTTGCGGCCTCCAGGTGGTGGATCCAATTTTTCATTAGG
TTTTGATGAACCAACAGAACAACCTGTGAGGAAGAACAAAATGGCCTCTA
ATATCTTTGGGACACCTGAAGAAAATCAAGCTTCTTGGGCCAAGTCAGCA
GGTGCCAAGTCTAGTGGTGGCAGGGAAGACTTGGAGTCATCTGGACTGCA
GAGAAGGAACTCCTCTGAAGCAAGCTCCGGAGACTTCTTAGATCTGAAGG
GAGAAGGTGATATTCATGAAAATGTGGACACAGACTTGCCAGGCAGCCTG
GGGCAGAGTGAAGAGAAGCCCGTGCCTGCTGCGCCTGTGCCCAGCCCGGT
GGCCCCGGCCCCAGTGCCATCCAGAAGAAATCCCCCTGGCGGCAAGTCCA
GCCTCGTCTTGGGTTAGCTCTGACTGTCTGAACGCTGTCGTTCTGTCTGT
NTCCTCCATGCTTGTGAACTGCACAATTGAGCTGATGTCATCTCTTGGAT
TTGTTTCATTAAAAGGAGCACTTATGTACTGCTGGCTTTTTTTTCTTGGA
CACCCGCTCCCCCTGCCTTGCTCTGGGTCTGGGGCAGCCTGGAGGGTCCA
GTAGAATGGGGAGGAACTGCGACCTTGCGGTTTCGAACAACTCGGCCCTG
TTACGACTTGCCGGGGTACCTCCTGGGTCCGCCCTGGCAGAAGCAGGGGT
CGCGCCCGAGCCTTGGCACACGGAACCTAGAAAGGCTCGCAATTCACGCC
GAGGGTCGGGGGGCTGGG
(CloneID 4559434)

SEQ ID NO:134

GGCTCCTCCAGCGGTGGTCGGCTGTGGGTGTGGAGTTTCCCAGCGCCCCT
CGGGTCCGACCCTTTGAGCGTTCTGCTCCGGCGCCAGCCTACCTCGCTCC
TCGGCGCCATGACCACAACCACCACCTTCAAGGGAGTCGACCCCAACAGC
AGGAATAGCTCCCGAGTCTTGCGGCCTCCAGGTGGTGGATCCAATTTTTC
TTTTAGGTTTGATGAACCAACAGAACAACCTGTGAGGAAGAACAAAATGG
CCTCTAATATCTTTGGGACACCTGAAGAAAATCAAGCTTCTTGGGCCAAG
TCAGCAGGTGCCCAGTCTAGTGGTGGCAGGGAAGACTTGGAGTCATCTGG
ACTGCAGAGAAGGAACTCCTCTGAAGCAAGCTCCGGAGACTTCTTAGATC
TGAAGGGAGAAGGTGATATTCATGAAAATGTGGACACAGACTTGCCAGGC
AGCCTCGGGCAGAGTGAAGAGAAGCCCGTGCCTGCTGCGCCTGTGCCCAG
CCCGGTGGCCCGGCCCCAGTGCCATCCAGAAGAAATCCCCCTGGCGGCAG
TCCAGCCTCGTCTTGGGTTACCTCTGACTGTTCTGAACGCTGTCGTCTGT
CTGTTTCCTTCCATGCTGTGAAACTGCACAACTTGGAGCCCTGACGTGTA
CCTCTCTGGGATCGTTCCATTCACCAGAAGCACTTTTATGTGCTGCTGCT
ATCTTACTCCTGTCGGAGACAGGGTTCTCTCGTCCCTTGACTTCGGGGTC
GGGGGCCCGGGCCCTGTCTGCTTCCTGACACATTGGGGGCGAGAGTCTGT
GTCCCTGCGACGGGTTCAGACAGAATCGGGCCAGTCGGTGGAGCGTGGTC
GGGACAACTCGGGAACAGCCAGGAGAATAACGACGCTCGGGCGCCCCAGG
CCTCGACTGCGCCTCTCCCGCAACCCCAGACACGCCGCGGAA
(CloneID 4547048)

SEQ ID NO:135

GCGGGTCCGACCCTTTGAGCGTTCTGCTCCGGCGCCAGCCTACCTCGCT
CCTCGGCGCCATGACCACAACCACCACCTTCAAGGGAGTCGACCCCAAC
AGCAGGAATAGCTCCCGAGTTTCGCGGCCTCCAGGTGGTGGATCCAATT
TTTCATTAGGTTTTGATGAACCAACAGAACAACCTGTGAGGAAGAACAA
AATGGCCTCTAATATCTTTGGGACACCTGAAGAAAATCAAGCTTCTTGG
GCCAAGTCAGCAGGTGCCAAGTCTAGTGGTGGCAGGGAAGACTTGGAGT
CATCTGGACTGCAGAGAAGGAACTCCTCTGAAGCAAGCTCCGGAGACTT
CTTAGATCTGAAGGCGAGAAGGTGATATTCATGAAAATGTGGACACAGA
CTTGCCAGGCAGCCTGGGGCAGAGTGAAGAGAAGCCCGTGCTGCTGCGC
CTGTGCCCAGCCCGGTGGCCCCGGCCCCAGTGCCATCCAGAAGAAATCC
CCTGGCCGGCACAGTCCAGCCTCGTCTCGGGTTAGCTCTGACTGTCCTG
AACGCTGTACGTTCTGTCTGTTTCCTCCATGCTTGTGAACTGCACAAG
CTTGAGCCTGACTGTACATCTCTTGGATTTGTTTCATTAAAACGAAGC
(CloneID 4276268)

SEQ ID NO:136

GAGCGGCGGCTCCTGCAGCGGTGGTCGGCTGTTGGGTGTGGAGTTTCCCA
GCGCCCCTCGGGTCCGACCCTTTGAGCGTTCTGCTCCGGCGCCAGCCTAC
CTCGCTCCTCGGCGCCATGACCACAACCACCACCTTCAAGGGAGTCGAC
CCCAACAGCAGGAATAGCTCCCGAGTTTTGCGGCCTCCAGGTGGTGGATCC
AATTTTTCATTAGGTTTTGATGAACCAACAGAACAACCTGTGAGGAAGAA
CAAAATGGCCTCTAATATCTTTGGGACACCTGAAGAAAATCAAGCTTCTT
GGGCCAAGTCAGCAGGTGCCAAGTCTAGTGGTGGCAGGGAAGACTTGGAG
TCATCTGGACTGCAGAGAAGGAACTCCTCTGGAGCAAGCTCCGGAGACTT
CTTAGATCTGAAGGGAGAAGGTGATATTCATGAAAATGTGGACACAGACT
TGCCAGGCAGCCTGGGGCAGAGTGAAGAGAAGCCCGTGCTGCTGCGCCTG
TGCCCAGCCGCTGGCCCCGGCCCAGTGCCATCCAGAAGAAATCCCCCTGG
CGGCAAGTCCAGCCTCGTCTTGGGTTAGCTCTGACTGTCCTGAACGCTGT
CGTTCTGTCTGTTCCTCCATGCTTGTGAACTGCACAATTTGAGCCTGACT
GTCATCTCTGGGTTTGGTTCTTTAAACAGAAGCCTTTGTGCGCGGCTTCT
TTCTCTCGAGACCACCGTTCCCTGCGCTGGCCCGGGCTCGGGGCCGGCCA
GGGTTTCCCCCACTGGCGGCGCCCTCTGGCCCCCCGGTGTCTCCACACAA
CATGGCGCCCGGTAGGCTCGTCGGCGTACCCTCGTGGCCCCCGG
(CloneID 3659047)

SEQ ID NO:137

GTTGGGTGTGGAGTTTCCCAGCGCCCCTCGGGTCCGACCCTTTGAGCGTT
CTGCTCCGGCGCCAGCCTACCTCGTCTCCTCGGCGCCATGACCACAACCA
CCACCTTCAAGGGATGTCTGACCCCAACAGCAGGAATAGCTCCCGAGTTT
CGCGGCCTCCAGGTGGTGGATCCAATTTTTCATATAGGTTTTGATGAACC
AACAGAACAACCTGTGAGGAAGAACAAAATGGCCTCTAATATCTTGGGGA
CACCTGAAGAAAATCAAGCTTCTTGGGCCAAGTCATGCACGTGCCAAGTC
TACGTGGTGGCAGGGAAGACTTGGAGTCATCTGGACTGCAGAGAAGGAAC
TCCTCTGAAGCAAGCTCCGGAGACTTCTTAGATCTGAACGGGAGAAGGTG
ATATTCATGAAAATGTGGACACGAGACTTGCCAGTCCAGCCTGGGGCAGA
GTGAAGAGAAGCCCGTGCCTGCATGCGCCTGTGCCCAGCCCGGTGTGCCC
CGTGCCCAGTGCCATCCAGAAGAAATCCCCCGTGGCGGCAAGTCCAGCCT
CGTGATTGGGTTAGCTCTGACTGTCCTGAACGCTGTCGTACTGTCTTGTT
CCTCCATGCTTGTGAACTGCACAACTTGAGCCTGACTGTACA
(CloneID 3634373)

SEQ ID NO:138

GGTGTGGAGTTTCCCAGCGCCCCTCGGGTCCGACCCTTTGAGCGTTCTGC
TCCGGCGCCAGCCTACCTCGCTCCTCGGCGCCATGACCACAACCACCACC
TTCAAGGGAGTCGACCCCAACAGCAGGAATAGCTCCCGAGTTTTGCGGCC
TCCAGGTGGTGGATCCAATTTTTCATTAGGTTTTGATGAACCAACAGAAC
AACCTGTGAGGAAGAACAAAATGGCCTCTAATATCTTTGGGACACCTGAA
GAAAATCAAGCTTCTTGGGCCAAGTCAGCAGGTGCCAAGTCTAGTGGTGG
CAGGGAAGACTTGGAGTCATCTGGACTGCAGAGAAGGAACTCCTCTGAAG
CAAGCTCCGGAGACTTCTTAGATCTGAAGGGAGAAGGTGATATTCATCTT
TTCATAGAAAATGTCGACACAGACTTGCCAGGCAGCCTGGGGCAGAGTGA
AGACAAGCCCGTGCCTGCTGCGCCTGTGCCCAGCCCGGTGGCCCGGCCCC
AGTGCCATCCAGAAGAAATCCCCCTGGCGGCAAGTCCAGCCTCGTCTTGG
GTTAGCTCTGACTGTCCTGAACGCTGTCGTTCTGTCTGTTTCCTCCATGC
TTGTGAACTGCACAATTGAGCCTGACTGTGCTCTCTGGATTTGTTCCTTA
ACAGAGCCTTTTGTCTGG
(CloneID 5741030)

SEQ ID NO:139

CTCCTGCAGCGGTGGTCGGCTGTTGGGTGTGGAGTTTCCCAGCGCCCCTC
GGGTCCGACCCTTTGAGCGTTCTGCTCCGGCGCCAGCCTACCTCGCTCCT
CGGCGCCATGACCACAACCACCACCTTCAAGGGAGTCGACCCCAACAGCA
GGAATAGCTCCCGAGTTTTGCGGCCTCCAGGTGGTGGATCCAATTTTTCA
TTAGGTTTTGATGAACCAACAGAACAACCTGTGAGGAAGAACAAAATGGC
CTCTAATATCTTTGGGACACCTGAAGAAAATCAAGCTTCTTGGGCCAAGT
CAGCAGGTGCCAAGTCTAGTGGTGGCAGGGAAGACTTGGAGTCATCTGGA
CTGCAGAGAAGGAACTCCTCTGAAGCAAGCTCCGGAGACTTCTTAGATCT
GAAGGGAGAAGGTGATATTCATGAAAATGTGGACACAGACTTGCCAGGCA
GCCTGGGGCAGAGTGAAGAGAAGCCCGTGCCTGCTGCGCCTGTGCCCAGC
CCGGTGGCCCCGGCCCCAGTGCCATCCAGAAGAAATCCCCCTGGCGGCAA
GTCCAGCCTCGTCTTGGGTTAGCTCTGACTGTCCTGAACGCTGTCGTTCT
GTCTGTTTCCTCCATGCTTGTGAACTGCACAACTTGAGCCTGACTGTACA
TCTCTTGGATTTGTTTCATTAAAAAGAAGCACTTTAAAAAAAAAAAAAAA
A
(CloneID 3870232)

SEQ ID NO:140

ATCCCATGACGCCCCTCGGGTCCGACCCTTTGAGACGTTCTGCTCCGGAC
GCCAAGCCTACCTCGCATCCTCGGCGCCATGACCACAACCAACCACCTTC
AAGGGAGTCGACCCCAACAGCAGTGAATACGCTCCCGAGATTTTGCGGCC
TCCAGTGTGGTGGATCCAATTTTTCATTAGGTTTGTGATGAACCAACGAG
AACAACCTGTGAGGAAGAACAAAATGGCCTCTAATATCTTTGGGACACCT
GAAGAAAATCAAGCTTCTTGGGCCAAGTCAGCAGGTGCCAAGTCTAGTGG
TGGCAGGGAAGACTTGGAGTCATCTCGACTGCAGAGAAGGAACTCCTCTG
AAGCAAGCTCCGGAGACTTCTTAGATCTGAAGGGAGAAGGTGATATTCAT
GAAAATGTGGACACAGACTTGCCAGGCACCCTGGGGCAGAGTGAAGAGAA
GCCCGTGCCTGCTGCGCCTGTGCCCAGCCCGGTGGACCCGGGCCCAGTGC
CATCCAGAAGAAATCCCCCTGGCGGCAAGTCCAGCCTCGTCTTGGGTTAG
CTCTGACTGTCCTGAACGCTGTCGTTCTGTCTGTTCCTCCATGCTTGTGA
ACTGCACAACTTGAGCCTTGACTGTACATCTCTTGGACTAGGTTCACTTA
AAAGAAGCACTTTATGTACTGCTGTCTACAGAAAAACACCCCGGCAGACC
GGGTACCCCTACAGGACGTGCACCTTGGGCCAGGGGCCACGGCCAGCCGG
GCCACCAGATCCCACACTGGCAGGCAACCCTGCGACACACCACCACCACG
GGAAACACAACACACACAACAGGCGACCGCACGGTAAGAAGGACCTCAGG
CCACGAGCACACACACAAAACAGGGGGCACAAACCACACGTGGACGACCA
AACACCACAGCGACCGAAAGGCGGCCGACAACAGAGACACTCCCCCACAC
ACCGGGTGCCCCAACACCAAACGCATGACAAACACGTTGAACACACACAA
GCCCC
(CloneID 3905523)

SEQ ID NO:141

GTGTGGAGTTTCCCAGCGCGGCCTCGGGTCCGACCCTTTGAGCGTTCTGC
TCCGGCGCCAGCCTACCTCGCTCCTCGGCGCCATGACCACAACCACCACC
TTCAAGGGAGTCGACCCCAACAGCAGGAATAGCTCCCGAGTTTTGCGGCC
TCCAGGTGGTGGATCCAATTTTTCATTAGGTTTTGATGAACCAACAGAAC
AACCTGTGAGGAAGAACAAAATGGCCTCTAATATCTTTGGGACACCTGAA
GAAAATCAAGCTTCTTGGGCCAAGTCAGCAGGTGCCAAGTCTAGTGGTGG
CAGGGAAGACTTGGAGTCATCTGGACTGCAGAGAAGGAACTCCTCTGAAG
CAAGCTCCGGAGACTTCTTAGATCTGAAGGGAGAAGGTGATATTCATGAA
ACATGTGGACACAGACTTGCCAGGCAGCCTGGGGCAGAGTGAAGAGAAGC
CCGTGCCTGCTGCGCCTGTGCCCAGCACCGGGTGGCCCCGGCCCCAGTGC
CATCCAGAAGAAATCCCCCTGGCGGCAAGTCCAGCCTCGTCTTGGGTTAG
CTCTGACTGTCCTGAACCGCTGTCGTTCTGTCTGTTTCCTCCATGCCTTG
TGAACTGCACAATTGAGCCTGACTGTACCTCTCTTGGATCCGGTTGCCAT
GCACACAGAAGCGCTGGCGAAAAAGACACCGAGCCGACCACCGACGC
(CloneID 5517974)

SEQ ID NO:142

CGGACGCGTGGGCGGCTGTTGGGTGTGGAGTTTCCCAGCGCCCCTCGGGT
CCGACCCTTTGAGCGTTCTGCTCCGGCGCCAGCCTACCTCGCTCCTCGGC
GCCATGACCACAACCACCACCTTCAAGGGAGTCGACCCCAACAGCAGGAA
TAGCTCCCGAGTTTTGCGGCCTCCAGGTGGTGGATCAAATTTTTCATTAG
GTTTTGATGAACCAACAGAACAACCTGTGAGGAAGAACAAAATGGCCTCT
AATATCTTTGGGACACCTGAAGAAAATCAAGCTTCTTGGGCCAACTCAGC
AGGTGCCAAGTCTAGTGGTGGCAGGGAAGACTTGGAGTCATCTGGACTGC
AGAGAAGGAACTCCTCTGAAGCAAGCTCCGGAGACTTCTTAGATCTGAAG
GGAGAAGGTGATATTCATGAAAATGTGGACACAGACTTGCCAGGCACCCT
GGGGCAGAGTGAAGAGAAGCCCGTGCCTGCTGCGCCTGTGCCCAGCCCGG
TGGCCCCGGCCCCAGTGCCATCCAGAAGAAATCCCCCTCGCGGCAAGTCC
AGCCTCGTCTTGGGTTAGCTCTGACTGTCCTGAACGCTGTCGTTCTGTCT
GTTTCCTCCCATGCTTGTGAACTGCACAACTTGAGCCTGACTGTACATCT
CTTGGATTTGTTTCATTAAAAAGAAGCACTTTATGCACTGCTGTCTTTTT
TTTTTTCTTTTGAAGAACAGGTTTCTCTCTGGCCTTGACTCTTGGGGTCT
GTGGGCCCTGGCATGAGTGTTTTCCTAGAATAGATTGGAGGGAAAGCTTT
GTGACACTTAATACTGTGGTTTCAAAAAAAAAATAATTTGGTTCCCAATG
TGTTAAAAGGAACTTTTGGTACTGGAGGTTTTTAAAACCTTTAATTTGGG
TTTAACCAAGGCCTAACCTGGTACGAACCTTAAAACGTGTCCCACGGGCC
CCCGTTTCCTTGGCACGGGCCCAAACCCCACCGGGGGAGGCCTCTCTCAC
CAAAAACCCTTC
(CloneID 4124775)

SEQ ID NO:143

GCTCCGGCGCCAGCCTACCTCGCTCCTCGGCCCC
ATGACCACAACCACCACCTTCAAGGGAGTCGACCCCAACAGCAGGAATAG
CTCCCGACTTTGCGGCCTCCAGGTGGTGGATCCAATTTTTCATTAGGTTT
GATGAACCAACAGAACAACCTGTGAGGAAGAACAAAATGGCCTCTAATAT
CTTTGGGACACCTGAAGAAAATCAAGCTTCTTGGGCCAAGTCAGCAGGTG
CCAAGTCTAGTGGTGGCAGGGAAGACTTGGAGTCATCTGGACTGCAGAGA
AGGAACTCCTCTGAAGCAAGCTCCGGAGACTTCTTAGATCTGAAGGGAGA
AGGTGATATTCATGAAAATGTGGACACAGACTTGCCAGGCAGCCTGGGGC
AGAGTGAAGAGAAGCCCGTGCCTGCTGCGCCTGTGCCCAGCCCGGTGGCC
CCGGCCCCAGTGCCATCCAGAAGAAATCCCCCTGGCGGCAAGTCCAGCCT
CGTCTTGGGTTAGCTCTGACTGTCCTGAACGCTGTCGTTCTGTCTGTTT
CCTCCATGCTTGTGAACTGCACAACTTGAGCCTGACTGTACATCTCTTGG
ATTTGTTTCATTAAAAGAAGCACTTTATGTACTGATGTACTTCTTATTTT
TCCTTTGAAAGAACCAGGTTTCTCTCTGTCCCTGATCTGGTCTGTGGCCC
ATGGCACCGAGTGTTCCTTGTAGTACGATTGCGAGGGCAAGCTTGTGAAC
TATAGTACGGGTTTACGACGAACAATATCTGGCTCCCCAGGTCTCCACGA
CCTTGACGGGCGTTTCACACCTACTGGGTTCCCAGCTAGTGGCAGCACAA
ACGCACAGCCCGGTCGCCAGCCACCCAGAGTTCGGGACCCTGGTGTGCTA
TGCAGGCTGGGACACCCTGGATGTCCAAGGGGCCTGCTGCGGAAAGGTTT
TATTAGCTAAG
(CloneID 5832873 in 3′ to 5′ orientation)

SEQ ID NO:144

TTTTTTTTTTTTTTTTTTAAAGTGCTTCTTTTTAATGAAACAAATCCAAG
AGATGTACAGTCAGGCTCAAGTTGTGCAGTTCACAAGCATGGAGGAAACA
GACAGAACGACAGCGTTCAGGACAGTCAGAGCTAACCCAAGACGAGGCTG
GACTTGCCGCCAGGGGGATTTCTTCTGGATGGCACTGGGGCCGGGGCCAC
CGGGCTGGGCACAGGCGCACCAGGCACGGGCTTCTCTTCACTCTGCCCCA
GGCTGCCTGGCAAGTCTGTGTCCACATTTTCATGAATATCACCTTCTCCC
TTCAGATCTAAGAAGTCTCCGGAGCTTGCTTCAGAGGAGTTCCTTCTCTG
CAGTCCAGATGACTCCAAGTCTTCCCTGCCACCACTAGACTTGGCACCTG
CTGACTTGGCCCAAGAAGCTTGATTTTCTTCAGGTGTCCCAAAGATATTA
GAGCCCATTTTGTTCTTCCTCACAGGTTGTTCTGTTGGTTCATCAAAACC
TAATGAAAAATTGGATCCACCACCTNGGAGGCCGCAAAACTCGGGAGCTA
TTCCTGCTGTTGGGGTCCACTCCCTTGAAGGTGGTGGTTGTGGTCATGGC
GCCGAGGAGCGAGGT
(CloneID 4277604)

SEQ ID NO:145

GAGCGGCGGCTCCTGCAGCGGTGGTCGGCTGTTGGGTGTGGAGTCTCCCA
GCGCCCCTCGGGTCCGACCCTTTGACCGTTCTGCTCCGGCGCCAGCCTAC
CTCGCTCCTCGGCGCCATGACCACAACCACCACCTTCAAGGGAGTCGACC
CCAACAGCAGGAATAGCTCCCGAGTTTGGCGGCCTCCAGGTGGTGGATCC
AATTTTTCATTAGGTTTGATGAACCAACAGAACAACCTGTGAGGAAGAAC
AAAATGGCCTCTAATATCTTTGGGACACCTGAAGAAAATCAAGCTTCTTG
GGCCAAGTCAGCAGGTGCCAAGTCTAGTGGTGCCAGGAAGACTTGGAGTC
ATCTGGGACTGCAGAGAAGGAACTCCTCTGAAGCAAGCTCCGGAGACTTC
TTAGATCTGAAGGGAGAAGGTGATATTCATGAAAATGTGGACACAGACTT
GCCAGGCAGCTGGGGCAGAGTGAAGAGAAGCCGTGGCTGCTGCCCCTGTG
CCCAGCCGGTGGCCCGGGCCCAGTGCATCCAGAAGAAATCCCCTGGGGAA
GTCCAGCCTCGTTTTGGGTTAGTCTGATGTCCTGAAGCTCTCGTTCTGTC
TGTTCCTCAAGGTTGTGACTGACATTGAGCTGATGTACTTTCTGGATTGT
TCATTAAAAGAGCTTTTGAAAAAAAAAAAAAAAAAATTTGGGGGCCGGCC
CGAAATTTAACATGGGGGGGGGCCGGGTAGAATGGACGGCAGAAACGGGA
AGGGGGTCACAAAACGGGGAGGTGGAACGGGGGGTAGGTAAGGCGTACAG
(CloneID 5834676 in 3′ to 5′ orientation)

SEQ ID NO:146

TTTTTTTTTTTTTTTTTTAAAGAGCTTCTTTTTAATGAAACAAATCCAAG
AGATGTACAGTCAGGCTCAAGTTGTGCAGTTCACAAGCATGGAGGAAACA
GACAGAACGACAGGGTTCAGGACAGTCAGAGCTAACCCAAGACGAGGCTG
GACTTGCCGCCAGGGGGATTTCTTCTGGATGGCACTGGGGCCGGGGCCAC
CGGGCTGGGCACAGGCGCAGCAGGCACGGGCTTCTCTTCACTCTGCCCCA
GGCTGCCTGGCAAGTCTGTGTCCACATTTTCATGAATATCACCTTCTCCC
TTCAGATCTAAGAAGTCTCCGGAGCTTGCTTCAGAGGAGTTCCTTCTCTG
CAGTCCAGATGACTCCAAGTCTTCCCTGCCACCACTAGACTTGGCACCTG
CTGACTTGGCCCAAGATGCTTGATTTTCTTCAGGTGTCCCAAAGATATTA
GAGGCCATTTTGTTCTTCCTCACACGTTGTTCTGTTGGTTCATCAAAACC
TAATGAAAAATTGGATCCACCACCTGGCAGGCCGCAAAACTCGGGAGCTA
TTCCTGCTGTTGGGGTCGACTCCCTTGAAGGTGGTGGTTGTGGTCATGGC
GCCGAGGAGCCAG
(CloneID 5405407)

SEQ ID NO:147

GCGGTGGTCGGCTGTTGGGTGTGGAGTTTCCCAGCGCCCCTCGGGTCCGA
CCCTTTGAGCGTTCTGCTCCGGCGCCAGCCTACCTCGCTCCTCGGCGCCA
TGACCACAACCACCACCTTCAAGGGAGTCGACCCCAACAGCAGGAATAGC
TCCCGAGTTTTGCGGCCTCCAGGTGGTGGATCCAATTTTTCATTAGGTTT
TGATGAACCAACAGAACAACCTGTGAGGAAGAACAAAATGGCCTCTAATA
TCTTTGGGACACCTGAAGAAAATCAAGCTTCTTGGGCCAAGTCAGCAGGT
GCCAAGTCTAGTCGTGGCAGGGAAGACTTGGAGTCATCTGGACTGCAGAG
AAGGAACTCCTCTGAAAGCAACGCTTCGGAAGACTTCCTTAAAATTCCGA
CAGGACAACGGGAGAATATCACAGAGAAATCTGTGGCACACAAGATTGGC
CAGGGCACCCGTGGGGCAGATGTGAAGAGAAGCCCGTGCCTGCTGCGCCT
GTGCCCAGCCCGGGTGGCCCCGGTCCCAGTTGCCATCCAGAAGAGATCCC
CCTGGCGGCAAGTCCCAGCCTCGTCTTGGGTTAGCTCTGACTGGTCCTGA
ACCGCTGTCGTTCTGTCTGTGTCCTCCATGCTTGTGACAGTGCACACACT
CGAGCCTGACTGTACATCTCTCGGATTCGTTACATTCAGAAACAACGCAC
TTCTATGTACTGCCTGTCCTTTCTTTCCTTCGCTTTTGGCAGGACCCTGG
GTTTCTCTCCTGCCCCTCCACTCTGGGCCTCCGCTGGCCCCAGGCCCTGT
GCTGTCTCCCTACGGCCTCCGCATGCCAGCGATTCGCCTGGTCGTGACCC
TTAGGCCTGGCGTTTTACCCCCC
(CloneID 4563923)

SEQ ID NO:148

GCTGCAGCGGTGGTCGGCTGTTGGGTGTGGAGTTTCCCAGCGCCCCTCGG
GTCCGACCCTTTGAGCGTTCTGCTCCGGCGCCAGCCTACCTCGCTCCTCG
GCGCCATGACCACAACCACCACCTTCAAGGGAGTCGACCCCAACAGCAGG
AATAGCTCCCGAGTTTTGCGGCCTCCAGGTGGTGGATCCAATTTTTCATT
AGNGTTTTGNATGAACCAANCAGAACAACCTGTGAGGAAGAACAAAATGG
CCTCTAATATCTTTGGGACACCTGAAGAAAATCAAGCTTCTTGGGCCAAG
TCAGCAGGTGCCAAGTCTAGTGGTGGCAGGGAAGACTTGGAGTCATCTGG
ACTGCAGAGAAGGAACTCCTCCCTGAAGCAAGCTCCGGAGACTTCTTAGA
TCTGAAGGGAGAAGGTGATATTCATGAAAATGTGGACACAGACTTGCCAG
GCAGCCTGGGGCAGAGTGAAGAGAAGCCCGTGCCTGCTGCGCCTGTGCCC
AGCCCGGTGGCCCCGGCCCCAGTGCCATCCAGAAGAAATCCCCCTGGCGG
CAAGTCCAGCCTCGTCTTGGGTTAGCTCTGACTGTCCTGAACGCTGTCGT
TCTGTCTGTTTCCTCCAT
(CloneID 5533034)

SEQ ID NO:149

CGGCTGTTGGGTGTGGAGTTTCCCAGCGCCCCTCGGGTCCGACCCTTTGA
GCGTTCTGCTCCGGCGCCAGCCTACCTCGCTCCTCGGCGCCATGACCACA
ACCACCACCTTCAAGGGAGTCGACCCCAACAGCAGGAATAGCTCCCGAGT
TTTGCGGCCTCCAGGTGGTGGATCCAATTTTTCATTAGGTTTTGATGAAC
CAACAGAACAACCTGTGAGGAAGAACAAAATGGCCTCTAATATCTTTGGG
ACACCTGAAGAAAATCAAGCTTCTTGGGCCAAGTCAGCAGGTGCCAAGTC
TAGTGGTGGCAGGGAAGACTTGGAGTCATCTGGACTGCAGAGAAGGAACT
CCTCTGAAGCAAGCTCCGGAGACTTCTTAGATCTGAAGGGAGAAGGTGAT
ATTCATGAAAATGTGGACACAGACTTGCCAGGCAGCCTGGGGCAGAGTGA
AGAGAAGCCCGTGCCTGCTGCGCCTGTGCCCAGCCCGGTGGCCCCGGCCC
CAGTGCCATCCAGAAGAAATCCCCCTGGCGGCAAGTCCAGCCTCGTCTTG
GGTTAGCTCTGACTGTCCTGAACGCTGTCGTTCTGTCTGTTTCCTCCATG
CTTGTGAACTGCACAACTTGAGCCTGACTGTACATCTCTTGGATTTGTTT
CATTAAAAAGAAGCACTTTATGTACTGCTGTCTTTTTTTTTTCTTTTGAA
GAACAGGGTTTCTCTCTGGCCTTTGACTCTTGGGGTCTGTGGGGCCATGG
CAATGAATGGTTTTCCTAAGTAGTAAGAATTTGGAGGGAAAAGCTTTTGT
TGACCCCTTAATCAACTGGGGTTTTTTAAGAACGAAAATTAATTTGGGGG
TCCCACAAATGGTGTTTAAAAAGGAATCTTTTTTGGGACCTGGGGGGGGT
TTTTTTAACCCCCTTTTTACTTTGGGGGGTTTTTACCCAAGGCCCTTCAA
A
(CloneID 6060969)

SEQ ID NO:150

AGCGGTGGTCGGCTGTTGGCTCTGGAGTTTCCCAGCGCCCCTCGGGTCCG
ACCCTTTGAGCGTTCTGCTCCGGCGCCAGCCTACCTCGCTCCTCGGCGCC
ATGACCACAACCACCACCTTCAAGGGAGTCGACCCCAACAGCAGGAATAG
CTCCCGAGTTTTGCGGCCTCCAGGTGGTGGATCCAATTTTTCATTAGGTT
TTGATGAACCAACAGAACAACCTGTGAGGAAGAACAAAATGGCCTCTAAT
ATCTTTGGGACACCTGAAGAAAATCAAGCTTCTTGGGCCAAGTCAGCAGG
TGCCAAGTCTAGTGGTGGCAGGGAAGACTTGGAGTCATCTGGACTGCAGA
GAAGGAACTCCTCTGAAGCAAGCTCCGGAGACTTCTTAGATCTGAAGGGA
GAAGGTGATATTCATGAAAATGTGGACACAGACTTGCCAGGCAGCCTGGG
GCAGAGTGAAGAGAAGCCCGTGCCTGCTGCGCCTGTGCCCAGCCCGGTGG
CCCCGGCCCCAGTGCCATCCAGAAGAAATCCCCCTGGCGGCAAGTCCAGC
CTCGTCTTGGGCTAGCTCTGACTGTCCTGAAACGCTGTCGNTCTGTCTG
TTTCCTCCATGCTTGTGAACTGCACAACTTGAGCCTGACTGTACATCTCT
TGGATTTGTTTCATTAAAAAAGAAGCACTTTATGTACTGCTGTCTTTTTT
TTTTTTTTCCTTTGAAGAACAGGTTTTCTCTCTGTCCTTTGACTCTTGGG
GTCTGTGGGCCATGGGCATGAATTGTTTTTCTAGTAGTAGAATTGGAGGG
AAAAGCTTTTGGGACACTTAANACTGGGGTTTTTTAAGAAGAAAATAAT
(CloneID 3347594)

SEQ ID NO:151

TGGAGTTTCCCAGCGCCCCTCGGGTCCGACCCTTTGAGCGTTCTGCTCCG
GCGCCAGCCTACCTCGCTCCTCGGCGCCATGACCACAACCACCACCTTCA
AGGGAGTCGACCCCAACAGCAAATAGCTCCCGAGTTTTGCGGCCTCCAGG
TGGTGGATCCAATTTTTCATTAGGTTTTGATGAACCAACAGAACAACCTG
TGAGGAAGAACAAAATGGCCTCTAATATCTTTGGGACACCTGAAGAAAAT
CAAGCTTCTTGGGCCAAGTCAGCAGGTGCCAAGTCTAGTGGTGGCAGGGA
AGACTTGGAGTCATCTGGACTGCAGAGAAGGAACTCCTCTGAAGCAAGCT
CCGGAGACTTCTTAGATCTGAAGGGAGAAGGTGATATTCATGAAAATGTG
GACACAGACTTGCCAGGCAGCCTGGGGCAGAGTGAAGAGAAGCCCGTGCC
TGCTGCGCCTGTGCCCAGCCCGGTGGCCCCGGCCCCAGTGCCATCCAGAA
GAAATCCCCCTGGCGGCAAGTCCAGCCTCGTCTTGGGTTAGCTCTGACTG
TCCTGAACGCTGTCGTTCTGTCTGTTTCCTCCATGCTTGTGAACTGCACA
A
(CloneID 3531894)

SEQ ID NO:152

TGTCGGTGTGGAGTTTCCAGCGCCCCTCGGGTCAGACCCTTTGAGCGTTC
TGCTCCGGCGCCAGTCTACCTCGCTCCTCGGCGCCATGACCACAACCACC
ACCTTCAAGGGAGTCGACCCCAACAGCAGGAATAGCTCCCGAGTTATGCG
GCCTCCAGGTGGTGGATCCAATTTTTCATTAGGTTTTGATGAACCAACAG
AACAACCTGTGAGGAAGAACAAAATCGCCTCTAATATCTTAGGGACACCT
GAAGAAAATCAAGCTTCTTGGGCCAAGTCAGCAGGTGCCAAGTCTAGTGG
TGGCAGGGAACGACTTGGAGTCATCTGGACTGCAGAGAACGGAACTCCTC
TGAAGCAAGCTCCGGAGACTTCTTAGATCTGAAGGGAGAAGGTGATATTC
ATGAAAATGTGGACACAGACTTGCCAGGCAGCCTGGGGCAGAGTGAAGAG
AAGCCCGTGCCTGCTGCGCCTGTGCCCAGCCCGGTGGCCCCGGGCCAGTG
CCATCCAGAAGAAATCCCCCTGGCGGAAGTCCAGCCTCGTCTTGGGTTAG
CTCTGACTGTCCTGAACGCTGTCGTTCTGTCTGTTTACTCCATGCTTGTG
AACTGACCATCCGAGACTGAATG
(CloneID 4276267)

SEQ ID NO:153

GAGGGGGGCGTCCTGTAGGGTGGTCGGTGTTGGGTGTGGAGTGTCCCAGC
GCCCCTCGGGTCCGACCCTTGAGCGTCTGCTCCGGGCCAGCTACCTCGCT
CCTCGGCGCCATGAGCACAATCCAGCACCTTCAAGGGAGTCGACCCCAAC
AGCAGGAATAGCTCCCGAGTTTTGCGGCTCCAGGTGGTGGATCCAATTTT
TCATTAGGTGTGATGAACCAACAGAACAACCTGTGAGGAAGAACAAAATG
GGCTCTAATATCTTTGGGACACCTGAAGAAAATCAAGCTTCTTGGGCCAG
TCAGCAGGTGCCAAGTCTAGTGGTGGCAGGGAAGACTTGGAGTCATCTGG
ACTGCAGACGAAGGAACTCCTCTGGAAGCAAGGCTCCGGAGACTTCTTAG
ATCTGAAGGGAAGAAGGTGATATTCATGAACAATGTGGACACAGACTTGT
CCAGGCACGCCTGGGGCAAGAGTGAAGAGAAGCCCGTGCTTGTGGGCTGT
GCCATCCGGTGGGCCCGGCTCCAGTGCTATCCAGAAGAAATCCCCCTGGG
GGGAAGTCCAGCCTCGTCTTGGGGTTAGCTCTGACTGTCCTGAACGCTGT
CCGTCCTGTCGTGGTTACATCCATGCTTGTTGAACTTGGACCAACTTGGA
GTCTGATGGGAGATCTCCTGGATTGGTTCATTAACCAAAACGCGGTTAGG
GAATGGCCGGCCTTGGGGGAGTCTTCTGACAGACGAAGGTTCACCACTGG
GCGAAATTCGGGGGTCGGGGGACAGGAACACAGGGTCCACAGGCAATGGG
GCAGACCCATTGTC
(CloneID 4547025)

SEQ ID NO:154

GGCGGGTCCGACCCTTTGAGCGTTCCTGCTCCGGCGCCAGCCTACCTCGC
TCCTCGGCGCCATGACCACAACCACCACCTTCAAGGGAGTCGACCCCAAC
AGCAGGAATAGCTCCCGAGTTTGCGGCCTCCAGGTGGTGGATCCAATTTT
TCATTAGGTTTTGATGAACCAACAGAACAACCTGTGAGGAAGAACAAAAT
GGCCTCTAATATCTTTGGGACACCTGAAGAAAATCAAGCTTCTTGGGCCA
AGTCAGCAGGTGCCAAGTCTAGTGGTGGCAGGGAAGACTTGGAGTCATCT
GGACTCCAGAGAAGGAACTCCTCTGAAGCAAGCTCCGGAGACTTCTTAGA
TCTGAAGGGAGAAGGTGATATTCATGAAAATGTGGACACAGACTTGCCAG
GCAGCCTGGGGCAGAGTGAAGAGAAGCCCGTGCCTGCTGCGCCTGTGCCC
AGCCCGGTGGCCCGGCCCCAGTGCCATCCAGAAGAAATCCCCCTGGCGGC
AAGTCCAGCTCGTTCTTGCGTTAGCTCTGACTGTCTGAACGCTGTTCGTT
CTGTACTTCTTTCCTCCATGCTTGTGAACTCCACAACTTGAGCTCACTGT
ACGATCTCTAGGATCTGTCTCATTAAAAAGAAGCCACTTATGTACTGGTG
TCTTTTTCACCTTAGCAAAAACAGGTTCCCTCAGGCCTTGACCACTTGAG
GTCTGGAGCCAAGCGCATGAAAGGTCCCTAGGCAGCCGATCGGAGGGAAA
CGCCTGCGACCTTACTACCGCATCTCACACGACACAAGTGCCGTCCGACC
TGTCAAAGCACACGTGACCGAGGTCCAACCCATACCGGTTGCCGCCTAAT
TGCAGACCAAACGCCCCGACCAGCGCGCCCACACGGACTCGACACTGCTG
CAAGGCGACCGCAACCCCTCACACAAACCCCCCTCCCAACAGATATCCAG
CGAACCCACCACGCAGCAAAACGTCCATCACAAAACGTC
(CloneID 5314132)

SEQ ID NO:155

AGCGGAGCGCCGGCTCCTGCACCGGTGGTCCGCTGTCGGTGTGGAGTTTC
CCACCGCCCCTCGGGTCCGACCCTTTGAGCGTTCTGCTCCGGCGCCAGCC
TACCTCGCTCCTCGGCGCCATGACCACAACCACCACCTTCAAGGGAGTCG
ACCCCAACAGCAGGAATAGCTCCCGAGTTTTGCGGCCTCCAGGTGGTGGA
TCCAATTTTTCATTAGGTTTGATGAACCAACAGAACAACCTGTGAGGAAG
AACAAAATGGCCTCTAATATCTTTGGGACACCTGAAGAAAATCAAGCTTC
TTGGGCCAAGTCAGCAGGTGCCAAGTCTAGTGGTGGCAGGGAAGACTTGG
AGTCATCTGGACTGCACAGAAGGAACTCCTCTGAAGCAAGCTCCGGAGAC
TTCTTAGATCTGAAGGGAGAAGGTGATATTCATGAAAATGTGGACACAGA
CTTGCCAGGCAGCCTGGGGCAGAGTGAAGAGAAGCCCGTGCCTGCTGCGC
CTGTGCCCAGCCCGGTGGCCCCGGCCCCAGTGCCATCCAGAACAAATCCC
CCTGGCGGCAAAGTCCCAGCCTCGTCTTGGGTTAGCTCTGACTGTCCTGA
ACCGCTGTCCGTTCCTGTCCTGGTTTCCTCCCATGCTTTTTTTTTCTCGT
CATCTCGTACGTGAAGGGTGACAGTCACCGACAATACCGTCCGCTGCCGA
GAAGACCTCAACGCGACGGAGTAACACAACATCAGCATATACCAATGAAC
CTTCCCGATGGCGTCAGACTCTCCACGCGACATATACTAGA
(CloneID 5576026)

SEQ ID NO:156

| | |
|---|---|
| CTCCTGCAGCGGTGGTCGGCTGTTGGGTGTGGAGTTTCCCAGCGCCCCTC | |
| GGGTCCGACCCTTTGAGCGTTCTGCTCCGGCGCCAGCCTACCTCGCTCCT | |
| CGGCGCCATGACCACAACCACCACCTTCAAGGGAGTCGACCCCAACAGCA | |
| GGAATAGCTCCCGAGTTTTGCGGCCTCCAGGTGGTGGATCCAATTTTTCA | |
| TTAGGTTTTGATGAACCAACAGAACAACCTGTGAGGAAGAACAAAATGGC | |
| CTCTAATATCTTTGGGACACCTGAAGAAAATCAAGCTTCTTGGGCCAAGT | |
| CAGCAGGTGCCAAGTCTAGTGGTGGCAGGGAAGACTTGGAGTCATCTGGA | |
| CTGCAGAGAAGGAACTCCTCTGAAGCAAGCTCCGGAGACTTCTTAGATCT | |
| GAAGGGAGAAGGTGATATTCATGAAAATGTGGACACAGACTTGCCAGGCA | |
| GCCTGGGGCAGAGTGAAGAGAAGCCCGTGCCTGCTGCGCCTGTGCCCAGC | |
| CCGGTGGCCCCGGCCCCAGTGCCATGCAGAAGAAATCCCCCTGGCGGCAA | |
| GTCCAGCCTCGTCTTGGGTTAGCTCTGACTGTCCTGAACGCTGTCGTTCT | |
| GTCTGTTTCCTCCATGCTTGTGAACTGCCCAACTTGAGCCTGACTGTACA | |
| TCTCTTGGATTTGTTTCATTAAAAAGAAACACTTTATGTACTGCTGGCCT | |
| TTTTTTTTTTCCTTTTGGAAAACAGGTTTCCTCCTCTGTCCTTGAACTCC | |
| TGGGTCTTGGGGGCCCAGGGCCTGAAGGGGTTTTAATTATCAAACTTGGA | |
| | GGGAAAACCTTTTGGACACTCTTTACTTGGGCCTCTTCACAAAACACCA |
| CCTCGGTCCCCACAAGGGGTTCGACGGCCTCTTTTGTCACGAAGGTTTCC | |
| CCAATTTAATATTTGGGCGTTCCATAAACCTCCATTGGCGACGGCCCAAT | |
| ACACCGGCCATACGGCCGCCGCTCCTTGTCTAATCCCGCCCCCGGATGGG | |
| AGGCTCTTCTTCCAGAGGCCCTCATTGGGGCGGCCCCCGCTCTATCACAG | |
| C | |
| (CloneID 5165195) | |

SEQ ID NO:157

GCAGGACAGGCGGCAGCGGCGGCTCCTGCAGCGGTGGTCTGGCTGTTGGG
TGTGGAGTTGCGGAGCGCCCCTCGGGTCCGAGCCTTTGAGCTGTTCTGCT
CCGGATGCCAGCCTACCTCGCTCCTCGGCGCCATGACCACAACCACCACC
TTCAAGGGAGTCAGAACCCAACAGTCAGCAATAGCTCCCGAGTTTTGCGC
GCCTCCAGGTGTGTGGATCCAATTTTTCATTAGGTTTTGATGAACCAACA
GAACAACCTGTGACGAAGAACAAAATGGTCTCTAATATCTTTGGGACACC
TGAAGAAAATCAAGCTTCTATGGGCCAAAGTCACGCAGGTGCCAAGTCTA
GTGGTGGCAGGGAAGACTTGGAGTCATCTGGACTGCAGAGAAGGAACTCC
TCTCAAGCAAGCATCCGGAGACTTCTTAGATCTGAAGGGAGAAGGTGACT
ATTCATGAAAATGTGGACACAGACTTGCCAGGCAGCCTGGGGCAGAGTGA
AGAGAAGCCCGTGCCTGCTGCGCCTGTGCCCAGCCCTGGTGGACCCTGTG
CCCAAGTGCCATCCAGAAGAAATCCCCCTGGCGGCAAGTCCAGAACTCGT
CTTGGGTAAGCTCTGAACTGTACCTGAACCGCTGTCGTTCTGTCTGTATC
CTCCATGCTTGTGAACTGCACAACTTGAGCCTGACGCTGTACATTCTCCT
GGGGATTTCGCTTCCCACTTAAAAAAGACGCCACTTCACTGTTAGCAGCG
AAAAAAGGCAGCAGCGAGAACAGAACAAACAATTGGTGAG
(CloneID 3449557)

SEQ ID NO:158

GTTGGGTGTGGAGTTTCCCAGCGCCCCTCGGGTCCGACCCTTTGAGCGTT
CTGCTCCGGCGCCAGCCTACCTCGCTCCTCGGCGCCATGACCACAACCAC
CACCTTCAAGGGGAGTCGACCCCAACAGCAGGAATAGCTCCCGAGTTTTG
CGGCCTCCAGGTGGTGGATCCAATTTTTCATTAGGTTTGATGAACCAACA
GAACAACCTGTGAGGAAGAACAAAATGGCCTCTAATATCTTTGGGACACC
TGAAGAAAATCAAGCTTCTTGGGCCAAGTCAGCAGGTGCCAAGTCTAGTG
GTGGCAGGGAAGACTTGCAGTCATCTGGACTGCAGAGAAGGAACTCCTCT
GAAGCAAGCTCCGGAGACTTCTTAGATCTGAAGGGAGAAGGTGATATTCA
TGAAAATGTGGACACAGACTTGCCAGGCAGCCTGGGGCAGAGTGAAGAGA
AGCCCGTGCCTGCTGCGCCTGTGCCCAGCCCGGTGGCCCCGGCCCAGTGC
CATCCAGAAGATATCCCCCTGGCGGCAAGTCCAGCCTCCTCTTCGGTTAG
CTCTCACTCTCCTGAACGCTGTCCTTCTTGTCTTGTTTCCTCCCATCCTT
TCTTCAACCTCCACAAACTTCAACCCCTTCACGTGGTACAATCTCCTTCC
AGTTTCTCTCCACTTTAAAACGAACCACTTTTTAAATAG
(CloneID 3351305)

SEQ ID NO:159

CGTCTCCACTTTCCCACCGCCCCTCCCCTCCGACCCTTTCACCGTTCTCC
TCCCTGCGCCACCCTACCTCGCTCCTCCCCCCCATGACCACAACCACCAC
CTTCAAGGCAGTCGACCCCAACACCAGCAATAGCTCCCCAGTTTTGCGGC
CTCCACCTCCTGGATCCAATTTTTCATTAGGTTTTGATGAACCAACACAA
CAACCTCTGAGGAAGAACAAAATGGCCTCTAATATCTTTCGGACACCTGA
ACAAAATCAAGCTTCTTCCCCCAACTCAGCAGGTCCCAAGTCTACTCCTC
CCAGCCAACACTTGCAGTCATCTGGACTGCAGAGAAGCAACTCCTCTCAA
GCAAGCTCCGGACACTTCTTAGATCTGAAGGCAGAACCTCATATTCATGA
AAATGTGGACACAGACTTCCCACGCAGCCTCGCCCACAGTCAAGAGAAGC
CCGTGCCTCCTGCCCCTGTCCCCACCCCGGTCCCCCCGCCCCCAGTCCCA
TCCACAACAAATCCCCCTGCCGGCAAGTCCACCCTCCTCTTCCGTTAGCT
CTGACTGTCCTGAACCCTGTCGTTCTGTCTGTTTCCTCCATCCTTCTGAA
CTGCACAATTTCACCTGACTCC
(CloneID 3029189)

SEQ ID NO:160

TTCGGTGTCGAGTTTCCCAGCGCCCCTCGGCTCCCACCCTTTGAGCGTTC
TGCTCCGGCGCCAGCCTACCTCGCTCCTCCCCGCCATGACCACAACCACC
ACCTTCAAGGGAGTCGACCCCAACAGCAGGAATAGCTCCCGAGTTTTGCG
GCCTCCAGGTGGTGGATCCAATTTTTCATTAGGTTTTGATCAACCAACAG
AACAACCTGTCAGCAACAACAAAATGCCCTCTAATATCTTTCCCACACCT
CAACAAAATCAAGCTTCTTCCCCCAACTCACCACCTCCCAACTCTAGTCC
TCCCACCCAACACTTCCACTCATCTGCACTCCACACAACCAACTCCTCTC
AACCAACCTCCCCACACTTCTTACATCTCAACCGACAACCTCATATTCAT
CAAAATCTCCACACACACTTCCCACCCACCCTCCCCCACACTCAAGACAA
CCCCCTCCCTCCTCCCCCTCTCCCCACCCCGCTCCCCCCCCCCCCACTCC
CATCCACAACAAATCCCCCTCCCCCCAACTCCACCCTCCTCTTCCCTTAC
CTCTCACTCTCCTCAACCCTCTCCTTCTCTCTCTTTCCTCCATCCTT
(CloneID 5465786)

SEQ ID NO:161

GCTGTTGGGTGTGGAGTTTCCCAGCGCCCCTCGGGTCCGACCCTTTGAGC
GTTCTGCTCCGGCGCCAGCCTACCTCGCTCCTCGGCGCCATGACCACAAC
CACCACCTTCAAGGGAGTCGACCCCAACAGCAGGAATAGCTCCCGAGTTT
TGCGGCCTCCAGGTGGTGGATCCAATTTTTCATTAGGTTTTGATGAACCA
ACAGAACAACCTGTGAGGAAGAACAAAATGGCCTCTAATATCTTTGGGAC
ACCTGAAGAAAATCAAGCTTCTTGGGCCAAGTCAGCAGGTGCCAAGTCTA
GTGGTGGCAGGGAAGACTTGGAGTCATCTGGACTGCAGAGAAGGAACTCC
TCTGAAGCAAGCTCCCGAGACTTCTTAGATCTGAAGGGAGAAGGTGATAT
TCATGAAAATGTGGACACAGACTTGCCAGGCAGCCTGGGGCAGAGTGAAG
AGAAGCCCGTGCCTGCTGCGCCTGTGCCCAGCCCGGTGGCCCCGGCCCCA
GTGCCATCCAGAAGAAATCCCCCTGGCGGCAAGTCCAGCCTCGTCTTGGG
TTAGCTCTGACTGTCCTGAACGCTGTCGTTCTGTCTGTTTCCTCCATGC
TTGTGAACTGCACAACTTGAGCCTGACTGTACATCTCTTGGATTTGTTTC
ATTAAAAAGAAGCACTTTAAAAACAAAAAAAAAAAAAACTCGA
(CloneID 2819762)

SEQ ID NO:162

GGCACGAGGACAACCACCACCTTCAAGGGAGTCGACCCCAACACCAGGAA
TAGCTCCCGAGTTTTGCGGCCTCCAGGTGGTGGATCCAATTTTTCATTAG
CTTTTGATGAACCAACAGAACAACCTGTGAGGAAGAACAAAATGGCCTCT
AATATCTTTGGGACACCTGAAGAAAATCAAGCTTCTTGGGCCAAGTCAGC
AGGTGCCAAGTCTAGTGGTGGCAGGGAAGACTTGGAGTCATCTGGACTGC
AGAGAAGGAACTCCTCTGAAGCAACCTCCGGAGACTTCTTACATCTGAAG
GGAGAAGGTGATATTCATGAAAATGTGGACACAGACTTGCCAGGCAGCCT
GGGCCAGACTGAAGAGAACCCCGTGCCTGCTGCGCCTGTGCCCAGCCCGG
TGGCCCCCGCCCCAGTGCCATCCAGAAGAAATCCCCCTGGCGGCAAGTCC
AGCCTCCTCTTGGGTTAGCTCTGACTGTCCTGAACCCTGTCGGTCTGTCT
GTTTCCTCCATGCTTGTGAACTGCACAACTTGAGCCTGACTGTACATCTC
TTGCATTTGTTTCATTAAAAAGAAGCACTTTANNAAAAANAAAAANNAAA
(CloneID 2819762 in 3′ to 5′ orientation)

SEQ ID NO:163

TTTTTTTTTTAAAGTGCTTCTTTTTAATGAAACAAATCCAAGAGATGTAC
AGTCAGGCTCAAGTTGTGCAGTTCACAAGCATGGAGGAAACAGACAGAAC
GACAGCGTTCAGGACAGTCAGAGCTAACCCAAGACGAGGCTGGACTTGCC
GCCAGGGGGATTTCTTCTGGATGGCACTGGGCCCGGGGCCACCGGGCTGG
GCACAGGCGCAGCAGGCACGGGCTTCTCTTCACTCTGCCCCAGGCTGCCT
GGCAAGTCTGTGTCCACATTTTCATGAATATCACCTTCTCCCTTCAGATC
TAAGAAGTCTCCGGAGCTTGCTTCAGAGGAGTTCCTTCTCTGCAGTCCAG
ATGACTCCAAGTCTTCCCTGCCACCACTAGACTTGGCACCTGCTGACTTG
GCCCAACAAGCTTGATTTTCTTCAGGTGTCCCAAAGATATTAGAGGCCAT
TTTGTTCTTCCTCACAGGTTGTTCTGTTGGTTCATCAAAACCTAATGAAA
AATTTGGATCCACCACCTGGAGGCCGCAAAACTCGGGAGCTATTCCTGCT
GTTGGGGTCGACTCCCT
(CloneID 2819763)

SEQ ID NO:164

GGCACGAGGACAACCACCACCTTCAAGGCAGTCGACCCCAACAGCAGGAA
TAGCTCCCGAGTTTTGCGGCCTCCAGGTGGTGGATCCAATTTTTCATTAG
GTTTTGATCAACCAACAGAACAACCTGTGAGGAAGAACAAAATGGCCTCT
AATATCTTTGGGACACCTGAAGAAAATCAAGCTTCTTGGGCCAAGTCAGC
AGGTGCCAAGTCTAGTGGTGGCAGGGAAGACTTGGAGTCATCTGGACTGC
AGAGAAGGAACTCCTCTGAAGCAAGCTCCGGAGACTTCTTAGATCTGAAG
GGAGAAGGTGATATTCATGAAAATGTGGACACAGACTTGCCAGGCAGCCT
GGGGCAGAGTGAAGAGAAGCCCGTGCCTGCTGCGCCTGTGCCCAGCCCGG
TGGCCCCGGCCCCAGTGCCAT
(CloneID 2819763 in 3′ to 5′ orientation)

SEQ ID NO:165

TTTTTTTTTTAAAGTGCTTCTTTTTAATGAAACAAATCCAAGAGATGTAC
AGTCAGGCTCAAGTTGTGCAGTTCACAAGCATGGAGGAAACAGACAGAAC
GACAGCGTTCAGGACAGTCAGAGCTAACCCAAGACGAGGCTGGACTTGCC
GCCAGGGGGATTTCTTCTGGATGGCACTGGGGCCGCGGCCACCGGGCTGG
GCACAGGCGCAGCAGGCACGGGCTTCTCTTCACTCTGCCCCAGGCTGCCT
GGCAAGTCTGTGTCCACATTTTCATGAATATCACCTTCTCCCTTCAGATC
TAAGAAGTCTCCGGAGCTTGCTTCAGAGGAGTTCCTTCTCTGCAGTCCAG
ATGACTCCAAGTCTTCCCTGCCACCACTAGACTTGGCACCTGCTGACTTG
GCCCAAGAAGCTTGATTTTCTTCAGGTGTCCCAAAGATATTAGAGGCCAT
TTTGTTCTTCCTCACAGGTTGTTCTGTTGGTTCATCAAAACCTAATGAAA
AATTGGATCCACCACCTGGAGGCCGCAAAACTCGGGAGCTATTCCTGCTG
TTGGGGTCGACTCCCT
(CloneID 3912245)

SEQ ID NO:166

GGTGTGGAGTTTCCCAGCGCCCCTCGGGTCCGACCCTTTGAGCGTTCTGC
TCCGGCGCCAGCCTACCTCGCTCCTCGGCGCCATGACCACAACCACCACC
TTCAAGGGAGTCGACCCCAACAGCAGGAATAGCTCCCGAGTTTTGCGGCC
TCCAGGTGGTGGATCCAATTTTTCATTAGGTTTTGATGAACCAACAGAAC
AACCTGTGAGGAAGAACAAAATGGCCTCTAATATCTTTGGGACACCTGAA
GAAAATCAAGCTTCTTGGGCCAAGTCAGCAGGTGCCAAGTCTAGTGGTGG
CAGGGAAGACTTGGAGTCATCTGGACTGCAGAGAAGGAACTCCTCTGAAG
CAAGCTCCGGAGACTTCTTAGATCTGAAGGGAGAAGGTGATATTCATGAA
AATGTGGACACAGACTTGCCAGGCAGCTGGGGCAGAGTGAAGAGAAGCCC
GTGCCTGCTGCGCCTGTGCCCAGCCCGGTGGCCCCGGCCCCAGTGCCATC
CAGAAGAAATCCCCCTGGGCGGCAAGTCCAGGCTCCCTCTTGGGGTTAGC
TCTGACTGGTCCTGAACGCTGTCGTTCTGTCTGGTGTCCTCCATGCTTTG
TGAACTGGGCCCAATTTGAGGCCTGGAGTGGTGCCTCTCTTGGGTTTTGG
TGCCTCTTAACGAAGCCTTTTTTGTCCGGCGGCCCTTGCCCTTTGCGGCA
CCCGGTGTTCTCCTGCCCTACACCGCGGGCGGGGGCCCGG
(CloneID 5164014)

SEQ ID NO:167

CCGGCGGGCTCCTTACCCTCGCCGTGCCCCCACTCCTTATTCGGACCTAC
CTCACAGAGTTTTGCGGCCTCCAGGTGGTGGATCCAATTTTTCATTAGGT
TTTGATGAACCAACAGAACACCTGTGAGGAAGAACAAAATGGCCTCTAAT
ATCTTTGGGACACCTGAAGAAAATCAAGCTTCTTGGGCCAAGTCAGCAGG
TGCAAGTCTAGTGGTGGCAGGGAAGACTTGGAGTCATCTGGACTGCAGAG
AAGGAACTCCTCTGAAGCAAGCTCCGGAGACTTCTTAGATCTGAAGGGAG
AAGGTGATATTCATGAAAATGTGGACACAGACTTGCCAGGCAGCCTGGGG
CAGAGTGAAGAGAAGCCCGTGCCTGCTGCGCCTGTCCCCAGCCCGGTGGC
CCCGGCCCCAGTGCCATCCAGAAGAAATCCCCCTGGCGGCAAGTCCAGCT
CGTCTTGGGTTAGCTCTGACTGTCCTGAACGCTGTCGTTCTGTCTGTTTC
CTCCATGCTTGTGAACTGCACAACTTCAGCCTGACTGTACATCTCTTGGA
TTTCTTTCATTAAAAACAGAAGGCACCCTTTAATAAACAAACACAACAAC
ACAGAACAAAACACAAGAAAAAGAAAACAACGGGGGG
(CloneID 4284754)

SEQ ID NO:168

GAGCGGGGCTCCTGCAGCGCTGGTCGGCTGTGGGTGTGGAGTTTCCCAGC
GCCCCTCGGGTCCGACCCTTTGAGCGTTCTGCTCCGGCGCCAGCCCTACC
TCGCTCCTCGGGCCATGGACCACAACCACCACCTTCAAGGGAGTCGACCC
CAACAGCAGGAATAGCTCCCGAGTTTGGCGGCCTCCAGGTGGTGGATCCA
ATTTTTCATTAGGTTTGATGAACCAACAGAACAACCTGTGAGGAAGAACA
AAATGGCCTCTAATATCTTGTGGGACACCTGAAGAAAATCAAGCTTCTGC
GGCCAAGTCAGCAGCTGCCAAGTCTAGTGGTGGCAGGGAAGACTTGGAGT
CATCTGGACTGCAGAGAACCAACTCCTCTGCAGCAAGCTCCGGAGACTTC
TTACATCTGAAGGGAGAAGGTGATATTCATGAACAATGTGGACACAGACT
TGCCAGGCCGCCTGGGGGCACGAGTGAAGAGAAGCCCGTGCCTGCTGCGC
CTGTGCCCAGCCCGGTGGCCCGGCCCCAGTGCCATCCAAGAATGAAATTC
CCCTGGGGGCAGAGTCCAGGCCTCGTCTTGGGTTACGCTCTGACTGTCTG
AACCCCTGTCGTTCTGGTCTTGTTTTCTTCCATGCTGTGTGAACGTGGAC
AACTTTGAGGCCTTGATGTACATCTCTGGATGTGTTCACTTAAAAAGAGC
AATTATGTATCGGTGGCTTTTCATTTCCTTGGACAAACCGGTCTCTCTGA
CTTGAACTGGGGTTGGGGCAGGGACGACGATGTTTCAACACAATGGAGCA
CCN
(CloneID 428337)

SEQ ID NO:169

GAGCGGCGGCTCCTGCAGCGGTGGTCGGCTGTTGGGTGTGGAGTTTCCCA
GCGCCCCTCGGTCCGACCCTTTGAGCGTCTGCTCCGGCGCCAGCCTACCT
CGCTCCTCGGCGCCATCACCACAACCACCACCTTCAAGGGAGTCGACCCC
AACAGCAGGAATAGCTCCCGAGTTTTGCGGCCTCCAGGTGGTGGATCCAA
TTTTTCATTAGGTTTTGATGAACCAACAGAACAACCTGTGAGGAAGAACA
AAATGGCCTCTAATATCTTTGGGACACCTGAAGAAAATCAAGCTTCTTGG
GCCAAGTCAGCACGTGCCAAGTCTAGTGGTGGCAGGAAGACTTGGAGTCA
TCTGGACTGCAGAGAAGGAACTCCTCTGAAGCAAGCTCCGGAGACTTCTT
AGATCTGAAGGGAGAAGGTGATATTCATGAACCATGTGGACACAAGACTT
GCCAGGCAGCCTGGGGGCAGAGTGAAGAAGAAGCCCGTGCCTGCTTGCGC
CTGTGCCCAGCCCGGGTGGCCCCGGCCCCCAGTGCATCCAGAAGAAATCC
CCCTGGGCGGCAAGTCCAGCCTCGTCTTGGGTAGCTCTGACTGTCTGACG
CTGGCGTCTGGCTGTTCCTCCTGCTTGTGAACTGCACACTTGAGCCTGAC
TGGACATCTCTTGGATTCGTTCATAAAAGGAGCTTGCAAACAAAAAAAAA
TTTGCGCGCCCCCCAAATTTTAACTCTTGGCGCCCCGGTGAATGGCGCGC
CAGACCGCACAGGGGTCACAAACCGGGCGGCGAAACAGGGGTTGTCGCCA
CACCCCGG
(CloneID 5936059)

SEQ ID NO:170

AGCGAGGCCTGGGTGGCGATCCAAGACGAGGCTGCAGCGGTGGTCGTCTG
TTGGGTGTGGAGTTTCCCAGCGCCCCTCGGGTCCGACCCTTTGAGCGTTC
TGCTCCGGCGCCAGCCTACCTCGCTCCTCGGCGCCATGACCACAACCACC
ACCTTCAAGGGAGTCGACCCCAACAGCAGGAATAGCTCCCGAGTTTTGCG
GCCTCCAGGTGGTGGATCCAATTTTTCATTAGGTTTTGATGAACCAACAG
AACAACCTGTGAGGAAGAACAAAATGGCCTCTAATATCTTTGGGACACCT
GAAGAAAATCAAGCTTCTTGGGCCAAGTCAGCAGGTGCCAAGTCTAGTGG
TGGCAGGGAAGACTTGGAGTCATCTGGACTGCAGAGAAGGAACTCCTCTG
AAGCAAGCTCCGGAGACTTCTTAGATCTGAAGGGAGAAGGTGATATTCAT
GAAAATGTGGACACAGACTTGCCAGGCAGCCTGGGGCAGAGTGAAGAGAA
GCCCGTGCCTGCTGCGCCTGTGCCCAGCCCGGTGGCCCCGGCCCCAGTGC
CATCCAGAAGAAATCCCCCTGGCGGCAAGTCCAGCCTCGTCTTGGGTTAG
CTCTGACTGTCCTGAACGCTGTCGTTCTGTCGGTTTCCCTCCATGCTTGT
GAACTGCACAACTTGAGCCTGACCTGTACATCTCTTGGATTTGTTTCATT
AAAAAGAAGCACTTTAAGGACTGCTGGCCTTTTTTTTTTTTCTTTTGAAA
AACAGGTTTCCCCCCGGCCCTTTGATCCTTGGGGTCTGGGGGCCCATGGG
AATGAAATGGTCTTCTAACTAACAACATTGGGAAGGAAAAAGCCTTGGGG
ACACCTCAACAACGTGGGGTTTTTAAAAAGAAAAAAAATTTGGGTTCCCA
CAGGGGGTTAAAAGGAATCTTTTTGTACCGGAAGGGTTTTTAAACCCTTT
TACCTGGGGGGTTACCCAGGCCCTCCCACTTGGGACCGGAACCATTTAAC
CGGTCTCCAGAGGGGCCCCGTTTCCCGGGCCAGAGCCCCCCACCCTCACA
GGGGAAGTCTCTCCTCCCCAAAAAACTCTCCCTTGGGGGGTGGGCTCCCC
TACCTTTCGCCCGTGGGGCCCTTTTGTCACAAAAA
(CloneID 4280999)

SEQ ID NO:171

GAGCGGCGGCTCCTGCAGCGGTGGTCGGCTGTTGGGTGTGGAGTCTCCCA
GCGCCCCTCGGGTCCGACCCTTTGAGCGTTCTGCTCCGGCGCCAGCCTAC
CTCGCTCCTCGGCGCCATGACCACAACCACCACCTTCAAGGGAGTCGACC
CCAACAGCAGGAATAGCTCCCGAGTTTTGCGGCCTCCAGGTGGTGGATCC
AATTTTTCATTAGGTTTGATGAACCAACAGAACAACCTGTGAGGAAGAAC
AAAATGGCCTCTAATATCTTTGGGACACCTGAAGAAAATCAAGCTTCTTG
GGCCAAGTCAGCAGGTGCCAAGTCTAGTGGGTGGCAGGGGAACGACTTGG
AGTCATCTGGACTGCAGAGAAGGAACTCCTCTGAAGCAAGCTCCGGAGAC
TTCTTAGATCTGAAGGGAGACGGTGATATTCATGAAAATGTGGACACAGA
CTTGCCAGGCAGCCTGGGGCAGAGTGAAGAGAAGCCCGTGCCTGCTGCGC
CTGTGCCCAGCCCGGTGGGCCCGGCCCAGTGCCATCCAGAAGAAAATCCC
CCTGGCGGGCAAGTCCCAGCCTTCCGTCTGTGGGGTTAAGCTCTTGACTG
GGTCCCTGAACGCTGTCCGTTCCTGTCTGTTTTCCCTCCCATCGCTGGTG
AACTGGACCAACTTTGAGCCTGACTTGTACATTCTCCTGGGATTCGTCTC
CTTTCACCCAGAAGGCCTTTATGACACACAACAACACTCCAAACACACCT
TGGGCGCCGCGCGCCCCTCAGACATTTTAAACCCTCTGTGCGGCCGGCCC
ACGGTAGGACACGCCTAGGCCCGGCAACGGGAGGGACGGTGGTTACCAAA
ACCCGGGCGGCCTGCCCCACGGTGCGCTCG
(CloneID 6154219)

SEQ ID NO:172

GCACGCGTGGGCGGACGCGTGGGCTGTTGGGTGTGGAGTTTCCCAGCGCC
CCTCGGGTCCGACCCTTTGAGCGTTCTGCTCCGGCGCCAGCCTACCTCGC
TCCTCGGCGCCATGACCACAACCACCACCTTCAAGGGAGTCGACCCCAAC
AGCAGGAATAGCTCCCGAGTTTTGCGGCCTCCAGGTGGTGGATCCAATTT
TTCATTAGGTTTTGATGAACCAACAGAACAACCTGTGAGGAAGAACAAAA
TGGCCTCTAATATCTTTGGGACACCTGAAGAAAATCAAGCTTCTTGGGCC
AAGTCAGCAGGTGCCAAGTCTAGTGGTGGCAGGGAAGACTTGGAGTCATC
TGGACTGCAGAGAAGGAACTCCTCTGAAGCAAGCTCCGGAGACTTCTTAG
ATCTGAAGGGAGAAGCTGATATTCATGAAAATGTGGACACAGACTTGCCA
GGCAGCCTGGGGCAGAGTGAAGAGAAGCCCGTGCCTGCTGCGCCTGTGCC
CAGCCCGGTGGCCCCGGCCCCAGTGCCATCCAGAAGAAATCCCCCTGGCG
GCAAGTCCAGCCTCGTCTTGGGTTAGCTCTGACTGTCCTGAACGCTGTCG
TTCTGTCTGTTTCCTCCATGCTTGTGAACTGCACAACTTCAGCCTGACTG
TACATCTCTTGGGGATTTGTTTCATTAAAAAAGAAGCACTTTTATGTACT
GCTGGNCTTTTTTTTTTTTTTCTTTTTTGAAGAACAGGTTTTCTCTCCTG
TCCCTTGACTCCTTTGGGGTCTGTTGGGCCCATGGGGCATGGAGTTGGTT
TTCTAAGGTAAAGTAGAAATTGGGGAGGGGGAAAAGCCTTTTGGTCGACA
CCCTTTT
(CloneID 4045037)

SEQ ID NO:173

GGCGGGGCTCACTGACAGCGGTGGTCGGGTGTGGGTGTGGAGTTTCCCAG
CTGGCCCCTCGGGTGCGACCCTTGTGAGCGTTTCTGCTCCGGTCGCCAGT
CGTTACCTCGCTCCTCGGGCCATGACCACAAGCACCAGCTTCAAGGGAGT
CGATCCCAACAGCAGGAATAGCTCCCGAGTGTAGCGGTCTCCAGGTGGTG
GATCCAATTCACATCAGGTTTGATGAACCAAGAGAACAACCTGTGAGGAA
GAACAAAATGGCCTCTAATATCTTTGGGACACCTGAAGAAAAACAAGCTT
CTTGGGCCAAGTCACCAGGTGCCAAGTCTAGTGGTGGCAGGGAAGACTTG
GAGTCATCTGGACTGCAGAGAAGGAACTCCTCTGAAGCAAGCTCCGGAGA
CTTCTTAGATCTGAAGGGAGACGGTGATATTCATGACAATGTGGACACAG
ACTTGCCAGGCAGCCTGGGGCCACAGTGAAGAGAAGCCCGTGCTGCTGCG
CCTGTGCCAGCCCGGNTGGCCCGGCCCAGTGCCATCCAGAAGAATCCCCT
TGGGGAGTCAGCTCGTCTGGGTA
(CloneID 6048028)

SEQ ID NO:174

CCACGCGTCCGCTGCAGCGGTGGTCGGCTGTTGGGTGTGGAGTTTCCCAG
CGCCCCTCGGGTCCGACCCTTTGAGCGTTCTGCTCCGGCGCCAGCCTACC
TCGCTCCTCGGCCATGACCACAACCACCACCTTCAAGGGAGTCGACCCCA
ACAGCAGGAATAGCTCCCGAGTTTTGCGGCCTCCAGGTGGTGGATCCAAT
TTTTCATTAGGTTTTGATGAACCAACAGAACAACCTGTGAGGAAGAACAA
AATGGCCTCTAATATCTTTGGGACACCTGAAGAAAATCAAGCTTCTTGGG
CCAAGTCAGCAGGTGCCAAGTCTAGTGGTGGCAGGGAAGACTTGGAGTCA
TCTGGACTGCAGAGAAGGAACTCCTCTGAAGCAAGCTCCGGAGACTTCTT
AGATCTGAAGGGAGAAGGTGATATTCATGAAAATGTGGACACAGACTTCC
CAGGCAGCCTGGGGCAGAGTGAAGAGAAGCCCGTGCCTGCTGCGCCTGTG
CCCAGCCCGGTGGCCCCGGCCCCAGTGCCATCCCGAAAAAATCCCCCTGG
CGGCAAGTCCAGCCTCGTCTTGGGTTAACTCTGACTGTCCTGAACGATGT
CGGTCTGGCCTGTTTCCTCCATGCTTGGGAACTGCACAACTGGAGCCTGA
CTGTACATCTCTTGGAATTGGTTTCATTAAAAAGAAACACCTTTAAAAAA
AAAAAAAAAAAGGGGGGGCCGCTCCAAAAGGAT
(CloneID 2518214)

SEQ ID NO:175

GAGCTCAAGGATCCTTAATTAAATTAATTCCCCCCCCCCCCGAGCGGCGG
CTCCTGCAGCGGTGGTCGGCTGTTGGGTGTGGAGTTTCCCAGCGCCCCTC
GGGTCCGACCCTTTGAGCGTTCTGCTCCGGCGCCAGCCTACCTCGCTCCT
CGGCGCCATGACCACAACCACCACCTTCAAGGGAGTCGACCCCAACAGCA
GGAATAGCTCCCGAGTTTTGCGGCCTCCAGGTGGTGGATCCAATTTTTCA
TTAGGTTTTGATGAACCAACAGAACAACCTGTGAGGAAGAACAAAATGGC
CTCTAATATCTTTGGGACACCTGAAGAAAATCAAGCTTCTTGGGCCAAGT
CAGCAGGTGCCAAGTCTAGTGGTGGCAGGGAAGACTTGGAGTCATCTGGA
CTGCAGAGAAGGAACTCCTCTGAAGCAAGCTCCGGAGACTTCTTAGATCT
GAAGGGAGAAGGTGATATTCATGAAAATGTGGACACAGACTTGCCAGGCA
GCCTGNGGCAGAGTGAAGAGAAGCCCGTGCCTGCTGCGCCTGTGCCCAGC
CCAGTGGCCCCGGTCCCAGTGCCATCCAGAAGAAATCCC
(CloneID 2820804)

SEQ ID NO:176

GGCACGAGGCCTGCAGCGGTGGTCGGCTGTTGGGTGTGGAGTTTCCCAGC
GCCCCTCGGGTCCGACCCTTTGAGCGTTCTGCTCCGGCGCCAGCCTACCT
CGCTCCTCGGCGCCATGACCACAACCACCACCTTCAAGGGAGTCGACCCC
AACAGCAGGAATAGCTCCCGAGTTTTGCGGCCTCCAGGTGGTGGATCCAA
TTTTTCATTAGGTTTTGATGAACCAACAGAACAACCTGTGAGGAAGAACA
AAATGGCCTCTAATATCTTTGGGACACCTGAAGAAAATCAAGCTTCTTGG
GCCAAGTCAGCACGTGCCAAGTCTAGTGGTGGCAGGGAAGACTTGGAGTC
ATCTGGACTGCAGAGAAGGAACTCCTCTGAAGCAAGCTCCGGAGACTTCT
TAGATCTGAAGGGAGAAGGTGATATTCATGAAAATGTGGACACAGACTTG
CCAGGCAGCCTGGGGCAGAGTGAAGAGAAGCCCGTGCCTGCTGCGCCTGT
GCCCAGCCCGGTGGCCCCGGCCCCAGTGCCATC
(CloneID 2519633)

SEQ ID NO:177

AGCGGCGGCTCCTGCAGCGGTGGTCGGTTGTTTGGTGTGGAGTTTCCCAG
CGCCCCTCGGGTCCGACCCTTTGAGCGTTCTGCTCCGGCGCCAGCCTACC
TCGCTCCTCGGCGCCATGACCACAACCACCACCTTCAAGGGAGTCCACCC
CAACAGCAGGAATAGCTCCCGAGTTTTGCGGCCTCCAGGTGGTGGATCCA
ATTTTTCATTAGGTTTTGATCAACCAACCAGAACAACCTGTGAGGAAGAA
CAAAATGGCCTCTAATATCTTTGGGACACCTGAAGAAAATCAAGCTTCTT
GGGCCAAGTCAGCAGGTGCCAAGTCTAGTGGTGGCAGGGAAGACTTGGAG
TCATCTGGACTGCAGAGAAGGAACTCCTCTGAAGCAAGCTCCGGAGACTT
CTTAGATCTGAAGGGAGAAGGTGATATTCATGAAAATGTGGACACAGACT
TGCCAGGCAGCCTG
(CloneID 2518293)

SEQ ID NO:178

AGGGGATCCTTATATTAAATTAATTCNCTCCCCCCCCCCCAGAGCCGGCG
NNNGCTCCTGCGGAGCGTGTGTGTCGGCTTTGTTGGGATGTGCGAGCTTT
CCCCAGCTGCACCCTTCGGAGTCCCGACGCCTTTTGANNGCGTTCTGCAT
CCCGGCGCCCAGCCCCTACTCTCGCTCCTCTGGCGCCCATGANCCACTAT
ACCACCACCTTCAAGTGTGAGTCGACCCCAACNATGCAGGAATAGCTCCC
GAGTTTTGGCGGCCTCCAGGTGGTGGATCCAATTTTTCATTAGGTTTTGA
TGAACCAACAGAACAACCTGTGAGGAAGAACAAAATGGCCTCTAATATCT
TTGGGACACCTGAAGAAAATCAAGCTTCTTGGGCCAAGTCAGCAGGTGCC
AAGTCTAGTGGTGGCAGGGAAGACTTGGAGTCATCTGGACTGCAGAGAAG
GAACTCCTCTGAAGCAAGCTCCGGAGACTTCTTAGATCTGAAGGGAGAAG
GTGATATTCATGAAAATGTGGACACAGACTTGCCAGGCAGCCTGNGGCAG
AGTGAAGAGAAGCCCGTGCCTGCTGCGCCTGTGCCCAGCCCAGTGGCCCC
GGTCCCAGTGCCATCCAGAAGAGATCCCCCTTGCGGCAAGTCCAGCCTCG
TCTTG
(CloneID 2819423)

SEQ ID NO:179

GGCACGAGGCCTGCAGCGGTGGTCGGCTGTTGGGTGTGGAGTTTCCCAGC
GCCCCTCGCGTCCGACCCTTTGAGCGTTCTGCTCCGCCGCCAGCCTACCT
CGCTCCTCGGCGCCATGACCACAACCACCACCTTTAAGGGAGTCGACCCC
AACAGCAGGAATAGCTACCGAGTTTTGCGGCCTCCAGGTGGTGGATCCAA
TTTTTCATTAGGTTTTGATGAACCAACAGAACAACCTGTGAGGAAGAACA
AAATGGCCCCTAATATCTTTGGGACACCTGAAGAAAATCAAGCTTCTTGG
GCCAAGTCAGCAGGTGCCAAGTCTAGTGGTGGCAGGGAAGACTTGGAGTC
ATCTGGACTGCAGAGAAGGAACTCCTCTGAAGCAAGCTCCCGAGACTTCT
TAGATCTGAAGGGAGAAGGTGATATTCATGAAAATGTGGACACAGACTTG
CCAGGCACCCTGGGGCAGAGTGAAGAGAAGCCCGTGCCTGCTGCGCCTGT
GCCC
(CloneID 2819397)

SEQ ID NO:180

GGCACGAGGCCTGCAGCGGTGGTCGGCTGTTGGGTGTGGAGTTTGCCAGC
GCCCCTCGGGTCCGACCCTTTGAGCGTTCTGCTCCGGCGCCAGCCTACCT
CGCTCCTCGGCGCCATGACCACAACCACCACCTTCAAGGGAGTCGACCCC
AACAGCAGGAATAGCTCCCGAGTTTTGCGGCCTCCAGGTGGTGGATCCAA
TTTTTCATTAGGTTTTGATGAACCAACAGAACAACCTGTGAGGAAGAACA
AAATGGCCCCTAATATCTTTGGGACACCTGAAGAAAATCAAGCTTCTTGG
GCCAAGTCAGCAGGTGCCAAGTCTAGTGGTGGCAGGGAAGACTTGGAGTC
ATCTGGACTGCAGAGAAGGAACTCCTCTGAAGCAAGCTCCGGAGACTTCT
TATATCTGAAGGGAGAAGGTGATATTCATGAAAATGTGGACACAGACTTG
GCC
(CloneID 2783324)

SEQ ID NO:181

CGAGCGGCGGCTCCTGGAGCGGTGGTCGGCTGTTGGGTGTGGAGTTTCCC
AGCGCCCCTCGGGTCCGACCCTTTGAGCGTTCTGCTCCGGCGCCAGCCTA
CCTCGCTCCTCGGCGCCATGACCACAACCACCACCTTCAAGGGAGTCGAC
CCCAACAGCAGGAATAGCTCCCGAGTTTTGCGGCCTCCAGGTGGTGGATC
CAATTTTTCATTAGGTTTTGATGAACCAACAGAACAACCTGTGAGGAAAA
ACAAAATGGCCTCTAATATCTTTGGGACACCTGAAGAAAATCAAGCTTCT
TGGGCCAAGTCAGCAGGTGCCAAGTCTAGTGGTGGCAGGGAAGACTTGGA
GTCATCTGGACTGCAAAGAATGAACTCCTCTGAAGCAAGCTCCGGAGACT
TCTTAGATCTGAAGGGAGAAGGTCATATTCATTAAAATGTGGACACAGAC
TAGCCATGCAGCCTGTGGCATAGTGAATAGAAGCCCGTGCCTGCTGCTCC
TGTGCCTATTTCAGTTCATCCAGCACACGCACCGTCCACAACAAATCCCG
CTGGGGCAAGTACAGGCTTGATT
(CloneID 471568 in 3′ to 5′ orientation)

SEQ ID NO:182

TTACATAAAGTGCTTCTTTTTAATGAAACAAATCCAAGAGATGTACAGTC
AGGCTCAAGTTGTGCAGTTCACAAGCATGGAGGAAACAGACAGAACGACA
GCGTTCAGGACAGTCAGAGCTAACCCAAGACGAGGCTGGACTTGCCGCCA
GGGGATTTCTTCTGGATGGCACTGGGGCCGGGGCACCGGGCTTGGGCACA
GGCGCANAGGCACGGGCTTCTCTTCACTCTGCCCCAGGCTGCCTGGCAAG
TCTGTGTCCACATTTTCATGAATATCACCTTCTCCCTTCAGATCTAAGAA
AGTCTCCGGAGCTTGCTTCAGAGGAGTTCCTTCTCTTGCAGTNCAGATTG
ACTCCAAGTCTTCCCTGCCACCANTAGACTTTGGCANCTGCNGACTTTGG
NCCAAGAAAGTTGAATTTCTTCAAGNGGTCCCAAAGATTT
(CloneID 471568)

SEQ ID NO:183

CTGCAGCGGTGGTCGGCTGTTGGGTGTGGAGTTTCCCAGCGCCCCTCGGG
TCTCGACCCTTTGAGCGTTCTGCTCCGGCGCCACTACCTCGCTCCTCGGC
GCCATGACCACAACCACCACCTTCAAGGGAGTCGACCCCAACAGCAGGAA
TAGCTCCCGAGTTTTGCGGCCTCCAGGTGGTGGATCCAATTTTTCATTAG
GTTTTGATGAACCAACAGAACAACCTGTTG
(CloneID 4080434)

SEQ ID NO:184

GACCCCAACAGCAGGAATAGCTCCCGAGTTTTGCGGCCTCCAGGTGGTGG
ATCCAATTTTTCATTAGGTTTTGATGAACCAACAGAACAACCTGTGAGGA
AGAACAAAATGGCCTCTAATATCTTTGGGACACCTGAAGAAAATCAAGCT
TCTTGGGCCAAGTCAGCAGCTGCCAAGTCTAGTGGTGGCAGGGAAGACTT
GGAGTCATCTGGACTGCAGAGAAGGAACTCCTCTGAAGCAAGCTCCGGAG
ACTTCTTAGATCTGAAGGGAGAAGGTGATATTCATGAAAATGTGNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNA
ACGCTGTCGTTCTGTCTGTTTCCTCCATGCTTGTGAACTGCACAACTTGA
GCCTGACTGTACATCTCTTGGATTTGTTTCATTAkkAAGAAGCACTTTAT
GTACTGCTGTCTTTTTTTTTTTCCTTTGGAAGAACAGGTTTCTCTCTGTC
CTTGACTCTGGGGTCTGTGGGCCATGGCATGAGTGTTTCTAGTAGTAATG
GGAGGGAAACCTTGGTGACACTTAGTACTGTGTTTTTAAGACGAAATAAT
TGGGTTTCCAGATCTGTCACAGGATCTTCGTACTGAGGTTTAACACTTTA
CTGGGGTTTACCAAGCCTCAATGGAAGACATAACAGTCACAGGCCCGTCT
GCAGGCCAACCAAGGGAGTTCTCCGAGAGCTCTGTTGTGCCTAATGCCAT
GGCTTGTCAAAGCCCTCTGGCTGGC
(CloneID 4748522)

SEQ ID NO:185

CGAGTTTTGCGGCTCCAGGTGGTGGATCCAATTTTTCATTAGGTTTTGAT
GAACCAACAGAACAACCTGTGAGGAAGAACAAAATGGCCTCTAATATCTT
TGGGACACCTGAAGAAAATCAAGCTTCTTGGGCCAAGTCAGCAGGTGCCA
AGTCTAGTGGTGGCAGGGAAGACTTGGAGTCATCTGGACTGCAGAGAAGG
AACTCCTCTGAAGCAAGCTCCGGAGACTTCTTAGATCTGAAGGGAGAAGG
TGATATTCATGAAAATGTGGACACAGACTTGCCAGGCAGCCTGGGGCAGA
GTGAAGAGAAGCCCGTGCCTGCTCCGCCTGTGCCCAGCCCGGTGGCCCCG
GCCCCAGTGCCATCCAGAAGAAATCCCCCTGGCGGCAAGTCCAGCCTCGT
CTTGGGTTAGCTCTGACTGTCCTGAACGCTGTCGTTCTGTCTGTTTCCTC
CATGCTTGTGAACTGCACAACTTGAGCCTGACTGTACATCTCTTGGATTT
GTTTCATTAAAAAGAAGCACTTTATGTACTGCTGTCTTTTTTTTTATTCC
TTTGGAAGAACAGGTTTCTCTCTGTCCTTGACTCTGTGGGTCTGTGGGCC
ATGGCATGAGTGTTTCCTAGTAGTAGATGGGAGGGAAAGCTTGGTGACAC
TTAGTACTGTGTTTTCAAGAAGAAATAATTGGGTCCCAGATCTGTTAGAG
GATCTTGGTACTGAGGTTTTTAACACTTTACTGTGGGTTTACCAAGCCTC
AACTGGACAGACCATAAA
(CloneID 5187032)

SEQ ID NO:186

CTAATATCTTTGGGACACCTGAAGAAAATCAAGCTTCTTGCGCCAAGTCA
GCAGGTGCCAAGTCTAGTGGTGGCAGGGAAGACTTGGAGTCATCTGCACT
GCAGAGAAGGAACTCCTCTGAAGCAAGCTCCGGAGACTTCTTAGATCTGA
AGGGAGAAGGTGATATTCATGAAAATGTGGACACAGACTTGCCAGGCAGC
CTGGGGCAGAGTGAAGAGAAGCCCGTGCCTGCTGCGCGTGTGCCCAGCCC
GGTGGCCCCGGCCCCAGTGCCATCCAGAAGAAATCCCCCTGGCGGCAAGT
CCAGCCTCGTCTTGGGTTAGCTCTGACTGTCCTGAACGCTGTCGTTCTGT
CTGTTTCCTCCATGCTTGTGAACTGCACAACTTGAGCCTGACTGTACATC
TCTTGGATTTGTTTCATTAAAAAGAAGCACTTTATGTACTGCTGTCTTTT
TTTTTTTTCCTTTGGAAGAACAGGTTTCTCTCGTGTCCTTGACTCTGGGG
TCTGGGGGCCAGGGCATGAGTGTTCCATAGTAGTAGATGGGAGGGAAAGC
TTGGGGACACTTAGTACGGGGTTTTCAAGAAGAAATAATCGGGTTCCAGA
GTGTGTAAGAGGACTCTTGGGTACGGAGGTTAAAACACTTAACTGGGGTA
TACCAAGCCTCAACGGGACAGACCATAAACGGTCCACAGGCACCGTTCCG
TGCAGGGCCCAAGCCAAAGGGAGTCTCTCCGAAGAGCCTTCTGGGAGTAG
CCCTAACTTTGCCAGGTGGCCTTGGGTCAAGAGCCTCCTCCTGTGACCTC
GTGA
(CloneID 3895688)

SEQ ID NO:187

AATATCTTGGGACACCTGAAGAAAATCAAGCTTCTTGGGCCAAGTCAGCA
GGTGCCAAGTCTAGTGGTGGCAGGGAAGACTTGGAGTCATCTGGACTGCA
GAGAAGGAACTCCTCTGAAGCAAGCTCCGGAGACTTCTTAGATCTGAAGG
GAGAAGGTGATATTCATGAAAATGTGGACACAGACTTGCCAGGCAGCCTG
GGGCAGAGTGAAGAGAAGCCCGTGCCTGCTGCGCCTGTGCCCAGCCCGGT
GGCCCCGGCCCCAGTGCCATCCAGAAGAAATCCCCCTGGCGGCAAGTCCA
GCCTCGTCTTGGGTTAGCTCTGACTGTCCTGAACGCTGTCGTTCTGTCTG
TTTCCTCCATGCTTGTGAACTGCACAACTTGAGCCTGACTGTACATCTCT
TGGATTTGTTTCATTAAAAAGAAGCACTTTATGTACTGCTGTCTTTTTTT
TTTTCCTTTGGAAGAACAGGTTTCTCTCTGTCCTTGACTCTGGGGTCTGT
GGGCCAGGGCATGAGTGTCTCTAGTAGTAGATGGGAGCGAAAGCTTGGTG
ACACACTTTACTGGGGTTTACCAAGCCTCAACTGACAGACCATAAACAGT
CCACAGGCACCGTTCCTGGCAGGCCCAACCCAAGGGAGTCTCTCCGAGAG
CCTTCTGGGTGTGGCCTAACTTGCCAGGGGCCTGGCTCAGAGCTCCTCTG
TGACTGTGACACTGAGCAG
(CloneID 3938961)

SEQ ID NO:188

AATCAAGCTTCTTGGGCCAAGTCAGCAGGTGCCAAGTCTAGTGGTGGCAG
GAAGACTTGGAGTCATCTGGACTGCAGAGAAGGAACTCCTCTGAAGCAAG
CTCCGGAGACTTCTTAGATCTGAAGGGAGAAGGTGATATTCATGAAAATG
TGGACACAGACTTGCCAGGCAGCCTGGGGCAGAGTGAAGAGAAGCCCGTG
CCTGCTGCGCCTGTGCCCAGCCCGGTGGCCCCGGCCCCAGTGCCATCCAG
AAGAAATCCCCCTGGCGGCAAGTCCAGCCTCGTCTTGGGTTAGCTCTGAC
TGTCCTGAACGCTGTCGTTCTGTCTGTTTCCTCCATGCTTGTGAACTGCA
CAACTTGAGCCTGACTGTACATCTCTTGGATTTGTTTCATTAAAAAGAAG
CACTTTATGTACTGCTGTCTTTTTTTTTTTCTTTTGAAGAACAGGTTTCT
CTCTGTCCTTGACTCTGTGGGTCTGTGGGCCAGTGGCATGAGTGTTTCTA
GTAGTAGATGGGAGGGAAAGCTTGGTGACACTTAGTACTGTGTTTTTAAC
GAAGAAATAATTGCTTCCAGATGTGTTAGAGGATCTTTGTACTGAGGTTT
TTAACACTTTACTGTGGGTTTACCAAGCCTCAATGGACAGACCATAAACA
GTCCACGAGGCACCGTTCCTGCCAGGCCCAACCCAAGAGGGAGTCTCTCC
GAAGAGCCTTCTGTGGTGTTGGCCCTAATTTGCCAGGTGGCCTTGGTCAG
AGCTCCTCTGTGACTTGTGGACCATGGAAGGGCTGGGCTCTTGCTTGCGG
CTGGAAAGGAAACAATGGCTTTATTTTTAACCTTAAAGTAGCAGATATGT
AACAAGATGGTGCCGTGTTACAA
(CloneID 4345766)

SEQ ID NO:189

GAAGAAAATCAAGCTTCTTGGGCCAAGTCAGCAGGTCCCAAGTCTAGTGG
TGGCAGGGAAGACTTGGAGTCATCTGGACTGCAGAGAAGGAACTCCTCTG
AAGCAAGCTCCGGAGACTTCTTAGATCTGAAGCGAGAAGGTGATATTCAT
GAAAATGTGGACACAGACTTGCCACGCAGCCTGGGGCAGAGTGAAGAGAA
GCCCGTGCCTGCTGCGCCTGTGCCCAGCCCGGTGGCCCCGGCCCCAGTGC
CATCCAGAAGAAATCCCCCTGGCGGCAAGTCCAGCCTCGTCTTGGGTTAG
CTCTGACTGTCCTGAACGCTGTCGTTCTGTCTGTTTCCTCCATGCTTGTG
AACTGCACAACTTGAGCCTGACTGTACATCTCTTGGATTTGTTTCATTAA
AAAGAAGCACTTTATGTACTGCTGTCTTTTTTTTTTTCCTTTTGAAGAAC
AGCTTTCTCTCTGTCCTTGACTCTGGGGTCTGTGGGCCATGGCATGAGTG
TTTCCTAGTAGTAGATGGGAGGGAAAGCTTTGTGACACTTAGTACTGTGT
TTTTAAGAAGAAATAATTGGGTCCCAGATGTGTCAGAGGATCTTTGGTAC
TGAGGTTTTTAACACCTTTACTGTGGGTTTACCAAGCCTCAACTGGACAG
ACCATAAACAGTCGACAAGGCACCGTCCTGCCAGGGCCGAACCACAGGGA
GTCTCTCCGCAGAGGCTTCTGGTGTTGCCTACTTGCCAGTGGCCTTGTCA
GAGCCTCTCCTGTGACATGTGAACACAGGAAAAGCCTGGGCCACTGACTT
GAGTGTGAAGA
(CloneID 3889866)

SEQ ID NO:190

TCGAGTCATCTGGACTGCAGAGAAGGAACTCCTCTGAAGCAAGCTCCGGA
GACTTCTTAGATCTGAAGGGAGAAGGTGATATTCATGAAAATGTGGACAC
AGACTTGCCAGGCAGCCTGGGGCAGAGTGAAGAGAAGCCCGTGCCTGCTG
CCCCTGTGCCCAGCCCGGTGGCCCCGGCCCCAGTGCCATCCAGAAGAAAT
CCCCCTGGCGGCAAGTCCAGCCTCGTCTTGGGTTAGCTCTGACTGTCCTG
AACGCTGTCGTTCTGTCTGTTTCCTCCATGCTTGTGAACTGCACAACTTG
AGCCTGACTGTACATCTCTTGGATTTCTTTCATTAAAAAGAAGCACTTTA
TGTACTGCTGTCTTTTTTTTTTTTCTTTGGAAGAACAGGTTTCTCTCTGT
CCTTGACTCTGGGGTCTGTGGGCCATGGCATGAGTGTTTCCTAGTAGTAG
ATGGGAGGGAAAGCTTGGTGACACTTAGTACTGTGTTTTAAGAAGAAATA
ATTGGGTTCCAGATGTGTCAGAGGATCTTNGTACTGAGGTTTTAACACTT
TACTTGGGTTTACCAAGCCTCAATGGACAGACCATAAACAGTCCACCAGG
CACCGTTCCTGCCAGGCCAACCCAGAGGGAGTCTCTCCGCGAGAGCCTTC
TGTGGTGGTGGCCCTAACTTGCAGTGGCTGGTCAGGAGCCCTCCCCTGTG
ACCTGTGACCCATGAAAGGCTGGGCTCTGCTGCCGCTGAAGAAAAACGCT
CTTATTTTACCTAAAAGTCCATTTACCGACGCGCGGTGACCCACTGTCCC
CCAGAGGCGGGGTCACAACGCCCGACACACCGGCACGGGGCCCGAGGCGC
CCAGGCCCGTCGAAACATGTGCGGCGAAACACCGGCGCAGGCGA
(CloneID 4450834)

SEQ ID NO:191

TCTTAGATCTGAAGGGAGAAGGTGATATTCATGAAAATGTGGACACAGAC
TTGCCAGGCAGCCTGGGGCAGAGTGAAGAGAAGCCCGTGCCTGCTGCGCC
TGTGCCGAGCCCGGTGGCCCCGGCCCCAGTGCCATCCAGAAGAAATCCCC
CTGGCGGCAAGTCCAGCCTCGTCTTGGGTTAGCTCTGACTGTCCTGAACG
CTGTCGTTCTGTCTGTTTCCTCCATGCTTGTGAACTGCACAACTTGAGCC
TGACTGTACATCTCTTGGATTTGTTTCATTAAAAAGAAGCACTTTATGTA
CTGCTGTCTTTTTTTTTTTTCTTTTGAAGAACAGGTTTCTCTCTGTCCTT
GACTCTGGGGTCTGTGGGCCAGTGGCATGAGTGTTTTCTAGTAGTAGATG
GGAGGGAAAGCTTGGTGACACTTAGTACTGTGTTTTTAAGAAGAAATAAT
TGGGTTCCAGATGTGTTAGAGGATCTTTGGTACTGAGGTTTTTAACACTT
TACTGGGGTTTACCAAGCCTCAACTGGACAGACCATAAACAGTCCACAGG
CACCGTTCCTGCCAGGCCCAACCCACAGGGAGTCTCTCCGAGAGCCTTCT
GTGGTGTGCCCTAAATTGCAGTGGCTTGTGTCAGAGCTCCTCCTGTGACA
TGTGACATGAAGAGGGCTGGGCTCCTGGCTTGGCGGCTGAAAGGAAAGAA
ACTGGCTTTATTTTTTAACCTTTAAAAGGTTGCGGATAGTAACAAGATTG
GGTGGGTGATGAGAAGGCTTGGTCTCAGCAAGAGAGGTGGTTCAATGAAA
TGCTGGATAAAAGCGTGACAG
(CloneID 5521426)

SEQ ID NO:192

TTACCAACAGAACAACCTGTGAGGAAGAACAAAATGGCCTCTAATATCTT
NTGGGACACNCTGAAGAAAATCAAGCTTCTTGGGCCAAGTCAGCAGGTGC
CAAGTCTAGTGGTGGCAGGGAAGACTTGGAGTCATCTGGACTGCAGAGAA
GGAACTCCTCTGAAGCAAGCTCCGGAGACTTCTTAGATCTGAAGGGAGAA
GGTGATATTCATGAAAATGTGGACACAGACTTGCCAGGCAGCCTGGGGCA
GAGTGAAGAGAAGCCCGTGCCTGCTGCGCCTGTGCCCAGCCCGGTGGCCC
CGGCCCCAGTGCCATCCAGAAGAAATCCCCCTGGCGGCAAGTCCAGCCTC
GTCTTGGGTTAGCTCTGACTGTCCTGAACGCTGTCGTTCTGTCTGTTTCC
TCCATGCTTGTGAACTGCACAACTTGAGCCTGACTGTACATCTCTTGGAT
TTGTTTCATTAAAAAGAAGCACTTTATGTACTGCTGTCTTTTTTTTTTTT
CTTTTGAAGAACAGGTTTCTCTCTGTCCTTGACTCTTGGGTCTGTGGGCC
ATGGCATGAGTGTTTTCTAGTAGTAGATTGGAGGGAAAGCTTTGTGACAC
TTAGTACTGTGTTTTTAAGAAGAAATAATTTGGTTCCAGATGTGTTAGAG
GATCTTTTGTACTGAGGTTTTTAACACTTTACTTGGGTTTACCAAGCCTC
AACTGGACAGACCATAAACAGTCCACAGGCACCGTTCCTGCCAGGCCCCA
ACCCACAGGGAGTCTCTCCGCAAGCCCTTCTTGGTGTTGCCCTAACTTGC
CAGTGGCCCTTTGCTCAGAGCCTCCCTCCTGTGACATGTGGAAACATGAA
AGAGGCCCTGCGCCCTCCCTGGCTTTTGCCGGCTTGCAAAGAAAAGAAAC
TGGCCCTTTTTATTTTTTTAACCCTTTAAAAAGGAGGCCAGAATAAGTAA
CCAGAACTTGGCCTGGGCTTGATGTAAACCAAAGCCCTTTGGTTCTCCCA
CCCCAAAAGGAAAAGGCTTTTGGGT
(CloneID 5267723)

SEQ ID NO:193

AGCGGGCGTAGCCATTGGATCCGGAGACCGGATCGATCAGAGTTTTGCCG
CCTCCAGGTGGTGGATCCAATTTTTCATTAGGTTTTGATGAACCAACAGA
ACAACCTGTGAGGAAGAACAAAATGGCCTCTAATATCTTTGGGACACCTG
AAGAAAATCAAGCTTCTTGGGCCAAGTCAGCAGGTGCCAAGTCTAGTGGT
GCCAGGGAAGACTTGGAGTCATCTGGACTGCAGAGAAGGAACTCCTCTGA
AGCAAGCTCCGGAGACTTCTTAGATCTGAAGGGAGAAGGTGATATTCATG
AAAATGTGGACACAGACTTGCCAGGCAGCCTGGGGCAGAGTGAAGAGAAG
CCCGTGCCTGCTGCGCCTGTGCCCAGCCCGGTGGCCCCGGCCCCAGTGCC
ATCCAGAAGAAATCCCCCTGGCGGCAAGTCCAGCCTCGTCTTGGGTTAGC
TCTGACTGTCCTGAACGCTGTCGTTCTGTCTGTTTCCTCCATGCTTGTGA
ACTGCACAACTTGAGCCTGACTGTACATCTCTTGGATTTGTTTCATTAAC
AAAGAAGCACTTTATGTACTGCTGTCTTTTTTTTTTTCCTTTGGAAGAAC
AGGTTTCTCTCTGTCCTTGACTCTGTGGGTCTGTGGGCCATGGCATGAGT
GTTTCCTAGTAGTAGAT
(CloneID 4441495)

SEQ ID NO:194

CAGGTGCCAAGTCTAGTGGTGGCAGGGAAGACTTGGAGTCATCTGGACTG
CAGAGAAGGAACTCCTCTGAAGCAAGCTCCGGAGACTTCTTAGATCTGAA
GGGAGAAGGTGATATTCATGAAAATGTGGACACAGACTTGCCAGGCAGCC
TGGGGCAGAGTGAAGAGAAGCCCGTGCCTGCTGCGCCTGTGCCCAGCCCG
GTGGCCCCCGCCCCAGTGCCATCCAGAAGAAATCCCCCTGGCGGCAAGTC
CAGCCTCGTCTTGGGTTAGCTCTGACTGTCCTGAACGCTGTCGTTCTGTC
TGTTTCCTCCATGCTTGTGAACTGCACAACTTGAGCCTGACTGTACATCT
CTTGGATTTGTTTCATTAAAAAGAAGCACTTTATGTACTGCTGTCTTTTT
TTTTTTCTTTTGAAGAACAGGTTTCTCTCTGTCCTTGACTCTTGGGTCTG
TGGGCCATGGCATGAGTGTTTTCTAGTAGTAGATTGGAGGGAAAGCTTTG
TGACACTTAGTACTGTGTTTTTAAGAAGAAATAATTTGGTTCCAGATGTG
TTAGAGGATCTTTGTACTGAGGTTTTTAACACTTTACTTGGGTTTACCAA
GCCTCAATCTGGACAGACGATAAAACAGTCCACAGGCACCNGTTCCTTGC
CAGGGCCCCAACCCACAGGGAGTCTCTCCCGGCAGAGCCTTTCTTGGTGT
TTGCCTTAAATTGGCAAGTGGGCCTTGGTCAAAGCCTCTTCCTGTGCATG
TGAACACTGGAAAGAGGCTGGGGCCTCCTGGCGTTGGCGGCTGGCAAAGA
AAAAAGCCGTTTTTTTTACCTTAAAGTGCCCAAGTTAACAACGGGTGGTC
TTGGAAACCTTGTTTCCCCAGAAGGGGGTGTGAAAAGGGGGGTACACAGG
AACAGGGGAAGCACAGGTCTCTCAACAGAGCACG
(CloneID 5395070)

SEQ ID NO:195

TGACCACAACCACCACCTTCAAGGGAGTCGACCCCAACAGCGAGGAATAG
CTCCCGAGTTTTGCGGCCTCCAGGTGGTGGATCCAATTTTTCATTAGGTT
TTGATGAACCAACAGAACAACCTGTGAGGAAGAACAAAATGGCCTCTAAT
ATCTTTGGGACACCTGAAGAAAATCAAGCTTCTTGGGCCAAGTCAGCAGG
TGCCAAGTCTAGTGGTGGCAGGGAAGACTTGGAGTCATCTGGACTGCAGA
GAAGGAACTCCTCTGAAGCAAGCTCCGGAGACTTCTTAGATCTGAAGGGA
GAAGGTGATATTCATGAAAATGTGGACACAGACTTGCCAGGCAGCCTGGG
GCAGAGTGAAGAGAAGCCCGTGCCTGCTGCGCCTGTGCCCAGCCCGGTGG
CCCCGGCCCCAGTGCCATCCAGAAGAAATCCCCCTGGCGGCAAGTCCAGC
CTCGTCTTGGGTTAGCTCTGACTGTCCTGAACGCTGTCGTACTGTCTGTT
TCCTCCATGCTTGTGAACTGCACAACTTGAGCCTGACTCTACATCTCTTG
GATTCGTTTCATTAAAAAGAAGCACTTTATGTACTGCTGTGTTTATTCTT
TCCCTTTTGAAGACCAGGTTTCTCTCTGTCCTTGACTCTGGGCTCTGTGG
GCCATTGGCATGCAGTTGTCTCCTAGTACGTAGCATGGGCGGGAAACGCT
TTGTGACCCCTTACGTACTGCGCTTCCAAGAACGACATCATGGGGTCGCA
GATTGTCGTAGAGGATCTTTGGGTACCTGCCGCGTTCTTCACCGCCTTTT
AACTGTGTGGTTATCCCAAGCCCTCCACTTGGACAGAACCATTAACCATG
CTCCACAGCGCACCGTCACTTGCAGGCCCAACCCCCAGGGGGGTCTTCCG
AGAGACCTTTTGGTGTGCCTATTTGCCCAGGGCGTTGTCAAACCCCCG
(CloneID 3445695)

SEQ ID NO:196

GCAGAGTGAAGAGAAGCCCGTGCCTGCTGCGCCTGTGCCCAGCCCGGTGG
CCCCGGCCCCTAGTGCCATCCAGAAGAAATCCCCCTGGCGGCAAGTCCAG
CCTCGTCTTGGGTTAGCTCTGACTGTCCTGAACGCTGTCGTTCTGTCTGT
TTCCTCCATGCTTGTGAACTGCACAACTTGAGCCTGACTGTACATCTCTT
GGATTTGTTTCATTAAAAAGAAGCACTTTATGTACTGCTGTCTTTTTTTT
TTCCTTTTGGAGAACAGGTTTCTCTCTGTCCTTGACTCTGGGGTCTGTGG
GCCATGGCATGAGTGTTTTCTAGTAGTAGATTGGCGGGAAAGCTTTGTGA
CACTTAGTACTGTGTTTTAAGAAGAAATAATTGGGTCCCAGATGTGTTAG
AGGATCTTTGGTACTGAGGTTTTTAACACTTTACTTGGCTTTACCAAGCC
TCAACTGGACAGACCATAAACAGTCCACAGGCACCGTTCCTGCCAGGCCC
CAACCCACAGGGAGTCTCTCCGCAGAGCCTTCTTGGTGTTGCCCTAACTT
GCCAGTGGCCTTGGCTCAGAGCCTCCTCCTGTGACCTGTGAACAATGAAG
AGGCCTGTGCCTCTGCCTTGCCGCTGCAGGCACAGAACTGCCTTTACTTT
TTAACCTTAAAAAGTAGCCGATAGTACAAGCTGGTTGGCTGTTGAAGCCA
AGCCTGGGTCTCACGCCGAGTAAGGCTGCCTGTTCCCTGCACACTGGCGG
GACCAAAAGCCGTGAACCAGGGGGGCATCCACTGTAAGCGGGTCTCCCGG
AACGGGCCCCAAGGGTGTTGTTACACTACCCGCGGGGGTGCATAGCTGTG
GCTGCCCTCAGGAAGCTCCTAACAGGTTTGCTCACCGGCGGGGCCTGAAA
ATC
(CloneID 5519819)

SEQ ID NO:197

TCAGCAGGTGCCAAGTCTAGTGGTGGCAGGGAAGACTTGGAGTCATCTGG
ACTGCAGAGAAGGAACTCCTCTGAAGCAAGCTCCGGAGACTTCTTAGATC
TGAAGGGAGAAGGTGATATTCATGAAAATGTGGACACAGACTTGCCAGGC
AGCCTGGGGCAGAGTGAAGAGAAGCCCGTGCCTGCTGCGCCTGTGCCCAG
CCCGGTGGCCCCGGCCCCAGTGCCATCCAGAAGAAATCCCCCTGGCGGCA
AGTCCAGCCTCGTCTTGGGTTAGCTCTGACTGTCCTGAACGCTGTCGTTC
TGTCTGTTTCCTCCATGCTTGTGAACTGCACAACTTGAGCCTCACTGTAC
ATCTCTTGGATTTGTTTCATTAAAAAGAAGCACTTTATGTACTGCTGTCT
TTTTTTTTTTTCTTTTGAAGAACAGGTTTCTCTCTGTCCTTGACTCTTGG
GTCTGTGGGCCATGGCATGAGTGTTTTCTAGTAGTAGATTGGAGGGAAAG
CTTTGTGACACTTAGTACTGTGTTTTTAAGAAGACATAATTTGGTTCCAG
ATGTGTTAGAGGATCTTTTGTACTGAGGTTTTTAACACTTTACCTTGGGT
TTACCAAGCCTTCCACTGGACAGGACCATAAACAGGTCCACAGGCCCCCG
TTTCTTGCCCAGGCCCCAAACCCACAGGGGAAGTCTCTTCCGCCACAAAC
CCTTCCTTTGGTGGCCCGCCCTTACCACCTGCCCCCGGCGGCGCTCTTTA
TGCTTTGGCGGAGCTCTCTCCTCTCCTTCATTCGAAGACATGCTGACCAA
CAACCACGGCCACACCAACGCCCCTCTCTATCTTTCTCATTGCGCCCCTG
CGCCCTGTATTGAGCGCGCCCTCAGAATTCGCGGGGCATACAGGGCGCGC
TGTCAATCCCCGAAACTCCAATATCGGTCTCCCCCCCGCTTTATATTAGC
GCGCCCCGTCGCTTGTAACGAGCCTAACTCAATTTTTTTTTCCCGCACTC
ACGCCCCGCTCTCGGTGTTACCAACCAACAGAACAGTCCTCCTCCTTCAT
AATATTCCCCAGACTCCCCCTCGCCATCTTACTCTTTTATTTNCCCCGCG
CGCGTGTTAAAAGCGGCAAAGGNNCCCCCACCGCACACACGTATATTGCC
CGGATTTTAATCATGAATTTTCCGCACCGGGCAAGACCACCCCCAGCTGC
CCCATCGTGTCCCCCGCCGTNCTGCAGAATTCCTCANATCTGGGCCCTCC
GCGATAGGTCACTCCATCCTCAGGAGAAAACACCCTCTTGTCCCACGCCG
TCCACTCTTAATACCGCCGTACCCTCATGTCCCCTCCGCCACTTCTTTTT
ACCCCCTCNGCGCTGTAGTTTTGCGGCGCCCCGACTACACTCACACCGAT
TATTTATTATCCCCCTTCANCCGACGCCGGNNGCCCCGAAAAAATGCCGC
CCCGGATCGATAACAGCTCACCCCCGCCCCCGCCAGGATGCCCCGCCGCC
AGCTAAAAGGCGCTCAACCA
(CloneID 2989794)

SEQ ID NO:198

CACGAGGACTGATGCCCAAGAGTTAAATGGCCTCTAATATCTTTGGGACA
CCATGAAGAAAATCAAGACTTCTTGGGCCAAGTCAACAGGTGCCAAGTCT
AGTGCTGGAAGGGAAGACTTGTGAGTCATCTGGACTGCAGAGAAGGAACT
CCTCTGAAGCAAGCTCCGGAGACTTCTTAGATCTGAAGGGAGAAGGTGAT
ATTCATGAAAATGTGAGACACAGACTTGCCAGTGCAGCCTGGGGCAGAGT
GAAGAGAAGCCCTGTGCCTGCTGCTGCCTGTGCCCAGCCCTGGTGGACCC
AGGCCCCAGTGCCATCCAGAAGAAATCCCCACTGGCGGCAAGTCCAGCCT
CGTCTTGGGTTAGCTCTGACTGTCCTGAACTGCTAGTCAGATATCTAGTC
TGTAACCTCCATGCTTGTGAACTGCACAACTTGAGCCTCACTGTACATCT
CTTGGAATTTGTTTCATTAAAAAGAAGCACTTATATGTACTGCTGTCTTT
ATTATTAATATACTTTGTGCAAGAACAGGTAAACACATCTGACACATTGA
CCTCTGTGGACTCTGTGAGAGCCATAGGCATTCAGTTGTGAACTAGCTAG
ATACATGGCGAGGGAACAGCTTTGTGAACACTAAGAACTGAGATCATAAC
GACAAAATACATGGGTCCAGATGTGTAGAGGATCTAAGGAAATGAGGTAT
AAAACTAACTTGGTTTCCAAGCTCAATGGGCGACA
(CloneID 1637898 in 3′ to 5′ orientation)

SEQ ID NO:199

AAAGTGCTTCTTTTTAATGAAACAAATCCAAGAGATGTACAGTCAGGCTC
AAGTTGTGCAGTTCACAAGCATGGAGGAAACAGACAGAACGACAGCGTTC
ACGACAGTCAGAGCTAACCCAAGACGAGGCTGGACTTGCCGCCAGGGGGA
TTTCTTCTGGATGGCACTGGGGCCGGGGCCACCGGGCTGGGCACAGGCGC
AGCAGGCACGGGCTTCTCTTCACTCTGCCCCAGGCTGCCTGGCAAGTCTG
TGTCCACATTTTCATGAATATCACCTTCTCCCTTCAGATCTAAGAAGTCT
CCGGAGCTTGCTTCAGAGGAGTTCCTTCTCTGCAGTCCAGATGACTCCAA
GTCTTCCCTGCCACCACTAGACTTGGCACCTGCTGACTTGGCCCAAGAAG
CTTGATTTTCTTCAGGTGTCCCAAAGATATTAGAGGCCATTTTGTTCTTC
CTCACAGGTTGTTCTGTTGGTTCATCAAAACCTAATGAAAAATTGGATCC
ACCACCTGGAGGCCGCAAAACTCGGGAGCTATTCCTGCTGTTGGGGTCGA
CTCCCTTGAAGGTGGT
(CloneID 1132042 in 3′ to 5′ orientation)

SEQ ID NO:200

TTTTTTTAAAGTGCTTCTTTTTAATGAAACAAATCCAAGAGATGTACAGT
CAGGCTCAAGTTGTGCAGTTCACAAGCATGGAGGAAACAGACAGAACGAC
AGCGTTCAGGACAGTCAGAGCTAACCCAAGACGAGGCTGGACTTGCCGCC
AGGGAGATTTCTTCTGGATGGCACTGGGGCCGGGGCACCGGGCTGGGCAC
AGGCGCACGAGGCACGGGCTTCTCTTCACTCTGCCCCAGGCTGCCTGGCA
AGTCTGTGTCCACATTTTCATGAATATCACCTTCTCCCTTCAGATCTAAG
AAGTCTCCGGAGCTTGCTTCAGACGAGTTCCTTCTCTGCAGTCCAGATGA
CTCCAAGTCT

In another set of preferred embodiments of the invention, the sequence, or a unique portion thereof, of the ESE-2/ELF5 sequences identified by the I.M.A.G.E. Consortium CloneIDs 1864302 and 4480123 as well as clusters C019657 and NM_{—}001422 may be used. Similarly, sequences from the UniGene Homo sapiens cluster Hs.11713, may be used. ESE-2/ELF5 sequences are useful in discriminations between normal and ADH or DCIS or IDC breast cells based upon decreased expression in non-normal breast cells. Human ESE-2/ELF5 has been mapped to 11p13-p15.

ESE-2/ELF5 related sequences are as follows, with the assigned coding region (ending with a termination codon) underlined and preceded by the 5′ untranslated and/or non-coding region and followed by the 3′ untranslated and/or non-coding region. The 3′ untranslated and/or non-coding regions are presented as the complementary strand in the 3′ to 5′ direction for some of the sequences.

SEQ ID NOS:204-209 are found in the 3′ untranslated and/or non-coding regions of the disclosed ESE-2/ELF5 sequences.

(CloneID 4480123, ESE-2b/ELF5)

SEQ ID NO:201

CACAAGGCTACAGGTGTCTTTATTTCCACTGCACGCTGGTGCTGGGAGCG
CCTGCCTTCTCTTGCCTTGAAAGCCTCCTCTTTGGACCTAGCCACCGCTG
CCCTCACGGTAATGTTGGACTCGGTGACACACAGCACCTTCCTGCCTAAT
GCATCCTTCTGCGATCCCCTGATGTCGTGGACTGATCTGTTCAGCAATGA
AGAGTACTACCCTGCCTTTGAGCATCAGACAGCCTGTGACTCATACTGGA
CATCAGTCCACCCTGAATACTGGACTAAGCGCCATGTGTGGGAGTGGCTC
CAGTTCTGCTGCGACCAGTACAAGTTGGACACCAATTGCATCTCCTTCTG
CAACTTCAACATCAGTGGCCTGCAGCTGTGCAGCATGACACAGGAGGAGT
TCGTCGAGGCAGCTGGCCTCTGCGGCGAGTACCTGTACTTCATCCTCCAG
AACATCCGCACACAAGGTTACTCCTTTTTTAATGACGCTGAAGAAAGCAA
GGCCACCATCAAAGACTATGCTGATTCCAACTGCTTGAAAACAAGTGGCA
TCAAAAGTCAAGACTGTCACAGTCATAGTAGAACAAGCCTCCAAAGTTCT
CATCTATGGGAATTTGTACGAGACCTGCTTCTATCTCCTGAAGAAAACTG
TGGCATTCTGGAATGGGAAGATAGGGAACAAGGAATTTTTCGGGTGGTTA
AATCGGAAGCCCTGGCAAAGATGTGGGGACAAAGGAAGAAAAATGACAGA
ATGACATATGAAAAGTTGAGCAGAGCCCTGAGATACTACTATAAAACAGG
AATTTTGGAGCGGGTTGACCGAAGGTTAGTGTACAAATTTGGAAAAAATG
CACACGGGTGGCAGGAAGACAAGCTATGATCTGCTCCAGGCATCAAGCTC
ATTTTATGGATTTCTGTCTTTTAAAACAATCAGATTGCAATAGACATTCG
AAAGGCTTCATTTTCTTCTCTTTTTTTTTTAACCTGCAAACATGCTGATA
AAATTTCTCCACATCTCAGCTTACATTTGGATTCAGAGTTGTTGTCTACG
GAGGGTGAGAGCAGAAACTCTTAAGAAATCCTTTCTTCTCCCTAAGGCGA
TGAGGGGATGATCTTTTGTGGTGTCTTGATCAAACTTTATTTTCCTAGAG
TTGTCGAATGACAACAGCCCATGCCATTGATGCTGATCAGAGAAAAACTA
TTCAATTCTGCCATTAGAGACACATCCAATGCTCCCATCCCAAAGGTTCA
AAAGTTTTCAAATAACTGTGGCAGCTCACCAAACGTGGGGGAAAGCATGA
TTAGTTTGCAGGTTATGGTAGGAGAGGGTGAGATATAAGACATACATACT
TTAGATTTTAAATTATTAAAGTCAAAAATCCATAGAAAAGTATCCCTTTT
TTTTTTTTTTGAGACGGGTTCTCACTATGTTGCCCAGGGCTGGTCTTGAA
CTCCTATGCTCAAGTGATCCTCCCACCTCGGCCTCCCAAAGTACTGTGAT
TACAAGCGTGAGCCACGGCACCTGGGCAGAAAAGTATCTTAATTAATGAA
AGAGCTAAGCCATCAAGCTGGGACTTAATTGGATTTAACATAGGTTCACA
GAAAGTTTCCTAACCAGAGCATCTTTTTGACCACTCAGCAAAACTTCCAC
AGACATCCTTCTGGACTTAAACAGTTAACATTAACCACATTATTAATTGT
TGCTGAGTTTATTCCCCCTTCTAACTGATGGCTGGCATCTGATATGCAGA
GTTAGTCAACAGACACTGGCATCAATTACAAAATCACTGCTGTTTCTGTG
ATTCAAGCTGTCAACACAATAAAATCGAAATTCATTGATTCCATCTCTGG
TCCAGATGTTAAACGTTTATAAAACCGGAAATGTCCTAACAACTCTGTAA
TGGCAAATTAAATTGTGTGTCTTTTTTGTTTTGTCTTTCTACCTGATGTG
TATTCAAGTGCTATAACACGTATTTCCTTGACAAAAATAGTGACAGTGAA
TTCACACTAATAAATGTTCATAGGTTAAAGTCTGCACTGACATTTTCTCA
TCAATCACTGGTATGTAAGTTATCAGTGACTGACAGCTAGGTGGACTGCC
CCTAGGACTTCTGTTTCACCAGAGCAGGAATCAAGTGGTGAGGCACTGAA
TCGCTGTACAGGCTGAAGACCTCCTTATTAGAGTTGAACTTCAAAGTAAC
TTGTTTTAAAAAATGTGAATTACTGTAAAATAATCTATTTTGGATTCATG
TGTTTTCCAGGTGGATATAGTTTGTAAACAATGTGAATAAAGTATTTAAC
ATGTAAAAAAAAAAAAAAA
(ESE-2a)

SEQ ID NO:202

GGCTGAGTGGTTTGCTCCTTCCCCTCTCTCTGGGAGGCTGAGCAGGGGTG
CCTGGGTTGCTCAGGCCATGGGAGCCACACCTGTTATTGCTGCCTCTGAT
TTGTGTGACACTGAGAAGCCCACAGGCCTGTCCCTCCAACTCGGTGGACC
CTCTCTGTGTGCATTTGGTGTGTGAGCCAGCTCTGAGAAGGGTTCAGAAG
CCACTGGAGGCATCTGGGGACCTCAGCTTCCATGCCATCTCTGCCTCACT
CCCACAGGGTAATGTTGGACTCGGTGACACACAGCACCTTCCTGCCTAAT
GCATCCTTCTGCGATCCCCTGATGTCGTGGACTGATCTGTTCAGCAATGA
AGAGTACTACCCTGCCTTTGAGCATCAGACAGCCTGTGACTCATACTGGA
CATCAGTCCACCCTGAATACTGGACTAAGCGCCATGTGTGGGAGTGGCTC
CAGTTCTGCTGCGACCAGTACAAGTTGGACACCAATTGCATCTCCTTCTG
CAACTTCAACATCAGTGGCCTGCAGCTGTGCAGCATGACACAGGAGGAGT
TCGTCGAGGCAGCTGGCCTCTGCGGCGAGTACCTGTACTTCATCCTCCAG
AACATCCGCACACAAGGTTACTCCTTTTTTAATGACGCTGAAGAAAGCAA
GGCCACCATCAAAGACTATGCTGATTCCAACTGCTTGAAAACAAGTGGCA
TCAAAAGTCAAGACTGTCACAGTCATAGTAGAACAAGCCTCCAAAGTTCT
CATCTATGGGAATTTGTACGAGACCTGCTTCTATCTCCTGAAGAAAACTG
TGGCATTCTGGAATGGGAAGATAGGGAACAAGGAATTTTTCGGGTGGTTA
AATCGGAAGCCCTGGCAAAGATGTGGGGACAAAGGAAGAAAAATGACAGA
ATGACGTATGAAAAGTTGAGCAGAGCCCTGAGATACTACTATAAAACAGG
AATTTTGGAGCGGGTTGACCGAAGGTTAATGTACAAATTTGGAAAAAATG
CACACGGGTGGCAGGAAGACAAGCTATGATCTGCTCCAGGCATCAAGCTC
ATTTTATGGATTTCTGTCTTTTAAAACAATCAGATTGCAATAGACATTCG
AAAGGCTTCATTTTCTTCTCTTTTTTTTTAACCTGCAAACATGCTGATAA
AATTTCTCCACATCTCAGCTTACATTTGGATTCAGAGTTGTTGTCTACGG
AGGGTGAGAGCAGAAACTCTTAAGAAATCCTTTCTTCTCCCTAAGGGGAT
GAGGGGATGATCTTTTGTGGTGTCTTGATCAAACTTTATTTTCCTAGAGT
TGTGGAATGACAACAGCCCATGCCATTGATGCTGATCAGAGAAAAACTAT
TCAATTCTGCCATTAGAGACACATCCAATGCTCCCATCCCAAAGGTTCAA
AAGTTTTCAAATAACTGTGGCAGCTCACCAAAGGTGGCGGAAAGCATGAT
TAGTTTGCAGGTTATGGTAGGAGAGGGTGAGATATAAGACATACATACTT
TAGATTTTAAATTATTAAAGTCAAAAATCCATAGAAAAGTATCCCTTTTT
TTTTTGAGACGGGTTCTCACTATGTTGCCCAGGGCTGGTCTTGAACTCCT
ATGCTCAAGTGATCCTCCCACCTCGGCCTCCCAAAGTACTGTGATTACAA
GCGTGAGCCACGGCACCTGGGCAGAAAAGTATCTTAATTAATGAAAGAGC
TAAGCCATCAAGCTGGGACTTAATTGGATTTAACATAGGTTCACAGAAAG
TTTCCTAACCAGAGCATCTTTTTGACCACTCAGCAAAACTTCCACAGACA
TCCTTCTGGACTTAAACACTTAACATTAACCACATTATTAATTGTTGCTG
AGTTTATTCCCCCTTCTAACTGATGGCTGGCATCTGATATGCAGAGTTAG
TCAACAGACACTGGCATCAATTACAAAATCACTGCTGTTTCTGTCATTCA
AGCTGTCAACACAATAAAATCGAAATTCATTGATTCCATCTCTGGTCCAG
ATGTTAAACGTTTATAAAACCGGAAATGTCCTAACAACTCTGTAATGGCA
AATTAAATTGTGTGTCTTTTTTGTTTTGTCTTTCTACCTGATGTGTATTC
AAGCGCTATAACACGTATTTCCTTGACAAAAATAGTGACAGTGAATTCAC
ACTAATAAATGTTCATAGGTTAAAGTCTGCACTGACATTTTCTCATCAAT
CACTGGTATGTAAGTTATCAGTGACTGACAGCTAGGTGGACTGCCCCTAG
GACTTCTGTTTCACCAGAGCAGGAATCAAGTGGTGAGGCACTGAATCGCT
GTACAGGCTGAAGACCTCCTTATTAGAGTTGAACTTCAAAGTAACTTGTT
TTAAAAAATGTGAATTACTGTAAAATAATCTATTTTGGATTCATGTGTTT
TCCAGGTGGATATAGTTTGTAAACAATGTGAATAAAGTATTTAACATGCG
AA
(alternative ELF5 encoding sequence)

SEQ ID NO:203

CAAGGCTACAGGTGTCTTTATTTCCACTGCACGCTGGTGCTGGGAGCGCC
TGCCTTCTCTTGCCTTGAAAGCCTCCTCTTTGGACCTAGCCACCGCTGCC
CTCACGGTAATGTTGGACTCGGTGACACACAGCACCTTCCTGCCTAATGC
ATCCTTCTGCGATCCCCTGATGTCGTGGACTGATCTGTTCAGCAATGAAG
AGTACTACCCTGCCTTTGAGCATCAGACAGCCTGTGACTCATACTGGACA
TCAGTCCACCCTGAATACTCGACTAAGCGCCATGTGTGGGAGTGGCTCCA
GTTCTGCTGCGACCAGTACAAGTTGGACACCAATTGCATCTCCTTCTGCA
ACTTCAACATCAGTGGCCTGCAGCTGTGCAGCATGACACAGGAGGAGTTC
GTCGAGCCAGCTGGCCTCTGCGGCGAGTACCTGTACTTCATCCTCCAGAA
CATCCGCACACAAGGTTACTCCTTTTTTAATGACGCTGAAGAAAGCAAGG
CCACCATCAAAGACTATGCTGATTCCAACTGCTTGAAAACAAGTGGCATC
AAAAGTCAAGACTGTCACAGTCATAGTAGAACAAGCCTCCAAAGTTCTCA
TCTATGGGAATTTGTACGAGACCTGCTTCTATCTCCTGAAGAAAACTGTG
GCATTCTGGAATGGGAAGATAGGGAACAAGGAATTTTTCGGGTGGTTAAA
TCGGAAGCCCTGGCAAAGATGTGGGGACAAAGGAAGAAAAATGACAGAAT
GACATATGAAAAGTTGAGCAGAGCCCTGAGATACTACTATAAAACAGGAA
TTTTGGAGCGGGTTGACCGAAGGTTAGTGTACAAATTTGGAAAAAATGCA
CACGGGTGGCAGGAAGACAAGCTATGATCTGCTCCAGGCATCAAGCTCAT
TTTATGGATTTCTGTCTTTTAAAACAATCAGATTGCAATAGACATTCGAA
AGGCTTCATTTTCTTCTCTTTTTTTTTAACCTGCAAACATGCTGATAAAA
TTTCTCCAGATCTCAGCTTACATTTGGATTCAGAGTTGTTGTCTACGGAG
GGTGAGAGCAGAAACTCTTAAGAAATCCTTTCTTCTCCCTAAGGGGATGA
GGGGATGATCTTTTGTGGTGTCTTGATCAAACTTTATTTTCCTAGAGTTG
TGGAATGACAACAGCCGATGCCATTGATGCTGATCAGAGAAAAACTATTC
AATTCTGCCATTAGAGACACATCCAATGCTCCCATCCCAAAGGTTCAAAA
GTTTTCAAATAACTGTGGCAGCTCACCAAAGGTGGGGGAAAGCATGATTA
GTTTGCAGGTTATGGTAGGAGAGGGTGAGATATAAGACATACATACTTTA
GATTTTAAATTATTAAAGTCAAAAATCCATAGAAAAGTATCCCTTTTTTT
TTTGAGACGGGTTCTCACTATGTTGCCCAGGGCTGGTCTTGAACTCCTAT
GCTCAAGTGATCCTCCCACCTCGGCCTCCCAAAGTACTGTGATTACAAGC
GTGAGCCACGGCACCTGGGCAGAAAAGTATCTTAATTAATGAAAGAGCTA
AGCCATCAAGCTGGGACTTAATTGGATTTAACATAGGTTCACAGAAAGTT
TCCTAACCAGAGCATCTTTTTGACCACTCAGCAAAACTTCCACAGACATC
CTTCTGGACTTAAACACTTAACATTAACCACATTATTAATTGTTGCTGAG
TTTATTCCCCCTTCTAACTGATGGCTGGCATCTGATATGCAGAGTTAGTC
AACAGACACTGGCATCAATTACAAAATCACTGCTGTTTCTGTGATTCAAG
CTGTCAACACAATAAAATCGAAATTCATTGATTCCATCTCTGGTCCAGAT
GTTAAACGTTTATAAAACCGGAAATGTCCTAACAACTCTGTAATGGCA
(CloneID 377520)

SEQ ID NO:204

CAGACACTGGCATCAATTACAAAATCACTGCTGTTTCTGTGATTCAAGCT
GTCAACACAATAAAATCGAAATTCATTGATTCCATCTCTCGTCCAGATGT
TAAACGTTTATAAAACCGGAAATCTCCTAACAACTCTGTAATGGCAAATT
AAATTGTGTGTCTTTTTTGTTTTGTCTTTCTACCTGATGTGTATTCAAGC
GCTATAACACGTATTTCCTTGACAAAAATAGTGACAGTGAATTCACACTA
ATAAATGTTCATAAGGTTAAAGTCTGCACTGACATTTTCTCATCAATCAC
TGGTATGTAAGTTATCAGTGACNGACAGCTAAGGTGNGACTGG
(CloneID 377520 in 3′ to 5′ orientation)

SEQ ID NO:205

GAACATGTTAAATACTTTATTCACATTGTTTACAAACTATATCCACCTGG
AAAACACATGAATCCAAAATAGATTATTTTACAGTAATTCACATTTTTTA
AAACAAGTTACTTTGAAGTTCAACTCTAATAAGGAGGTCTTCAGCCTGTA
CAGCGATTCAGTGCCTCACCACTTGATTCCTGCTCTGGTGAAACAGAAGT
CCTAGGGGCAGTCCACCTAG
(CloneID 81671)

SEQ ID NO:206

TGATTCAAGCTGTCAACACAATAAAATCGAAATTCATTGATTCCATCTCT
GGTCCAGATGTTAAACGTTTATAAAACCGGAAATGTCCTAACAACTCTGT
AATGGCAAATTAAATTGTGTGTCTTTTTTGTTTTGTCTTTCTACCTGATG
TGTATTCAAGCGCTATAACAC
(CloneID 81671 in 3′ to 5′ orientation)

SEQ ID NO:207

GCGGTGAAATACTTTATTCACATTGTTTACAAACTATATCCACCTGGAAA
ACACATGAATCAAAATAGATTATTTTACAGTAATNACATTTTTTAAAACA
AGTTACTTTGAAGTCAACTCTAATAAGGAGGTCTTCAGCCTGTACAGCGA
TTCAGTGCCTCACCACTTGATTCCTGCTCTGGTGAACAGAAGTCCTAGGG
GCAATCCACCTAGCTGTCAGTCACTGATAACTTACATACCAGTGATTGAT
GAGAAAATGTCAGTGCAGACTTTAACCTATGAACATTTATTAGTGTGAAT
TCACTGTCACTATTTTTGTCAAGGAAATACGTGTTATAGCGCTTGAATAC
ACATCAGGTAGAAAGACAAANCAAAAAAGNCACACAATTTAATTTGCCAT
TACAGAGTTGTTAGGGCCATTTCCGGTTTTATAAACGTTTACATCTGGGC
CNGAGATGCGATCAATGAATTTCGNTTTTATTGTGTTGACCGCTTGNATC
ACCTCGTGCCGATTTCCTGCGGCCCGGGGNTCCCCTAGTT
(CloneID 2956497 in 3′ to 5′ orientation)

SEQ ID NO:208

AGACGGGTTCTCACTATGTTGCCCAGGGCTGGTCTTGAACTCCTATGCTC
AAGTGATCCTCCCACCTCGGCCTCCCAAAGTACTGTGATTACAAGCGTGA
GCCACGGCACCTGGGCAGAAAAGTATCTTAATTAATGAAAGAGCTAAGCC
ATCAAGCTGGGACTTAATTGGATTTAACATAGGTTCACAGAAAGTTTCCT
AACCAGAGCATCTTTTTGACCACTCAGCAAAACTTCCACAGACATCCTTC
TGGACTTAAACACTTAACATTAACCACATTATTAATTGTTGCTGAGTTTA
TTCCCCCTTCTAACTGATGGCTGGCATCTGATATGCAGAGTTAGTCAACA
GACACTGGCATCAATTACAAAATCACTGCTGTTTCTGTGATTCAAGCTGT
CAACACAATAAAATCGAAATTCATTGATTCCATCTCTGGTCCAGATGTTA
AACGTTTATAAAACCGG
(CloneID 1864302 in 3′ to 5′ orientation)

SEQ ID NO:209

TGAACATGTTAAATACTTTATTCACATTGTTTACAAACTATATCCACCTG
GAAAACACATGAATCCAAAATAGATTATTTTACAGTAATTCACATTTTTT
AAAACAAGTTACTTTGAAGTTCAACTCTAATAAGGAGGTCTTCAGCCTGT
ACAGCGATTCAGTGCCTCACCACTTGATTCCTGCTCTGGTGAAACAGAAG
TCCTAGGGGCAGTCCACCTAGCTGTCAGTCACTGATAACTTACATACCAG
TGATTGATGAGAAAATGTCAGTGCAGACTTTAACCTATGAACATTTATTA
GTGTGAATTCACTGTCACTATTTTTGTCAAGGAAATACGTGTTATAGCAC
TTGAATACACATCAGGTAGAAAGACAAAACAAAAAAGACACACAATTTAA
TT
(ESE-2 promoter 1, positions 1-1046, and 5′
untranslated region of ESE-2b, positions 1047 to
>1106)

SEQ ID NO:210

TTCAATCCCACTTCCTCCTTTTGCCACTGGGGAAAAATGAAGCCCAGAGA
AGTCCAGGTTACCCAGCCAAGATAGGGGTCTAGGCAGTCACCTATCTTCC
TTCAACAATTTTTCCATGATGACACCCCGAATCCTCTCACGAACTGCCTT
TTGTTTCTGCAGACTATATGCGTATTTGTATTTGTGTGTTTGTAACCATG
CCCGGTGATCCCAAGAAAACATAACAAACCAGTCCTTGCTTTCGCTTTAA
AGCTTGGACTCTGCCATTTGAATACAACATCTCGGCTGCCCAAGATGGCT
AGAAGCAGAATGCAAAAAGGCACAAGGGTTATAAATACCTGTCTCATAGA
TGACCCGGGACACTTGTGCTTTGCAGCCTAAATTAGGCAGAGTTTCTGTT
GTCACGGAGAAGTACTAAAAAGCGGGCAGTTCTCAGCGAGACACCTTGAG
AGGCTGGCATCCACATGAGGAGAGGCCCCATCACTTACATTACACTCATG
AAGCCCAACAGATGTTAAGCTACATTTTTCTAGGTAGCACAGCTAGGAAA
TGATGGACACTGAATTTTGAATCTAAAGGCAACTAGCTGCAAAACCTGAG
GTCTCAACACTGGGCCTATAATGCTCTTCCTTCTCACCACATGGGAAAAA
CGGAGAAAGACATGATCTTACAAAAGCACTGGGCTCAGCCAGCCTGGGAG
AGAGGCAGCGCAAACCGGCACCAGGGTGGAATCTCTGTGTTTCCCCTCCA
GCTTGCACACAGGAAAATTCCTCACCAAACGCACGATCCCCGCACAGCTT
CCCAGGGACAAGAATTTTCTGCTTGTTTACTGAGTCCCCTGGGCTCGGAG
TGGGGGGTTTGCAGGGCAGGGGTGAGCTGCGCACAAAAGCAGGATAAAGG
TAAACTTTCTGCATATGAGAACCATTTCCCCCCCTCCATAGGAGCCGTGT
CACACTGTATGTCACCGTCATCAAAGGGGCTGTGCGTAAACCTGAAAAAC
CAAACGGACCTGTCTGTAGGTGTCACTTATATCACAAGGTACAGGTGTCT
TTATTTCCACTGCACGCTGGTGCTGGGAGCGCCTGCCTTCTCTTGCCTTG
AAAGCC
(ESE-2 promoter 2, positions 1-1879, and 5′
untranslated region of ESE-2a, positions 1080 to
>1975)

SEQ ID NO:211

GTAAGCGCCTTTTCTTTGCTTTTTTGGATGAATCGTAAGGGGTCTCAGGG
CTCAGAGGCTCGGCCTGGGGCCTGGGTCTGCTCGTCCATCTCCAGCTCCA
GCGCCGGCACCTGCGCTGGGTGCAGTCCCAGGGCCTCCGGGAGCTGTTTG
ACACGGATGTGACGGCTTGCCTCAGAGAGACCTGCTTGCCTCCGCAGGCC
GTGTGGCCACCAGCCCTCCTCACAGCCACCACTCCCTCTGGATCCCTTAG
CTGAACCAAGGGGAATTAAGAGAACGCGCCCAGGCGCTCAGTTCTCTATC
CTCCCTAAAGCTTCTTTAGGAAGGGAAGCTGGATTTTACTCAGAAAAATA
TTCAGCTTCAAGGAAAGCCCCCCACCTACCCTTCTCCCCTGACTGAATTC
CTTTCCCCACCGGCCTGCCCACTGTCCCCCACCCCCTCGATACAAACAAC
TCCCTTATTATAGTTTGGGGCATTTTCAGGAATTTGTACAAAACAGTTTT
AAAAGAAACCCATTTGCCCTCTGACATCTTCCCCAGAAGAGAATGCTTCC
AGAAAAGCAATGGTTAGCGTTTTTCCACTGTGTTCTCAGGCCTTGTCAGA
CTCTGCAGTGTGTGGGGTTTCTGTGGGGTTGCCCCCTGCTGACACCGACA
GGTAGCACAAGCTCCCTCCTCAGGCTGAAGCCGGGCCTGCGTGTGGATGC
CTTTTGTACGCCCGAAAGTGTGTCAGACATCACTAGGTGCAATCGCTTCA
TGTTACAGATGAGGAAACTGAGGCCAGAAAGGCTAGATGACTTAGGGAGG
CAGCTCTGTGGTTCAGTCTAGTCCAGAGACCCAGGGAAAGGGCTGGTTTG
CCCATCCGAGTGAACGGCTTGATTCTGTTGTCTTCGTGTTCTCTGCAATA
CTGTTTTTCATAATCTAAGGTATTCTCGGGCCGGAGTTTTCAGTTTGGTC
ATCTCCCTTTTTCAGAAGTCACCAGAGTGGCAAATTTTTAGATTCATTGT
TGATGGCTGATTTTTCCCTCTAAAACAGGATTGAATATATGTATTATGCC
CCGTCCTTTGCATATATCGGATTGCATGATTTAAAAAAAAAAAAATCCCC
ATTTTAGAGATGAGGGAAACCGTAGTTCGAAGAGGGCCATGAATATTTAT
ATACCACATCTATATGAAAAAAAGCTTAAGAATTTCAGCAGTCTGGGTAG
TAAGAAATGTGCCCCACGTTCGCAGCAGCAGAGGTAGGATTTAACCCCTG
ATATATGTAATCCAAAACTGGTAAGCTTTGCATCGAAGCCTACAGCAAAC
TACATGGCATACCTTTTTGGATCTTCGCTGCACCTTCAGAAAAACAGCTA
CAAACAAACAAACACGCAAACTCCCTAAGCGAGCTAAAGAAAAATGAGGA
ATCACTTGACAAGAGGTCAGCAAATTGGTTCTCACAGTTGCTAGGGTTCC
TCTCAGAAGAACAAACCCTCAAGCACCCACCCTCAATCTTTCTTTAGCAG
TAAGTGTTGAAATACTCCACTGGGTTCTCCTCAGTGGGCCCCTTCTTAGG
GAGGTCCAAGGGCTGGGAAATTGACCCTCCCTTGCTCACAAGTCTTGGGA
CGGAGTGGACATTTGGGTATCACTGAGGCTTTAAGGAAGGAAAACCGATT
TTCTTCCCAATGATACCACTCAGAGGGTGGGGTTGAGGAAGCCCAATCTT
GGCCACTTTTTTCTATATTTTGCACCCTATGGCCTAGTTCTGCCCAGTGA
TGATTCGGCCCGTAAACAGCCAATGTGTAGATGCTTAATTGGGCCAATTT
TTGGTCACATGCCCAGAGTGAAGTTGATGATCACCACCAGAGTCAGGAAG
GAATTTTCCTCCTCTGGCAAACTGGCCAAGGCTGAGTGGTTTGCTCCTTC
CCCTCTCTCTGGGAGGCTGAGCAGGGGTGCCGGGTTGCTCAGGCCATGGG
AGCCACACCTGTTATTGCTGCCTCT

All sequences are provided using conventional representations of a DNA strand starting from the 5′ phosphate linked end to the 3′ hydroxyl linked end. The above assignment of coding regions is generally by comparison to available consensus sequence(s) and therefore may contain inconsistencies. These have no effect on the practice of the invention because the invention can be practiced by use of shorter segments (or combinations thereof) of sequences unique to each of the three sets described above and not affected by inconsistencies. As a non-limiting example, a segment of CRIP1 composed of a 3′ untranslated region sequence and a sequence from the 3′ end of the coding region may be used as a probe for the detection of CRIP1 expression without being affected by the presence of any inconsistency in the representations of the coding regions provided above. Similarly, the use of an antibody which specifically recognizes CRIP1 protein to detect its expression would not be affected by the presence of any inconsistency in the representation of the coding regions provided above.

As will be appreciated by those skilled in the art, some of the above sequences include 3′ poly A (or poly T on the complementary strand) stretches that do not contribute to the uniqueness of the disclosed sequences. The invention may thus be practiced with sequences lacking the 3′ poly A (or poly T) stretches. The uniqueness of the disclosed sequences refers to the portions or entireties of the sequences which are found only in CRIP1, HN1, and ESE-2/ELF5 nucleic acids. Preferred unique sequences for the practice of the invention are those which contribute to the consensus sequences for each of the three sets. These preferred unique sequences are of the lengths of polynucleotides of the invention as discussed herein.

To determine the (increased or decreased) expression levels of the above described sequences in the practice of the present invention, any method known in the art may be utilized. In one preferred embodiment of the invention, expression based on detection of RNA which hybridizes to polynucleotides containing the above described sequences is used. This is readily performed by any RNA detection or amplification+detection method known or recognized as equivalent in the art such as, but not limited to, reverse transcription-PCR (optionally real-time PCR), the methods disclosed in U.S. patent application Ser. No. 10/062,857 entitled “Nucleic Acid Amplification” filed on Oct. 25, 2001 as well as U.S. Provisional Patent Application 60/298,847 (filed Jun. 15, 2001) and 60/257,801 (filed Dec. 22, 2000), the methods disclosed in U.S. Pat. No. 6,291,170, and quantitative PCR. Methods to identify increased RNA stability (resulting in an observation of increased expression) or decreased RNA stability (resulting in an observation of decreased expression) may also be used. These methods include the detection of sequences that increase or decrease the stability of mRNAs containing the CRIP1, HN1, and ESE-2/ELF5 sequences disclosed herein. These methods also include the detection of increased mRNA degradation.

In particularly preferred embodiments of the invention, polynucleotides having sequences present in the 3′ untranslated and/or non-coding regions of the above disclosed sequences are used to detect expression or non-expression of CRIP1, HN1, and ESE-2/ELF5 sequences in breast cells in the practice of the invention. Such polynucleotides may optionally contain sequences found in the 3′ portions of the coding regions of the above disclosed sequences. Polynucleotides containing a combination of sequences from the coding and 3′ non-coding regions preferably have the sequences arranged contiguously, with no intervening heterologous sequence(s).

Alternatively, the invention may be practiced with polynucleotides having sequences present in the 5′ untranslated and/or non-coding regions of the above CRIP1, HN1, and ESE-2/ELF5 sequences in breast cells to detect their levels of expression. Such polynucleotides may optionally contain sequences found in the 5′ portions of the coding regions. Polynucleotides containing a combination of sequences from the coding and 5′ non-coding regions preferably have the sequences arranged contiguously, with no intervening heterologous sequence(s).

Preferred polynucleotides contain sequences from 3′ or 5′ untranslated and/or non-coding regions of at least about 20, at least about 22, at least about 24, at least about 26, at least about 28, at least about 30, at least about 32, at least about 34, at least about 36, at least about 38, at least about 40, at least about 42, at least about 44, or at least about 46 consecutive nucleotides. The term “about” as used in the previous sentence refers to an increase or decrease of 1 from the stated numerical value. Even more preferred are polynucleotides containing sequences of at least or about 50, at least or about 100, at least about or 150, at least or about 200, at least or about 250, at least or about 300, at least or about 350, or at least or about 400 consecutive nucleotides. The term “about” as used in the preceding sentence refers to an increase or decrease of 10% from the stated numerical value.

Sequences from the 3′ or 5′ end of the above described coding regions as found in polynucleotides of the invention are of the same lengths as those described above, except that they would naturally be limited by the length of the coding region. The 3′ end of a coding region may include sequences up to the 3′ half of the coding region. Conversely, the 5′ end of a coding region may include sequences up the 5′ half of the coding region. Of course the above described sequences, or the coding regions and polynucleotides containing portions thereof, may be used in their entireties.

Polynucleotides combining the sequences from a 3′ untranslated and/or non-coding region and the associated 3′ end of the coding region are preferably at least or about 100, at least about or 150, at least or about 200, at least or about 250, at least or about 300, at least or about 350, or at least or about 400 consecutive nucleotides.

In another embodiment of the invention, polynucleotides containing deletions of nucleotides from the 5′ and/or 3′ end of the above disclosed sequences may be used. The deletions are preferably of 1-5, 5-10, 10-15, 15-20, 20-25, 25-30, 30-35, 35-40, 40-45, 45-50, 50-60, 60-70, 70-80, 80-90, 90-100, 100-125, 125-150, 150-175, or 175-200 nucleotides from the 5′ and/or 3′ end, although the extent of the deletions would naturally be limited by the length of the disclosed sequences and the need to be able to use the polynucleotides for the detection of expression levels.

In yet another embodiment of the invention, polynucleotides containing portions of the above disclosed sequences including the 3′ end may be used in the practice of the invention. Such polynucleotides would contain at least or about 50, at least or about 100, at least about or 150, at least or about 200, at least or about 250, at least or about 300, at least or about 350, or at least or about 400 consecutive nucleotides from the 3′ end of the disclosed sequences.

The invention thus also includes polynucleotides used to detect CRIP1, HN1, and ESE-2/ELF5 expression in breast cells. The polynucleotides may comprise a shorter polynucleotide consisting of sequences found in the above provided SEQ ID NOS in combination with heterologous sequences not naturally found in combination with CRIP1, HN1, and ESE-2/ELF5 sequences. As a non-limiting example, a polynucleotide of the invention may comprise a polynucleotide consisting of the sequence of SEQ ID NO:29, with a deletion of one or more nucleotides from the 5′ and/or 3′ end, in combination with one or more non-HN1 sequences.

Other polynucleotides for use in the practice of the invention include those that have sufficient homology to those described above to detect expression by use of hybridization techniques. Such polynucleotides preferably have about or 95%, about or 96%, about or 97%, about or 98%, or about or 99% identity with CRIP1, HN1, or ESE-2/ELF5 sequences as described herein. Identity is determined using the BLAST algorithm, as described above. The other polynucleotides for use in the practice of the invention may also be described on the basis of the ability to hybridize to polynucleotides of the invention under stringent conditions of about 30% v/v to about 50% formamide and from about 0.01M to about 0.15M salt for hybridization and from about 0.01M to about 0.15M salt for wash conditions at about 55 to about 65° C. or higher, or conditions equivalent thereto.

In a further embodiment of the invention, a population of single stranded nucleic acid molecules comprising one or both strands of a human CRIP1 or HN1 sequence is provided as a probe such that at least a portion of said population may be hybridized to one or both strands of a nucleic acid molecule quantitatively amplified from RNA of a non-normal or abnormal breast cell. The population may be only the antisense strand of a human CRIP1 or HN1 sequence such that a sense strand of a molecule from, or amplified from, a non-normal or abnormal breast cell may be hybridized to a portion of said population. The population preferably comprises a sufficiently excess amount of said one or both strands of a human CRIP1 or HN1 sequence in comparison to the amount of expressed (or amplified) nucleic acid molecules containing a complementary CRIP1 or HN1 sequence from a normal breast cell. This condition of excess permits the increased amount of nucleic acid expression in a non-normal or abnormal cell to be readily detectable as an increase.

Alternatively, the population of single stranded molecules is equal to or in excess of all of one or both strands of the nucleic acid molecules amplified from a non-normal or abnormal breast cell such that the population is sufficient to hybridize to all of one or both strands. Preferred non-normal cells are ADH, DCIS, or IDC cells. The single stranded molecules may of course be the denatured form of any CRIP1 and/or HN1 sequence containing double stranded nucleic acid molecule or polynucleotide as described herein.

The population may also be described as being hybridized to CRIP1 or HN1 sequence containing nucleic acid molecules at a level of at least twice as much as that by nucleic acid molecules of a normal breast cell. As in the embodiments described above, the nucleic acid molecules may be those quantitatively amplified from a breast cell such that they reflect the amount of expression in said cell.

The population is preferably immobilized on a solid support, optionally in the form of a location on a microarray. A portion of the population is preferably hybridized to nucleic acid molecules quantitatively amplified from a non-normal or abnormal breast cell by real time PCR. The real time PCR may be practiced by use of amplified RNA from a breast cancer cell, as long as the amplification used was quantitative with respect to CRIP1 and/or HN1 containing sequences.

In another embodiment of the invention, expression based on detection of DNA status may be used. Detection of the ESE-2/ELF5 DNA as methylated, deleted or otherwise inactivated, may be used as an indication of decreased expression as found in non-normal breast cells. This may be readily performed by PCR based methods known in the art. The status of the promoter regions (SEQ ID NOS:210 and 211) of the ESE-2/ELF5 may also be assayed as an indication of decreased expression of ESE-2/ELF5 sequences. A non-limiting example is the methylation status of sequences found in the promoter region.

Conversely, detection of the DNA of a sequence as amplified may be used for as an indication of increased expression as found in non-normal breast cells. This may be readily performed by PCR based, fluorescent in situ hybridization (FISH) and chromosome in situ hybridization (CISH) methods known in the art.

A preferred embodiment using a nucleic acid based assay to determine expression is by immobilization of one or more of the sequences identified herein on a solid support, including, but not limited to, a solid substrate as an array or to beads or bead based technology as known in the art. Alternatively, solution based expression assays known in the art may also be used. The immobilized sequence(s) may be in the form of polynucleotides as described herein such that the polynucleotide would be capable of hybridizing to a DNA or RNA corresponding to the sequence(s).

The immobilized polynucleotide(s) may be used to determine the state of nucleic acid samples prepared from sample breast cell(s) for which the pre-cancer or cancer status is not known or for confirmation of a status that is already assigned to the sample breast cell(s). Without limiting the invention, such a cell may be from a patient suspected of being afflicted with, or at risk of developing, breast cancer. The immobilized polynucleotide(s) need only be sufficient to specifically hybridize to the corresponding nucleic acid molecules derived from the sample.

In embodiments where only one or a few sequences are to be analyzed, the nucleic acid derived from the sample breast cancer cell(s) may be preferentially amplified by use of appropriate primers such that only the sequences to be analyzed are amplified to reduce contaminating background signals from other sequences present in the breast cell. Alternatively, and where the disclosed sequences are to be analyzed in combination with other sequences or where very few cells (or one cell) is used, the nucleic acid from the sample may be globally amplified before hybridization to the immobilized polynucleotides. Of course RNA, or the cDNA counterpart thereof may be directly labeled and used, without amplification, by methods known in the art.

Sequence expression based on detection of a presence, increase, or decrease in protein levels or activity may also be used. Detection may be performed by any immunohistochemistry (IHC) based, bodily fluid based (where a CRIP1, HN1, and/or ESE-2/ELF5 polypeptide is found in a bodily fluid, such as but not limited to blood), antibody (including autoantibodies against the protein where present) based, ex foliate cell (from the cancer) based, mass spectroscopy based, and image (including used of labeled ligand where available) based method known in the art and recognized as appropriate for the detection of the protein. Antibody and image based methods are additionally useful for the localization of tumors after determination of cancer by use of cells obtained by a non-invasive procedure (such as ductal lavage or fine needle aspiration), where the source of the cancerous cells is not known. A labeled antibody or ligand may be used to localize the carcinoma(s) within a patient.

Antibodies for use in such methods of detection include polyclonal antibodies, optionally isolated from naturally occurring sources where available, and monoclonal antibodies, including those prepared by use of CRIP1, HN1, and/or ESE-2/ELF5 polypeptides as antigens. Such antibodies, as well as fragments thereof (including but not limited to F_{ab} fragments) function to detect or diagnose non-normal or cancerous breast cells by virtue of their ability to specifically bind CRIP1, HN1, or ESE-2/ELF5 polypeptides to the exclusion of other polypeptides to produce a detectable signal. Recombinant, synthetic, and hybrid antibodies with the same ability may also be used in the practice of the invention. Antibodies may be readily generated by immunization with a CRIP1, HN1, or ESE-2/ELF5 polypeptide, and polyclonal sera may also be used in the practice of the invention.

Antibody based detection methods are well known in the art and include sandwich and ELISA assays as well as Western blot and flow cytometry based assays as non-limiting examples. Samples for analysis in such methods include any that contain CRIP1, HN1, or ESE-2/ELF5 polypeptides. Non-limiting examples include those containing breast cells and cell contents as well as bodily fluids (including blood, serum, saliva, lymphatic fluid, as well as mucosal and other cellular secretions as non-limiting examples) that contain the polypeptides.

The above assay embodiments may be used in a number of different ways to identify or detect the presence of non-normal breast cells or breast cancer in a breast cancer cell sample from a patient. In some cases, this would reflect a secondary screen for the patient, who may have already undergone mammography or physical exam as a primary screen. If positive from the primary screen, the subsequent needle biopsy, ductal lavage, fine needle aspiration, or other analogous methods may provide the sample for use in the assay embodiments described herein. The present invention is particularly useful in combination with non-invasive protocols, such as ductal lavage or fine needle aspiration, to prepare a breast cell sample. The current analysis of ductal lavage samples is by cytological examination by a trained pathologist who classifies the samples in terms that are at least partly subjective: unsatisfactory (too few cells), benign (including fibrocystic change), atypical (or mild atypia), suspicious (or marked atypia), or malignant.

The present invention provides a more objective set of criteria, in the form of gene expression levels of discrete gene sequences, to discriminate (or delineate) between normal and non-normal breast cells.

In one embodiment of the invention, the isolation and analysis of a breast cancer cell sample may be performed as follows:
(1) Ductal lavage or other non-invasive procedure is performed on a patient to obtain a sample.
(2) Sample is prepared and coated onto a microscope slide. Note that ductal lavage results in clusters of cells that are cytologically examined as stated above.
(3) Pathologist or image analysis software scans the sample for the presence of atypical cells.
(4) If atypical cells are observed, those cells are harvested (e.g. by microdissection such as LCM).
(5) RNA is extracted from the harvested cells.
(6) RNA is assayed for the expression of CRIP1, HN1, and/or ESE-2/ELF5 sequences.

A specific example of the above method would be performing ductal lavage following a primary screen, observing and collecting non-normal cells (or cells suspected of being non-normal) for analysis. Alternatively, the sample may permit the collection of both normal and non-normal cells (or cells suspected of being non-normal) for analysis. The expression levels of CRIP1, HN1, and/or ESE-2/ELF5 sequences in each of these two populations may be compared to each other. This approach can be significantly more powerful than one using the non-normal cells only approach because it utilizes information from the normal cells and the differences between normal and non-normal cells to determine the status of the non-normal cells from the sample.

While many clinical settings focus on identification of the highest stage or grade of breast cancer, the detection of non-normal breast cells of any stage or grade is also important to identify with confidence the presence of, or susceptibility to, breast cancer as early as possible. With use of the present invention, skilled clinicians will be apprised of the presence of non-normal cells quickly and may begin treatment or additional testing based on such information.

The present invention may also be used with solid tissue biopsies. As a non-limiting example, a solid biopsy may be collected and prepared for visualization followed by determination of increased CRIP1 and/or HN1 expression to identify or diagnose the presence of non-normal cells. One preferred means is by use of in situ hybridization with polynucleotide or protein identifying probe(s) for assaying expression of said gene(s). An analogous method may be used to detect decreased expression of ESE-2/ELF5 sequences.

In an alternative method, the solid tissue biopsy may be used to extract molecules followed by analysis for expression of the disclosed sequence(s). This provides the possibility of leaving out the need for visualization and collection of only those cells suspected of being non-normal. This method may of course be modified such that only cells suspected of being non-normal are collected and used to extract molecules for analysis. This would require some form of selection as a prerequisite to gene expression analysis.

In a further modification of the above, both normal cells and cells suspected of being non-normal are collected and used to extract molecules for analysis of sequence expression. The approach, benefits and results are as described above using non-invasive sampling.

In a further alternative to all of the above, the sequence(s) identified herein may be used as part of a simple PCR or array based assay simply to determine the presence of non-normal cells in a sample from a non-invasive sampling procedure. If normal expression levels of the disclosed sequences are identified, no further examination may be necessary. If non-normal expression levels are detected, a more comprehensive analysis may follow.

The detection of sequence expression from samples may be by use of a single microarray able to assay expression of the disclosed sequences as well as other sequences, including sequences known not to vary in expression levels between normal and non-normal breast cells, for convenience and improved accuracy.

Other uses of the present invention include providing the ability to identify breast cancer cell samples as being non-normal for further research or study. This provides a particular advantage in many contexts requiring the identification of non-normal or cancerous cells based on objective genetic or molecular criteria rather than cytological observation.

The materials and methods of the present invention are ideally suited for preparation of kits produced in accordance with well known procedures. The invention thus provides kits comprising agents (like the polynucleotides and/or antibodies described herein as non-limiting examples) for the detection of expression of the disclosed sequences. Such kits, optionally comprising the agent with an identifying description or label or instructions relating to their use in the methods of the present invention, are provided. Such a kit may comprise containers, each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and one or more primer complexes of the present invention (e.g., appropriate length poly(T) or random primers linked to a promoter reactive with the RNA polymerase). A set of instructions will also typically be included.

The methods provided by the present invention may also be automated in whole or in part. All aspects of the present invention may also be practiced such that they consist essentially of a subset, or subregion, of the disclosed sequences to the exclusion of material irrelevant to the identification of non-normal or cancerous breast cells.

To identify changes in sequence expression in non-normal or cancerous breast cells, abnormal epithelium from ADH, DCIS and IDC and phenotypically normal epithelium (henceforth referred to as normal) from 36 breast cancer patients and 3 healthy mammoplasty reduction patients were isolated via laser capture microdissection (LCM). The resulting 300 independently microdissected samples were used to interrogate a microarray containing approximately 12,000 human genes.

One important advantage provided by LCM use is the ability to procure both normal and diseased cell populations from the same biopsy. Therefore, the expression level of each gene in a disease state (ADH or DCIS or IDC) is represented as the ratio to the patient-matched normal, which highlights differences due to disease state as opposed to the genetic background of a particular patient. Unsupervised hierarchical clustering revealed one sequence cluster demonstrating increased expression in a majority of the diseased samples. CRIP1 was included in the cluster and identified as displaying increased expression levels in ADH with persistence in DCIS and IDC samples. Its increased expression may thus be a potential biomarker for the detection of breast cancer including the pre-malignant stage of ADH.

Sequences of another cluster demonstrated decreased expression in all three pathological stages. The epithelium-specific transcription factor ELF5 was included, and loss of expression of ELF5 in ADH may be an important first step in the initiation of breast malignancy.

Additional experiments resulted in the identification of increased expression of HN1 sequences in grade III DCIS and IDC cells.

Having now generally described the invention, the same will be more readily understood through reference to the following examples which are provided by way of illustration, and are not intended to be limiting of the present invention, unless specified.

### EXAMPLES

### Example I

### Materials and Methods

Clinical specimen collection and clinicopathological parameters. All breast specimens were obtained from the Massachusetts General Hospital between 1998 and 2001. Thirty-six breast cancer patients were selected, 31 of which were diagnosed with two or more pathological stages of breast cancer progression, and 5 of which were diagnosed with pre-invasive disease only. Three healthy women who underwent elective mammoplasty reduction were selected as disease-free normal controls. Tissue specimens that demonstrated one or more pathological lesions (ADH, DCIS and IDC) were selected for the study. Cases of ADH were selected as proliferative epithelial lesions that possessed some, but not all, of the features of carcinoma in situ (Page, D. L. et al. (1992)) and most closely resemble those lesions described as CAPSS (Oyama, T. et al. and Fraser, J. L. et al.). DCIS and IDC were classified (histological grade) according to the European classification (Holland, R. et al.) and by the Nottingham combined histological grade (Elston, C. W. et al.), respectively. ER and PR expression were determined by immunohistochemical staining (negative when none of the tumor cell nuclei showed staining), and Her-2 expression determined by immunohistochemistry or FISH. This study was approved the Massachusetts General Hospital human research committee in accordance with NIH human research study guidelines.

LCM and RNA isolation and amplification. Each component (Normal, ADH, DCIS or IDC) was laser capture microdissected in triplicate (from consecutive tissue sections) as described (Sgroi et al.) using a PixCell II LCM system (Arcturus Engineering Inc., Mountain View, Calif.). Total RNA was extracted from the captured cells using the Picopure™ RNA Isolation Kit (Arcturus). T7-based RNA amplification was carried out using the RiboAmp™ kit (Arcturus). Briefly, the RNA from each sample was primed with an oligo-dT primer containing a T7 promoter sequence, reverse transcribed and then converted to double stranded cDNA. The cDNA templates were then used in an in vitro transcription reaction using T7 RNA polymerase to generate amplified RNA (aRNA). To obtain enough aRNA for a microarray experiment, a second round of RNA amplification was performed on all samples. To serve as reference in microarray hybridizations, a human universal reference RNA from Stratagene (La Jolla, Calif.) was amplified identically.

Fabrication of microarrays. Sequence-verified human cDNA clones were obtained from Research Genetics (Huntsville, Ala.). cDNA clones (from the I.M.A.G.E. Consortium via Research Genetics) inserts were amplified by PCR, gel-purified, and spotted onto a 1×3-inch SuperAmine™ (TeleChem International, Sunnyvale, Calif.) glass microscope slide using an OmniGrid™ robotic arrayer (GeneMachines, San Carlos, Calif.). As used herein, the I.M.A.G.E. Consortium CloneID, or the IMAGE CloneID, lists the identifiers of the cDNA clones on the microarrays according to the I.M.A.G.E. Consortium and Research Genetics (www.resgen.com/). This provides a unique single identifier for each clone. Descriptive names of clones (or genes) use the UniGene symbols and titles (www.ncbi.nlm.nih.gov/UniGene/).

Probe labeling and hybridization. cDNA was transcribed from aRNA in the presence of 5-(3-aminoallyl)-2′-deoxyuridine 5′-triphosphate (aminoallyl dUTP) using Stratagene's FairPlay kit™ (La Jolla, Calif.). Cy3 or Cy5 mono-reactive dye (Amersham, Piscataway, N.J.) was conjugated onto purified cDNA and the residual dye was removed using QiaQuick PCR Purification columns (Qiagen, Valencia, Calif.). Each Cy5-labeled cDNA was hybridized together with the Cy3-labeled reference probe to a microarray in 40 μL hybridization solution (5×SSC, 0.1 μg/μL COT I, 0.2% SDS, 50% formamide) at a concentration of 25 ng/μL per channel for 17 hrs at 42° C. in >60% relative humidity.

Washing, scanning and image analysis. After hybridization, slides were washed as follows: 1×SSC, 0.2% SDS at 42° for 5 min (two times), 1×SSC, 0.2% SDS at 55° C. for 5 min, 0.1×SSC, 0.2% SDS at 55° C. for 5 min and 0.1×SSC at RT for 2 min. Washed slides were scanned using ScanArray 5000 (PerkinElmer, Billerica, Mass.), and Cy5/Cy3-signals were quantitated using ImaGene 4.2 (BioDiscovery, Los Angeles, Calif.).

Data processing. Fluorescent intensities of Cy5 and Cy3 channels on each slide were subjected to spot filtering and normalization. Spots flagged by ImaGene were excluded from further analysis. Normalization was performed using a robust nonlinear local regression method (Yang, Y. H. et al.). The normalized ratios of Cy5/Cy3 were used to represent the relative gene expression levels in the experimental samples. Measurements from replicate samples were averaged after normalization.

Cluster and discriminant analysis. Hierarchical cluster analysis was performed in GeneMaths (v1.5, Applied-Maths, Austin, Tex.) using the cosine correlation coefficient as a measure of similarity between two genes or samples and complete linkage. Linear discriminant analysis with variance was performed within GeneMaths.

### Example II

### Analysis of Over and Under Expression

Quantitative real-time PCR analysis of CRIP1 and ELF5 was conducted to confirm their over and under expression in non-normal breast cells.

For the non-amplified RNA RT-PCR validation study, independently laser captured (˜40,000) normal breast epithelial cells from case 215, and ˜40,000 abnormal epithelial cells from DCIS (from cases 89, 178, 179) or IDC (from cases 97, 169, 170) were used. Total RNA was isolated and converted to double-stranded cDNA. For studies using amplified RNA (aRNA), 2 mgs of aRNA from each microdissected sample was converted into double-stranded cDNA. In all cases (cDNA derived from non-amplified and amplified RNA), the double-stranded cDNA was quantitated with PicoGreen (Molecular Probes) using a spectrofluorometer (Molecular Devices) and quantitative analysis of gene expression performed (RT-PCR) was performed with an ABI 7900HT (Applied Biosystems, Foster City, Calif.) as described (Sgroi et al., 1999).

Each reaction was performed in triplicate using 2.5 ng of double stranded cDNA from each sample as template. The relative standard curve method was used for linear regression analysis of unknown samples and data presented as fold change between samples. The sequences of the PCR primer pairs and fluorogenic probe (5′ to 3′), respectively, that were used for each gene are as follows:

CRIP1:
CCTGCTACGCAGCCATGTT,
GGATGGGTCTCCACCACCT,
VIC-CGGAGCCGAGAGCCACACTTTCAAGT-TAMRA;
ELF5:
TGATTCCTGCTCTGGTGAAACA,
ACATTTTCTCATCAATCA CTGGTATGT,
VIC-CAGTCCACCTAGCTGTCAGTCACTGATA-TAMRA;

In agreement with microarray results used to initial identify sequences that were over and under expressed in non-normal breast cells, RT-PCR demonstrated over-expression of CRIP1 (>2-fold) in 7 of 8 ADH, 27 of 30 DCIS, and 23 of 25 IDC cases, and under-expression of ELF5 (>2-fold) in 7 of 8 ADH, 28 of 30 DCIS, and 25 of 25 IDC cases (FIG. 1).

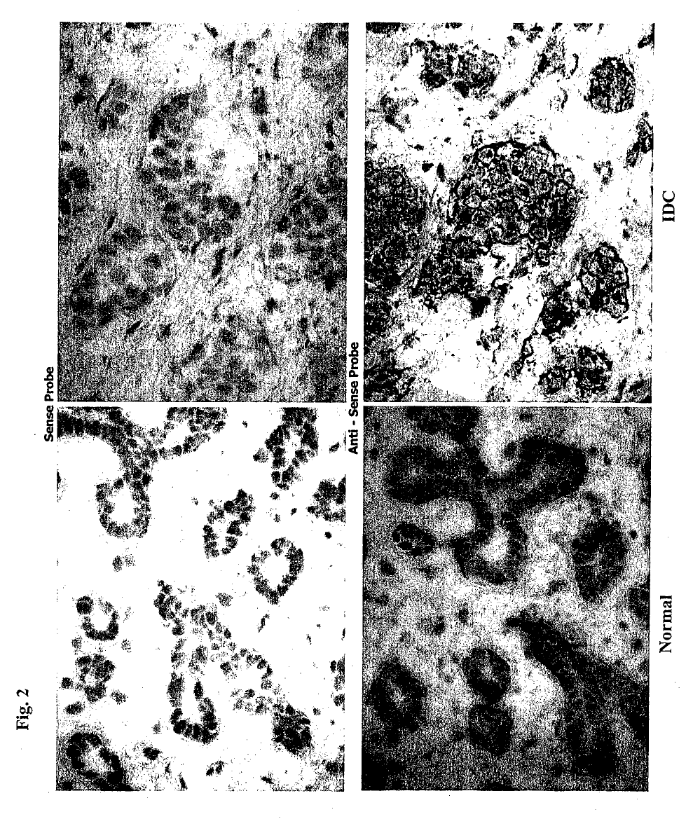

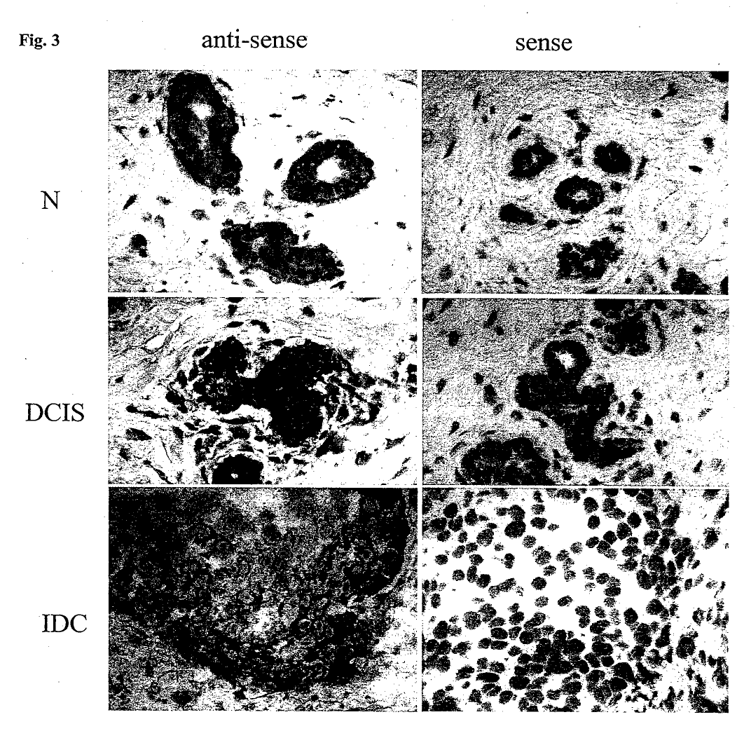

In addition, we performed in-situ hybridization for CRIP1 to confirm its cellular specificity. As expected from the use of LCM, CRIP1 signal localized to the epithelial cells, and its intensity was markedly increased in the IDC compartment of the same biopsy (FIGS. 2 and 3), thus verifying the microarray-derived results at the level of cellular resolution.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without undue experimentation. This application is intended to cover any variations, uses, or adaptations of the invention, following in general the principles of the invention, that include such departures from the present disclosure as come within known or customary practice within the art to which the invention pertains and as may be applied to the essential features hereinbefore set forth.

We claim:

1. A method to determine the presence of pre-malignant cells in a sample from a human subject comprising assaying a sample suspected of containing pre-malignant cells for decreased expression of human ESE-2/ELF5 sequences.

2. The method of claim 1 wherein said sample is from a subject afflicted with, or suspected of having, atypical ductal hyperplasia (ADH) or breast cancer.

3. The method of claim 1 wherein said sample is obtained by solid tissue biopsy or a non-invasive procedure.

4. The method of claim 3 wherein said non-invasive procedure is selected from ductal lavage, fine needle aspiration, or a needle biopsy.

5. The method of claim 4 wherein microdissection is used to isolate breast cells from said sample before assaying for nucleic acid expression.

6. The method of claim 5 wherein said assaying is by hybridization to a polynucleotide comprising sequences of at least 24 nucleotides from the 3′ untranslated region, the coding region, or the 5′ untranslated region, of human ESE-2/ELF5.

7. The method of claim 1 wherein said assaying is by PCR amplification of said ESE-2/ELF5 sequence.

8. The method of claim 7 wherein said assaying is by quantitative PCR.

9. The method of claim 1 wherein said assaying is for inactivation or methylation of ESE-2/ELF5 sequences.

10. The method of claim 1 wherein said assaying comprises detection of increased mRNA degradation.

11. A method to determine the presence of non-normal or abnormal breast cells in a sample from a human subject comprising assaying said sample for increased expression of one or more human CRIP1 or HN1 sequences.
